(19) United States
(12) Patent Application Publication
(10) Pub. No.: US 2007/0059711 A1
(43) Pub. Date: Mar. 15, 2007

(54) Microarray for evaluation of stress-related genes in skin

(76) Inventors: Andrzej Slominski, Germantown, TN (US); Alexander Pisarchik, Lauderdale, MN (US)

Correspondence Address: Benjamin Aaron Adler;ADLER & ASSOCIATES, 8011 Candle Lane, Houston, TX, 77071, US

(21) Appl. No.: 11/193,801

(22) Filed: Jul. 29, 2005

Related U.S. Application Data
(60) Provisional application No. 60/592,875, filed on Jul. 30, 2004.

(51) Int. Cl. C12Q 1/68; G06F 19/00; C12P 19/34; C12M 1/34

(52) U.S. Cl. 435/6; 435/91.2; 435/287.2; 702/20; 977/924

(57) ABSTRACT

The present invention provides a novel DNA microarray chip that can be used for simultaneous testing of transcriptional responses to cutaneous stressors in the context of neuro-endocrine-immune functions of the skin. The transcriptional responses to ultraviolet radiation in epidermal keratinocytes were tested using such microarray chip containing more than 700 neuro-endocrine-immune related genes. The gene expression pattern was non-random and time dependent; it included increased expression of genes involved in water and salt balance, prostaglandin synthesis, keratinocyte differentiation as well as genes coding for stress effectors, cytokines and metalloproteinases. In contrast, expression was decreased for genes coding for growth factors and their receptors, and for elements of extracellular matrix. This stochastic pattern suggests that transcriptional responses are coordinated and aimed at preservation of epidermal barrier function, prevention of early carcinogenic events and remodeling of extracellular matrix.

### CROSS-REFERENCE TO RELATED APPLICATION

This non-provisional application claims benefit of provisional patent applications Ser. No. 60/592,875, filed Jul. 30, 2004, now abandoned.

### FEDERAL FUNDING LEGEND

This invention was produced in part using funds obtained through a grant (AR047079-01A2) from the National Institutes of Health. Consequently, the federal government has certain rights in this invention.

### BACKGROUND OF THE INVENTION

1. Field of the Invention

The present invention relates generally to the molecular biology and genomics technology. More specifically, the present invention relates to microarrays for the investigation of skin neuro-endocrine-immune functions.

2. Description of the Related Art

As a biological barrier, the skin is continuously subjected to the environmental stresses such as ultraviolet radiation, pathogens and thermal and chemical insults (Slominski and Wortsman, 2000). To provide systemic protection, skin cells have numerous defense mechanisms, presumably evolving from a network of intracellular communications coordinated by messages from the local neuro-endocrine-immune system (Slominski and Wortsman, 2000).

This concept, of skin as a peripheral neuroendocrine organ, would imply that local homeostasis (and by inference systemic homeostasis) is regulated by the expression of hormones, neurotransmitters, neuropeptides, cytokines and their receptors. Accordingly, expression of the respective genes should be hierarchical, coordinated and organized into patterns defining functional pathways after exposure to environmental stressors and/or pathological events. The predominant environmental stressor for the skin is solar radiation, especially its UV wavelengths (200-320 nm) (Slominski and Pawelek, 1998). Primary responses to UV radiation (UVR) involve induction of melanin pigmentation and immunomodulation and endocrine activity represented by production of vitamin D and neuropeptides (Slominski and Pawelek, 1998; Slominski and Wortsman, 2000).

Chronic UVR exposure has potent carcinogenic and aging effects. However, the prior art is deficient in the development of a microarray assay to obtain further insight into the cutaneous stress response under chronic UVR exposure. The present invention fulfills this long-standing need and desire in the art by developing microarrays that could be used for simultaneous testing of transcriptional responses to cutaneous stressors in context of neuroendocrine-immune functions of the skin.

### SUMMARY OF THE INVENTION

The present invention is directed to a microarray comprising a human molecular chip composed of approximately 700 genes developed to study peripheral response to stress. The chip was designed to evaluate hierarchical and coordinated gene expression patterns organized into functional pathways in response to environmental stress, dyshomeostatic stimuli or pathology. These human microarrays included oligonucleotides corresponding to genes encoding enzymes involved in hormonal production or degradation or neuropeptide processing, precursors to neuropeptide, cytokines, corresponding receptors, other immune markers. For completeness of such molecular chips, several structural genes governing melanogenesis (specific for melanocytes) or epidermal barrier formation by keratinocytes (different cytokeratins) were also included to allow evaluation of the neuroendocrine and immune pathways in relation to differentiated state of prevalent cells of the epidermal component. The oligonucleotides for genes in the microarray chip not limited to include the ones shown in Table 1.

This technology was then applied to the testing of transcriptional activity in keratinocytes exposed to UVR. Significant expression changes of genes arranged in patterns defining functional pathways were found. Thus, this chip could be used as an efficient molecular technology to study neuroendocrine and immune functions of the skin and other organs on a comprehensive molecular level in relation to external and internal stress or any dyshomeostatic signal. It would further allow shaping the modern concept of skin as a neuroendocrine organ requiring that local homeostasis and by inference systemic homeostasis is dependent on the regulated local expression of hormones, neurotransmitters, neuropeptides, cytokines and their receptors.

In one aspect of the present invention, there is provided a DNA microarray chip for measuring transcriptional responses to stress in a cell. The chip comprises 761 oligonucleotides for genes related to production and metabolism of hormones, neurotransmitters, neuropeptides, cytokines, biological modulators, growth factors, corresponding receptors and normal skin metabolism genes.

In another aspect of the present invention, there is provided a method of detecting transcriptional responses to stress in a cell that comprises following steps. The cell is challenged with stress and nucleic acid samples are isolated from the cell. This is followed by measuring gene expression levels of a group of genes. Subsequently, the gene expression levels are analyzed statistically and compared to those without stress, where a statistically significant upregulation of the gene expression indicates transcriptional responses to stress.

In yet another aspect of the present invention, there is provided a method of detecting transcriptional responses to stress in a cell that comprises the same steps of challenging, isolating nucleic acid and measuring gene expression levels. However, in this case the gene expression levels are analyzed statistically and compared to those without stress, where a statistically significant downregulation of the gene expression indicates transcriptional responses to stress.

Other and further aspects, features, and advantages of the present invention will be apparent from the following description of the presently preferred embodiment of the invention. This embodiment is given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows detection of differentially expressed genes. Green dots represent genes with higher level of A546 labeled cDNA. Red dots represent genes with higher level of A647 labeled cDNA. Two green spots on the top are landing marks.

FIGS. 2A and 2B show relative expression of genes in UV-irradiated keratinocytes. FIG. 2A shows real-time RT-PCR results that were calculated by comparing UV-irradiated keratinocytes (incubated 12 hours after irradiation) against controls (no treatment). Data was normalized to GAPDH expression and represented mean ±SD (N=3). Ratios statistically significantly different from 1 (no difference) are denoted by asterisks (p<0.05). FIG. 2B shows RT-PCR of human gastrin. The samples were loaded as follows: immortalized keratinocytes (HaCaT; lane 1); epidermal keratinocytes (HeKa; lane 2); epidermal fibroblasts (lane 3); different melanomas (lanes 4-9); melanocytes (lane 10). M denotes molecular marker.

### DETAILED DESCRIPTION OF THE INVENTION

The following abbreviations are used herein: AQP, aquaporin; CYP4F8, cytochrome P450, subfamily IVF, polypeptide 8; COX-2, prostaglandin-endoperoxide synthase 2; IL-6, interleukin 6; IL-1, interleukin 1; IL-2, interleukin 2; IL-8, interleukin 8; TNFSF9, tumor necrosis factor superfamily member 9; BMP3, bone morphogenetic protein 3; IGFBP6, insulin-like growth factor binding protein 6; VGEF, vascular endothelial growth factor; EGFR, epidermal growth factor receptor; KGF, keratinocyte growth factor; KGFR, keratinocyte growth factor receptor; TNFSF10 (TRAIL), tumor necrosis factor superfamily member 10; MMPs, metalloproteinases; CRF-R2, corticotropin releasing factor receptor type 2.

The present invention used microarray technology to detect several genes with changed expression levels (up- or down-regulated) in a time-dependent manner afetr exposure to UVR. Although the affected genes were mostly involved in water and salt balance and prostaglandin metabolism, they also included interleukins and growth factors, metalloproteinases, markers of keratinocyte differentiation and genes involved in stress response. Functional assessment revealed that majority of the detected genes represented defense mechanisms against malignant transformation. Thus, activation of interleukins, of keratinocyte differentiation markers and down-regulation of EGFR, KGF and KGFR may all represent anti-cancer defense mechanisms. In contrast, the stimulation of metalloproteinases, the enzymes responsible for the extracellular matrix rearrangement or the down-regulation of TRAIL might promote tumor progression in the human skin. Among all the genes that were analyzed, stimulation of COX-2, TNFSF9 and down-regulation of KGF and KGFR were the most interesting observation. Many of the gene expression changes induced by UVR have been already described. Thus, confirming the results obtained by using the microarray chip of the present invention with that already known made the data more reliable. The data was also verified independently using real-time PCR and sequencing. Thus, the microarray chip can be reliably used for the simultaneous assessment of the effect of different agents on the expression of a large set of endocrine-related genes.

In one aspect of the present invention, there is provided a DNA microarray chip for measuring transcriptional responses to stress in a cell. The chip comprises 761 oligonucleotides for genes related to production and metabolism of hormones, neurotransmitters, neuropeptides, cytokines, biological modulators, growth factors, corresponding receptors and normal skin metabolism genes. The oligonucleotides for genes in the DNA microarray chip have SEQ ID Nos. shown in SEQ ID No. 25-785.

In another aspect of the present invention, there is provided a method of detecting transcriptional responses to stress in a cell, comprising the steps of: challenging the cell with stress; isolating nucleic acid samples from the cell; measuring gene expression levels of a group of genes; and analyzing the gene expression levels statistically as compared to those without stress, wherein a statistically significant upregulation of the gene expression indicates transcriptional responses to stress. The gene expression is measured by DNA microarray and further verified by RT-PCR. Representative examples of the stress are ultraviolet radiation, thermal stress, chemical stress, or immune stress. The preferred cell is epidermal keratinocyte, melanocyte, fibroblast, or immune cell. Representative examples of the statistical analysis that can be performed are one-way analysis of variance, post-hoc test or student t test. The statistically significant upregulation is atleast a 1.5-fold increase of expression of the genes. The group of genes not limited to include genes involved in water and salt balance, genes involved in prostaglandin metabolism, interleukin genes, growth factor genes, markers of keratinocyte differentiation genes, metalloproteinases genes, and stress response genes. The upregulated genes not limited to include the genes selected from the group of genes encoding for gastrin, vasopressin, aquaporin, prostaglandin-endoperoxide synthase 2, CYP4F8, IL-1, IL-2, IL-6, IL-8, tumor necrosis factor superfamily member 9, bone morphogenetic protein 3, insulin-like growth factor binding protein 6, vascular endothelial growth factor B, stromelysins 1 and 2, corticotropin releasing factor receptor type 1 and 2, and aldo-keto reductase.

In yet another aspect of the present invention, there is provided a method of detecting transcriptional responses to stress in a cell that comprises the same steps of challenging, isolating nucleic acid samples, measuring gene expression levels and statistical analysis of the gene expression levels as compared to those without stress as described supra. However in this case, a statistically significant downregulation of the gene expression indicates transcriptional responses to stress. All other aspects regarding the assay to measure gene expression and to verify its expression, the types of stress, type of cell and the genes whose expression is measured is the same as described supra. The down-regulated genes not limited to include genes selected from the group of genes encoding for epidermal growth factor receptor, keratinocyte growth factor, keratinocyte growth factor receptor, activin A receptor type I, and tumor necrosis factor superfamily member 10.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the cells and methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### EXAMPLE 1

Cell Lines and Cell Culture

Human adult epidermal keratinocytes and melanocytes were purchased from Cascade Biologics and cultured according to the company's protocol (Cascade Bio, Portland, Oreg.). Melanocytes were grown in Medium 154 and keratinocytes in EpiLife medium supplemented with appropriate growth factors and antibiotics. For co-cultivation experiments, keratinocytes and melanocytes were mixed in the ratio 20:1 and incubated in Medium 154 supplemented with HKGS (Cascade Bio, Portland, Oreg.).

### EXAMPLE 2

Ultraviolet Light Treatment

Ultraviolet irradiation of cells was performed as previously described (Pisarchik and Slominski, 2001). Briefly, cells were cultured in 75 cm² flasks at 85% confluency. The flasks were placed on the UV transilluminator 2000 (BioRad) and irradiated with 50 mJ/cm² UVB (Pisarchik and Slominski, 2001). Time of exposure, corresponding doses and UV spectrum had been established previously; after UV exposure (50 mJ/cm²) the morphology of the cells did not change significantly (Pisarchik and Slominski, 2001). Irradiated cells were further incubated in culture media for 4, 12 and 24 hours, detached and processed for RNA isolation.

### EXAMPLE 3

Microarray Slides

Oligonucleotides for genes related to production and metabolism of hormones, neurotransmiters, neuropeptides, cytokines, biological modulators, growth factors, corresponding receptors and normal skin metabolism genes were designed (http://www.utmem.edu/pathology/molecular-endocrinology.htm). The location of the oligos for each gene was chosen according to the structure of the corresponding gene obtained from the GeneBank. Oligonucleotides were synthesized by Integrated DNA Technologies (Coralville, Iowa) and printed on superamine slides (TeleChem, Sunnyvale, Calif.) at the University of Tennessee Molecular Resource Center.

### EXAMPLE 4

RNA extraction, cDNA Preparation and Fluorescent Labeling

Total RNA was extracted using Trizol isolation kit (Gibco-BRL, Gaithersburg, Md.). The cDNA was synthesized, hybridized and labeled with Genisphere Array 50 kit according to the manufacturer protocol (Genisphere, Hatfield, Pa.).

### EXAMPLE 5

Hybridization Protocol

Slides were washed by 0.2% SDS (2 min), water (two times, 2 min), dipped in absolute ethanol (1 min), dried by centrifugation (300×g, 2 min) followed by the injection of 100 μl of the hybridization mixture under the cover slip (LifterSlip cover glass, Erie Scientific Company, Portsmouth, N.H.). Slide hybridization and washing were done according to the protocol of the Genisphere Array 50 kit manufacture (Genisphere, Hatfield, Pa.). Slides were incubated overnight at 53° C. in the dark.

### EXAMPLE 6

Data Acquisition and Analysis

At least four arrays were used in every experiment (every time-point). Fluorescence corresponding to A546 and A647 dyes was measured by Axon scanner at The University of Tennessee Molecular Resource Center. Program Fine PixPro 4.0 was used to identify spots and subtract background. Data was normalized by LOWES method (intensity-dependent) using the BRB-Array Tools 2.1 program written by Dr. Richard Simon (Biometrics Research Branch, National Cancer Institute) and Amy Peng (EMMES Corporation). The output Excel spreadsheet contained normalized logarithms (base 2) of ratios for each gene on the slide.

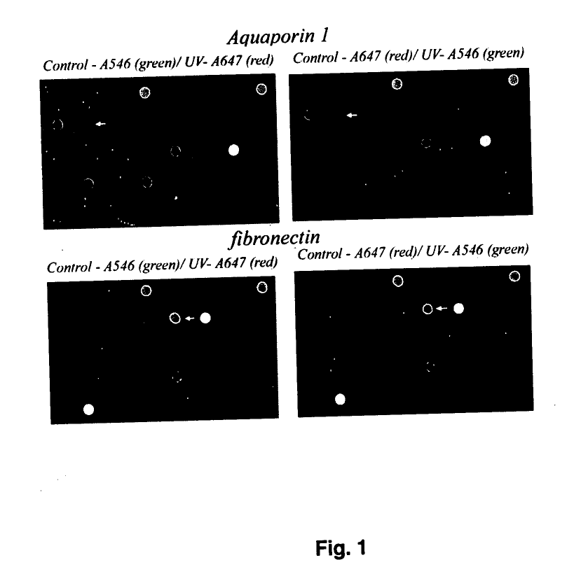

For easy interpretation, logarithms were recalculated into ratios. The factor of 1.5 was chosen as the cut-off for increase/decrease of gene expression, instead of the generally-used factor of 2. This was done because some genes that did show statistically significant stimulation of expression did not attain the 2-fold difference. To avoid false positives, only those genes that were consistently up- or down-regulated on several slides including dye-flip slides as presented on FIG. 1 were selected. Table 2 lists the average ratios for these genes. It is apparent from the table that the majority of selected genes were stimulated or inhibited at least 2 fold.

### EXAMPLE 7

RT-PCR and Real-Time RT-PCR

Total RNA (10 μg) was treated with DNAse (Invitrogen), purified by Rneasy MinElute Cleanup kit (Quigen, Valencia, Calif.) and reverse transcribed by Superscript First-Strand Synthesis System (Invitrogen). The resulting cDNA was diluted 100 times in water and aliquots were subjected to quantitative real-time PCR in an iCycler (BioRad). The PCR mixture (25 μl) contained 5 μl of the diluted cDNA, 2.5 μl of each primer (0.04 μM) and 12.5 μl of SYBR Green (Applied Biosystems, Warrington, UK). Each sample was amplified in triplicate. Reactions were performed with initial hot start incubation at 95° C. for 10 min, followed by 40 cycles of denaturation at 95° C. for 10 sec and 60° C. for 1 min. The mixture was then incubated at 60° C. for 10 min, heated gradually in 0.4 increments and the melting curve was then analyzed. Relative gene expression was recalculated by ΔΔCt method. Briefly, the difference (ΔCt) between the Ct values of the target and the house-keeping gene (GAPDH or β-actin) were calculated:
ΔCt=Ct(target)−Ct(GAPDH)
ΔΔCt is the difference between UV-irradiated and control samples:
ΔΔCt=ΔCt(UV)−ΔCt(control)
The fold-time stimulation of gene expression is calculated as =2^{ΔΔCt}

Primers used for the PCR were as follows: prostaglandin-endoperoxide synthase 2 (COX-2) P898 (ATGAGTGTGGGATTTGACCAG, forward, SEQ ID No. 1) and P899 (TTCCGGTGTTGAGCAGTTTTC, reverse, SEQ ID No. 2); TNRSF9 P900 (AGCTACAAAGAGGACACGAAG, forward, SEQ ID No. 3) and P901 (CGCAGCTCTAGTTGAAAGAAG, reverse, SEQ ID No. 4); Keratinocyte growth factor receptor P904 (GATGGTGCGGAAGATTTTGTC, forward, SEQ ID No. 5) and P905 (GCCGCTTTTCCATCTTTTCTG, reverse, SEQ ID No. 6); stromelysin 2 P908 (ATTTTGGCCCTCTCTTCCATC, forward, SEQ ID No. 7) and P909 (TGATGGCCCAGAACTCATTTC, reverse, SEQ ID No. 8); stromelysin 1 P910 (GGAAATCAATTCTGGGCCATC, forward, SEQ ID No. 9) and P911 (CATCGATTTTCCTCACGGTTG, reverse, SEQ ID No. 10); aquaporin 3 P912 (TTTGGCTTTGCTGTCACTCTG, forward, SEQ ID No. 11) and P913 (AAGCACATGGCAAAGGTCAC, reverse, SEQ ID No. 12); aquaporin 9 P916 (TTTGCTGGTGGAAAACTGCTG, forward, SEQ ID No. 13) and P917 (AAATGCGTTCGCCAGAGATAG, reverse, SEQ ID No. 14); keratin 1 K1-L (TGACCAAGGTGGACCTTCAG, forward, SEQ ID No. 15) and K1-R (ATGATGCTGTCCAGGTCGAG, reverse, SEQ ID No. 16); involucrin IVL-L (TCAGCCTTACTGTGAGTCTGG, forward, SEQ ID No. 17) and IVL-R (AGTCTTGAGGAGCTCCTGAC, reverse, SEQ ID No. 18); GAPDH G-L (TGATGACATCAAGAAGGTGGTGAAG, forward, SEQ ID No. 19) and G-R (TCCTTGGAGGCCATGTAGGCCAT, reverse, SEQ ID No. 20); β-actin BA-L (GCCCTGGCACCCAGCACAAT, forward, SEQ ID No. 21) and BA-R (TCATAGTCCGCCTAGAAGCA, reverse, SEQ ID No. 22).

The 283 bp long band corresponding to human Gastrin was amplified by primers P432 (TGCAGACGAGATGCAGCGAC, SEQ ID No. 23) and P433 (GTCCATCCATCCATAGGCTTC, SEQ ID No. 24). The reaction mixture (25 μl) contained 2.0 mM MgCl₂, 0.25 of each dNTP, 0.4 μM of each primer, 75 mM Tris-HCl (pH 8.8), 20 mM (NH₄)2SO₄, 0.01% Tween 20 and 1.25 u of Taq polymerase (Promega). The mixture was heated to 94° C. for 2.5 minutes and then amplified for 25 cycles: 94° C. for 10 seconds (denaturation), 55° C. for 10 seconds (annealing) and 72° C. for 30 seconds (extension). Amplification products were separated by agarose electrophoresis and visualized by ethidium bromide staining. The identified PCR products were excised from the agarose gel and purified using a GFX PCR DNA and gel band purification kit (Amersham-Pharmacia-Biotech). Sequencing was performed at the Molecular Resource Center at the University of Tennessee HSC (Memphis) using Applied Biosystems 3100 Genetic Analyzer and BigDye™ Terminator Kit.

### EXAMPLE 8

Statistical Analysis

Data was analyzed using one-way analysis of variance and appropriate post-hoc test or by Student t test using Prism 4.00 software (GraphPad Software, San Diego, Calif.).

### EXAMPLE 9

Technological Considerations

Exposure to sunlight is the leading cause of skin cancer due to its UV component (Ichihashi et al., 2003). A number of genes have been reported to be involved in the response to UVR including those coding for the cytokines IL-1β, IL-6, IL-10 (Kimbauer et al., 1991; Grewe et al., 1995). These data, generated by conventional approach were, however, concentrated on a limited number of well-known genes and consequently limited in scope. To overcome such constraints, DNA microarrays searching for a range of genes that might be differentially expressed in human keratinocytes in response to UV irradiation were developed. The known limitation of this technology was the small number of experimental samples that could be tested simultaneously versus the large number of variables (probes); thus statistically significant effects could reflect experimental biasing while lack thereof might reflect low sensitivity, rather than absence of biological effect. To minimize possible bias, detected effects were verified by RT-PCR.

Another important aspect of microarray technology is data normalization; for which there are several approaches (Bilban et al., 2002). The most widely accepted method was used herein: LOWES normalization (intensity dependent). The strongest factors affecting data reliability were the technical quality of the slides and intrinsic precision and consistency of the individual techniques. Sometimes it might be impossible to eliminate false positives due to nonspecific dye binding, but this could be solved by dye-flip experiments, as presented in FIG. 1. Briefly, if the dot is green on the first slide it should be red on the second with the opposite dye combination. This was exemplified in FIG. 1 where both dye combinations showed identical changes of gene expression (up- or down-regulation).

DNA microarray with 761 probes was specifically designed herein to investigate neuroendocrine functions in the skin, including probes for the barrier function (Slominski and Wortsman, 2000). Genes were chosen to reflect functional pathways activated in response to environmental stressors or pathological events; e.g. carbohydrate metabolism-related hormones; pituitary and thyroid hormones; steroidogenic enzyme receptors. Genes coding for neurotransmitters, catecholamines, growth factors, cytokines, housekeeping genes and others involved in the protective function of the skin were also added. The full list of the genes as well as oligonucleotide sequences are listed on Table 1.

The oligonucleotide design approach used herein was a modification of the commonly used technique of searching for oligos with desired melting temperature, preferably at the 3′ end of the gene. Instead the oligos were placed on the borders between different exons to avoid unspecific labeling of DNA impurities. It is well known that human introns contain a wide range of simple repetitive sequences of which polyA sequences are most common. Hence, the use of total RNA and poly(dT) sequence to synthesize cDNA for microarray experiments may result in some unspecific labeling since poly(dT) sequence may hybridize to introns. This concept was tested by hybridizing cDNA synthesized from total and polyA RNA of HaCaT cell line (data not shown). The polyA RNA sample showed lower signal but, when normalized the conditions showed identical results.

TABLE 1
DNA Microarray Chips for Neuroendocrine Functions in the Skin

| | Short | Full | | | |
|---|---|---|---|---|---|
| Primer | gene | gene | Accession | | |
| name | name | name | number | Primer sequence | Comments |
| M001 | GCG | Glucagon | NM_002054 | TTCAAGACACAGAGGAGAAATC [symbol] | 50 bp; e2-3 |
| | | | | AGATCATTCTCAGCTTCCCAGG [symbol] | |
| | | | | GAC | |
| | | | | (SEQ ID NO: 25) | |
| M002 | GCGR | glucagon | NM_000160 | TGGTACCTGCCTTGGCACCACAA [symbol] | 50 bp; e4-5 |
| | | receptor | | GTGCAACACCGCTTCGTGTTCAA [symbol] | |
| | | | | AG | |
| | | | | (SEQ ID NO: 26) | |
| M003 | CRH-R1 | | | GTGCCAGGAGATCCTCAATGAG [symbol] | 50 bp; e4-5 |
| | | | | AGAAAAAAAGCAAGGTGCACTA [symbol] | |
| | | | | CATG | |
| | | | | (SEQ ID NO: 27) | |
| M004 | | | | AGGAGATCCTCAATGAGGAGAA [symbol] | 40 bp; e4-5 |
| | | | | AAAAGCAAGGTGCACTA | |
| | | | | (SEQ ID NO: 28) | |
| M005 | | | | ATCCTCAATGAGGAGAAAAAAA [symbol] | 30 bp; e4-5 |
| | | | | CAAGGTG | |
| | | | | (SEQ ID NO: 29) | |
| M006 | | | | GTGCCAGGAGATCCTCAATGAG [symbol] | 50 bp, 62% GC, |
| | | | | AGGAGCCTGGGACTCAGGCACA [symbol] | e4-cryptic; |
| | | | | CCTG | detects CRF-R1h |
| | | | | (SEQ ID NO: 30) | |
| M007 | | | | AGGAGATCCTCAATGAGGAGGA [symbol] | 40 bp, 62% GC |
| | | | | CCTGGGACTCAGGCACA | e4-cryptic; |
| | | | | (SEQ ID NO: 31) | detects CRF-R1h |
| M008 | | | | ATCCTCAATGAGGAGGAGCCTG [symbol] | 30 bp, 62% GC |
| | | | | GACTCAG | e4-cryptic; |
| | | | | (SEQ ID NO: 32) | detects CRF-R1h |
| M009 | | | | GTTCTACTGTTTCCTCAATAGTG [symbol] | 50 bp; e13-14; |
| | | | | GGTCCGTTCTGCCATCCGGAAGA [symbol] | |
| | | | | GT | |
| | | | | (SEQ ID NO: 33) | |
| M010 | | | | ACTGTTTCCTCAATAGTGAGGT [symbol] | 40 bp; e13-14; |
| | | | | GTTCTGCCATCCGGAA | |
| | | | | (SEQ ID NO: 34) | |
| M011 | | | | CAACTCCTTCCTGGAATCCTTCC [symbol] | 50 bp; e13-15; |
| | | | | GGTCCGTTCTGCCATCCGGAAGA [symbol] | detects deletion |
| | | | | GT | of exon 13 |
| | | | | (SEQ ID NO: 35) | |
| M012 | | | | CCTTCCTGGAATCCTTCCAGGT [symbol] | 40 bp; e13-15; |
| | | | | GTTCTGCCATCCGGAA | detects deletion |
| | | | | (SEQ ID NO: 36) | of exon 13 |
| M013 | 5HTR2B | Serotonin | XM002541 | ATTGCCCTCTTGACAATAATGTT [symbol] | 50 bp, e1-2; |
| | | receptor 2B | | GAGGCTATGTGGCCCCTCCCACT [symbol] | full-length |
| | | | | GT | |
| | | | | (SEQ ID NO: 37) | |
| M014 | | | | CCTCTTGACAATAATGTTTGAG [symbol] | 40 bp, e1-2; |
| | | | | TATGTGGCCCCTCCCA | full-length |
| | | | | (SEQ ID NO: 38) | |
| M015 | | | | TGACAATAATGTTTGAGGCTAT [symbol] | 30 bp, e1-2; |
| | | | | GGCCCC | full-length |
| | | | | (SEQ ID NO: 39) | |
| M016 | | | | ATTGCCCTCTTGACAATAATGTT [symbol] | 50 bp, e1-3; |
| | | | | GGCATTGCCATTCCAGTCCCTAT [symbol] | 5HTR2 [symbol] detects |
| | | | | AA | deletion of exon 2 |
| | | | | (SEQ ID NO: 40) | |
| M017 | | | | CCTCTTGACAATAATGTTTGGCA [symbol] | 40 bp, e1-3; 5HTR2 [symbol] |
| | | | | TGCCATTCCAGTCCCT | detects deletion of |
| | | | | (SEQ ID NO: 41) | exon 2 |
| M018 | | | | TGACAATAATGTTTGGCATTGC [symbol] | 30 bp, e1-3; 5HTR2 [symbol] |
| | | | | TTCCAG | detects deletion of |
| | | | | (SEQ ID NO: 42) | exon 2 |
| M019 | 5HTR7 | Serotonin | XM015676 | CCCTGTGCGTGATCAGCATTGA [symbol] | 50 bp, e1-2; 58% GC |
| | | receptor 7 | | GGTACCTTGGGATCACAAGGCC [symbol] | |
| | | | | TC | |
| | | | | (SEQ ID NO: 43) | |
| M020 | | | | TGCGTGATCAGCATTGACAGGTA [symbol] | 40 bp, e1-2; 58% GC |
| | | | | CTTGGGATCACAAGGC | |
| | | | | (SEQ ID NO: 44) | |
| M021 | | | | GATCAGCATTGACAGGTACCTT [symbol] | 30 bp, e1-2; 58% GC |
| | | | | GATCAC | |
| | | | | (SEQ ID NO: 45) | |
| M022 | CRF-R2 | | | CACCCCTGGACCCCGAGGGTCC [symbol] | 40 bp; e4-5; 65% GC; |
| | alpha | | | ACTCCTACTGCAACAC | detects alpha isoform |
| | | | | (SEQ ID NO: 46) | |
| M023 | CRF-R2 | | | CATGACCCTCACCAACCTCTCA [symbol] | 50 bp; e2-5; 65% GC; |
| | beta | | | TCCCTACTCCTACTGCAACACGA [symbol] | detects beta isoform |
| | | | | CT | |
| | | | | (SEQ ID NO: 47) | |
| M024 | CRF-R2 | | | CCCTCACCAACCTCTCAGGTCC [symbol] | 40 bp; e2-5; 65% GC; |
| | beta | | | ACTCCTACTGCAACAC | detects beta isoform |
| | | | | (SEQ ID NO: 48) | |
| M025 | CRF-R2 | | | CTTTCCTCAGCTCTTCTGCCAAG [symbol] | 50 bp; e3-5; 54% GC; |
| | gamma | | | TCCCTACTCCTACTGCAACACGA [symbol] | detects gamma isoform |
| | | | | CT | |
| | | | | (SEQ ID NO: 49) | |
| M026 | CRF | Corticotropin | | CGAGCAGTTAGCACAGCAAGCT [symbol] | 50 bp |
| | | releasing | | ACAGCAACAGGAAACTCATGGA [symbol] | |
| | | factor | | ATTA | |
| | | | | (SEQ ID NO: 50) | |
| M027 | Uro | Urocortin | | CTTCTCTGTCCATTGACCTCACC [symbol] | 50 bp |
| | | | | TCACCTGCTGCGGACCCTGCTG [symbol] | |
| | | | | G | |
| | | | | (SEQ ID NO: 51) | |
| M028 | CRFBP | Corticotropin | | ATAAAGACAGACCCCAACCTCT [symbol] | 50 bp; est E1-2 |
| | | releasing | | CCTTGCAATGTCATTTCTCAGA [symbol] | |
| | | factor | | CC | |
| | | binding | | (SEQ ID NO: 52) | |
| | | protein | | | |
| M029 | FSH | Follicle | NM_000510 | TGTGCTGGCTACTGCTACACCA [symbol] | 49 bp, e2-3, 55% GC |
| | beta | stimulating | | GATCTGGTGTATAAGGACCCAG [symbol] | |
| | | hormone, | | A | |
| | | p-p b [symbol] | | (SEQ ID NO: 53) | |
| M030 | FSHR | Follicle | NM_000145 | GAACCTTCCCAACCTTCAATAT [symbol] | 50 bp, e3-4, 38% GC |
| | | stimulating | | GTTAATATCCAACACAGGTATTA [symbol] | |
| | | hormone | | GC | |
| | | receptor | | (SEQ ID NO: 54) | |
| M031 | CGB | chorionic | NM_000737 | CACCAAGGATGGAGATGTTCCA [symbol] | 48 bp, e1-2, 58% GC; |
| | | gona [symbol] | | GGGCTGCTGCTGTTGCTGCTGCT [symbol] | It should also detect |
| | | tropin, beta | | A | CGB7, CGB8, proba [symbol] |
| | | polypeptide | | (SEQ ID NO: 55) | also CGB1, CGB5, |
| | | | | | CGB6 |
| M032 | GLYCA | glycoprotein | AH007338 | CTCCATTCCGCTCCTGATGTGCA [symbol] | 50 bp, e2-3, 56% GC; |
| | | alpha subunit | | GATTGCCCAGAATGCACGCTAC [symbol] | |
| | | | | GGA | |
| | | | | (SEQ ID NO: 56) | |
| M033 | | | | CTGTGTAGCTAAATCATATAACA [symbol] | 50 bp, e3-4, 42% GC; |
| | | | | GGTCACAGTAATGGGGGGTTTC [symbol] | |
| | | | | AAG | |
| | | | | (SEQ ID NO: 57) | |
| M034 | LHB | Luteinizing | NM_000894 | TGTGCCGGCTACTGCCCCACCAT [symbol] | 47 bp, e1-2, 67% GC; |
| | | hormone beta | | ATGCGCGTGCTGCAGGCGGTCC [symbol] | high homology to C [symbol] |
| | | p-p | | (SEQ ID NO: 58) | |
| M035 | LHCGR | Luteinizing | NM_000233 | TCAATGGGACGACACTGACTTCA [symbol] | 50 bp, e6-7, 46% GC; |
| | | hormone | | TGGAGCTAAAGGAAAACGTACA [symbol] | |
| | | receptor | | CTG | |
| | | | | (SEQ ID NO: 59) | |
| M036 | GNRH1 | Gonadotropin- | NM_000825 | CCTCCGAGACCTGAAAGGAGCT [symbol] | 50 bp, e2-3, 50% GC; |
| | | releasing | | GGAAAGTCTGATTGAAGAGGAA [symbol] | |
| | | hormone | | CTG | |
| | | | | (SEQ ID NO: 60) | |
| M037 | GNRH2 | Gonadotropin- | NM_001501 | CCCAGAATGCCCTTAGGCCCCCA [symbol] | 47 bp, e2-3, 67% GC; |
| | | releasing | | GAAGGGCCCTGGACACTGCAGC [symbol] | |
| | | hormone | | (SEQ ID NO: 61) | |
| M038 | GNRHR | gonadotropin- | NM_000406 | CGGGTCCTTCATCAGGACCCCCA [symbol] | 50 bp, e2-3, 52% GC; |
| | | releasing | | GAACTACAACTGAATCAGTCCA | |
| | | hormone | | GAA | |
| | | | | (SEQ ID NO: 62) | |
| M039 | GNRH2 | gonadotropin- | NM_057163 | TAGTTTCCTGCTTGCCTTCCCCC [symbol] | 50 bp, e2-3, 56% GC; |
| | | releasing | | GCTGTTCCTGTTCCACACGGTCC [symbol] | |
| | | horomone | | CT | |
| | | type 2 | | (SEQ ID NO: 63) | |
| M040 | GH2 | growth | | TTCCAACAGGGTGAAAACGCAG [symbol] | 50 bp, e3-43, 50% GC [symbol] |
| | | horomone | | AGAAATCTAACCTAGAGCTGCT [symbol] | more specific for GH [symbol] |
| | | | | GCA | than GH1, but can |
| | | | | (SEQ ID NO: 64) | detect all GH, CSH |
| M041 | GH1 | growth | NM_000515 | CAGGGAGGAAACACAACAGAAA [symbol] | 50 bp, e3-4, 54% GC, |
| | | horomone | | CCAACCTAGAGCTGCTCCGCAT [symbol] | will also detect CSH [symbol] |
| | | | | CCC | CSH2, GH2 |
| | | | | (SEQ ID NO: 65) | |
| M042 | | | | AGCCTGGTGTACGGCGCCTCTGA [symbol] | 47 bp, e4, more speci [symbol] |
| | | | | AGCAACGTCTATGACCTCCTAAA [symbol] | for GH1 than to CSH |
| | | | | (SEQ ID NO: 66) | |
| M043 | GHR | growth | NM_000163 | AAGTGTTTCTCTGTTGATGAAAT [symbol] | 50 bp, e4-5, 44% GC; |
| | | horomone | | GTGCAACCAGATCCACCCATTG [symbol] | |
| | | receptor | | CT | |
| | | | | (SEQ ID NO: 67) | |
| M044 | GHRH | growth | NM_021081 | CCACCTCCCCCTTTGACCCTCAG [symbol] | 47 bp, e1-2, 62% GC; |
| | | horomone | | ATGCGGCGGTATGCAGATGCCA [symbol] | |
| | | releasing | | (SEQ ID NO: 68) | |
| | | horomone | | | |
| M045 | GHRHR | growth | NM_000823 | TTCTCTCACTTCAGCTCAGAGTC [symbol] | 50 bp, e3-4, 48% GC; |
| | | horomone | | GGGGCTGTGAAACGGGATTGTA [symbol] | |
| | | releasing | | TAT | |
| | | horomone | | (SEQ ID NO: 69) | |
| | | receptor | | | |
| M046 | ghrelin | ghrelin | AB029434 | GAGCCCTGAACACCAGAGAGTC [symbol] | 50 bp, e1-2, 56% GC; |
| | | | | AGCAGAGAAAGGAGTCGAAGAA [symbol] | homol to motilin |
| | | | | CCAC | (the same?) |
| | | | | (SEQ ID NO: 70) | |
| M047 | MC1R | melanocortin | XM_047456 | AACCTCTTTCTCGCCCTCATCAT [symbol] | 50 bp, only 1 exon, |
| | | 1 receptor | | GCAATGCCATCATCGACCCCCT [symbol] | 52% GC; specific for |
| | | | | T | MC1R |
| | | | | (SEQ ID NO: 71) | |
| M048 | MC2R | melanocortin | NM_000529 | GATCGTCCTGCTGGCTGTGTTCA [symbol] | 50 bp, only 1 exon, |
| | | 2 receptor | | GAATAAGAATCTCCAGGCACCC [symbol] | 50% GC; specific for |
| | | | | TGT | MC2R |
| | | | | (SEQ ID NO: 72) | |
| M049 | POMC | proopiomelano [symbol] | NM_000939 | CTCACCACGGAAAGCAACCTGC [symbol] | 46 bp, e2-3, 63% GC; |
| | | tin | | GGAGTGCATCCGGGCCTGCAAG [symbol] | |
| | | | | (SEQ ID NO: 73) | |
| M050 | | | | TCAGAGAGCAGCCTCCCGAGAC [symbol] | 46 bp, e1-2, 60% GC; |
| | | | | GAGCCTCAGCCTGCCTGGAAGA [symbol] | |
| | | | | (SEQ ID NO: 74) | |
| M051 | LOC5105 | preproprolactin | NM_015893 | ACTCCATGGAGATCCGCACCCCT [symbol] | 48 bp, e1-2, 58% GC; |
| | | releasing | | ACATCAATCCTGCCTGGTACGC [symbol] | palindrom at 5′ end |
| | | peptide | | (SEQ ID NO: 75) | |
| M052 | PrRPR | prolactin | AB048946 | TGCTGCTGGTCACCTACCTGCTC [symbol] | 49 bp, only 1 exon, |
| | | releasing | | CTCTGCTGGTCATCCTCCTGTCT [symbol] | 57% GC; |
| | | peptide | | (SEQ ID NO: 76) | |
| | | receptor | | | |
| M053 | PRL | prolactin | XM_033558 | GGGCATGGAGCTGATAGTCAGC [symbol] | 50 bp, e4-5, 48% GC; |
| | | | | AGGTTCATCCTGAAACCAAAGA [symbol] | may detect peptides |
| | | | | AATG | similar to prolactin |
| | | | | (SEQ ID NO: 77) | |
| M054 | PRLR | prolactin | NM_000949 | GGATTTGATGCTCATCTGTTGGA [symbol] | 50 bp, e9-10, 46% GC [symbol] |
| | | receptor | | AAGGGCAAGTCTGAAGAACTA [symbol] | should be absent whe [symbol] |
| | | | | GAG | is alternatively |
| | | | | (SEQ ID NO: 78) | spliced in cancer |
| | | | | | (PRLR1b) |
| M055 | SST | somatostatin | NM_001048 | TGGCTGCTGCCGCGGGGAAGCA [symbol] | 47 bp, e1-2, 62% GC; |
| | | | | GAACTGGCCAAGTACTTCTTGG [symbol] | have a palindron |
| | | | | (SEQ ID NO: 79) | |
| M056 | SSTR1 | somatostatin | NM_001049 | CCTCTATGGCTTTCTCTCAGACA [symbol] | 50 bp, only 1 exon, |
| | | receptor 1 | | CTTCAAGCGCTCTTTCCAACGCA [symbol] | 50% GC; specific for |
| | | | | CC | SSTR1 |
| | | | | (SEQ ID NO: 80) | |
| M057 | SSTR2 | somatostatin | XM_085745 | GGCTATCCATTCCATTTGACCTC [symbol] | 50 bp, only 1 exon, |
| | | receptor 2 | | ATGGCTCTGTGGTGTCAACCAA [symbol] | 50% GC; specific for |
| | | | | CC | SSTR2 |
| | | | | (SEQ ID NO: 81) | |
| M058 | | | | TCAATCAGTTCACCAGCATCTT [symbol] | 50 bp, only 1 exon, |
| | | | | GCCTGACAGTCATGAGCATCGA [symbol] | 50% GC; less specific |
| | | | | GA | may also detect SST [symbol] |
| | | | | (SEQ ID NO: 82) | 3, 5 genes |
| M059 | SSTR3 | somatostatin | NM_001051 | CGCTCTTCGTGCTCTGCTGGATG [symbol] | 50 bp, only 1 exon, |
| | | receptor 3 | | CCTTCTACGTGCTCAACATCGTC [symbol] | 56% GC; specific for |
| | | | | AC | SSTR3 |
| | | | | (SEQ ID NO: 83) | |
| M060 | SSTR4 | somatostatin | XM_009594 | GTCTTCGTGGTCTACACTTTCCT [symbol] | 47 bp, only 1 exon, |
| | | receptor 4 | | CTGGGCTTCCTGCTGCCCGTGCT [symbol] | 59% GC; may also |
| | | | | (SEQ ID NO: 84) | detect SSTR1 |
| M061 | SSTR5 | somatostatin | NM_001053 | TCACCAACATCTACATTCTCAA [symbol] | 50 bp, only 1 exon, |
| | | receptor 5 | | TGGCAGTGGCCGACGTCCTGTA [symbol] | 59% GC; may also |
| | | | | TG | detect SSTR2, 3, 4 |
| | | | | (SEQ ID NO: 85) | |
| M062 | TRH | thyrotropin- | XM_002795 | AAGGGGACCAGGGTGAGCACT [symbol] | 48 bp, e2-3, 56% GC; |
| | | releasing | | GCGTCCCAGATCTITTCAATCTGA [symbol] | |
| | | horomone | | T | |
| | | | | (SEQ ID NO: 86) | |
| M063 | TRHR | thyrotropin- | XM_017795 | CAGCACAGTATCTTCAAGGAAG [symbol] | 50 bp, e1-2, 52% GC; |
| | | releasing | | AGGTCACCAAGATGCTGGCAGT [symbol] | |
| | | horomone | | GTTG | |
| | | receptor | | (SEQ ID NO: 87) | |
| M064 | TSHb | Thyroid stimu- | NM_000549 | CTGTGCTGGATATTGTATGACA [symbol] | 50 bp, e1-2, 42% GC; |
| | | lating hormon, | | GGATATCAATGGCAAACTGTTT [symbol] | |
| | | beta subunit | | TC | |
| | | | | (SEQ ID NO: 88) | |
| M065 | TSHR | Thyroid stimu- | NM_000369 | GCTTACCGCCCAGTACGCAGACT [symbol] | 50 bp, e1-2, 52% GC; |
| | | lating hormon | | TGAAGCTTATTGAGACTCACCT [symbol] | |
| | | receptor | | GA | |
| | | | | (SEQ ID NO: 89) | |
| M066 | DIO1 | type I 5′ | S48220 | TGCAACATTTGGGAGTTTATGCA [symbol] | 50 bp, e1-2, 42% GC; |
| | | iodothyronine | | GGTAATAGGCCACTGGTGCTGA [symbol] | |
| | | deiodinase | | TT | |
| | | | | (SEQ ID NO: 90) | |
| M067 | DIO2 | type 2 5′ | NM_000793 | CTGGAAGAGCTTCCTCCTCGAT [symbol] | 50 bp, e2-3, 52% GC; |
| | | iodothyronine | | CTACAAACAGGTGAAATTGGGT [symbol] | |
| | | deiodinase v2 | | AGG | |
| | | | | (SEQ ID NO: 91) | |
| M068 | DIO3 | type 3 5′ | NM_001362 | GCTACCAGGTCTCTGAGCTGCG [symbol] | 50 bp, only 1 exon |
| | | iodothyronine | | CTTGGTTGGAACGCTATGATGA [symbol] | 54% GC; |
| | | deiodinase | | AA | |
| | | | | (SEQ ID NO: 92) | |
| M069 | SLC26A4 | Iodine | NM_000441 | AGAGCACTGGAGGAAAGACACA [symbol] | 50 bp, e9-10, 52% GC [symbol] |
| | | transport | | GTTGCTGGCATCATCTCTGCTG [symbol] | |
| | | | | ATT | |
| | | | | (SEQ ID NO: 93) | |
| M070 | THRA | thyroid | XM_008585 | ACTTGTGAGGGCTGCAAGGGCT [symbol] | 50 bp, e4-5, 54% GC; |
| | | horomone | | TTTCGCCGCACAATCCAGAAGAA [symbol] | |
| | | receptor, | | CT | |
| | | alpha | | (SEQ ID NO: 94) | |
| M071 | THRB | thyroid | NM_000461 | ATCTATGTTGGCATGGCAACAGA [symbol] | 50 bp, e6-7, 48% GC; |
| | | hormone | | TTGGTGCTGGATGACAGCAAGA [symbol] | |
| | | receptor, bet | | GCT | |
| | | | | (SEQ ID NO: 95) | |
| M072 | TG | thyroglobulin | NM_003235 | TAGGGAAGCCCAAGAAATGCCC [symbol] | 50 bp, e9-10 (the |
| | | | | ACGCCCTGTCAAUACAGGCTGA [symbol] | biggest), 54% GC; |
| | | | | CAA | |
| | | | | (SEQ ID NO: 96) | |
| M073 | | | | CAAGGGCGGGAACTGGCTGAGA [symbol] | 50 bp, e7-8 (Assuming |
| | | | | AGGTTTGGAGTTGTTACTGGAT [symbol] | that it can also be |
| | | | | AAT | shorter protein), |
| | | | | (SEQ ID NO: 97) | 50% GC |
| M074 | AG | Androgen | L29496 | TATGAAGCAGGGATGACTCTGG [symbol] | 50 bp, e3-4, 50% GC; |
| | | receptor | | AGCCCGGAAGCTGAAGAAACTT [symbol] | |
| | | | | GTAA | |
| | | | | (SEQ ID NO: 98) | |
| M075 | SBP | plasma sex | X05792 | AGGGACTCAGGCAGAATTCAAT [symbol] | 50 bp, 50% GC; will |
| | | steroid- | | TCCGAGACATTCCCCAGCCTCAT [symbol] | also detect SHGB, |
| | | binding | | CAG | SHGB-2 |
| | | protein | | (SEQ ID NO: 99) | |
| M076 | ESR1 | estrogen | XM_045967 | TGCTACGAAGTGGGAATGATGA [symbol] | 50 bp, e3-4, 48% GC; |
| | | receptor 1 | | AGGTGGGATACGAAAAGACCGA [symbol] | |
| | | | | GAGG | |
| | | | | (SEQ ID NO: 100) | |
| M077 | ESRRB o [symbol] | estrogen | AB006590 | TTACGAAGTGGGAATGGTGAAG [symbol] | 50 bp, e3-4, 53% GC; |
| | ESR2 | receptor | | GTGGCTCCCGGAGAGAGAGATG [symbol] | |
| | | beta | | GGGT | |
| | | | | (SEQ ID NO: 101) | |
| M078 | ESRRA | estrogen- | NM_004451 | ATCAGCTGGGCCAAGAGCATCC [symbol] | 50 bp, e3-4, 58% GC; |
| | | related | | AGGCTTCTCATCGCTGTCGCTGT [symbol] | |
| | | receptor | | TGA | |
| | | alpha | | (SEQ ID NO: 102) | |
| M079 | | estrogen | L25275 | GACTCTGTTGGATCAGAAGGTCA [symbol] | 50 bp, e4-5, 50% GC; |
| | | sulfotrans- | | GGTGGTCTATGTTGCCCGAAAC [symbol] | may also detect phen [symbol] |
| | | ferase | | AA | and aryl |
| | | | | (SEQ ID NO: 103) | sulphotransferase |
| M080 | REA | repressor of | NM_007273 | GAGCAGCCCAGAATATCTCCAA [symbol] | 50 bp, e6-7, 50% GC |
| | | estrogen | | ACGATCGCCACATCACAGAATC [symbol] | |
| | | receptor | | ATC | |
| | | activity | | (SEQ ID NO: 104) | |
| M081 | | glucocorticoid | X03225 | TGAAATGGGCAAAGGCAATAC [symbol] | 50 bp, e5-6, 46% GC; |
| | | receptor | M10901 | GGTTTCAGGAACTTACACCTGGA [symbol] | detects both alpha and |
| | | | X03348 M11 | GAG | beta receptors |
| | | | | (SEQ ID NO: 105) | |
| M082 | | alpha- | X03225 | GACGATGAGTATTGAATTCCCC [symbol] | 50 bp, e10, 42% GC; |
| | | glucocorticoid | M10901 | GATGTTAGCTGAAATCATCACCA [symbol] | detects only alpha |
| | | receptor | | TC | receptor = NR3C1 |
| | | | | (SEQ ID NO: 106) | |
| M083 | LOC1432 | similar to | XM_089709 | ACAAAGCAGGGAAGAGAAACTT [symbol] | 50 bp, e2-3, 42% GC |
| | | glucocorticoid | | AGGTGGGAGCATGTGTCAGTCTT [symbol] | |
| | | receptor | | CCC | |
| | | | | (SEQ ID NO: 107) | |
| M084 | MR | mineralocorticoid | M16801 | CTTCAAGCTGGAATGAATTTAG [symbol] | 50 bp, e4-5, 40% GC; |
| | | receptor | | GCACGAAAGTCAAAGAAGTTG [symbol] | detects full-length = |
| | | | | AAA | NR3C2 |
| | | | | (SEQ ID NO: 108) | |
| M085 | | mineralocorticoid | AJ315514 | CTTCAAGCTGGAATGAATTTAG [symbol] | 50 bp, e4-?, 44% GC; |
| | | receptor delta | | GAGAGAAGATGCATCAGTCTGC [symbol] | should detect delta |
| | | | | ATG | isoform |
| | | | | (SEQ ID NO: 109) | |
| M086 | BZRP | benzodiazapine | NM_000714 | TTCTTTGGTGCCCGACAAATGG [symbol] | 50 bp, e2-3, 58% GC; |
| | | receptor | | TGGGCCTTGGTGGATCTCCTGCT [symbol] | |
| | | | | GT | |
| | | | | (SEQ ID NO: 110) | |
| M087 | NR1H2 | nuclear | NM_007121 | CCTCCTGAAGGCATCCACTATC [symbol] | 50 bp, e7-8, 56% GC; |
| | | receptor | | GATCATGCTGCTAGAGACAGCC [symbol] | |
| | | subfamily 1, | | GGC | |
| | | group H | | (SEQ ID NO: 111) | |
| | | member 2 | | | |
| M088 | NR1I3 | nuclear | NM_005122 | GTGAGGGCTGCAAGGGTTTCTT [symbol] | 50 bp, e3-4, 56% GC; |
| | | receptor | | GGAGAACAGTCAGCAAAAGCAT [symbol] | |
| | | subfamily 1, | | GGT | |
| | | group I, | | (SEQ ID NO: 112) | |
| | | member | | | |
| M089 | NR2C1 | nuclear | NM_003297 | TGATCTGTCTGCACAACACCTG [symbol] | 50 bp, e2-3, 48% GC; |
| | | receptor | | GCTCCTAACAGATAATTCTCCA [symbol] | |
| | | subfamily 2, | | CC | |
| | | group C | | (SEQ ID NO: 113) | |
| | | member 1 | | | |
| M090 | NR2C2 | nuclear | NM_003298 | TTCAGACACACACGTCACATTTA [symbol] | 50 bp, e10-11 46% G [symbol] |
| | | receptor | | GCTAACAATGCCCAGTCCAATG [symbol] | may detect some |
| | | subfamily 2, | | AG | orphan receptors |
| | | group C, | | (SEQ ID NO: 114) | |
| | | member 2 | | | |
| M091 | NR2F2 | nuclear | NM_021005 | AAGGCCATAGTCCTGTTCACCT [symbol] | 50 bp, e2-3, 52% GC; |
| | | receptor | | GATGCCTGTGGTCTCTCTGATGT [symbol] | |
| | | subfamily 2, | | GC | |
| | | group F, | | (SEQ ID NO: 115) | |
| | | member 2 | | | |
| M092 | NR4A1 | nuclear | NM_002135 | TGGCGGTGGGCATGGTGAAGGA [symbol] | 47 bp, e3-4, 57% GC; |
| | | receptor | | GTTGTCCGAACAGACAGCCTGA [symbol] | |
| | | subfamily 4, | | G | |
| | | group A, | | (SEQ ID NO: 116) | |
| | | member 1 | | | |
| M093 | NR0B1 | nuclear | NM_000475 | CGCTTCTGTACCGCTGCTGCTTT [symbol] | 44 bp, e1, 60% GC; |
| | | receptor | | GCGGTGAAGACCACCCGCAGCA [symbol] | |
| | | subfamily 0, | | (SEQ ID NO: 117) | |
| | | group B, | | | |
| | | member 1 | | | |
| M094 | NR2F1 | nuclear | NM_005654 | TCAAAGTGGGCATGAGGCGGGA [symbol] | 47 bp, e1-2, 57% GC; |
| | | receptor | | GCGGTTCAGCGAGGAAGAATG [symbol] | has homology to orphan |
| | | subfamily 2, | | T | receptor (COUP-TFI) |
| | | group F, | | (SEQ ID NO: 118) | |
| | | member 1 | | | |
| M095 | NR1H4 | nuclear | NM_005123 | GCACTGACCTGTGAGGGGTGTA [symbol] | 50 bp, e4-5, 48% GC |
| | | receptor | | AGGTTTCTTCAGGAGAAGCATTA [symbol] | farnesol receptor |
| | | subfamily 1, | | CAA | |
| | | group H, | | (SEQ ID NO: 119) | |
| | | member 4 | | | |
| M096 | NR2E1 | nuclear | AF411525 | TGAGCAAGCCAGCCGGATCAAC [symbol] | 50 bp, e1-2, 50% GC |
| | | receptor | | AGCCGCATTTTAGATATCCCCTG [symbol] | |
| | | subfamily 2 | | AAA | |
| | | group E | | (SEQ ID NO: 120) | |
| | | member 1 | | | |
| M097 | NR4A2 | nuclear | NM_006186 | TGTITTGTCCTTCGATTAGCATAC [symbol] | 50 bp, e6-7, 48% GC; |
| | | receptor | | GGTCCAACCCAGTGGAGGGTAA [symbol] | almost the same as |
| | | subfamily 4, | | CTC | NGFI-B/nur77 (acc |
| | | group A, | | (SEQ ID NO: 121) | S77154)-has several |
| | | member 2 | | | exons deleted |
| M098 | NR4A3 | nuclear | NM_006981 | CTCAGTGTTGGAATGGTAAAAG [symbol] | 50 bp, e6-7, 42% GC |
| | | receptor | | AGTTGTCCGTACAGATAGTCTGA [symbol] | neuron derived orphan |
| | | subfamily 4, | | AGG | receptor (NOR-1, |
| | | group A, | | (SEQ ID NO: 122) | #D78579) |
| | | member 3 | | | |
| M099 | NR5A1 | nuclear | NM_004959 | TGCTCACGTGTGAGAGCTGCAA [symbol] | 56 bp, e1-2, 58% GC = |
| | | receptor | | GGCTTCTTCAAGCGCACGGTGCA [symbol] | steroidogenic factor |
| | | subfamily 5, | | (SEQ ID NO: 123) | 1(#U76388) |
| | | group A, | | | |
| | | member 1 | | | |
| M100 | | | | AAGTTCATCATCCTCTTCAGCCT [symbol] | 50 bp, e5-6, 40% GC |
| | | | | GATTTGAAGTTCCTGAATAACCA [symbol] | |
| | | | | AT | |
| | | | | (SEQ ID NO: 124) | |
| M101 | NR6A1 | nuclear | NM_033334 | CGCCCTCCGATGAAGAACTACA [symbol] | 50 bp, e5-6, 50% GC = |
| | | receptor | | AGATTTAGTGATGAAGGGATGG [symbol] | gem cell nuclear |
| | | subfamily 6, | | GGTG | receptor, GCNF |
| | | group A, | | (SEQ ID NO: 125) | (S83309) |
| | | member 1 | | | |
| M102 | PGR | progesterone | NM_000926 | CTTCTTTAAGAGGGCAATGGAA [symbol] | 50 bp, e2-3, 44% GC |
| | | receptor | | GGCAGCACAACTACTTATGTGCT [symbol] | |
| | | | | GAA | |
| | | | | (SEQ ID NO: 126) | |
| M103 | | Delta | | GCTGTCAGGCTGGCATGGTCCTT [symbol] | 50 bp, e2-3, 46% GC |
| | | | isoform | | GAGGTTTTCGAAACTTACATATT [symbol] |
| | | | | AT | |
| | | | | (SEQ ID NO: 127) | |
| M104 | PGRMC1 | progesterone | NM_006667 | AGTGACTGGGAGTCTCAGTTCA [symbol] | |
| | | receptor | | TTCAAGTATCATCACGTGGGCAA [symbol] | |
| | | membrane | | CT | |
| | | component 1 | | (SEQ ID NO: 128) | |
| M105 | PGRMC2 | progesterone | NM_006320 | CGAGAATGGGAAATGCAGTTTA [symbol] | 50 bp, e2-3, 36% GC |
| | | receptor | | AGAAAAATATGATTATGTAGGC [symbol] | |
| | | membrane | | GACT | |
| | | component 2 | | (SEQ ID NO: 129) | |
| M106 | RARA | retinoic acid | NM_000964 | TTTGAAGTGGGCATGTCCAAGGA [symbol] | 50 bp, e4-5, 46% GC |
| | | receptor, | | GTCTGTGAGAAACGACCGAAAC [symbol] | |
| | | alpha | | AGAA | |
| | | | | (SEQ ID NO: 130) | |
| M107 | RARB v1 | retinoic acid | NM_000965 | GGTCAGCGCCTGTGAGGGATGT [symbol] | 50 bp, e2-3, 52% GC; |
| | (NR1B2) | receptor, | | AGGGCTTTTTCCGCAGAAGTATT [symbol] | detects both v1 and |
| | | beta, v [symbol] | | AGA | v [symbol] isoforms |
| | | | | (SEQ ID NO: 131) | |
| M108 | RARG | retinoic acid | NM_00096 | ACCTCCGGGGCATCAGCACTAA [symbol] | 49 bp, e6-7, 55% GC |
| | | receptor, | | GGAGCTGAAAGGGCCATTACTC [symbol] | |
| | | gamma | | GAA | |
| | | | | (SEQ ID NO: 132) | |
| M109 | RXRA | retinoid X | NM_002957 | ATCTGCGGGGACCGCTCCTCAG [symbol] | 46 bp, e3-4, 58% GC |
| | | receptor, | | AAGCACTATGGAGTGTACAGCT [symbol] | |
| | | alpha | | (SEQ ID NO: 133) | |
| M110 | RXRB | retinoid X | NM_021976 | CAATCTGCGGGGACAGAAGCTC [symbol] | 48 bp, e1-2, 50% GC |
| | | receptor, | | GGCAAACACTACGGGGTTTACA [symbol] | |
| | | beta | | CT | |
| | | | | (SEQ ID NO: 134) | |
| M111 | AKR1D1 | aldo-keto | NM_005989 | TGGTACCTACTCAGAACCTAAAT [symbol] | 50 bp, e1-2, 52% GC |
| | | reductase | | GACCCCTAAGGGAGCCTGTGCA [symbol] | |
| | | family 1 | | CAT | |
| | | member D1 | | (SEQ ID NO: 135) | |
| M112 | CYP11A | cytochrome P45 [symbol] | NM_000781 | TCCAGAAGTATGGCCCGATTTA [symbol] | 48 bp, e1-2, 54% GC |
| | | subfamily X1A | | GGGAGAAGCTCGGCAACGTGGA [symbol] | P450 scc |
| | | | | T | |
| | | | | (SEQ ID NO: 136) | |
| M113 | CYP11B1 | cytochrome P4 [symbol] | NM_000497 | AAATGTGGCGTGTTCTTGCTGAA [symbol] | 50 bp, e2-3, 50% GC |
| | | subfamily X1B | | GGGCCTGAATGGCGCTTCAACC [symbol] | (steroid 11-beta- |
| | | polypeptide 1 | | ATT | hydroxylase) will also |
| | | | | (SEQ ID NO: 137) | detect CYP11B2 |
| M114 | CYP17 | cytochrome P4 [symbol] | NM_000102 | ACTTCTCTGGGCGGCCTCAAAT [symbol] | 48 bp, e2-3, 54% GC |
| | | subfamily XVI [symbol] | | CAACTCTAGACATCGCGTCCAA [symbol] | Steroid 17-alpha- |
| | | | | (SEQ ID NO: 138) | hydroxylase |
| M115 | CYP19 | cytochrome P4 [symbol] | NM_000103 | GCTCTTCTTGAGGATCCCTTTGG [symbol] | 50 bp, e5-6, 46% GC |
| | | subfamily XIX | | CGAAAGTGCTATCGTGGTTAAA [symbol] | aromatization of |
| | | | | CC | androgens |
| | | | | (SEQ ID NO: 139) | |
| M116 | CYP21A2 | cytochrome P4 [symbol] | NM_000500 | TGTGGACGTGATTCCCTTTCTCA [symbol] | 50 bp, e5-6, 54% GC |
| | | subfamily XXI [symbol] | | GTTCTTCCCCAATCCAGGTCTCC [symbol] | Steroid 21-hydroxylase |
| | | | | GA | |
| | | | | (SEQ ID NO: 140) | |
| M117 | CYP24 | cytochrome P4 [symbol] | XM_030593 | CTCAAGAAACAGCACGACACCC [symbol] | 50 bp, e1-2, 50% GC |
| | | subfamily XXI [symbol] | | GGTGGAGTACCACAAGAAGTAT [symbol] | vitamin D 24- |
| | | | | GCAA | hydroxylase |
| | | | | (SEQ ID NO: 141) | |
| M118 | CYP27A1 [symbol] | cytochrome P4 [symbol] | NM_000784 | GGTTGGAATGCCATCTTTTCCTT [symbol] | 50 bp, e4-5, 44% GC |
| | | subfamily | | GGGAAGAAGCTGATTGATGAGA [symbol] | Steroid 27-hydroxylase |
| | | XXVIIA | | GCT | |
| | | | | (SEQ ID NO: 142) | |
| M119 | CYP2C9 | cytochrome P4 [symbol] | NM_000771 | CTGCTGAAGCACCCAGAGGTCA [symbol] | 48 bp, e6-7, 50% GC |
| | | subfamily IIC | | AGCTAAAGTCCAGGAAGAGATT [symbol] | Mephenytoin 4- |
| | | | | AA | hydroxylase; will also |
| | | | | (SEQ ID NO: 143) | detect CYP2C8, 17, [symbol] 19 |
| M120 | CYP3A4 | cytochrome P4 [symbol] | NM_017460 | AGTCGCCTCGAAGATACACAAA [symbol] | 50 bp, e6-7, 48% GC |
| | | subfamily IIIA | | GCACCGAGTGGATTTCCTTCAG [symbol] | niphedipine oxidase |
| | | | | GAT | will detect other CYP [symbol] |
| | | | | (SEQ ID NO: 144) | genes |
| M121 | HSD17B1 | 17beta- | AF037438 | AGTGTGGCTGCCTTCGAGGGTCA [symbol] | = ERAB (U96132) |
| | | hydroxysteroid | | GTTGGACAAGCTGCATACTCTG [symbol] | |
| | | dehydrogenase | | T | |
| | | type 10 | | (SEQ ID NO: 145) | |
| M122 | HSD17B3 | hydroxysteroid | NM_000197 | CCTGAACGCACCGGATGAAATC [symbol] | 50 bp, e5-6, 50% GC |
| | | (17-beta) | | AGAGCCTCATCCATTGTAACAT [symbol] | |
| | | dehydrogenase | | CCT | |
| | | | | (SEQ ID NO: 146) | |
| M123 | HSD17B8 | hydroxysteroid | NM_014234 | AACATCAGTAGCATCGTAGGAA [symbol] | 50 bp, e4-5, 51% GC |
| | | (17-beta) | | GGTGGGGAACGTGGGGCAGACA [symbol] | |
| | | dehydrogenase | | ACT | |
| | | | | (SEQ ID NO: 147) | |
| M124 | | adrenaline | E07358 | TTCGTGCTCTGCTGGTITCCCCTT [symbol] | 50 bp, 50% GC |
| | | alpha2CII | | TCTTCAGCTACAGCCTGTACGG [symbol] | |
| | | receptor | | T | |
| | | | | (SEQ ID NO: 148) | |
| M125 | | adrenaline | E14946 | AGGCTTTGCCAACGGCTCGACG [symbol] | 50 bp, e1-2, 57% GC |
| | | beta receptor | | GGCTTCTTGGGGAGTTTCTTAG [symbol] | |
| | | | | (SEQ ID NO: 149) | |
| M126 | PNMT | phenylethanola [symbol] | NM_002686 | AACATGCCTGCCTCATTGAGGG [symbol] | 46 bp, e2-3, 54% GC |
| | | ne N-methyl | | AGGGGGAATGCTGGCAGGATAA [symbol] | |
| | | transferase | | (SEQ ID NO: 150) | |
| M127 | SLC6A2 | solute carrier | NM_001043 | TTCTACCGCTTGAAAGAGGCCA [symbol] | 50 bp, e5-6, 50% GC |
| | | family 6 | | GTATGGATTGATGCCGCAACTCA [symbol] | neurotransmitter |
| | | | | AT | transporter, |
| | | | | (SEQ ID NO: 151) | noradrenalin |
| M128 | ADM | adrenomedullin | NM_001124 | TGTCGCGTCGGAGTTTCGAAAGA [symbol] | 50 bp, e2-3, 52% GC |
| | | | | GTGGAATAAGTGGGCTCTGAGT [symbol] | |
| | | | | GTG | |
| | | | | (SEQ ID NO: 152) | |
| M129 | ADMR | adrenomedullin | NM_007264 | TCCCAAGGGAAACTCAGGCGTG [symbol] | 46 bp, e1-2, 55% GC |
| | | receptor | | GCTGGTCCCAATGTCAGTGAAA [symbol] | |
| | | | | (SEQ ID NO: 153) | |
| M130 | ANGPT1 | angiopoietin 1 | NM_001146 | CAACCTTGTCAATCTTTGCACTA [symbol] | 50 bp, e4-5, 38% GC |
| | | | | AGAAGGTGTTTTACTAAAGGGA [symbol] | |
| | | | | GAA | |
| | | | | (SEQ IDNO: 154) | |
| M131 | ANGPT2 | angiopoietin 2 | XM_034835 | ATCCCAGTCCACCTGAGGA | 50 bp, e9, 46% GC |
| | | | | ACTGTCTCGAACTATTTTCAAA [symbol] | |
| | | | | CTTAAGC | |
| | | | | (SEQ ID NO: 155) | |
| M132 | ANGPT4 | angiopoietin 4 | NM_015985 | AAGAAAGGGCTAACGCCTCGG [symbol] | 47 bp, e4-5, 59% GC; |
| | | | | CCGGCCTTCATAATGGCAGGTGA [symbol] | angiopoetin-3 |
| | | | | (SEQ ID NO: 156) | |
| M133 | CDT6 | angiopoietin- | NM_021146 | AGACGGTCACTCAGACCTCCGC [symbol] | 47 bp, e1-2, 57% GC |
| | | like fact | | GATGCCATCTACGACTGCTCTT [symbol] | |
| | | | | (SEQ ID NO: 157) | |
| M134 | LOC1643 | similar to | XM_092738 | AACTGGATCTGAGCGCAAAAAG [symbol] | 50 bp, only 1 exon |
| | | Angiopoietin 1 | | AAGACACAGACGGTCAAAAAC [symbol] | 50% GC |
| | | receptor | | CCCC | |
| | | precursor | | (SEQ ID NO: 158) | |
| M135 | TEK | TEK tyrosine | XM_005480 | TGTGGAGTCAGCTTGCTCCTTTC [symbol] | 50 bp, e1-2, 50% GC |
| | | kinase | | GGAACTGTGGAAGGTGCCATGG [symbol] | |
| | | | | CTT | |
| | | | | (SEQ ID NO: 159) | |
| M136 | BDKRB1 | bradykinin | NM_000710 | CTGCTGCACAGAGTGCTGCCAA [symbol] | 50 bp, e2, 52% GC |
| | | receptor B1 | | TTTATCATCTCCATCTGTTTCTT [symbol] | |
| | | | | G | |
| | | | | (SEQ ID NO: 160) | |
| M137 | BDKRB2 | bradykinin | XM_040854 | TCCGTGCCCACCACGGCCTCTTT [symbol] | 46 bp, e1-2, 60% GC |
| | | receptor B2 | | AGCGCCGACATGCTCAATGTCA [symbol] | |
| | | | | (SEQ ID NO: 161) | |
| M138 | PTGIS | prostaglandin | NM_000961 | TGATGAAAAGGCCAGGATGAAA [symbol] | 50 bp, e3-4, 50% GC |
| | | I2 (prosta- | | TGACTCTTCTCCACAGAGAGCT [symbol] | |
| | | cyclin) | | AGG | |
| | | synthase | | (SEQ ID NO: 162) | |
| M139 | CT | Calcitonin | X00356 J001 | TCATGTTAGCATGCCCCAGAAT [symbol] | 50 bp, e3, 48% GC |
| | | | | CAACTAAACTCCTCCCTTTTCCTT [symbol] | |
| | | | | CT | |
| | | | | (SEQ ID NO: 163) | |
| M140 | CALCA | Calcitonin | M64486 | ATCATTGCCCAGAAGAGAGCCT [symbol] | 49 bp, e3, 54% G |
| | | with different | | GACACTGCCACCTGTGTGACTCA [symbol] | Detected in medullar |
| | | C-term | C | thyroid carcinoma | |
| | | | | (SEQ ID NO: 164) | |
| M141 | CALCB | calcitonin- | XM_113645 | TCAAGCCTGAGCAGATGAATGA [symbol] | 50 bp, e8-9, 50% G |
| | | related | | TCCAGGAAGAAGGTTATCATGA [symbol] | |
| | | polypeptide, | | ACTG | |
| | | beta | | (SEQ ID NO: 165) | |
| M142 | CALCR | calcitonin | NM_001742 | AGGGCCGTGTACTTCAATGACA [symbol] | |
| | | receptor | | TGCTGGCTGAGTGTGGAAACCCA [symbol] | |
| | | | | TT | |
| | | | | (SEQ ID NO: 166) | |
| M143 | ECE2 | endothelin | NM_014693 | AGCCACTGAGAGACCTCATTGA [symbol] | 49 bp, e5-6, 49% GC |
| | | converting | | AAGATTGGTGGTTGGAACATTA [symbol] | |
| | | enzyme 2 | | GG | |
| | | | | (SEQ ID NO: 167) | |
| M144 | ECE2 | Variants A | AF428263 | GGCTGCCTTGTGGCCCTAGGGGT [symbol] | 47 bp, e3-9, 55% GC |
| | | | AF428264 | TATGGGATGGATTATTFGCAGCA [symbol] | |
| | | | | (SEQ ID NO: 168) | |
| M145 | ECE1 | endothelin | NM_001397 | CTCTCATCAACACCACCGACAG [symbol] | 50 bp, e9-10, 50% G |
| | | converting | | GCCTGCTCAACAACTACATGAT [symbol] | |
| | | enzyme 1 | | GG | |
| | | | | (SEQ ID NO: 169) | |
| M146 | EDN1 | endotbelin 1 | XM_165812 | ACATCATTTGGGTCAACACTCCT [symbol] | 50 bp, e2-3, 50% GC |
| | | | | AGCACGTTGTTCCGTATGGACTT [symbol] | |
| | | | | GA | |
| | | | | (SEQ ID NO: 170) | |
| M147 | EDN2 | endothelin 2 | NM_001956 | ACATCATCTGGGTGAACACTCCT [symbol] | 50 bp, e2-3, 54% GC |
| | | | | AACAGACAGCTCCTTACGGCCT [symbol] | |
| | | | | GA | |
| | | | | (SEQ ID NO: 171) | |
| M148 | EDN3 | endothelin 3 | XM_030725 | ACATCATTTGGATCAACACTCC [symbol] | 49 bp, e2-3, 53% GC |
| | | | | AACAGACGGTGCCCTATGGACT [symbol] | |
| | | | | C | |
| | | | | (SEQ ID NO: 172) | |
| M149 | EDNRA | endothelin | NM_001957 | TGATCTCCCTATCAATGTATTTA [symbol] | 50 bp, e2-3, 50% GC |
| | | receptor | | GCTGCTGGCTGGGCGCTGGCCTT [symbol] | |
| | | type A | | TG | |
| | | | | (SEQ ID NO: 173) | |
| M150 | EDNRB | endothelin | XM_007108 | TGACATCCCTATCAATGTCTACA [symbol] | 50 bp, e1-2, 50% GC |
| | | receptor | | GCTGCTGGCAGAGGACTGGCCA [symbol] | very similar to M149 |
| | | type B | | TG | |
| | | | | (SEQ ID NO: 174) | |
| M151 | | | | GTCTATGTGCTCTGAGTATTGA [symbol] | 50 bp, e2-3, 50% GC |
| | | | | GATATCGAGCTGTTGCTTCTTGG [symbol] | sure only EDNRB |
| | | | | GT | |
| | | | | (SEQ ID NO: 175) | |
| M152 | CCK | cholecystokinin | NM_000729 | AGATACATCCAGCAGGCCCGGA [symbol] | 50 bp, e2-3, 54% GC |
| | | | | AGCTCCTTCTGGACGAATGTCCA [symbol] | |
| | | | | CGT | |
| | | | | (SEQ ID NO: 176) | |
| M153 | CCKBR | cholecystokinin | NM_000731 | TGCAAGGCGGTTTCCTACCTCAT [symbol] | 47 bp, e2-3, 57% GC; |
| | | receptor | | GGGGTGTCTGTGAGTGTGTCCA [symbol] | will also detect gastric |
| | | | | (SEQ ID NO: 177) | receptor |
| M154 | GAS | gastrin | NM_000805 | CCCACACCTCGTGGCAGACCCGT [symbol] | 41 bp, e1-2, 63% GC |
| | | | | CAAGAAGCAGGGACCAT | |
| | | | | (SEQ ID NO: 178) | |
| M155 | | | | CGACTATGTGTGTATGTGCTGAT [symbol] | 49 bp, e1, 53% GC |
| | | | | TTTGCACTGGCTCTGGCCGCCTT [symbol] | |
| | | | | T | |
| | | | | (SEQ ID NO: 179) | |
| M156 | GRP | gastrin- | NM_002091 | AAAGATGTAGGTTCAAAAGGCA [symbol] | 50 bp, e2-3, 44% GC |
| | | releasing | | AGTTGGTAGACTCTCTGCTCCA [symbol] | |
| | | peptide | | TTC | |
| | | | | (SEQ ID NO: 180) | |
| M157 | GRPR | gastrin- | NM_005314 | AGGGAATATACATGTCAAGAAG [symbol] | 50 bp, e1-2, 46% GC |
| | | releasing | | AGATTGAATCCCGGAAGCGACT [symbol] | |
| | | peptide | | GCCA | |
| | | receptor | | (SEQ ID NO: 181) | |
| M158 | LEP | leptin | NM_000230 | CAGGATCAATGACATTTCACACA [symbol] | 50 bp, e1-2, 46% GC |
| | | | | GCAGTCAGTCTCCTCCAAACAGA [symbol] | |
| | | | | AG | |
| | | | | (SEQ ID NO: 182) | |
| M159 | LEPR | Leptin | NM_002303 | ACCTTTTCATGGCCTATGAGCAA [symbol] | 50 bp, e15-16, 46% G [symbol] |
| | | | | GTAAATATCGTGCAGTCACTCA [symbol] | |
| | | | | GC | |
| | | | | (SEQ ID NO: 183) | |
| M160 | MCH-R2 | melanin- | AF399937 | TCATGACTGTAATGAGTGTGGA [symbol] | 50 bp, e2-3, 46% GC |
| | | concentrating | | GGTACTTTGCCCTCGTCCAACCA [symbol] | |
| | | hormone | | TT | |
| | | receptor | | (SEQ ID NO: 184) | |
| M161 | PMCH | pro-melanin- | XM_006590 | CGAAGAGGAAAATAAAGTTTCA [symbol] | 50 bp, e1-2, 34% GC |
| | | concentrating | | AGAACACAGGCTCCAAACATAA [symbol] | |
| | | hormone | | TTCT | |
| | | | | (SEQ ID NO: 185) | |
| M162 | | | | CATGCTCAGATGTATGCTGGGAA [symbol] | 50 bp, e3, 50% GC |
| | | | | AGTCTACCGACCTTGTTGGCAA [symbol] | |
| | | | | CT | |
| | | | | (SEQ ID NO: 186) | |
| M163 | | Something | | GTAGCGTTAAGATAGATCAGTA [symbol] | 50 bp, 36% GC |
| | | sim [symbol] from | | CCAAAATCATAGGCTTTTTCTGT [symbol] | |
| | | chromosome 5 | | CAT | |
| | | | | (SEQ ID NO: 187) | |
| M164 | CABP1 | calcium | NM_031205 | GGAACTGCGAGATGCTTTCCGA [symbol] | 50 bp, e5-6, 50% GC |
| | | binding | | AGTTTGACACCAATGGTGATGG [symbol] | |
| | | protein 1 v1 | | GAAA | |
| | | | | (SEQ ID NO: 188) | |
| M165 | | Variant 2 | | TCCACTGAGAAATCTCTCAAGGA [symbol] | 50 bp, e1-3, 46% GC |
| | | | | GGATAGATCACTGCGACCAGAG [symbol] | |
| | | | | AAA | |
| | | | | (SEQ ID NO: 189) | |
| M166 | CABP2 | calcium | NM_016366 | GCAGCATAAAGGACTCAAGGA [symbol] | 47 bp, e7, 55% GC |
| | | protein 2 v1 | | ACAGCCTCTGCCCACCAGCATGT [symbol] | |
| | | | | (SEQ ID NO: 190) | |
| M167 | CABP3 | calcium | NM_016367 | CTCAAGAAATGCTTCCCGCAGCT [symbol] | 50 bp, e1-2, 50% GC |
| | | binding | | TTCAATCTCATCTTGTCCCAGTG [symbol] | |
| | | protein 3 | | TC | |
| | | | | (SEQ ID NO: 191) | |
| M168 | CABP5 | calcium | NM_019855 | CACTGGGACAAGATGAGATTGA [symbol] | 50 bp, e1-2, 48% GC |
| | | binding | | GAGCTGCGGGAAGCATTTCTTGA [symbol] | |
| | | protein 5 | | TTC | |
| | | | | (SEQ ID NO: 192) | |
| M169 | CALM1 | calmodulin 1 | NM_006888 | AACTGTCATGAGGTCACTGGGT [symbol] | 50 bp, e2, 50% GC |
| | | | | GAACCCAACAGAAGCTGAATTG [symbol] | will also detect |
| | | | | AGG | calmodulin 2 |
| | | | | (SEQ ID NO: 193) | |
| M170 | CALM2 | calmodulin 2? | D45887 | AGAAGCATTCCGTGTGTTTGATA [symbol] | 50 bp, e3-4, 48% GC |
| | | | | GGATGGCAATGGCTATATTAGT [symbol] | |
| | | | | TG | |
| | | | | (SEQ ID NO: 194) | |
| M171 | CALM3 | calmodulin 3 | NM_005184 | ATATGATCAATGAGGTGGATGC [symbol] | 50 bp, e3-4, 48% GC |
| | | | | GATGACCTTCCAGGGAACGGGA [symbol] | |
| | | | | CATTGA | |
| | | | | (SEQ ID NO: 195) | |
| M172 | CALN1 | calneuron 1 | NM_031468 | CACGATAGACAGCATATTCTGG [symbol] | 50 bp, e3-4, 44% GC |
| | | | | GTTTGACATGCAAAGGATAACT [symbol] | |
| | | | | GG | |
| | | | | (SEQ ID NO: 196) | |
| M173 | CAMKK2 | calcium/calmo [symbol] | XM006592 | CACGTCTCCATCACGGGTATGCA [symbol] | 49 bp, e2-3, 51% GC |
| | | n-dependent | | GACTGTGTGCAGCTGAATCAGTA [symbol] | |
| | | protein kinase | | A | |
| | | kinase 2 | | (SEQ ID NO: 197) | |
| M174 | PDE1A | phosphodiester [symbol] | NM_005019 | GAAACAGTTTGCAGCAGCCTGA [symbol] | 50 bp, e10-11, 50% G [symbol] |
| | | 1A, calmodulin | | GGGATTGACAGAGCCAAAACCA [symbol] | |
| | | dependent | | GTCC | |
| | | | | (SEQ ID NO: 198) | |
| M175 | MLN | motilin | NM_002418 | CTATGGCGAACTCCAGAGGATG [symbol] | 50 bp, e2-3, 50% GC |
| | | | | AGGAAAAGGAACGGAATAAAG [symbol] | |
| | | | | CAAA | |
| | | | | (SEQ ID NO: 199) | |
| M176 | NPPB | natriuretic | NM_002521 | ACTTGGAAACGTCCGGGTTACA [symbol] | 46 bp, e2, 56% GC; |
| | | peptide | | GAGCAGCGCAACCATTTGCAGG [symbol] | |
| | | precursor B | | (SEQ ID NO: 200) | |
| M177 | NPPC | natriuretic | NM_024409 | CAAATACAAAGGAGCCAACAA [symbol] | 48 bp, e1-2, 52% GC |
| | | peptide | | AGGGCTTGTCCAAGGGCTGCTT [symbol] | |
| | | precursor C | | (SEQ ID NO: 201) | |
| M178 | NPR1 | natriuretic | NM_000906 | CGGATGTGGAACCGAAGCTTTC [symbol] | 50 bp, e3-4, 52% GC |
| | | peptide | | AGGTGTGACAGGATACCTGAAA [symbol] | |
| | | receptor A | | TTGA | |
| | | | | (SEQ ID NO: 202) | |
| M179 | | | | GGATCCCGAGAATGGTGCCTTC [symbol] | 50 bp, e5-6, 50% GC |
| | | | | GGTTGTACTGAACTACAATGGG [symbol] | |
| | | | | TT | |
| | | | | (SEQ ID NO: 203) | |
| M180 | NPR2 | natriuretic | NM_003995 | AAAGATGCAGGGACGAAGATAT [symbol] | 50 bp, e4-5, 50% GC |
| | | peptide | | ACGGTGTAACTGGGCTGGTTGT [symbol] | |
| | | receptor B | | TGG | |
| | | | | (SEQ ID NO: 204) | |
| M181 | | | | TTGGACGACCCATCCTGTGATAA [symbol] | 50 bp, e6-7, 46% GC |
| | | | | ACTCCACTTTCAACCCTGGCAAT [symbol] | |
| | | | | GT | |
| | | | | (SEQ ID NO: 205) | |
| M182 | NPR3 | natriuretic | NM_000 | CAATATCCAGGCCAGTGAGAGA [symbol] | 48 bp, e1-2, 52% GC |
| | | peptide | | TGGTGATCATGTGTGCGAGCAG [symbol] | |
| | | receptor C | | A | |
| | | | | (SEQ ID NO: 206) | |
| M183 | KIAA097 | netrin G1f | NM_014917 | CCCTCCTGAGACGTTCTGTGCAA [symbol] | 50 bp, e2-3, 50% GC |
| | | | | GGGCAATCCCTACATGTGCAATA [symbol] | |
| | | | | TG | |
| | | | | (SEQ ID NO: 207) | |
| M184 | KIAA185 | netrin G2 | NM_032536 | CCCTGAGAGGTTCTGCTCCCAT [symbol] | 48 bp, e1-2, 54% GC |
| | | | | GAATCCCTACCTATGCAGCAAC [symbol] | |
| | | | | (SEQ ID NO: 208) | |
| M185 | NTN1 | Netrin 1 | NM_004822 | CAGCAGCGTGGAGGAGCCTGAA [symbol] | 45 bp, e3-4, 59% GC |
| | | | | ACTGCGATTCCTACTGCAAGG | |
| | | | | (SEQ ID NO: 209) | |
| M186 | NTN4 | Netrin 4 | NM_02122 | TTCTCAGCTCCAGATGCTTGCAA [symbol] | 49 bp, e1-2, 51% GC |
| | | | | CCGTGTTCCTGCCATCCAGTAG [symbol] | |
| | | | | T | |
| | | | | (SEQ ID NO: 210) | |
| M187 | iNOS | nitric oxide | AF051164 | CAGGATGACCCTAAGAGTCACA [symbol] | 50 bp, 48% GC |
| | | synthase | | GCATCAAAATGGTTTCCCCCAGT [symbol] | will also detect NOS [symbol] |
| | | | | CCT | |
| | | | | (SEQ ID NO: 211) | |
| M188 | NOS1 | nitric oxide | NM_000620 | CAACTCTGTGCAGGAAGAAAGG [symbol] | 50 bp, e15-16, 50% G [symbol] |
| | | synthase 1 | | AGAGCTACAAGGTCCGATTCAA [symbol] | |
| | | | | AGCG | |
| | | | | (SEQ ID NO: 212) | |
| M189 | NOS2 | nitric oxide | | TGACACAGGATGACCTTCAGTA [symbol] | 50 bp, 48% GC in one |
| | | synthase 2 | | ACAACCTCAGCAAGCAGCAGAA [symbol] | exon will also detect |
| | | | | GAG | iNOS |
| | | | | (SEQ ID NO: 213) | |
| M190 | NOS3 | nitric oxide | | CACAGGAAATGTTCACCTACAT [symbol] | 50 bp, 46% GC |
| | | synthase 3 | | GCAACCACATCAAGTATGCCAC [symbol] | |
| | | | | AAC | |
| | | | | (SEQ ID NO: 214) | |
| M191 | PEDF | pigment | M76979 | GCCTCCCGGATCGTCTTTGAGA [symbol] A | 50 bp, e4-5, 50% GC |
| | | epithelium- | | AAGCTGCGCATAAAATCCAGCT [symbol] | |
| | | differenti- | | GT | |
| | | ation factor | | (SEQ ID NO: 215) | |
| M192 | NPY5R | neuropeptide | NM_006174 | GCTACTGTCTGGACACTAGGTTT [symbol] | 50 bp, single exon, |
| | | Y [symbol] receptor Y5 | | GCCATCTGTTCTCCCCTTCCAGT [symbol] | 50% GC |
| | | | | TT | |
| | | | | (SEQ ID NO: 216) | |
| M193 | | neuropeptide | U42389 | AATGGGTCCAATAGGTGCAGAG [symbol] | 50 bp, single exon, |
| | | y/peptide YY | | CTGATGAGAACCAGACAGTGGA [symbol] | 50% GC |
| | | receptor type 2 | | GAAA | |
| | | | | (SEQ ID NO: 217) | |
| M194 | PPY2 | pancreatic | NM_021092 | TTGGAGCCATTGTACCGAGGGG [symbol] | 44 bp, 54% GC |
| | | polypeptide 2 | | AATACCACACCGGAGCAGAT | |
| | | | | (SEQ ID NO: 218) | |
| M195 | PPYR1 | pancreatic | NM_005972 | TACACCATCATGGACTACTGGAT [symbol] | 50 bp, single exon, |
| | | polypeptide | | TTTGGAGAGACCCTCTGCAAGAT [symbol] | 48% GC |
| | | receptor 1 | | TC | |
| | | | | (SEQ ID NO: 219) | |
| M196 | PYY | peptide YY | NM_004160 | ACTACCTCAACCTGGTCACCCG [symbol] | 41 bp, e2-3, 54% GC |
| | | | | AGCGGTATGGGAAAAGA | |
| | | | | (SEQ ID NO: 220) | |
| M197 | PYY2 | peptide YY, 2 | NM_021093 | TACGCCTATCCTCGCCAGTACCT [symbol] | 46 bp, single exon, |
| | | | | ATCCTGGTCACTCAGCCGTCGT | 54% GC |
| | | | | (SEQ ID NO: 221) | |
| M198 | VIP | vasoactive | NM_003381 | TTACAGGGCACCTTCTGCTCT [symbol] | 50 bp, e1-2, 50% GC |
| | | intestinal | | GGTTGGGTGACAGAATACCCTTT [symbol] | |
| | | peptide | | AG | |
| | | | | (SEQ ID NO: 222) | |
| M199 | | | | AGTCTCTTATGGGAAAACGTGTT [symbol] | 50 bp, e3-4, 42% GC |
| | | | | GCAGTAACATCTCAGAAGACCC [symbol] | |
| | | | | GTA | |
| | | | | (SEQ ID NO: 223) | |
| M200 | PTH | parathyroid | NM_000315 | AAATCGGATGGGAAATCTGTTA [symbol] | 50 bp, e2-3, 38% GC |
| | | hormon | | GAAGAGATCTGTGAGTGAAATA [symbol] | |
| | | | | AGCT | |
| | | | | (SEQ ID NO: 224) | |
| M201 | PTHR1 | parathyroid | NM_000316 | GGTGCCCATCCTGGCCTCCATT [symbol] | 50 bp, e10-11, 50% G [symbol] |
| | | hormone | | GCTCAACTTTCATGCTCTTCATCA [symbol] | |
| | | receptor 1 | | TA | |
| | | | | (SEQ ID NO: 225) | |
| M202 | | | | CATATACTGTTTCTGCAATGGC [symbol] | 50 bp, e14-15, 40% G [symbol] |
| | | | | GGTACAAGCTGAGATCAAGAA [symbol] | |
| | | | | CTT | |
| | | | | (SEQ ID NO: 226) | |
| M203 | PTHR2 | parathyroid | NM_005048 | GGCATGACACAAGGAAGCAATA [symbol] | 50 bp, e10-11, 50% G [symbol] |
| | | hormone | | AGGAAACTGGCCAAATCGACA [symbol] | |
| | | receptor 2 | | GGTC | |
| | | | | (SEQ ID NO: 227) | |
| M204 | | | | GGCAACTTCTGTGGACAAATCA [symbol] | 50 bp, e6-7, 44% GC |
| | | | | ATATATCGGGTGCAAGATTGCT [symbol] | |
| | | | | TG | |
| | | | | (SEQ ID NO: 228) | |
| M205 | CYP4F8 | cytochrome P4 [symbol] | NM_007253 | GTCCGATCTGTCATCAATACCT [symbol] | 50 bp, e2-3, 44% GC |
| | | subfamily IVF, | | GATGCCATTACAGACAAGGACA [symbol] | |
| | | polypeptide 8 | | AGT | |
| | | | | (SEQ ID NO: 229) | |
| M206 | PTGDR | prostaglandin | XM_051711 | CACTATGTGTTCTCTGCCCGTAA [symbol] | 50 bp, e1-2, 42% GC |
| | | D [symbol] receptor | | TTATCGCGCTTACTATGGAGCAT [symbol] | |
| | | | | TA | |
| | | | | (SEQ ID NO: 230) | |
| M207 | CYP4F11 | cytochrome P4 [symbol] | NM_021187 | GCCTATCACCAGTGCCTCAGCT [symbol] | 47 bp, e1-2, 53% GC |
| | | subfamily IVF, | | TGTCGCACCCAAGGATATGATTT [symbol] | |
| | | polypeptide 11 | | (SEQ ID NO: 231) | |
| M208 | CYP4F12 | cytochrome P4 [symbol] | NM_023944 | AACAAGAGTGCAAACATCATGC [symbol] | 50 bp, e4-5, 50% GC |
| | | isoform 4F12 | | TGACAAGTGGCAGCACCTGGCC [symbol] | |
| | | | | AGA | |
| | | | | (SEQ ID NO: 232) | |
| M209 | CYP4F2 | cytochrome P4 | NM_001082 | ATCCGGTCTGTCATCAACGCCT [symbol] | 50 bp, e3-4, 52% GC |
| | | subfamily IVF, | | GCTGCCATTGCACCAAAGGACA [symbol] | |
| | | polypeptide 2 | | GTT | |
| | | | | (SEQ ID NO: 233) | |
| M210 | CYP4F3 | cytochrome P4 | XM_029072 | CATTGATGTACTCCTGCTGAGCA [symbol] | 50 bp, e6-7, 48% GC |
| | | subfamily IVF, | | GGATGAAGATGGGAAGAAGTT [symbol] | |
| | | polypeptide 3 | | CCG | |
| | | | | (SEQ ID NO: 234) | |
| M211 | PTGER1 | prostaglandin E | NM_000955 | CGTGCATCTGCTGGAGCCCAAT [symbol] | 40 bp, e2-3, 62% GC |
| | | receptor 1 | | TGGTGTTGGTGGCGCT | |
| | | | | (SEQ ID NO: 235) | |
| M212 | PTGER2 | prostaglandin E | NM_000956 | CGCCGTCTGCTCCTTGCCTTTCA [symbol] | 50 bp, e1-2, 44% GC |
| | | receptor 2 | | GATTTTTGCATATATGAATGAAA [symbol] | |
| | | | | CT | |
| | | | | (SEQ ID NO: 236) | |
| M213 | PTGER3 | prostaglandin E | NM_000957 | AGGACTTGGTGCAGTTCTCATGA [symbol] | 50 bp, e1-2, 48% GC |
| | | receptor 3 | | AGAGAACCCTGCAGTGTCCAGC [symbol] | |
| | | | | AAG | |
| | | | | (SEQ ID NO: 237) | |
| M214 | PTGER4 | prostaglandin E | NM_000958 | TCATCTGCTCCATCCCGCTCGTG [symbol] | 48 bp, e2-3, 52% GC |
| | | receptor 4 | | TGCGAGTATTCGTCAACCAGTTA [symbol] | |
| | | | | (SEQ ID NO: 238) | |
| M215 | PTGFR | prostaglandin F | NM_000959 | TCCTGTATTTGTTGGAGCCCATT [symbol] | 50 bp, e2-3, 42% GC |
| | | receptor | | CTGGTTACAATGGCCAACATTG [symbol] | |
| | | | | AT | |
| | | | | (SEQ ID NO: 239) | |
| M216 | PTGIR | prostaglandin | NM_000960 | TGTGCTCCCTGCCTCTCACGATC [symbol] | 37 bp, e2-3, 62% GC |
| | | I2 (prosta- | | GCTGCTTCACCCA | |
| | | cyclin) | | (SEQ ID NO: 240) | |
| | | receptor | | | |
| M217 | PTGS2 | prostaglandin- | NM_000963 | TTCTATGGAGAAAACTGCTCAA [symbol] | 50 bp, e2-3, 32% GC |
| | | endoperoxide | | CCGGAATTTTTGACAAGAATAA [symbol] | |
| | | synthase 2 | | ATT | |
| | | | | (SEQ ID NO: 241) | |
| M218 | | | | CAGACAAGCAGGCTAATACTGA [symbol] | 50 bp, e7-8, 38% GC |
| | | | | AGGAGAGACTATTAAGATTGTG [symbol] | |
| | | | | TTGA | |
| | | | | (SEQ ID NO: 242) | |
| M219 | TBXA2R | thromboxane A | NM_001060 | TCTCTGTCGCTTCATGGGCGTCG [symbol] | 44 bp, 54% GC |
| | | receptor | | CATGATCTTCTTCGGCCTGT | |
| | | | | (SEQ ID NO: 243) | |
| M220 | TBXAS1 | thromboxane A | NM_001061 | TTTCAGTCCTGAAAAGCTGAAC [symbol] | 50 bp, e4-6, 33% GC |
| | | synthase 1 | | GATGGTTCCCCTCATCAGCCAA [symbol] | |
| | | | | CT | |
| | | | | (SEQ ID NO: 244) | |
| M221 | | | | GGTAGACGTTTTTAAGGAGAAA [symbol] | 49 bp, e9-10, 51% GC |
| | | | | ACATGGCCCCTGAGTTCTGCAG [symbol] | |
| | | | | TC | |
| | | | | (SEQ ID NO: 245) | |
| M222 | AGTR1 | angiotensin | XM_051470 | TGTACGCTAGTGTGTTTCTACTC [symbol] | 50 bp, 46% GC |
| | | receptor 1 | | CGTGTCTCAGCATTGATCGATA [symbol] | |
| | | | | TG | |
| | | | | (SEQ ID NO: 246) | |
| M223 | AGTR2 | angiotensin | XM_030897 | ATTTACTCCTTTTGGCTACTCTT [symbol] | 50 bp, 36% GC |
| | | receptor 2 | | TCTATGGGCAACCTATTATTCTT [symbol] | |
| | | | | T | |
| | | | | (SEQ ID NO: 247) | |
| M224 | ACE | angiotensin I | XM_044372 | CTTCTACAACAGGAAAGACTTCA [symbol] | 50 bp, e6-7, 50% GC |
| | | converting | | GATCAAGCAGTGCACACGGGTC [symbol] | |
| | | enzyme | | CGA | |
| | | | | (SEQ ID NO: 248) | |
| M225 | AGT | angiotensinoge | NM_000029 | TTCAACACCTACGTCCACTTCCA [symbol] | 50 bp, e2-3, 52% GC |
| | | | | GGGAAGATGAAGGGCTTCTCCC [symbol] | |
| | | | | GCT | |
| | | | | (SEQ ID NO: 249) | |
| M226 | REN | renin | NM_000537 | CGACAGACACCACCACCTTTAAA [symbol] | 50 bp, e1-2, 50% GC |
| | | | | GGATCTTCCTCAAGAGAATGCC [symbol] | |
| | | | | CA | |
| | | | | (SEQ ID NO: 250) | |
| M227 | | | | GGAGCCAAGAAGAGGCTGTTTG [symbol] | 49 bp, e7-8, 51% GC |
| | | | | TTATGTCGTGAAGTGTAACGAG [symbol] | |
| | | | | CC | |
| | | | | (SEQ ID NO: 251) | |
| M228 | RENBP | renin binding | NM_002910 | AAGTATGTGTGGCTGCAGGGGA [symbol] | 49 bp, e3-4, 53% GC |
| | | protein | | GCAGGTATGGATGTATTGTCGC [symbol] | |
| | | | | GT | |
| | | | | (SEQ ID NO: 252) | |
| M229 | ACE2 | ACE-related | AF291820 | AGAACTGAAGTTGAAAAGGCCA [symbol] | 50 bp, e16-17, 48% G [symbol] |
| | | carboxypeptidase | | CAGGATGTCCCGGAGCCGTATC [symbol] | |
| | | | | ATGA | |
| | | | | (SEQ ID NO: 253) | |
| M230 | SCT | secretin | NM_021920 | CTGGTGGGGAAGCGCAGCGAG [symbol] | 47 bp, e2-3, 65% GC |
| | | | | GGACGCAGAGAACA [symbol] | |
| | | | | (SEQ ID NO: 254) | |
| M231 | SCTR | Secretin | NM_002980 | GGATGCTCACCAGCAGAAATGG [symbol] | 49 bp, e3-4, 51% GC |
| | | receptor | | TCCTTGTTCCGAAACTGCACACA [symbol] | |
| | | | | GA | |
| | | | | (SEQ ID NO: 255) | |
| M232 | UTS2 | Urotensin 2 | NM_021995 | ATGCTGGGTGCAGAAAGAGGG [symbol] | 50 bp, 48% GC |
| | | | | TATTCTCAGGAAAGCAGACTCA [symbol] | |
| | | | | GTAC | |
| | | | | (SEQ ID NO: 256) | |
| M233 | AQP4 | aquaporin 4 | NM_001650 | CAGTTATCATGGGAAATTGGGA [symbol] | 50 bp, e3-4, 44% GC |
| | | | | AACCATTGGATATATTGGGTTG [symbol] | |
| | | | | CCC | |
| | | | | (SEQ ID NO: 257) | |
| M234 | AVPR1A | arginine | XM_006934 | ACAGAACATTGTCTTACTTGAT [symbol] | 50 bp, e1-2, 42% GC |
| | | vasopressin | | TCCCGATGACCTCAACAACAGG [symbol] | |
| | | receptor 1A | | AAG | |
| | | | | (SEQ ID NO: 258) | |
| M235 | | | | AGATTTCAAGTCCAGCATCTCAA [symbol] | 50 bp, e2-3, 44% GC |
| | | | | GCGACAACCTTGGTAACTCTGCA [symbol] | |
| | | | | GA | |
| | | | | (SEQ ID NO: 259) | |
| M236 | AVPR1B | arginine | NM_000707 | CGGTGACCATGCTCACGGCCTG [symbol] | 41 bp, e2-3, 61% GC |
| | | vasopressin | | ACAGCCTCATCTGCCAT | |
| | | receptor 1B | | (SEQ ID NO: 260) | |
| M237 | | | | CCACCAATGTGGCTTTCACCAT [symbol] | 50 bp, e3, 50% GC |
| | | | | CTAGCTTTTGGGCAACCTCAA [symbol] | |
| | | | | GC | |
| | | | | (SEQ ID NO: 261) | |
| M238 | AVPR2 | arginine | NM_000054 | ATGCTCATGGCGTCCACCACTT [symbol] | 40 bp, e1-2, 62% GC |
| | | vasopressin | | GCTGTGCCTGGGCATC | |
| | | receptor 2 | | (SEQ ID NO: 262) | |
| M239 | OXTR | oxytocin | NM_000916 | ATGCCAACGCGCCCAAGGAAG [symbol] | 43 bp, e1-2, 58% GC |
| | | receptor | | TCGGCCTTCATCATCGTCAT | |
| | | | | (SEQ ID NO: 263) | |
| M240 | OXT | oxytocin | NM_000915 | AAAGGCCGCTGCTTCGGGCCCA [symbol] | 39 bp, e2, 61% GC |
| | | | | ATCTGCTGCGCGGAA | |
| | | | | (SEQ ID NO: 264) | |
| M241 | | oxytocinase | U62769 | CACCGCTTTATCAAATATGCCTA [symbol] | 50 bp, e3-4, 40% GC |
| | | | | GAAGTCATCAGTCGTTCTAGAT [symbol] | |
| | | | | TG | |
| | | | | (SEQ ID NO: 265) | |
| M242 | RLN1 | relaxin 1 | NM_006911 | CCTCAGACACCTAGACCAGTGG [symbol] | 50 bp, e1-2, 46% GC |
| | | | | AGAAATTGTACCATCCTTCATCA [symbol] | will also detect |
| | | | | CAA | relaxin 2 |
| | | | | (SEQ ID NO: 266) | |
| M243 | | | | CTATCTGAGAGGCAACCATCATT [symbol] | 50 bp, e2, 48% GC |
| | | | | CCAGAGCTACAGCAGTATGTAC [symbol] | specific for |
| | | | | GC | relaxin 1 |
| | | | | (SEQ ID NO: 267) | |
| M244 | RLN2 | relaxin 2 | NM_006911 | GAGAGTTCCTTGGTGCCCTTTC [symbol] | 50 bp, e2-3, 46% GC |
| | | | | AATTGTGCCATCCTTCATCAACA [symbol] | |
| | | | | AG | |
| | | | | (SEQ ID NO: 268) | |
| M245 | RLN3 | relaxin 3 | NM_080864 | ATCCTGGCCCACGAGGCTATGG [symbol] | 45 bp, e1-2, 57% GC |
| | | | | AGATACCTTCCCGGATGCAGAT [symbol] | |
| | | | | (SEQ ID NO: 269) | |
| M246 | | vasopressin | AF031476 | ATGTCCGACCTGGAGCTGAGAC [symbol] | 36 bp, e1-2, 63% GC |
| | | precursor | | GTGCCTCCCCTGC | |
| | | | | (SEQ ID NO: 270) | |
| M247 | | | | TTTGCTGCAACGACGAGAGCTG [symbol] | 34 bp, e2-3, 61% GC |
| | | | | TGACCGAGCC | |
| | | | | (SEQ ID NO: 271) | |
| M248 | CHRNA1 | cholinergic | NM_000079 | GACAACCAATGTGCGTCTGAAA [symbol] | 50 bp, e3-4, 42% GC |
| | | receptor, | | AGCAATGGGTGGATTACAACCT [symbol] | |
| | | nicotinic, | | AAAT | |
| | | alpha poly- | | (SEQ ID NO: 272) | |
| | | peptide 1 | | | |
| | | (muscle) | | | |
| M249 | | | | TATTCTACCTGCCCACAGACTCA [symbol] | 50 bp, e6-7, 42% GC |
| | | | | GGGAGAAGATGACTCTGAGCAT [symbol] | |
| | | | | TCT | |
| | | | | (SEQ ID NO: 273) | |
| M250 | CHRNA2 | cholinergic | NM_000742 | ACCACCAACGTCTGGCTAAAAC [symbol] | 49 bp, e4-5, 53% GC |
| | | receptor, | | GGAGTGGAGCGACTACAAACTG [symbol] | |
| | | nicotinic, | | GCT | |
| | | alpha poly- | | (SEQ ID NO: 274) | |
| | | peptide 2 | | | |
| | | (neuronal) | | | |
| M251 | CHRNA4 | cholinergic | NM_000743 | TCATGGAGACCAACCTGTGGCT [symbol] | 50 bp, e3-4, 48% GC |
| | | receptor, | | AGCAAATCTGGAATGACTACAA [symbol] | |
| | | nicotinic, | | CTG | |
| | | alpha poly- | | (SEQ ID NO: 275) | |
| | | peptide 3 | | | |
| M252 | CHRNA4 | cholinergic | NM_000744 | GTCCATCGCTCAGCTCATTGAC [symbol] | 50 bp, e2-3, 50% GC |
| | | receptor, | | GGATGAGAAGAACCAGATGAT [symbol] | |
| | | nicotinic, | | CCA | |
| | | alpha poly- | | (SEQ ID NO: 276) | |
| | | peptide 4 | | | |
| M253 | CHRNA5 | cholinergic | XM_007577 | CACCAGACATCGTTTTGTTTGAT [symbol] | 50 bp, e4-5, 44% GC |
| | | receptor, | | ATGCAGATGGACGTTTTGAAGG [symbol] | |
| | | nicotinic, | | ACC | |
| | | alpha poly- | | (SEQ ID NO: 277) | |
| | | peptide 5 | | | |
| M254 | CHRNA6 | cholinergic | CHRNA6 | GGAAACCAATTTGTGGCTGCGT [symbol] | 50 bp, e2-3, 42% GC |
| | | receptor, | | CATCTGGAATGATTATAAATTG [symbol] | |
| | | nicotinic, | | CT | |
| | | alpha poly- | | (SEQ ID NO: 278) | |
| | | peptide 6 | | | |
| M255 | CHRNA7 | cholinergic | NM_000746 | GAGCCTCCTGCAGATCATGGAC [symbol] | 50 bp, e1-2, 48% GC |
| | | receptor, | | GGATGAGAAGAACCAAGTTTTA [symbol] | |
| | | nicotinic, | | CCA | |
| | | alpha poly- | | (SEQ ID NO: 279) | |
| | | peptide 7 | | | |
| M256 | CHRNB1 | cholinergic | NM_000747 | ATCCTGGCGCAACTCATCAGCCT [symbol] | 49 bp, e2-3, 51% GC |
| | | receptor, | | AACGAGAAGGATGAAGAGATGA [symbol] | |
| | | nicotinic, | | CA | |
| | | beta poly- | | (SEQ ID NO: 280) | |
| | | peptide 1 | | | |
| | | (muscle) | | | |
| M257 | CHRNB2 | cholinergic | NM_000748 | AGCAGATCATGACCACCAATGT [symbol] | 50 bp, e3-4, 50% GC |
| | | receptor, | | GGCTGACCCAGGAGTGGGAAGA [symbol] | |
| | | nicotinic, | | TAT | |
| | | beta poly- | | (SEQ ID NO: 281) | |
| | | peptide 2 | | | |
| | | (neuronal) | | | |
| M258 | CHRNB3 | cholinergic | NM_000749 | TTCCTGACATAGTTCTCTTTGAA [symbol] | 50 bp, e1-2, 48% GC |
| | | receptor, | | ATGCTGACGGCCGCTTCGAAGG [symbol] | |
| | | nicotinic, | | CC | |
| | | beta poly- | | (SEQ ID NO: 282) | |
| | | peptide 3 | | | |
| M259 | CHRNB4 | Cholinergic | XM_039151 | AGAATGACGATGAAGACCAGA [symbol] | 50 bp, e1-2, 50% GC |
| | | receptor, | | GTCGTTGAGGACTGGAAGTACG [symbol] | |
| | | nicotinic, | | GGCT | |
| | | beta poly- | | (SEQ ID NO: 283) | |
| | | peptide 4 | | | |
| M260 | DRD1 | dopamine | XM_003966 | AGACTTTGCCCTGCGACGAATAA [symbol] | 50 bp, 44% GC |
| | | receptor | | GCCATAGAGACGGTGAGTATCA [symbol] | |
| | | D1 | | TAA | |
| | | | | (SEQ ID NO: 284) | |
| M261 | DRD2 | dopamine | NM_000795 | CTCCTCTTCGGACTCAATAACG [symbol] | 50 bp, e5-6, 50% GC |
| | | receptor | | GACCAGAACGAGTGCATCATTG [symbol] | |
| | | D2 | | CAA | |
| | | | | (SEQ ID NO: 285) | |
| M262 | DRD3 | dopamine | XM_113416 | TTCTGTTTGGCTTTAATACCACA [symbol] | 49 bp, e4-5, 51% GC |
| | | receptor | | GGGACCCCACTGTCTGCTCCAT [symbol] | |
| | | D3 | | C | |
| | | | | (SEQ ID NO: 286) | |
| M263 | DRD4 | dopamine | XM_006145 | TGGTGCTGCCGCTCTTCGTCTA [symbol] | 36 bp, e1-2, 63% GC |
| | | receptor | | CCGAGGTCCAGG | |
| | | D4 | | (SEQ ID NO: 287) | |
| M264 | AANAT | arylalkylamine | NM_001088 | CTTTGAGATCGAGCGTGAAGCC [symbol] | 44 bp, e2-3, 54% GC |
| | | acetyl- | | CATCTCCGTCTTGGGCGTCT | |
| | | transferase | | (SEQ ID NO: 288) | |
| M265 | | | | ACAAGGAGAGACTCATGCAGGA [symbol] | 39 bp, e2-3, 53% GC |
| | | | | TCACTGACGCTGCACA [symbol] | |
| | | | | (SEQ ID NO: 289) | |
| M266 | TPH | Tryptophan | X52836 | GATCTGAACTAGATGCAGACCA [symbol] | 50 bp, e4-5, 46% GC |
| | | hydroxylase | | CTGGCTTCAAAGACAATGTCTA [symbol] | |
| | | | | GT | |
| | | | | (SEQ ID NO: 290) | |
| M267 | | | | ATAAAAGCCCTGAAAATCTTTCA [symbol] | 49 bp, e1-3, 46% GC |
| | | | | GTATGGAAACTGTTCCTTGGTTT [symbol] | might detect deletion |
| | | | | C | exon 2 (regulatory |
| | | | | (SEQ ID NO: 291) | domain) |
| M268 | | | | CAGATCCCTTCTATACCCCAGA [symbol] | 50 bp, e6-7, 50% GC |
| | | | | CAGATACCTGCCATGAACTCTTA [symbol] | |
| | | | | GT | |
| | | | | (SEQ ID NO: 292) | |
| M269 | HIOMT | acetylserotonin | NM_004043 | AAGAACAGATTGACTTCCAGGA [symbol] | 50 bp, e7-8, 46% GC |
| | | methyl- | | GGGGATTTCTTCAAAGACCCTCT [symbol] | |
| | | transferase | | CCG | |
| | | | | (SEQ ID NO: 293) | |
| M270 | ABAT | 4-aminobutyrat [symbol] | NM_000663 | GGAAGTCCCAGGGCCTAGATCT [symbol] | 50 bp, e2-3, 44% GC |
| | | amino- | | AGGAGTTAATGAAACAGCTGAA [symbol] | |
| | | transferase | | ATAA | |
| | | | | (SEQ ID NO: 294) | |
| M271 | AOX1 | aldehyde | NM_001159 | GCCAGAATTTGAGGAAGGAAGT [symbol] | 50 bp, e6-7, 44% GC |
| | | oxidase 1 | | AGACAAGTCCAAAACTCTTCGC [symbol] | |
| | | | | GAAG | |
| | | | | (SEQ ID NO: 295) | |
| M272 | DBH | dopamine | NM_000787 | TACCACAACCCACTGGTGATAGA [symbol] | 48 bp, e5-6, 52% GC |
| | | beta- | | AGGACGAAACGACTCCTCAGG [symbol] | |
| | | hydroxylase | | TC | |
| | | | | (SEQ ID NO: 296) | |
| M273 | CHAT vN | choline | NM_020549 | CGTCATCGTAGCCTGCTGCAAT [symbol] | 50 bp, e6-7, 44% GC |
| | | acetyl- | | GTTCTTTGTCTTGGATGTTGTCA [symbol] | detects all variants |
| | | transferase | | A | |
| | | | | (SEQ ID NO: 297) | |
| M274 | | Variant N1 | | TGAGGCTTTGAGAAAGGAGTAG [symbol] | 50 bp, e1, 50% GC |
| | | | | AGCCTAGCATTCCGGCAGAGGA [symbol] | |
| | | | | GAAA | |
| | | | | (SEQ ID NO: 298) | |
| M275 | COMT | catechol-O- | NM_000754 | AGAGGGCTGGAGCCTGCTCAGA [symbol] | 42 bp, e1-2, 59% GC |
| | | methyl- | | GTGCTTTGAAGATGCCGGA | will detect only MB |
| | | transferase | | (SEQ ID NO: 299) | isoform |
| M276 | | | | CATGAACGTGGGCGACAAGAAA [symbol] | 46 bp, e3-4, 54% GC |
| | | | | GCAAGATCGTGGACGCCGTGAT [symbol] | will detect all |
| | | | | (SEQ ID NO: 300) | isoform |
| M277 | DDC | dopa | XM_033775 | AAAGGACTGCAGGCTTATATCC [symbol] | 49 bp, e9-10, 51% GC [symbol] |
| | | decarboxylase | | AAGCATGTCCAGCTGTCCCATGA [symbol] | |
| | | | | T | |
| | | | | (SEQ ID NO: 301) | |
| M278 | GAD2 | glutamate | NM_000818 | CTCTGCTCTCCTGGTTAGAGAA [symbol] | 50 bp, e12-13, 48% G [symbol] |
| | | decarboxylase | | GGGATTGATGCAGAATTGCAAC [symbol] | |
| | | 2 | | AAA | |
| | | | | (SEQ ID NO: 302) | |
| M279 | GAD2 | glutamate | NM_013445 | GACTTCTCTAATCTGTTTGCTAG [symbol] | 50 bp, e3-4, 46% GC |
| | | decarboxylase | | GATCTGCTTCCGGCTAAGAACG [symbol] | Detects both 25 and |
| | | | | GA | isoforms |
| | | | | (SEQ ID NO: 303) | |
| M280 | | glutamine | Y00387 | GACAGTGAGCCCAAGTGTGTGG [symbol] | 50 bp, e3-4, 52% GC; |
| | | synthetase | | AGAGTTGCCTGAGTGGAATTTC [symbol] | should detect also so [symbol] |
| | | | | TGG | gene on chr 11 |
| | | | | (SEQ ID NO: 304) | |
| M281 | | | | CATGCGGGAGGAGAATGGTCTG [symbol] | 50 bp, e7-8, 52% GC; |
| | | | | AGTACATCGAGGAGGCCATTGA [symbol] | should detect also so [symbol] |
| | | | | AAAC | genes on chr 2, 9 |
| | | | | (SEQ ID NO: 305) | |
| M282 | HNMT | histamine | NM_006895 | AAGATGCTCATTATTGTTGTGT [symbol] | 50 bp, e5-6, 48% GC |
| | | N-methyl | | GGAAGCAGTGGCTGGGACAAG [symbol] | |
| | | transferase | | GTG | |
| | | | | (SEQ ID NO: 306) | |
| M283 | MAOA | monoamine | NM_000240 | CTGGGTACAAGAACCTGAATCA [symbol] | 50 bp, e13-14, 50% G [symbol] |
| | | oxidase A | | AGGACGTTCCAGCGGTAGAAAT [symbol] | |
| | | | | ACCC | |
| | | | | (SEQ ID NO: 307) | |
| M284 | MAOB | monoamine | NM_000898 | GGGAGGCAGGACTTACACTCTT [symbol] | 50 bp, e2-3, 46% GC |
| | | oxidase B | | GGAACCAAAAGGTTAAATATGT [symbol] | |
| | | | | GACC | |
| | | | | (SEQ ID NO: 308) | |
| M285 | HRH1 | histamine | XM_052382 | CCGAGAGGACAAGTGTGAGACA [symbol] | 50 bp, 50% GC |
| | | receptor H1 | | ACTTCTATGATGTCACCTGGTTC [symbol] | |
| | | | | AGG | |
| | | | | (SEQ ID NO: 309) | |
| M286 | HRH2 | histamine | NM_022304 | AAGGGCAATCATACCACCTCTA [symbol] | 50 bp, 44% GC |
| | | receptor H2 | | GTGCAAAGTCCAGGTCAATGAA [symbol] | |
| | | | | TGTA | |
| | | | | (SEQ ID NO: 310) | |
| M287 | HRH3 | histamine | AF321910 | TCAACCTCGCCATCTCCGACTT [symbol] | 43 bp, e1-2, 60% GC |
| | | receptor H3 | | TCGTCGGCGCCTTCTGCAT | |
| | | | | (SEQ ID NO: 311) | |
| M288 | HRH4 | histamine | NM_021624 | GGCCATCTCTGACTTCTTTGTGG [symbol] | 50 bp, e1-2, 48% GC |
| | | receptor H4 | | TGTGATCTCCATTCCTTTGTACA [symbol] | |
| | | | | C | |
| | | | | (SEQ ID NO: 312) | |
| M289 | MTNR1A | melatonin | NM_005958 | GTATCGGAACAAGAAGCTCAGG [symbol] | 50 bp, e1-2, 48% GC |
| | receptor 1A | | ACGCAGGAAACATCTTTGTGGT [symbol] | | |
| | | | | GCT | |
| | | | | (SEQ ID NO: 313) | |
| M290 | MTNR1B | melatonin | NM_005959 | AGGAACCGCAAGCTCCGGAACG [symbol] | 48 bp, e1-2, 52% GC |
| | | | receptor 1B | | AGGTAATTTGTTCTTGGTGAGT [symbol] |
| | | | | G | |
| | | | | (SEQ ID NO: 314) | |
| M291 | NTS | neurotensin | NM_006183 | TTCAGCTCCTGGAGTCTGTGCT [symbol] | 50 bp, e1-2, 44% GC |
| | | | | GATTCAGAAGAGGAAATGAAA [symbol] | |
| | | | | ATT | |
| | | | | (SEQ ID NO: 315) | |
| M292 | NTSR1 | neurotensin | NM_002531 | CATCTCGGATGAGCAGTGGACT [symbol] | 50 bp, e2-3, 50% GC |
| | | receptor 1 | | GTTCCTCTATGACTTCTACCACT [symbol] | |
| | | (high | | CT | |
| | | affinity) | | (SEQ ID NO: 316) | |
| M293 | NTSR2 | neurotensin | NM_012344 | ACCGCGCTCCAAGTCTTTATCCA [symbol] | 47 bp, e1-2, 53% GC |
| | | receptor 2 | | GTGAATGTGCTGGTGTCCTTCGT [symbol] | |
| | | | | (SEQ ID NO: 317) | |
| M294 | ADRA1A | adrenergic, | NM_033303 | TTTTCTTAGTCATGCCCATTGGG [symbol] | 50 bp, e1-2, 44% GC |
| | | alpha | | CTTTCTTCCCTGATTTCAAGCCC [symbol] | |
| | | 1A-, receptor | | T | |
| | | | | (SEQ ID NO: 318) | |
| M295 | ADRA1B | adrenergic, | NM_000679 | TACCCTTCTTCATCGCTCTACCG [symbol] | 48 bp, e1-2, |
| | | alpha | | TGGCTCCTTGTTCTCCACCCTGA [symbol] | 54% GC = adral c? |
| | | 1B-, receptor | | (SEQ ID NO: 319) | |
| M296 | ADRA1D | adrenergic, | NM_000678 | TGCTGGTTCCCTTTCTTCTTTGT [symbol] | 45 bp, e1-2, 55% GC |
| | | alpha | | TGCCGCTCGGCTCCTTGTTC | |
| | | 1D-, receptor | | (SEQ ID NO: 320) | |
| M297 | ADRA2A | adrenergic, | NM_000681 | TCTCCCTACTCTCTCCCGCCGCT [symbol] | 50 bp, 5′UTR, 44% G [symbol] |
| | | alpha | | GAAATAAAACTTGGCTGTATTA [symbol] | |
| | | 2A-, receptor | | A | |
| | | | | (SEQ ID NO: 321) | |
| M298 | ADRA2B | adrenergic, | NM_000682 | TTCCTCATTCTCTTTACCATCTT [symbol] | 50 bp, 50% GC |
| | | alpha | | GCAACGCTCTGGTCATCCTGGCT [symbol] | |
| | | 2B-, receptor | | T | |
| | | | | (SEQ ID NO: 322) | |
| M299 | ADRA2C | adrenergic, | NM_000683 | CAACAGCTCGCTCAACCCGGTCA [symbol] | 50 bp, 50% GC; may |
| | | alpha | | CTACACGGTCTTCAACCAGGATT [symbol] | also detect ADRA 2A |
| | | 2C-, receptor | | (SEQ ID NO: 323) | and 2B |
| | | c | | | |
| M300 | ADRB1 | adrenergic, | NM_000684 | TAAGACCGATAGCAGGTGAACT [symbol] | 50 bp, 3′UTR, 48% G [symbol] |
| | | beta | | GAAGCCCACAATCCTCGTCTGA [symbol] | |
| | | 1-, receptor | | CAT | |
| | | | | (SEQ ID NO: 324) | |
| M301 | ADRB2 | adrenergic, | NM_000024 | GAGACCTGCTGTGACTTCTTCA [symbol] | 49 bp, 51% GC |
| | | beta | | AACCAAGCCTATGCCATTGCCT [symbol] | |
| | | 2-, receptor | | T | |
| | | | | (SEQ ID NO: 325) | |
| M302 | ADRB3 | adrenergic, | NM_000025 | CCTGAAGGACAAGAAGCAACAA [symbol] | 50 bp, 3′UTR, 46% G [symbol] |
| | | beta | | TCTGTTGATCAGAACCTGTGGAA [symbol] | |
| | | 3-, receptor | | ACC | |
| | | | | (SEQ ID NO: 326) | |
| M303 | HTR1A | serotonin | XM_003692 | AATTGGCTGGGCTACTCCAACT [symbol] | 50 bp, 48% GC |
| | | receptor | | CTGCTTAACCCCGTCATTTACG [symbol] | |
| | | 1A | | TA | |
| | | | | (SEQ ID NO: 327) | |
| M304 | HTR1B | serotonin | XM_004117 | TGGAAAAGAAGAAACTCATGG [symbol] | 49 bp, 51% GC |
| | | receptor | | GCTAGGGAGCGCAAAGCCACCA [symbol] | similarity to 1 D? |
| | | 1B | | (SEQ ID NO: 328) | |
| M305 | HTR1D | serotonin | XM_001542 | CCAAATCTTGTGTGACATCTGG [symbol] | 49 bp, 51% GC |
| | | receptor | | GTCCTCTGACATCACGTGCTGCA [symbol] | |
| | | 1D | | A | |
| | | | | (SEQ ID NO: 329) | |
| M306 | HTR1E | serotonin | XM_004134 | GGAAACATGAACATCACAAACT [symbol] | 50 bp, 46% GC |
| | | receptor | | TACCACAGAGGCCAGCATGGCT [symbol] | |
| | | 1E | | TAAG | |
| | | | | (SEQ ID NO: 330) | |
| M307 | HTR1F | serotonin | XM_003092 | ATTGTGTATATTGTGAGAGAGA [symbol] | 50 bp, 44% GC |
| | | receptor | | TGGATTATGGGGCAAGTGGTCT [symbol] | |
| | | 1F | | GA | |
| | | | | (SEQ ID NO: 331) | |
| M308 | HTR2A | serotonin | XM_007123 | CATGTTAACCATCCTGTATGGGT [symbol] | 45 bp, e1-255% GC |
| | | receptor | | CCGGTGGCCTCTGCCGAGCAA [symbol] | |
| | | 2A | | (SEQ ID NO: 332) | |
| M309 | HTR2C | serotonin | XM_013121 | CTGTCTCTCCTGGCAATCCTTTA [symbol] | 50 bp, e2-3, 44% GC |
| | | receptor | | GATTATGTCTGGCCACTACCTA [symbol] | |
| | | 2C | | TA | |
| | | | | (SEQ ID NO: 333) | |
| M310 | HTR3A | serotonin | XM_006444 | TCCCGGACATTCTCATCAATGA [symbol] | 50 bp, e4-5, 48% GC |
| | | receptor | | TCGTGGATGTGGGGAAGTCTCCA [symbol] | |
| | | 3A | | AT | |
| | | | | (SEQ ID NO: 334) | |
| M311 | HTR3B | serotonin | XM_006445 | ACCTTCAAGAGCATTCTGCATA [symbol] | 50 bp, e5-6, 48% GC |
| | | receptor | | GTGGAAGACGTAGACCTGGCCT [symbol] | |
| | | 3B | | CT | |
| | | | | (SEQ ID NO: 335) | |
| M312 | HTR4 | serotonin | NM_000870 | TTCCTGTAATGGACAAACTTGAT [symbol] | 50 bp, e5-6, 42% GC |
| | | receptor | | CTAATGTGAGTTCTGAGGAGGG [symbol] | |
| | | 4 | | TC | |
| | | | | (SEQ ID NO: 336) | |
| M313 | HTR5A | serotonin | XM_017055 | ACCCATATCCGAAGCTGTGGAG [symbol] | 44 bp, e5-6, 53% GC |
| | | receptor | | GAAGGACTCTGCCAAACAG | |
| | | 5A | | (SEQ ID NO: 337) | |
| M314 | HTR6 | serotonin | XM_001435 | TTTGTGGCCAACATAGTCCAGG [symbol] | 40 bp, e2-3, 55% GC |
| | | receptor | | GTGTGCGACTGCATCT | |
| | | 6 | | (SEQ ID NO: 338) | |
| M315 | EGF | epidermal | NM_001963 | AGAGAGATGGGAAAACATGTA [symbol] | 50 bp, e7-8, 48% GC |
| | | growth | | GGTTGTTCCTCACCCGATAATG [symbol] | |
| | | factor | | GGA | |
| | | | | (SEQ ID NO: 339) | |
| M316 | EGFR | epidermal | NM_005228 | AAGTGCGAAGGGCCTTGCCGCA [symbol] | 50 bp, e7-8, 48% GC |
| | | growth | | AGTGTGTAACGGAATAGGTATT [symbol] | |
| | | factor | | GTGA | |
| | | receptor | | (SEQ ID NO: 340) | |
| M317 | | | | ATACGCGGCAGGACCAAGCAA [symbol] | 49 bp, e10-11, 51% G [symbol] |
| | | | | TGGTCAGTTTTCTCTTGCAGTC [symbol] | |
| | | | | CA | |
| | | | | (SEQ ID NO: 341) | |
| M318 | ERBB2 | v-erb-b2 | NM_004448 | TGAACTGGTGTATGCAGATTGC [symbol] | 49 bp, e20-21, 51% G [symbol] |
| | | erytbroblastic | | AGGGGATGAGCTACCTGGAGGA [symbol] | |
| | | leukemia viral | | GT | |
| | | oncogene | | (SEQ ID NO: 342) | |
| | | homolog 2 | | | |
| M319 | FGF1 | fibroblast | NM_000800 | CACCGACGGGCTTTTATACGGCT [symbol] | 50 bp, e3-4, 48% GC |
| | | growth | | ACAGACACCAAATGAGGAATGT [symbol] | |
| | | factor 1 | | TGT | |
| | | | | (SEQ ID NO: 343) | |
| M320 | FGF2 | fibroblast | NM_002006 | GTCCGGGAGAAGAGCGACCCT [symbol] | 50 bp, e1-2, 52% GC |
| | | growth | | CATCAAGCTACAACTTCAAGCA [symbol] | |
| | | factor 2 | | AAGA | |
| | | | | (SEQ ID NO: 344) | |
| M321 | FGF3 | fibroblast | NM_005247 | TGGCCATGAACAAGAGGGGAC [symbol] | 46 bp, e1-2, 54% GC |
| | | growth | | CTCTATGCTTCGGAGCACTACA [symbol] | |
| | | factor 3 | | (SEQ ID NO: 345) | |
| M322 | FGF4 | fibroblast | NM_002007 | GGCAAGCTCTATGGCTCGCCCTT [symbol] | 45 bp, e2-3, 57% GC |
| | | growth | | TTCACCGATGAGTGCACGTTC | |
| | | factor 4 | | (SEQ ID NO: 346) | |
| M323 | FGFS | fibroblast | NM_004464 | GATCCCACGAAGCCAATATGTTA [symbol] | 50 bp, e1-2, 38% GC |
| | | growth | | GTGTTTTGGAAATATTTGCTGTG [symbol] | |
| | | factor 5 | | CT | |
| | | | | (SEQ ID NO: 347) | |
| M324 | FGF6 | fibroblast | NM_020996 | CAGTAAAGGAAGATTGTACGCA [symbol] | 50 bp, e2-3, 46% GC |
| | | growth | | CGCCCAGCTTCCAAGAAGAATG [symbol] | |
| | | factor 6 | | AAGT | |
| | | | | (SEQ ID NO: 348) | |
| M325 | FGF7 | fibroblast | NM_002009 | ACCCAAGAGATGAAGAATAATT [symbol] | 50 bp, e1-2, 38% GC |
| | | growth | | CAATATCATGGAAATCAGGACA [symbol] | |
| | | factor 7 | | TGGC | |
| | | | | (SEQ ID NO: 349) | |
| M326 | FGF8 | fibroblast | NM_033163 | AGAAGGGGAAGCTGATCGCCAA [symbol] | 45 bp, e1-2, 55% GC |
| | | growth | | AGCAACGGCAAAGGCAAGGACT [symbol] | will detect longer and |
| | | factor 8 | | (SEQ ID NO: 350) | shorter isoforms |
| M327 | FGF10 | fibroblast | NM_004465 | AGAAGGGGAAACTCTATGGCTC [symbol] | 50 bp, e2-3, 38% GC |
| | | growth | | AAAGAATTTAACAATGACTGTA [symbol] | |
| | | factor 10 | | GCTG | |
| | | | | (SEQ ID NO: 351) | |
| M328 | FGF11 | fibroblast | NM_004112 | ATGCTGAGGGACTGCTCTACAG [symbol] | 48 bp, e,3-4, 54% GC |
| | | growth | | CGCCGCATTTCACAGCTGAGTGT [symbol] | homol to fgf-3? |
| | | factor 11 | | (SEQ ID NO: 352) | |
| M329 | FGF12 | fibroblast | NM_021032 | ACCAAGGACGAAAACAGCGACT [symbol] | 50 bp, e2-3, 48% GC |
| | | growth | | CACTCTCTTCAATCTAATTCCCG [symbol] | |
| | | factor 12 | | GGG | |
| | | | | (SEQ ID NO: 353) | |
| M330 | FGF13 | fibroblast | NM_004114 | AAGAAGAGGCGCAGAAGAAGA [symbol] | 50 bp, e1-2, 48% GC |
| | | growth | | AGAGCCTCAGCTTAAGGGTATA [symbol] | |
| | | factor 13 | | TAC | |
| | | | | (SEQ ID NO: 354) | |
| M331 | FGF14 | fibroblast | NM_004115 | ACCAAGGATGACAGCACTAATT [symbol] | 50 bp, e2-3, 48% GC |
| | | growth | | ACACTCTTCAACCTCATACCAGT [symbol] | |
| | | factor 14 | | GG | |
| | | | | (SEQ ID NO: 355) | |
| M332 | FGF16 | fibroblast | NM_003868 | ACCTAGGAATGAATGAGCGAG [symbol] | 50 bp, 48% GC |
| | | growth | | GAACTCTATGGGTCGAAGAAAC [symbol] | |
| | | factor 16 | | CACA | |
| | | | | (SEQ ID NO: 356) | |
| M333 | FGF17 | fibroblast | NM_003867 | TTCTCTGCTGTCAAACTCAGGG [symbol] | 50 bp, e2-3, 48% GC |
| | | growth | | AGAATCACCCGTCTCCTAATTTT [symbol] | |
| | | factor 17 | | AC | |
| | | | | (SEQ ID NO: 357) | |
| M334 | FGF18 | fibroblast | NM_033649 | CCTCCGCCTGCACTTGCCTGTGT [symbol] | 46 bp, e1-2, 56% GC |
| | | growth | | TACACTTCCTGCTGCTGTGCTT | |
| | | factor 18 | | (SEQ ID NO: 358) | |
| M335 | FGF19 | fibroblast | NM_005117 | GACGGCAAGATGCAGGGGCTG [symbol] | 45 bp, e2-3, 57% GC |
| | | growth | | TCAGTACTCGGAGGAAGACTGT [symbol] | |
| | | factor 19 | | (SEQ ID NO: 359) | |
| M336 | FGF20 | fibroblast | NM_019851 | GAATGACAAAGGAGAAGTCTAT [symbol] | 50 bp, e2-3, 40% GC |
| | | growth | | GATCAGAGAAACTTACTTCCGAA [symbol] | |
| | | factor 20 | | GCA | |
| | | | | (SEQ ID NO: 360) | |
| M337 | FGF21 | fibroblast | NM_019113 | TGCTGACCAGAGCCCCGAAAGT [symbol] | 45 bp, e1-2, 55% GC |
| | | growth | | CCTGCAGCTGAAAGGCTTGAA | |
| | | factor 21 | | (SEQ ID NO: 361) | |
| M338 | FGF22 | fibroblast | NM_020637 | CCACGGCCAGGACAGCATCCTG [symbol] | 40 bp, e1-2, 62% GC |
| | | growth | | AGATCCGCTCTGTACAC | |
| | | factor 22 | | (SEQ ID NO: 362) | |
| M339 | FGF23 | fibroblast | NM_020638 | TTTCAGAGGCAACATTTTTGGAT [symbol] | 50 bp, e2-3, 44% GC |
| | | growth | | ACACTATTTCGACCCGGAGAAC [symbol] | |
| | | factor 23 | | CA | |
| | | | | (SEQ ID NO: 363) | |
| M340 | FGFR1 | fibroblast | NM_000604 | CGACTACTATAAAAAGACAACC [symbol] | 50 bp, e13-14, 50% G [symbol] |
| | | growth | | ACGGCCGACTGCCTGTGAAGTG [symbol] | |
| | | factor | | ATGG | |
| | | receptor [symbol] | | (SEQ ID NO: 364) | |
| M341 | FGFR2 | fibroblast | NM_000141 | TTCTTGGAGCCTGCACACAGGAT [symbol] | 50 bp, e13-14, 48% G [symbol] |
| | | growth | | GGCCTCTCTATGTCATAGTTGA [symbol] | detects almost all |
| | | factor | | AT | variants (tyrosine |
| | | receptor [symbol] | | (SEQ ID NO: 365) | kinase domains) |
| M342 | FGFR3 | fibroblast | XM_044120 | ACCGTAGCCGTGAAGATGCTGA [symbol] | 49 bp, e10-11, 53% G [symbol] |
| | | growth | | AGACGATGCCACTGACAAGGA [symbol] | |
| | | factor | | TGT | |
| | | receptor [symbol] | | (SEQ ID NO: 366) | |
| M343 | FGFR4 | fibroblast | NM_002011 | ACTGTGGCCGTCAAGATGCTCAA [symbol] | 48 bp, e11-12, 54% G [symbol] |
| | | growth | | GACAACGCCTCTGACAAGGACC [symbol] | |
| | | factor | | G | |
| | | receptor [symbol] | | (SEQ ID NO: 367) | |
| M344 | FIBP | fibroblast | NM_004214 | CACTACTCATCGAGAGGTACTAT [symbol] | 50 bp, 50% GC |
| | | growth | | CCTTTGATGAGGCCTITTGTTCGG [symbol] | |
| | | factor (acidic) | | AG | |
| | | intracellular | | (SEQ ID NO: 368) | |
| | | binding protein | | | |
| M345 | SPRY1 | sprouty homolog | XM_036349 | CTGTTCACAATCACACTGCTGCT [symbol] | 50 bp, 48% GC |
| | | 1, antagonist | | TAGATACCTGTGTATGGGAGCCA [symbol] | |
| | | of FGF | | GT | |
| | | signaling | | (SEQ ID NO: 369) | |
| M346 | SPRY2 | sprouty homolog | NM_005842 | CCCAGAACGTGATTGACTATGG [symbol] | 50 bp, 48% GC |
| | | 2, antagonist | | ACTTGTGTATGCTGTGTGAAAG [symbol] | |
| | | of FGF | | CTC | |
| | | signaling | | (SEQ ID NO: 370) | |
| M347 | IGF1R | insulin-like | NM_000875 | TACCGGCACAATTACTGCTCCAA [symbol] | 50 bp, e9-10, 46% GC [symbol] |
| | | growth factor | | GACAAAATCCCCATCAGGAAGT [symbol] | |
| | | 1 receptor | | TGC | |
| | | | | (SEQ ID NO: 371) | |
| M348 | IGF2 | insulin-like | NM_000612 | TCGACCCCTCCGACCGTGCTTC [symbol] | 41 bp, e3-4, 61% GC |
| | | growth factor | | GACAACTTCCCCAGATA | |
| | | 2 | | (SEQ ID NO: 372) | |
| M349 | IGF1 | insulin-like | X00173 | GACATGCCCAAGACCCAGAAGG [symbol] | 50 bp, e3-4, 48% GC |
| | | growth factor | | AGTACATTTGAAGAACGCAAGT [symbol] | |
| | | 1 | | GAGG | |
| | | | | (SEQ ID NO: 373) | |
| M350 | IGFBP1 | insulin-like | NM_000596 | GAATGGATTTTATCACAGCAGA [symbol] | 50 bp, e2-3, 47% GC |
| | | growth factor | | GTGTGAGACATCCATGGATGGA [symbol] | |
| | | binding protein | | AGGC | |
| | | | | (SEQ ID NO: 374) | |
| M351 | IGFBP2 | insulin-like | NM_000597 | AAGCATGGCCTGTACAACCTCA [symbol] | 50 bp, e3-4, 50% GC |
| | | growth factor | | ACAGTGCAAGATGTCTCTGAAC [symbol] | |
| | | binding protein | | GGCA | |
| | | | | (SEQ ID NO: 375) | |
| M352 | IGFBP3 | insulin-like | NM_000598 | AAGCGGGAGACAGAATATGG | 50 bp, e2-3, 50% GC |
| | | growth factor | | TCCCTGCCGTAGAGAAATGGAA [symbol] | |
| | | binding protein | | ACACACT | |
| | | | | (SEQ ID NO: 376) | |
| M353 | IGFBP4 | insulin-like | NM_001552 | AACGGCAACTTCCACCCCAAGC [symbol] | 42 bp, e2-3, 57% GC |
| | | growth factor | | GTGTCACCCAGCTCTGGAT | |
| | | binding protein | | (SEQ ID NO: 377) | |
| M354 | IGFBP5 | insulin-like | U20271 | CAAAGGATTCTACAAGAGAAAG [symbol] | 49 bp, e3-4, 51% GC |
| | | growth factor | | AGTGCAAACCTTCCCGTGGCG [symbol] | |
| | | binding protein | | AG | |
| | | | | (SEQ ID NO: 378) | |
| M355 | IGFBP6 | insulin-like | NM_002178 | GCGCGCCTGCTGTTGCAGAGGA [symbol] | 42 bp, e1-2, 54% GC |
| | | growth factor | | AATCCTAAGGAGAGTAAAC | |
| | | binding protein | | (SEQ ID NO: 379) | |
| M356 | IMP-1 | IGF-II mRNA- | AF117106 | ATGGTACAGTAGAGAACTGTGA [symbol] | 50 bp, e2-3, 50% GC |
| | | binding protein | | CAAGTGAACACCGAGAGTGAGA [symbol] | |
| | | | | GGCA | |
| | | | | (SEQ ID NO: 380) | |
| M357 | IMP-2 | IGF-II mRNA- | NM_006548 | GGACAGTGGAGAATGTGGAACA [symbol] | 50 bp, e4-5, 50% GC |
| | | binding protein | | GTCAACACAGACACAGAAACCG [symbol] | |
| | | | | CGTT | |
| | | | | (SEQ ID NO: 381) | |
| M358 | IMP-3 | IGF-II mRNA- | AF117108 | TATCCCGCCTCATTTACAGTGG [symbol] | 50 bp, e2-3, 48% GC |
| | | binding protein | | GGTGCTGGATAGTTTACTAGTC [symbol] | |
| | | | | GT | |
| | | | | (SEQ ID NO: 382) | |
| M359 | NGFB | nerve growth | NM_002506 | GTTCTACACTCTGATCACAGCTT [symbol] | 50 bp, 50% GC |
| | | factor, beta | | TCTGATCGGCATACAGGCGGAA [symbol] | |
| | | polypeptide | | CAC | |
| | | | | (SEQ ID NO: 383) | |
| M360 | NGF-2 | | X53655 | CAAGCTCTCCAAGCAGATGGTG [symbol] | 50 bp, 50% GC |
| | | | | ACGTTAAGGAAAATTACCAGAG [symbol] | |
| | | | | ACCC | |
| | | | | (SEQ ID NO: 384) | |
| M361 | NGFR | nerve growth | NM_002507 | TTGTGGCCTACATAGCCTTCAA [symbol] | 50 bp, e4-5, 50% GC |
| | | factor | | GGTGGAACAGCTGCAAGCAGAA [symbol] | |
| | | receptor | | AAG | |
| | | | | (SEQ ID NO: 385) | |
| M362 | TDGF1 | teratocarcinoma | NM_003212 | TCTTTGAACTGGGATTAGTTGC [symbol] | 44 bp, e2-3, 50% GC |
| | | derived growth | | GGCTGGGCCATCAGGAATTT | |
| | | factor 1 | | (SEQ ID NO: 386) | |
| M363 | PDGFA | platelet- | NM_002607 | AGATAGACTCCGTAGGGAGTGA [symbol] | 48 bp, e3, 52% GC |
| | | derived growth | | GATTCTTTGGACACCAGCCTGA [symbol] | |
| | | factor alpha | | G | |
| | | polypeptide | | (SEQ ID NO: 387) | |
| M364 | PDGFB | platelet- | NM_002608 | GACCTGTCCAGGTGAGAAAGAT [symbol] | 47 bp, e4-5, 51% GC |
| | | derived growth | | GAGATTGTGCGGAAGAAGCCAA [symbol] | |
| | | b polypeptide | | C | |
| | | | | (SEQ ID NO: 388) | |
| M365 | PDGFC | platelet- | NM_016205 | TTTCCAGCAACAAGGAACAGAA [symbol] | 50 bp, e1-2, 46% GC |
| | | derived growth | | GGAGTACAAGATCCTCAGCATG [symbol] | |
| | | factor C | | GAGA | |
| | | | | (SEQ ID NO: 389) | |
| M366 | PDGFD | platelet- | AF336376 | TCATACCATGACCGGAAGTCAA [symbol] | 50 bp, e5-6, 44% GC |
| | | derived growth | | AGTTGACCTGGATAGGCTCAAT [symbol] | |
| | | factor D | | TGA | |
| | | | | (SEQ ID NO: 390) | |
| M367 | PDGFRA | platelet- | NM_006206 | TCAATGGACTTACCCTGGAGAA [symbol] | 50 bp, e5-6, 48% GC |
| | | derived growth | | GAAAGGCAAAGGCATCACAAT [symbol] | |
| | | factor receptor, | | TGG | |
| | | alpha poly- | | (SEQ ID NO: 391) | |
| | | peptide | | | |
| M368 | PDGFRB | platelet- | NM_002609 | TGCCTACTATGTCTACAGACTC [symbol] | 50 bp, e4-5, 48% GC |
| | | derived growth | | GGTGTCATCCATCAACGTCTCT [symbol] | |
| | | factor receptor, | | GA | |
| | | beta polypeptide | | (SEQ ID NO: 392) | |
| M369 | VEGF | vascular | NM_003376 | TGAGGAGTCCAACATCACCATG [symbol] | 50 bp, e3-4, 48% GC |
| | | endothelial | | AGATTATGCGGATCAAACCTCA [symbol] | |
| | | growth factor | | AAG | |
| | | | | (SEQ ID NO: 393) | |
| M370 | VEGFB | vascular | NM_003377 | ACCAGAGGAAAGTGGTGTCATG [symbol] | 50 bp, 50% GC |
| | | endothelial | | ATAGATGTGTATACTCGCGCTA [symbol] | |
| | | growth factor | | TGC | |
| | | B | | (SEQ ID NO: 394) | |
| M371 | VEGFC | vascular | NM_005429 | CAGCACGAGCTACCTCAGCAAG [symbol] | 50 bp, e1-2, 46% GC |
| | | endothelial | | CGTTATTTGAAATTACAGTGCCT [symbol] | |
| | | growth factor | | TCT | |
| | | C | | (SEQ ID NO: 395) | |
| M372 | VEGFD | vascular | NM_004469 | CAGTAATGAACATGGACCAGTG [symbol] | 50 bp, e1-2, 46% GC |
| | | endothelial | | AGCGATCATCTCAGTCCACATT [symbol] | |
| | | growth factor | | AAC | |
| | | D | | (SEQ ID NO: 396) | |
| M373 | | similar to | XM_067723 | TTTGCAGAACTTGTGGAAAAAC [symbol] | 50 bp, e4-5, 34% GC |
| | | Vascular | | GAAAATAGTGGGTTTACATACT [symbol] | |
| | | endothelial | | AC | |
| | | growth factor | | (SEQ ID NO: 397) | |
| | | receptor | | | |
| M374 | ADAR | adenosine | NM_001111 | AAAGAAGGCCCTGCCCATGAA [symbol] | 48 bp, e4-5, 50% GC |
| | | deaminase, RN | | CAAGTTCCAATACTGTGTTGCA [symbol] | should detect all 3 |
| | | specific | | G | variants |
| | | | | (SEQ ID NO: 398) | |
| M375 | SPS | selenium donor | NM_012247 | ATCGTAGACGACCCTTACATGAT [symbol] | 48 bp, e2-3, 52% GC |
| | | protein | | GGCAGGATAGCGTGTGCCAATG [symbol] | may also detect |
| | | | | C | LOC158861 |
| | | | | (SEQ ID NO: 399) | |
| M376 | SPS2 | selenium donor | NM_012248 | ATGTTACTCAGCGTCAGCCAGA [symbol] | 50 bp, 50% GC |
| | | protein 2 | | ATGAGTGAGGAGGAACGCGAAA [symbol] | |
| | | | | GGT | |
| | | | | (SEQ ID NO: 400) | |
| M377 | LOC1235 | similar to | XM_063731 | GAACTGAAGGGCACAGGCTGCA [symbol] | 50 bp, e1-2, 50% GC |
| | | Selenide, water | | AGAGAACCACTTCCAAGAAGAT [symbol] | |
| | | dikinase 1 | | AGCA | |
| | | | | (SEQ ID NO: 401) | |
| M378 | LOC1295 | similar to | XM_065297 | TTAAAGATGCAGCCGAGGAAG [symbol] | 50 bp, e1-2, 42% GC |
| | | selenophosphat [symbol] | | GGAATATGTAGAGTTGACATAC [symbol] | |
| | | synthetase | | AAGA | |
| | | | | (SEQ ID NO: 402) | |
| M379 | LOC1505 | -//- | XM_086946 | AAAATATGAATCCCACACCTGG [symbol] | 50 bp, e1-2, 42% GC |
| | | | | CCACCTCTTAATCTAGACAGAAA [symbol] | |
| | | | | AGC | |
| | | | | (SEQ ID NO: 403) | |
| M380 | LOC1588 | -//- | XM_005845 | CAGAATGTGACAATATGCTGAT [symbol] | 50 bp, 40% GC |
| | | | | TCCTTGGAGTCAGTAATAAAAT [symbol] | |
| | | | | CC | |
| | | | | (SEQ ID NO: 404) | |
| M381 | LOC1670 | -//- | XM_094245 | TTACAGTGCTTTTGCTGAAAAT [symbol] | 50 bp, e2-3, 38% GC |
| | | | | GAATGCAGGAACTGAGACCAT [symbol] | |
| | | | | TTA | |
| | | | | (SEQ ID NO: 405) | |
| M382 | LOC1684 | -//- | XM_095122 | CAGAATGTGACAATATGCTGAT [symbol] | 50 bp, 40% GC |
| | | | | TCCTTGGAGTCAGTAATAAAAT [symbol] | |
| | | | | CC | |
| | | | | (SEQ ID NO: 406) | |
| M383 | LOC2020 | -//- | XM_116312 | ATATCACAGGCTTTGGCATTCTA [symbol] | 50 bp, 46% GC |
| | | | | GACACTCTCAGAACCTCGTGAA [symbol] | |
| | | | | AA | |
| | | | | (SEQ ID NO: 407) | |
| M384 | SCYA21 | small | NM_002989 | AAAGGAAAGGGCTCCAAAGGCT [symbol] | 44 bp, e3-4, 54% GC |
| | | inducible | | CAAGAGGACTGAGCGGTCACA | |
| | | cytokine | | (SEQ DI NO: 408) | |
| | | subfamily A | | | |
| M385 | ACVR1 | activin A | NM_001105 | CCAATGTTGGAGACAGCACTTTA [symbol] | 50 bp, e4-5, 40% GC |
| | | receptor type I | | CAGATTTATTGGATCATTCGTGT [symbol] | |
| | | | | CA | |
| | | | | (SEQ ID NO: 409) | |
| M386 | ACVR1B | activin A | NM_004302 | AGATTGCTCGAAGATGCAATTCT [symbol] | 50 bp, e7-8, 44% GC |
| | | receptor type IB | | GAGGAGTCCATGAAGAATATCA [symbol] | should detect only |
| | | | | CTG | isoform a |
| | | | | (SEQ ID NO: 410) | |
| M387 | ACVR2 | activin A | NM_001616 | GCTGATCACAGCATTTCATGAAA [symbol] | 50 bp, e4-5, 40% GC |
| | | receptor type 2 | | GGGTTCACTATCAGACTTTCTTA [symbol] | |
| | | | | GG | |
| | | | | (SEQ ID NO: 411) | |
| M388 | ACVR2B | activin A | NM_001106 | TGCTGGCTAGATGACTTCAACT [symbol] | 45 bp, e2-3, 51% GC |
| | | receptor type 2B | | TACGATAGGCAGGAGTGTGTG | |
| | | | | (SEQ ID NO: 412) | |
| M389 | INHBA | inhibin, beta | XM_042996 | ATCATCACGTTTGCCGAGTCAG [symbol] | 47 bp, e1-2, 53% GC; |
| | | A | | ACAGCCAGGAAGACGCTGCACT [symbol] | same as erythroid |
| | | | | (SEQ ID NO: 413) | differentiation |
| | | | | | protein |
| M390 | INHBB | inhibin, beta | NM_002193 | AATCATCAGCTTCGCCGAGACA [symbol] | 40 bp, e1-2, 60% GC |
| | | B | | ATGGCCTCGCCTCCTCC | |
| | | | | (SEQ ID NO: 414) | |
| M391 | INHBC | inhibin, beta | NM_005538 | GAAATCATCAGCTTTGCTGAGA [symbol] | 49 bp, e1-2, 51% GC |
| | | C | | GGCCTCTCCACCATCAACCAGA [symbol] | |
| | | | | C | |
| | | | | (SEQ ID NO: 415) | |
| M392 | BLC | B lymphocyte | AF044197 | ATGGTTGTCCAAGAAAAGAAAT [symbol] | 50 bp, e3-4, 34% GC |
| | | chemo- | | ATAGTCTGGAAGAAGAACAAGT [symbol] | |
| | | attractant | | AATT | |
| | | | | (SEQ ID NO: 416) | |
| M393 | TNFSF13 | tumor necrosis | NM_006573 | GCCGTTCAGGGTCCAGAAGAAA [symbol] | 50 bp, e2-3, 50% GC |
| | | factor (ligand) | | AGTCACTCAAGACTGCTTGCAA [symbol] | AF132600, AF11645 [symbol] |
| | | superfamily, | | GAT | AF186114, AF13471 [symbol] |
| | | member 13b | | (SEQ ID NO: 417) | |
| M394 | TNFRSF5 | tumor necrosis | NM_001250 | AAACAGTCAGTGCTGTTCTTTGT [symbol] | 50 bp, e2-3, 50% GC |
| | (CD40L) | factor receptor | | CCAGCCAGGACAGAAACTGGTG [symbol] | |
| | | superfamily, | | GTG | |
| | | member 5 | | (SEQ ID NO: 418) | |
| M395 | CNTF | ciliary | NM_000614 | CTGACTGCTCTTACGGAATCCTA [symbol] | 50 bp, e1-2, 48% GC |
| | | neurotrophic | | GTGAAGCATCAGGGCCTGAACA [symbol] | |
| | | factor | | GAA | |
| | | | | (SEQ ID NO: 419) | |
| M396 | CNTFR | ciliary | NM_001842 | ATCACCTTTGACGAGTTCACCAT [symbol] | 50 bp, e5-6, 44% GC |
| | | neurotrophic | | GTGAAGCCTGATCCTCCAGAAA [symbol] | |
| | | factor receptor | GT | | |
| | | | | (SEQ ID NO: 420) | |
| M397 | CSF1 | colony | NM_000757 | ACATTTGAGTTTGTAGACCAGGA [symbol] | 50 bp, 44% GC = |
| | | stimulating | | CAGTTGAAAGATCCAGTGTGCTA [symbol] | CSF4? |
| | | factor I | | CT | |
| | | | | (SEQ ID NO: 421) | |
| M398 | CSF1R | colony | NM_005211 | CTTCCTCCAACACAACAACACTA [symbol] | 50 bp, e4-5, 50% GC |
| | | stimulating | | GCTCGCAATCCCTCAACAATCT [symbol] | |
| | | factor 1 | | CT | |
| | | receptor | | (SEQ ID NO: 422) | |
| M399 | CSF2 | colony | NM_000758 | TGAACCTGAGTAGAGACACTGC [symbol] | 50 bp, e1-2, 44% GC |
| | | stimulating | | GCTGAGATGAATGAAACAGTAG [symbol] | |
| | | factor 2 | | AGTC | |
| | | | | (SEQ ID NO: 423) | |
| M400 | CSF2RA | colony | NM_006140 | CTAGTCTCAATGTGAGGTTTGA [symbol] | 50 bp, 46% GC |
| | | stimulating | | CCAGGACGATGAATTTAAGCTG [symbol] | |
| | | factor 2 | | GAC | |
| | | receptor, | | (SEQ ID NO: 424) | |
| | | alpha low- | | | |
| | | affinity | | | |
| M401 | CSF2RB | colony | NM_000395 | GGCAGAGAAACACATAAAGAG [symbol] | 49 bp, e7-8, 51% GC |
| | | stimulating | | CAGTGAACATCCAGATGGCCCC [symbol] | |
| | | factor 2 | | CA | |
| | | receptor, | | (SEQ ID NO: 425) | |
| | | beta low- | | | |
| | | affinity | | | |
| M402 | CSF3 | colony | NM_000759 | AGGAGAAGCTGGTGAGTGAGT [symbol] | 43 bp, e2-3, 55% GC |
| | | stimulating | | GCCACCTACAAGCTGTGCCA | |
| | | factor 3 | | (SEQ ID NO: 426) | |
| M403 | CTF1 | cardiotrophin 1 | XM_096076 | CATGTGTACATCTCAGCCTTAT [symbol] | 50 bp, 48% GC |
| | | | | CAAGGAGGTGACACCTTCTCTC [symbol] | |
| | | | | TG | |
| | | | | (SEQ ID NO: 427) | |
| M404 | CLC | cardiotrophin- | AY049779 | AAGCCTCAATGACAAACTGCGG [symbol] | 42 bp, 54% GC |
| | | li [symbol] cytokine | | TGACCCAGAACTACGAGGC | |
| | | | | (SEQ ID NO: 428) | |
| M405 | EMAPII | small inducible | NM_004757 | CAGGCAGAAATTCAAAATGGAG [symbol] | 50 bp, e2-3, 40% GC |
| | (SCYE1) | cytokine | | GAAGCAAATAGCATTTCCATCT [symbol] | |
| | | subfamily E, | | TAC | |
| | | member 1 | | (SEQ ID NO: 429) | |
| M406 | Epo | erytropoetin | X02157 | CCGAGAATATCACGACGGGCTG [symbol] | 49 bp, e2-3, 51% GC |
| | | | | GCTGAACACTGCAGCTTGAATGA [symbol] | |
| | | | | GAA | |
| | | | | (SEQ ID NO: 430) | |
| M407 | CCR3 | CC chemokine | AF247361 | CATGGCATGTGTAAGCTCCTCT [symbol] | 50 bp, 48% GC |
| | | receptor 3 | | GGGTTTTATCACACAGGCTTGTA [symbol] | |
| | | | | AG | |
| | | | | (SEQ ID NO: 431) | |
| M408 | eotaxin | eosinophil- | D49372 | TGGCAAATGTCCCCAGAAAGCT [symbol] | 50 bp, e2-3, 50% GC |
| | | selective CC | | GATCTTCAAGACCAAACTGGCC [symbol] | |
| | | chemokine | | AGG | |
| | | | | (SEQ ID NO: 432) | |
| M409 | osteopont [symbol] | osteopontin | AF052124 | CTAGGCATCACCTGTGCCATAC [symbol] | 50 bp, e2-3, 48% GC |
| | | | | GTTAAACAGGCTGATTCTGGAA [symbol] | |
| | | | | TC | |
| | | | | (SEQ ID NO: 433) | |
| M410 | GSN | gelsolin | NM_000177 | AAGATGGGAAAATCTTTGTCTG [symbol] | 50 bp, e7-8, 48% GC |
| | | | | AAGGCAAGCAGGCAAACACGGA [symbol] | |
| | | | | GAG | |
| | | | | (SEQ ID NO: 434) | |
| M411 | GZMB | granzyme B | NM_004131 | AGAGATTAAAAAGACTTCCTTTA [symbol] | 50 bp, e3-4, 46% GC |
| | | | | GGGGGACTCTGGAGGCCCTCTT [symbol] | |
| | | | | GT | |
| | | | | (SEQ ID NO: 435) | |
| M412 | CD44 | CD44 hyaluronic | X55150 | GACAGGACCTCTTTCAATGACAA [symbol] | 50 bp, e5-6, 46% GC |
| | | receptor | | GCAGCAGAGTAATTCTCAGAGC [symbol] | |
| | | (epithelial | | CT | |
| | | form) | | (SEQ ID NO: 436) | |
| M413 | | | | AGCACAGACAGAATCCCTCGTA [symbol] | 50 bp, e4-5, 46% GC |
| | | | | CAATATGGACTCCAGTCATAGTA [symbol] | |
| | | | | AAC | |
| | | | | (SEQ ID NO: 437) | |
| M414 | IFN-alpha | interferon | J00210 | ATTCTGCACCGAACTCTACCAG [symbol] | 50 bp, 50% GC will a [symbol] |
| | | alpha | | GCTGAATGACTTGGAAGCCTGT [symbol] | detect IFN-alpha 13, [symbol] |
| | | | | GA | may detect 2, F, 6, 5, [symbol] |
| | | | | (SEQ ID NO: 438) | |
| M415 | IFN-alpha | interferon | X02959 | TGCCTGAAGGACAGACATGACT [symbol] | 50 bp, 46% GC; may |
| | 14 | alpha | | GAATTTCCCCAGGAGGAATTTGA [symbol] | also detect IFN-alpha |
| | | | | GG | 1B, 21, 17, 5, F, 1B, |
| | | | | (SEQ ID NO: 439) | D, 2, 21 |
| M416 | IFN-alpha | interferon | X02957 | ACAAAGGATTCATCTGCTGCTT [symbol] | 50 bp, 46% GC; may |
| | 16 | alpha | | GATGAGACCCTCCTAGACAAAT [symbol] | also detect IFN-alpha |
| | | | | TA | 14, F, 5, 6, 7, 8, |
| | | | | (SEQ ID NO: 440) | |
| M417 | IFN-alpha | interferon | X02955 | AATACAGCCCTTGTGCCTGGGA [symbol] | 50 bp, 46% GC; will |
| | 4b | alpha | | TTGTCAGAGCAGAAATCATGAG [symbol] | also detect IFN-alpha |
| | | | | TCC | 21, 7, 13, 14, 1, 16, 1 [symbol] |
| | | | | (SEQ ID NO: 441) | d, N, F, H, N, M1, A |
| M418 | IFN-alpha | interferon | X02956 | GTAACAGGAGGACTTTGATGAT [symbol] | 50 bp, 40% GC; may |
| | | alpha | | ATGGCACAAATGGGAAGAATCT [symbol] | also detect IFN-alpha |
| | | | | TCCT | 14, H |
| | | | | (SEQ ID NO: 442) | |
| M419 | IFN-alpha | interferon | X02958 | ATGACTTCAGATTTCCCCAGGA [symbol] | 50 bp, 48% GC; may |
| | | alpha | | AGTTTGATGGCAACCAGTTCCA [symbol] | also detect IFN-alpha |
| | | | | AG | 16, F |
| | | | | (SEQ ID NO: 443) | |
| M420 | IFN-alpha | interferon | X02960 | AGCCTGCGTAATAGGAGGGCCT [symbol] | 50 bp, 50% GC; may |
| | | alpha | | GATACTCCTGGCACAAATGGGA [symbol] | also detect IFN-alpha |
| | | | | GAAT | 21, 17, 4, A, N, F, C, |
| | | | | (SEQ ID NO: 444) | 10, 8 |
| M421 | IFN-alpha | interferon | X03125 | GAGATGATCCAGCAGACCTTCA [symbol] | 50 bp, 50% GC; will |
| | | alpha | | CCTCTTCAGCACAAAGGACTCAT [symbol] | also detect IFN-alpha |
| | | | | TGC | 1B, 201, may detect [symbol] |
| | | | | (SEQ ID NO: 445) | 21, 5, 7, 17, 4, 16, 10 |
| M422 | IFNAR1 | interferon | NM_000629 | GACTCATTTACACCATTTCGCAA [symbol] | 50 bp, e3-4, 44% GC |
| | | (alph | | GCTCAGATTGGTCCTCCAGAAGT [symbol] | |
| | | beta and | | CA | |
| | | omega | | (SEQ ID NO: 446) | |
| | | receptor 1 | | | |
| M423 | IFNAR2 | interferon | NM_000874 | CACTTAATTTGGTTCTCATGGTG [symbol] | 50 bp, e3-4, 38% GC; |
| | | (alph | | ATATCAGCCTCGTGTTTGGTATT [symbol] | will detect both soluble |
| | | beta and | | CA | and membrane-bound |
| | | omega | | (SEQ ID NO: 447) | receptors |
| | | receptor 2 | | | |
| M424 | IFN-beta | fibroblast | M35591 | GATCAAGCAACTCCAGCAATTC [symbol] | 50 bp, 46% GC |
| | | interferon | | GAAAGAAGACGCAGCTCTGACT [symbol] | |
| | | | | TCT | |
| | | | | (SEQ ID NO: 448) | |
| M425 | IFN-beta | interferon- | X04430 | ATGCTTCCAATCTGGATTCAAT [symbol] | 50 bp, e3-4, 42% GC = |
| | (IL6) | beta- | | GGAGACTTGCCTGGTGAAAATC [symbol] | IL6 |
| | | (interleukin 6) | | CA | |
| | | | | (SEQ ID NO: 449) | |
| M426 | IFMBR | interferon | A26595 | GACTCATTTACACCATTTCGCAA [symbol] | 50 bp, e3-4, 44% GC |
| | | beta | | GCTCAGATTGGTCCTCCAGAAGT [symbol] | |
| | | receptor | | CA | |
| | | | | (SEQ ID NO: 450) | |
| M427 | | IFN-gamma | A25270 | ACCTTCTAAGTCACGTCACCAT [symbol] | |
| | | antagonist | NM_006083 | GATGCCAAGGGAGTACAATGAG [symbol] | |
| | | cytokine | | ATG | |
| | | | | (SEQ ID NO: 451) | |
| M428 | IFNG | interferon, | NM_000619 | CTTAGGCATTTTGAAGAATTGGA [symbol] | 50 bp, e2-3, 36% GC |
| | | gamma | | AGAGGAGAGTGACAGAAAAATA [symbol] | |
| | | | | TGC | |
| | | | | (SEQ ID NO: 452) | |
| M429 | IFNGR1 | interferon | NM_000416 | GTATGTGAGAATGAACGGAAGT [symbol] | 50 bp, e4-5, 40% GC |
| | | gamma | | AGATCCAGTATAAAATACTCAC [symbol] | |
| | | receptor 1 | | AGA | |
| | | | | (SEQ ID NO: 453) | |
| M430 | IFNGR2 | interferon | NM_005534 | TTGTCTACCGAGTGCAGTTTTAAA [symbol] | 50 bp, e2-3, 46% GC |
| | | gamma | | ACACCGACAGTAAATGGTTCAC [symbol] | |
| | | receptor 2 | | GCC | |
| | | | | (SEQ ID NO: 454) | |
| M431 | ILI8 | interleukin | NM_001562 | GATATGACTGATTCTGACTGTA [symbol] | 50 bp, e2-3, 40% GC |
| | | 18 | | GATAATGCACCCCGGACCATATT [symbol] | |
| | | | | AT | |
| | | | | (SEQ ID NO: 455) | |
| M432 | IL1A | interleukin | NM_000575 | GCATTACATAATCTGGATGAAG [symbol] | 50 bp, e4-5, 36% GC |
| | | 1, alpha | | GTGAAATTTGACATGGGTGCTTA [symbol] | |
| | | | | AA | |
| | | | | (SEQ ID NO: 456) | |
| M433 | IL1B | interleukin | NM_000576 | CCAGTGAAATGATGGCTTATTA [symbol] | 50 bp, e2-3, 40% GC |
| | | 1, b [symbol] | | GTGGCAATGAGGATGACTTGTT [symbol] | |
| | | | | TT | |
| | | | | (SEQ ID NO: 457) | |
| M434 | IL1E | interleukin-1 | AF206696 | GATGGTGGAGGAAGGGCCGTCT [symbol] | 50 bp, e2-3, 46% GC |
| | | epsilon | | TCAATCAATGTGTAAACCTATTA [symbol] | |
| | | | | TGG | |
| | | | | (SEQ ID NO: 458) | |
| M435 | IL1R1 | interleukin 1 | NM_000877 | GTAATAGAATTTATTACTCTAGA [symbol] | 50 bp, e4-5, 36% GC |
| | | receptor, | | GAAAACAAACCCACAAGGCCTG [symbol] | |
| | | type I [symbol] | | GAT | |
| | | | | (SEQ ID NO: 459) | |
| M436 | IL1R2 | interleukin 1 | NM_004633 | CTGGCACCTACGTCTGCACTACT [symbol] | 50 bp, e2-3, 44% GC |
| | | receptor, | | GAAATGCTTCTTACTGTGACAAA [symbol] | |
| | | type I [symbol] | | TG | |
| | | | | (SEQ ID NO: 460) | |
| M437 | IL10 | interleukin 10 | NM_000572 | CTTCAGCAGAGTGAAGACTTTCT [symbol] | 50 bp, e1-2, 42% GC |
| | | | | TCAAATGAAGGATCAGCTGGAC [symbol] | |
| | | | | ACT | |
| | | | | (SEQ ID NO: 461) | |
| M438 | IL10RA | interleukin 10 | NM_001558 | TACCTGCTATGAAGTGGCGCTC [symbol] | 50 bp, e2-3, 50% GC |
| | | receptor, alpha | | GAGGTATGGAATAGAGTCCTGG [symbol] | |
| | | | | ACT | |
| | | | | (SEQ ID NO: 462) | |
| M439 | IL10RB | interleukin 10 | NM_000628 | TTCTCTTCCACAGCACCTGAAA [symbol] | 50 bp, e6-7, 44% GC |
| | | receptor, beta | | GTTTTTGGGCCATCCTCATCATA [symbol] | |
| | | | | CA | |
| | | | | (SEQ ID NO: 463) | |
| M440 | IL11 | interleukin 11 | NM_000641 | GACATGAACTGTGTTTGCCGCCT [symbol] | 40 bp, e1-2, 57% GC |
| | | | | GTCCTGGTCGTGCTGA | |
| | | | | (SEQ ID NO: 464) | |
| M441 | IL10RA | interleukin 10 | NM_004512 | TATGAGAACTTCTCTTGCACTTG [symbol] | 49 bp, e5, 49% GC |
| | | receptor, alpha | | AGTCCCAGCCAGATCAGCGGTT [symbol] | |
| | | | | C | |
| | | | | (SEQ ID NO: 465) | |
| M442 | IL12 | interleukin 12 | AF180562 | GGCCGTCAGCAACATGCTCCAG [symbol] | 50 bp, e2-3, 50% GC |
| | | | | AGGCCAGACAAACTCTAGAATT [symbol] | |
| | | | | ACC | |
| | | | | (SEQ ID NO: 466) | |
| M443 | IL12RB1 | interleukin 12 | NM_005535 | TGACCCTGCAGCTCTACAACTCA [symbol] | 50 bp, e4-5, 50% GC |
| | | receptor, beta | | TTAAATATGAGCCTCCTCTGGGA [symbol] | |
| | | 1 | | A | |
| | | | | (SEQ ID NO: 467) | |
| M444 | IL12RB2 | interleukin 12 | NM_00155 | AGTAACAGCAGGCTCTGGAATA [symbol] | 50 bp, e6-7, 42% GC |
| | | receptor, beta | | GGTTAATGTTACAAAGGCCAAA [symbol] | |
| | | 2 | | GAAG | |
| | | | | (SEQ ID NO: 468) | |
| M445 | IL13 | interleukin 13 | NM_002188 | ATCACCCAGAACCAGAAGGCTC [symbol] | 46 bp, e1-2, 54% GC |
| | | | | GCTCTGCAATGGCAGCATGGTAT [symbol] | |
| | | | | (SEQ ID NO: 469) | |
| M446 | IL13RA1 | interleukin 13 | NM_001560 | CCAGAATTTGAGAGAAATGTGG [symbol] | 50 bp, e6-7, 42% GC |
| | | receptor, alpha | | GAATACATCTTGTTTCATGGTC [symbol] | |
| | | | | TGG | |
| | | | | (SEQ ID NO: 470) | |
| M447 | IL13RA2 | interleukin 13 | NM_000640 | TGGAGTGATAAACAATGCTGGG [symbol] | 50 bp, e8-9, 40% GC |
| | | receptor, alpha | | AGGTGAAGACCTATCGAAGAAA [symbol] | |
| | | | | CTTT | |
| | | | | (SEQ ID NO: 471) | |
| M448 | IL15 | interleukin 15 | NM_000585 | CATTCATGTCTTCATTTTGGGCT [symbol] | 50 bp, e4-5, 44% GC |
| | | | | TTCAGTGCAGGGCTUCCTAAAA [symbol] | |
| | | | | G | |
| | | | | (SEQ ID NO: 472) | |
| M449 | IL15RA | interleukin 15 | NM_002189 | TTCTGGAAAAGAGCCCGCAGCT [symbol] | 47 bp, e3-4, 51% GC |
| | | receptor, alpha | | ATCTCCCAGCTCAAACAACACA [symbol] | |
| | | | | (SEQ ID NO: 473) | |
| M450 | IL2 | interleukin 2 | NM_000586 | ATTTAAGTTTTACATGCCCAAGA [symbol] | 50 bp, e2-3, 40% GC |
| | | | | GGCCACAGAACTGAAACAGCTT [symbol] | |
| | | | | AGT | |
| | | | | (SEQ ID NO: 474) | |
| M451 | IL2RA | interleukin 2 | NM_000417 | AACCAATGTCAATGCACAAGCT [symbol] | 50 bp, e2-3, 44% GC |
| | | receptor, alpha | | GCCACTCGGAACACAACGAAA [symbol] | |
| | | | | AGT | |
| | | | | (SEQ ID NO: 475) | |
| M452 | IL2RB | interleukin 2 | NM_000878 | AACCTGATCCTCGGAGCCCCAGA [symbol] | 50 bp, e4-5, 50% GC |
| | | receptor, beta | | TCTCAGAAACTGACCACAGTTGA [symbol] | |
| | | | | AT | |
| | | | | (SEQ ID NO: 476) | |
| M453 | IL2RG | interleukin 2 | NM_000206 | TGGGAATGAAGACACCACAGCT [symbol] | 50 bp, e1-2, 50% GC |
| | | receptor, gamma | | ATTTCTTCCTGACCACTATGCCC [symbol] | |
| | | | | CTG | |
| | | | | (SEQ ID NO: 477) | |
| M454 | IL3 | interleukin 3 | NM_000588 | ATCAGCAATTGAGAGCATTCTTA [symbol] | 50 bp, e3-4, 46% GC |
| | | | | AAATCTCCTGCCATGTCTGCCC [symbol] | |
| | | | | GG | |
| | | | | (SEQ ID NO: 478) | |
| M455 | IL3RA | interleukin 3 | NM_002183 | TTGCCAACAGGCGTCAACAGTA [symbol] | 50 bp, 48% GC |
| | | receptor, alpha | | GAGTGTCTTCACTACAAAACGG [symbol] | |
| | | | | GCT | |
| | | | | (SEQ ID NO: 479) | |
| M456 | IL4 | interleukin 4 | NM_000589 | TAACAGACATCTTTGCTGCCTC [symbol] | 50 bp, e2-3, 44% GC |
| | | | | AGAACACAACTGAGAAGGAAA [symbol] | |
| | | | | TTC | |
| | | | | (SEQ ID NO: 480) | |
| M457 | IL4R | interleukin 4 | NM_000418 | TTGGAGTGAAAACGACCCGGCA [symbol] | 50 bp, e5-6, 46% GC |
| | | receptor | | ATTTCAGAATCTATAACGTGAC [symbol] | |
| | | | | ACC | |
| | | | | (SEQ ID NO: 481) | |
| M458 | IL5 | interleukin 5 | NM_000879 | TCGAACTCTGCTGATAGCCAAT [symbol] | 50 bp, e1-2, 48% GC |
| | | | | GACTCTGAGGATTCCTGTTCCTG [symbol] | |
| | | | | AC | |
| | | | | (SEQ ID NO: 482) | |
| M459 | IL5RA | interleukin 5 | NM_000564 | ACACGCAGTATTTTCTCTACTAT [symbol] | 50 bp, e7-8, 42% GC |
| | | receptor, alpha | | GGTATGGCTCTTGGACTGAAGAA [symbol] | |
| | | | | GC | |
| | | | | (SEQ ID NO: 483) | |
| M460 | IFNB1 | interferon, | NM_002176 | AGTGTCTCCTCCAAATTGCTCTC [symbol] | 50 bp, 48% GC |
| | | beta fibroblast | | TGTTGTGCTTCTCCACTACAGCT [symbol] | |
| | | | | TT | |
| | | | | (SEQ ID NO: 484) | |
| M461 | IL6R | interleukin 6 | NM_000565 | TCAAAGACATTCACAACATGGA [symbol] | 50 bp, e5-6, 46% GC |
| | | receptor | | GGTCAAGGACCTCCAGCATCAC [symbol] | |
| | | | | TGT | |
| | | | | (SEQ ID NO: 485) | |
| M462 | IL7 | interleukin 7 | NM_000880 | TAATGGTCAGCATCGATCAATTA [symbol] | 50 bp, e2-3, 38% GC |
| | | | | TGGACAGCATGAAAGAAATTGG [symbol] | |
| | | | | AGC | |
| | | | | (SEQ ID NO: 486) | |
| M463 | IL7R | interleukin 7 | NM_002185 | GGAGAAAGTGGCTATGCTCAAA [symbol] | 50 bp, e1-2, 46% GC |
| | | receptor | | TGGAGACTTGGAAGATGCAGAA [symbol] | |
| | | | | TGGA | |
| | | | | (SEQ ID NO: 487) | |
| M464 | IL8 | interleukin 8 | NM_000584 | CTGATTTCTGCAGCTCTGTGTGA [symbol] | 50 bp, e1-2, 48% GC |
| | | | | GGTGCAGTTTTGCCAAGGAGTG [symbol] | |
| | | | | AA | |
| | | | | (SEQ ID NO: 488) | |
| M465 | IL8RA | interleukin 8 | NM_000634 | CTTGGTCAAGTTTGTTTGTCTTG [symbol] | 50 bp, 46% GC |
| | | receptor, alpha | | CTGCTGGGGACTGTCTATGAAT [symbol] | |
| | | | | GT | |
| | | | | (SEQ ID NO: 489) | |
| M466 | IL8RB | interleukin 8 | NM_001557 | GGAAGATTTTAACATGGAGAGT [symbol] | 50 bp, 40% GC |
| | | receptor, beta | | ACAGCTTTGAAGATTTCTGGAAA [symbol] | |
| | | | | GTG | |
| | | | | (SEQ ID NO: 490) | |
| M467 | IL9 | interleukin 9 | NM_000590 | ACTAAAGAACAACAAGTGTCCA [symbol] | 50 bp, e4-5, 40% GC |
| | | | | ATTTTTCCTGTGAACAGCCATG [symbol] | |
| | | | | ACC | |
| | | | | (SEQ ID NO: 491) | |
| M468 | IL9R | interleukin 9 | NM_002186 | TGTTCAAGCTGTCGCCCAGGGT [symbol] | 46 bp, e8-9, 50% GC |
| | | receptor | | AGAGAATCTTCTAGCAGAACGT [symbol] | |
| | | | | (SEQ ID NO: 492) | |
| M469 | LIF | leukemia | NM_002309 | TGCCAATGCCCTCTTTATTCTCT [symbol] | 48 bp, e2-3, 53% GC |
| | | inhibitory | | TACACAGCCCAGGGGGAGCCGT [symbol] | |
| | | factor | | (SEQ ID NO: 493) | |
| | | receptor | | | |
| M470 | LIFR | leukemia | NM_002310 | GGAGCCCTGTGAAGAACATTTCT [symbol] | 50 bp, e6-7, 44% GC |
| | | inhibitory | | GGATACCTGATTCTCAGACTAA [symbol] | |
| | | factor | | TT | |
| | | receptor | | (SEQ ID NO: 494) | |
| M471 | BLT1 | leukotriene B4 | AB008193 | TCACTAGGTGTAGAGTTCATCT [symbol] | 50 bp, 48% GC |
| | | receptor | | CTGCTGGCTATCATCCTGCTGTC [symbol] | |
| | | | | GT | |
| | | | | (SEQ ID NO: 495) | |
| M472 | BLT2 | leukotriene B4 | AB008193 | TTGGCCTTCTTCAGTTCTAGCGT [symbol] | 44 bp, 52% GC |
| | | receptor | | AACCCGGTGCTCTACGTCTT | |
| | | | | (SEQ ID NO: 496) | |
| M473 | CYSLT1 | cysteinyl | NM_006639 | CAGGAAATCTGACAGTATCTTCT [symbol] | 50 bp, 44% GC |
| | | leukotriene | | CCACATGCCATGACACTATTGAT [symbol] | |
| | | receptor 1 | | AC | |
| | | | | (SEQ ID NO: 497) | |
| M474 | LTA4H | leukotriene A4 | NM_000895 | TGTGAAATTAACCTATACTGCA [symbol] | 50 bp, e4-5, 44% GC |
| | | hydrolase | | GGTGTCTGTCCCTAAAGAACTG [symbol] | |
| | | | | GG | |
| | | | | (SEQ ID NO: 498) | |
| M475 | LTC4S | leukotriene C4 | NM_000897 | GTCTACCGAGCCCAGGTGAACT [symbol] | 36 bp, e2-3, 58% GC |
| | | synthase | | AGCGAGTACTTC | |
| | | | | (SEQ ID NO: 499) | |
| M476 | LTB4DH | NADP depende [symbol] | XM_088569 | ATGATGGGGCAGGAAGTGGCCA [symbol] | 50 bp, e4-5, 46% GC |
| | | leukotriene b4 [symbol] | | AGTTGTGGAAAGTAAAAATGTA [symbol] | |
| | | hydroxydehydro- | | CCCT | |
| | | genase | | (SEQ ID NO: 500) | |
| M477 | SELL | selectin L | NM_000655 | TCATCTCCAGAACCAACCTGTCA [symbol] | 50 bp, e5-6, 46% GC |
| | | | | GTGATTCAGTGTGAGCCTCTAT [symbol] | |
| | | | | GC | |
| | | | | (SEQ ID NO: 501) | |
| M478 | SCYC1 | lymphotactin | NM_002995 | TCTCTCACTGCATACATTGTGGA [symbol] | 50 bp, e1-2, 44% GC |
| | | | | GGTGTAGGGAGTGAAGTCTCAG [symbol] | also detect SCYC2 = |
| | | | | TAA | SCYC1? |
| | | | | (SEQ ID NO: 502) | |
| M479 | CCXCR1 | chemokine (C | NM_005283 | CAAACTCCTCAATATGATCTTCT [symbol] | 50 bp, 44% GC |
| | | motif) XC | | CATCAGCCTCTACAGCAGCATCT [symbol] | |
| | | receptor 1 | | CT | |
| | | | | (SEQ ID NO: 503) | |
| M480 | SCYA22 = | small inducible | NM_002990 | TTCAAGCAACTGAGGCAGGCCC [symbol] | 42 bp, e1-2, 58% GC |
| | MDC? | cytokine | | ACGGCGCCAACATGGAA | |
| | | subfamily A | | (SEQ ID NO: 504) | |
| | | (C [symbol] | | | |
| | | Cys), | | | |
| | | member 2 | | | |
| M481 | CCR2 | chemokine (C- [symbol] | NM_000648 | AGTGCTTGACTGACATTTACCT [symbol] | 50 bp, 48% GC shoul [symbol] |
| | | motif) receptor | | TCAACCTGGCCATCTCTGATCT [symbol] | detect both isoforms |
| | | | | TT | may also detect CCR |
| | | | | (SEQ ID NO: 505) | |
| M482 | SCYA3 | small inducible | NM_002983 | AAGCCCGGTGTCATCTTCCTAA [symbol] | 41 bp, e2-3, 58% GC |
| | | cytokine A3 | | AAGCGAAGCCGGCAGGT [symbol] | |
| | | | | (SEQ ID NO: 506) | |
| M483 | SCYA4 | small inducible | NM_002984 | GCTCCCAGCCAGCTGTGGTATT [symbol] | 50 bp, e2-3, 50% GC |
| | | cytokine A4 | | AAACCAAAAGAAGCAAGCAAGT [symbol] | |
| | | | | TGT | |
| | | | | (SEQ ID NO: 507) | |
| M484 | AMH | anti-Mullerian | NM_000479 | AGCCCCGCGGAGAGGACTCCCG [symbol] | 30 bp, e3-4, 70% GC |
| | | hormone | | CTGAGTA | |
| | | | | (SEQ ID NO: 508) | |
| M485 | AMHR2 | anti-Mullerian | NM_020547 | ATGAGGAGCGCTGGCAGAATG [symbol] | 48 bp, e7-8, 52% GC |
| | | hormone receptor | | CAATATAAACCAGGTATTGCCCA [symbol] | |
| | | typeII | | C | |
| | | | | (SEQ ID NO: 509) | |
| M486 | SCYD1 | neurotactin | NM_002996 | TGCGGCAAACGCGCAATCATCT [symbol] | 47 bp, e2-3, 55% GC |
| | | | | GAGACGAGACAGCACAGGCTGT [symbol] | |
| | | | | (SEQ ID NO: 510) | |
| M487 | NRTN | neurturin | NM_004558 | TCAGTGCTCTGCAGCTCCGTGCT [symbol] | 40 bp, e1, 57% GC |
| | | | | TCCATCTGGATGTGTC | |
| | | | | (SEQ ID NO: 511) | |
| M488 | BMP-8 | bone morpho- | NM_001720 | AGGTGGTCCAGGAGCAGTCCAA [symbol] | 50 bp, e2-3, 50% GC |
| | | genetic | | AGGGAGTCTGACTTGTTCTTTTT [symbol] | |
| | | protein 8 | | GAT | |
| | | | | (SEQ ID NO: 512) | |
| M489 | FAAH | fatty acid | NM_001441 | TGCTCTTCACCTATGTGGGAAA [symbol] | 49 bp, e2-3, 51% GC |
| | | amide hydrolase | | CCTGGGAAGTGAACAAAGGGA [symbol] | |
| | | | | AA | |
| | | | | (SEQ ID NO: 513) | |
| M490 | OSM | oncostatin M | NM_020530 | ATCTCATGCAGGACACCAGCAG [symbol] | 50 bp, e2-3, 50% GC |
| | | | | CTCCTGGACCCCTATATACGTAT [symbol] | |
| | | | | CAA | |
| | | | | (SEQ ID NO: 514) | |
| M491 | OSMR | oncostatin M | NM_003999 | TCCTTGAGGACTTACCAGAGTGA [symbol] | 50 bp, e2-3, 46% GC |
| | | receptor | | GTCTTGGCTGAACGTTTACCATT [symbol] | |
| | | | | AC | |
| | | | | (SEQ ID NO: 515) | |
| M492 | SCYA5 | RANTES | NM_002985 | AAGTGCTCCAACCCAGCAGTCG [symbol] | 44 bp, e2-3, 54% GC |
| | | | | TTTGTCACCCGAAAGAACCG | |
| | | | | (SEQ ID NO: 516) | |
| M493 | | RANTES | L10918 | TGTACTCCTTGGTATTTGTCATT [symbol] | 50 bp, 46% GC |
| | | Receptor | | GCCTGGTTGGAAACATCCTGGT [symbol] | |
| | | | | TC | |
| | | | | (SEQ ID NO: 517) | |
| M494 | | relaxin H1 | X00949 | CCTCAGACACCTAGACCAGTGG [symbol] | 50 bp, e1-2, 46% GC |
| | | | | AGAAATTGTACCATCCTTCATCA [symbol] | should detect also |
| | | | | CAA | relaxin H2 (1 bp |
| | | | | (SEQ ID NO: 518) | different) |
| M495 | SDF1 | stromal cell- | XM_165565 | CTCCAAACTGTGCCCTTCAGATT [symbol] | 50 bp, e2-3, 50% GC |
| | | derived factor | | TAGCCCGGCTGAAGAACAACAA [symbol] | |
| | | 1 | | AGA | |
| | | | | (SEQ ID NO: 519) | |
| M496 | SDF2 | stromal cell- | NM_006923 | CTTCACCTCTTTCTGGAAACCA [symbol] | 50 bp, e2-3, 48% GC |
| | | derived factor | | AAGTGAGTGCTTTTGGTGAGGA [symbol] | |
| | | | | GGT | |
| | | | | (SEQ ID NO: 520) | |
| M497 | SDFR1 | stromal cell | XM_015963 | AGAATGCCAGCAACATGGAGTA [symbol] | 50 bp, e3-4, 48% GC |
| | | derived factor | | AGGATCAATAAGCCGAGAGCTG [symbol] | |
| | | receptor 1 | | GGAT | |
| | | | | (SEQ ID NO: 521) | |
| M498 | TGF-alpha | transforming | M31172 | TTTTTGGTGCACGAGGACAAGC [symbol] | 48 bp, e2-3, 52% GC |
| | | growth factor- | | GCATGTGTCTGCCATTC TGGGTA [symbol] | |
| | | alpha | | (SEQ ID NO: 522) | |
| M499 | TGFB1 | transforming | NM_000660 | GTGCTAATGGTGGAAACCCACA [symbol] | 50 bp, e1-2, 44% GC |
| | | growth factor, | | CGAAATCTATGACAAGTTCAAG [symbol] | |
| | | beta 1 | | AGAG | |
| | | | | (SEQ ID NO: 523) | |
| M500 | TGFB2 | transforming | M19154 | GAAGAACTAGAAGCAAGATTTG [symbol] | 50 bp, e3-4, 42% GC |
| | | growth factor, | M22045 | AGGTATTGATGGCACCTCCACAT [symbol] | |
| | | beta 2 | M22046 | TAG | |
| | | | | (SEQ ID NO: 524) | |
| M501 | TGFB3 | transforming | J03241 | GAGGTGATGGAAATCAAATTCA [symbol] | 50 bp, e4-5, 46% GC |
| | | growth factor, | | AGGCGTGGACAATGAGGATGA [symbol] | |
| | | beta 3 | | ATGG | |
| | | | | (SEQ ID NO: 525) | |
| M502 | TGFBR1 | transforming | NM_004612 | ATGACAACGTCAGGTTCTGGCT [symbol] | 50 bp, e3-4, 46% GC |
| | | growth factor, | | GGTTTACCATTGCTTGTTCAGAG [symbol] | |
| | | beta receptor | | AC | |
| | | 1 | | (SEQ ID NO: 526) | |
| M503 | TGFBR2 | transforming | NM_003242 | CACAGGAAGTCTGTGTGGCTGTA [symbol] | 50 bp, e2-3, 44% GC |
| | | growth factor, | | GGAGAAAGAATGACGAGAACAT [symbol] | |
| | | beta receptor | | ACA | |
| | | 2 | | (SEQ ID NO: 527) | |
| M504 | TGFBR3 | transforming | NM_003243 | TGGGGTCTCCAGACTGTTTTTGG [symbol] | 50 bp, e3-4, 50% GC |
| | | growth factor, | | GTCTGAGGGTTCTGTGGTCCAGT [symbol] | |
| | | beta receptor | | TT | |
| | | 3 | | (SEQ ID NO: 528) | |
| M505 | THPO | thrombopoietin | NM_000460 | TTTAGCTTGGGAGAATGGAAAA [symbol] | 50 bp, e3-4, 50% GC |
| | | | | CCAGATGGAGGAGACCAAGGCA [symbol] | |
| | | | | AGGA | |
| | | | | (SEQ ID NO: 529) | |
| M506 | TNF | tumor necrosis | NM_000594 | CGAGTGACAAGGCTGTAGCCCA [symbol] | 46 bp, e2-3, 52% GC |
| | TNFSF2 | factor | | GTTGTAGCAAACCCTCAAGCTGA [symbol] | |
| | | | | (SEQ ID NO: 530) | |
| M507 | TNFSF1 | lymphotoxin | NM_000595 | AAACCTGCTGCTCACCTCATTG [symbol] | 45 bp, e3-4, 53% GC |
| | (LTA) | alpha | | GACCCCAGCAAGCAGAACTCA | |
| | | | | (SEQ ID NO: 531) | |
| M508 | TNFSF3 | lymphotoxin | NM_002341 | CTTAGTGCCCCAGGATCAGGGA [symbol] | 39 bp, e1-2, 59% GC |
| | (LTB) | be [symbol] | | GACTGGTAACGGAGAC | |
| | | | | (SEQ ID NO: 532) | |
| M509 | TNFSF4 | tumor necrosis | NM_003326 | TGCCTGCACTTCTCTGCTCTTCA [symbol] | 50 bp, e1-2, 48% GC |
| | | factor (ligand) | | GTATCACATCGGTATCCTCGAAT [symbol] | |
| | | superfamily, | | CA | |
| | | member 4 | | (SEQ ID NO: 533) | |
| M510 | TNFSF5 | tumor necrosis | NM_000074 | GAGGCCAGCAGTAAAACAACAT [symbol] | 50 bp, e2-3, 50% GC |
| | | factor (ligand) | | TGTGTTACAGTGGGCTGAAAAA [symbol] | |
| | | superfamily, | | GATA | |
| | | member 5 | | (SEQ ID NO: 534) | |
| M511 | TNFSF6 | tumor necrosis | NM_000639 | GAGCTGAGGAAAGTGGCCCATT [symbol] | 50 bp, e3-4, 50% GC |
| | | factor (ligand) | | AACAGGCAAGTCCAACTCAAGG [symbol] | |
| | | superfamily, | | CCAT | |
| | | member 6 | | (SEQ ID NO: 535) | |
| M512 | TNFSF7 | tumor necrosis | NM_001252 | TGCAGCTGAATCACACAGGACC [symbol] | 43 bp, e2-3, 55% GC = |
| | | factor (ligand) | | AGCAGGACCCCAGGCTATA | CD27? |
| | | superfamily, | | (SEQ ID NO: 536) | |
| | | member 7 | | | |
| M513 | TNFSF8 | tumor necrosis | NM_001244 | ATTATGGTGTTGGTCGTTCAGA [symbol] | 50 bp, e1-2, 50% GC |
| | | factor (ligand) | | ACGGACTCCATTCCCAACTCAC [symbol] | |
| | | superfamily, | | GA | |
| | | member 8 | | (SEQ ID NO: 537) | |
| M514 | TNFSF9 | tumor necrosis | NM_003811 | AGGGCATGTTTGCGCAGCTGGT [symbol] | 50 bp, e2-3, 50% GC |
| | | factor (ligand) | | CCCAAAATGTTCTGCTGAT | |
| | | superfamily, | | (SEQ ID NO: 538) | |
| | | member 9 | | | |
| M515 | TNFSF10 | tumor necrosis | NM_003810 | GTACTTTACCAACGAGCTGAAG [symbol] | 50 bp, e1-2, 46% GC |
| | | factor (ligand) | | GATGCAGGACAAGTACTCCAAA [symbol] | |
| | | superfamily, | | GTG | |
| | | member 10 | | (SEQ ID NO: 539) | |
| M516 | TNFSF11 | tumor necrosis | NM_003701 | GCCTTTCAAGGAGCTGTGCAAA [symbol] | 50 bp, e2-3, 42% GC |
| | | factor (ligand) | | GGAATTACAACATATCGTTGGAT [symbol] | |
| | | superfamily, | | ACA | |
| | | member 11 | | (SEQ ID NO: 540) | |
| M517 | TNFSF12 | tumor necrosis | NM_003809 | CTCTACTACCTGTACTGTCAGGT [symbol] | 47 bp, e6-7, 51% GC |
| | | factor (ligand) | | CACTTTGATGAGGGGAAGGCTG [symbol] | |
| | | superfamily, | | (SEQ ID NO: 541) | |
| | | member 12 | | | |
| M518 | TNFSF13 | tumor necrosis | NM_003808 | TGGAGTTTATCTGCTGTATAGC [symbol] | 50 bp, e4-5, 44% GC |
| | | factor (ligand) | | GGTCCTGTTTCAAGACGTGACTT [symbol] | |
| | | superfamily, | | CA | |
| | | member 13 | | (SEQ ID NO: 542) | |
| M519 | TNFSF14 | tumor necrosis | NM_003807 | AGCTGATACAAGAGCGAAGGT [symbol] | 42 bp, e2-3, 54% GC |
| | | factor (ligand) | | CACGAGGTCAACCCAGCAG | |
| | | superfamily, | | (SEQ ID NO: 543) | |
| | | member 14 | | | |
| M520 | TNFSF15 | tumor necrosis | NM_005118 | TCACCAAGAACCGAATGAACTA [symbol] | 50 bp, 44% GC |
| | | factor (ligand) | | ACCAACAAATTCCTGCTGATCC [symbol] | may also detect |
| | | superfamily, | | GAG | transmembrane LIGH [symbol] |
| | | member 15 | | (SEQ ID NO: 544) | mRNA (AY028261) |
| M521 | TNFSF18 | tumor necrosis | NM_005092 | AAGGAGCCCTGTATGGCTAAGT [symbol] | 50 bp, e2-3, 44% GC |
| | | factor (ligand) | | GGACCATTACCCTCAAAATGGCA [symbol] | |
| | | superfamily, | | AAT | |
| | | member 18 | | (SEQ ID NO: 545) | |
| M522 | TNFRSF10B | tumor necrosis | NM_003842 | TTTTCTGCTTGCGCTGCACCAGG [symbol] | 50 bp, e2-3, 50% GC |
| | | factor receptor | | GTGATTCAGGTGAAGTGGAGCT [symbol] | |
| | | superfamily, | | AGT | |
| | | member 10B | | (SEQ ID NO: 546) | |
| M523 | TNFRSF11A | tumor necrosis | XM_036978 | ACAAATGCAGACCCTGGACCAA [symbol] | 50 bp, e4-5, 50% GC |
| | | factor receptor | | TGTACCTTCCTTGGAAAGAGAGT [symbol] | |
| | | superfamily, | | GAA | |
| | | member 11A | | (SEQ ID NO: 547) | |
| M524 | TNFRSF11B | tumor necrosis | NM_002546 | TGGATTTGGAGTGGTGCAAGCT [symbol] | 50 bp, e2-3, 50% GC |
| | | factor receptor | | AACCCCAGAGCGAAATACAGTT [symbol] | |
| | | superfamily, | | GCA | |
| | | member 11B | | (SEQ ID NO: 548) | |
| M525 | TNFRSF12 | tumor necrosis | NM_003790 | CACACACGGCTACTCTGTTCCC [symbol] | 45 bp, e4-5, 53% GC |
| | | factor receptor | | AGAGATACTGACTGTGGGA | |
| | | superfamily, | | (SEQ ID NO: 549) | |
| | | member 12 | | | |
| M526 | TNFRSF14 | tumor necrosis | NM_003820 | GTGAAAAGAAGAAAGCCAAGG [symbol] | 50 bp, e6-7, 48% GC |
| | | factor receptor | | TGATGTAGTCAAGGTGATCGTCT [symbol] | |
| | | superfamily, | | CGT | |
| | | member 14 | | (SEQ ID NO: 550) | |
| M527 | TNFRSF17 | tumor necrosis | NM_001192 | CATGTCAGCGTTATTGTAATGCA [symbol] | 50 bp, e1-2, 42% GC |
| | | factor receptor | | GTGTGACCAATTCAGTGAAAGG [symbol] | |
| | | superfamily, | | ACG | |
| | | member 17 | | (SEQ ID NO: 551) | |
| M528 | TNFRSF18 | tumor necrosis | NM_004195 | ACTGCAAACCTTGGACAGACTG [symbol] | 49 bp, e3-4, 51% GC |
| | | factor receptor | | ACCCAGTTCGGGTTTCTCACTGT [symbol] | |
| | | superfamily, | | TT | |
| | | member 18 | | (SEQ ID NO: 552) | |
| M529 | TNFRSF19 | tumor necrosis | NM_018647 | GTATTACTAGGCTATTTGTCATG | 50 bp, e2-3, 38% GC |
| | | factor receptor | | TAAAGTGACTTGTGAATCAGGAG | |
| | | superfamily, | | ACTG | |
| | | member 19 | | (SEQ ID NO: 553) | |
| M530 | TNFRSF21 | tumor necrosis | NM_014452 | ACTTATGTTCCCAAAGGCATGAA | 50 bp, e2-3, 44% GC |
| | | factor receptor | | CTCAACAGAATCCAACTCTTCTG | |
| | | superfamily, | | CCTC | |
| | | member 21 | | (SEQ ID NO: 554) | |
| M531 | TNFRSF3 | tumor necrosis | NM_002342 | ACTGAAGCCGAGCTCAAAGATG | 50 bp, e4-5, 50% GC |
| | | factor receptor | | AAGTTGGGAAGGGTAACAACCA | |
| | | superfamily, | | CTGCGT | |
| | | member 3 | | (SEQ ID NO: 555) | |
| M532 | TNFRSF4 | tumor necrosis | NM_003327 | TGCACGTGGTGTAACCTCAGAAG | 41 bp, e2-3, 56% GC |
| | | factor receptor | | TGGGAGTGAGCGGAAGCA | |
| | | superfamily, | | (SEQ ID NO: 556) | |
| | | member 4 | | | |
| M533 | TNFRSF6 | tumor necrosis | NM_000043 | AGATTGTGTGATGAAGGACATGG | 50 bp, e3-4, 44% GC = |
| | | factor receptor | | CTTAGAAGTGGAAATAAACTGCA | Apo1 antigen? |
| | | superfamily, | | CCCG | |
| | | member 6 | | (SEQ ID NO: 557) | |
| M534 | TNFRSF6B | tumor necrosis | NM_003823 | TCAGCACCAGGGTACCAGGAGC | 38 bp, e2-3, 60% GC |
| | | factor receptor | | TGAGGAGTGTGAGCGT | |
| | | superfamily, | | (SEQ ID NO: 558) | |
| | | member 6B | | | |
| M535 | TNFRSF7 | tumor necrosis | NM_001242 | CATCAACGAAGGAAATATAGAT | 50 bp, e5-6, 42% GC |
| | | factor receptor | | CAAACAAAGGAGAAAGTCCTGT | |
| | | superfamily, | | GGAGCC | |
| | | member 7 | | (SEQ ID NO: 559) | |
| M536 | TNFRSF8 | tumor necrosis | NM_001243 | CACGGAGCACACCAATAACAAG | 50 bp, e9-10, |
| | | factor receptor | | ATTGAGAAAATCTACATCATGAA | 42% GC |
| | | superfamily, | | GGCTG | |
| | | member 8 | | (SEQ ID NO: 560) | |
| M537 | TNFRSF9 | tumor necrosis | NM_001561 | TGTGACATATGCAGGCAGTGTAA | 50 bp, e3-4, 48% GC |
| | | factor receptor | | AGGTGTTTTCAGGACCAGGAAGG | |
| | | superfamily, | | AGTG | |
| | | member 9 | | (SEQ ID NO: 561) | |
| M538 | GAPD | glyceraldehyde- | NM_002046 | CCCTTCATTGACCTCAACTACAT | 50 bp, e2-3, 40% GC |
| | | 3-phosphate | | GGTTTACATGTTCCAATATGATT | |
| | | dehydrogenase | | CCAC | |
| | | | | (SEQ ID NO: 562) | |
| M539 | | | | CATTGACCTCAACTACATGGTTT | 40 bp, e2-3, 35% GC |
| | | | | ACATGTTCCAATATGAT | |
| | | | | (SEQ ID NO: 563) | |
| M540 | | | | ACCTCAACTACATGGTTTACATGTTCC | 30 bp, e2-3, 32% GC |
| | | | | AAT | |
| | | | | (SEQ ID NO: 564) | |
| M541 | | | | AGTATGACAACAGCCTCAAGATCATC | 50 bp, e5-6, 50% GC |
| | | | | AGCATGCCTCCTGCACCACCAAC | |
| | | | | (SEQ ID NO: 565) | |
| M542 | | | | GACAACAGCCTCAAGATCATCAGCAA | 40 bp, e5-6, 52% GC |
| | | | | TGCCTCCTGCACCA | |
| | | | | (SEQ ID NO: 566) | |
| M543 | | | | CAGCCTCAAGATCATCAGCAAT | 30 bp, e5-6, 53% GC |
| | | | | GCCTCCTG | |
| | | | | (SEQ ID NO: 567) | |
| M544 | | | | AACTTTGGTATCGTGGAAGGACT | 50 bp, e6-7, 50% GC |
| | | | | CATGACGACAGTCCATGCCATC | |
| | | | | ACTGC | |
| | | | | (SEQ ID NO: 568) | |
| M545 | ACTB | actin, beta | NM_001101 | AGATCACTGCCCTGGCACCCAG | 50 bp, e4-5, 50% GC |
| | | | | CAATGAAGATCAAGATCATTGC | |
| | | | | CT | |
| | | | | (SEQ ID NO: 569) | |
| M546 | | | | GCCCTGGCACCCAGCACAATGA | 40 bp, e4-5, 52% GC |
| | | | | GATCAAGATCATTGCTC | |
| | | | | (SEQ ID NO: 570) | |
| M547 | | | | TGGCACCCAGCACAATGAAGAT [symbol] | 30 bp, e2-3, 46% GC |
| | | | | AAGATCA | |
| | | | | (SEQ ID NO: 571) | |
| M548 | Negative | Negative | NM_002046 | ATGACAACAGCCTCAAGATCAT [symbol] | 50 bp, 44% GC |
| | control | control | NM_001101 | AGGAAGATCAAGATCATTGCTC [symbol] | e5 of GAPD + e5 |
| | | | | CCT | ofACTB |
| | | | | (SEQ ID NO: 572) | |
| M549 | Negative | Negative | NM_002046 | AACAGCCTCAAGATCATCAGGA [symbol] | 40 bp, 42% GC |
| | control | control | NM_001101 | GATCAAGATCATTGCTC | e5 of GAPD + e5 |
| | | | | (SEQ ID NO: 573) | ofACTB |
| M550 | Negative | Negative | NM_002046 | CCTCAAGATCATCAGGAAGATC [symbol] | 30 bp, 40% GC |
| | control | control | NM_001101 | AGATCAT | e5 of GAPD + e5 |
| | | | | (SEQ ID NO: 574) | ofACTB |
| M551 | PTGDS | prostaglandin | NM_000954 | AACCTGACCTCCACCTTCCTCAG [symbol] | 50 bp, e2-3, 52% GC |
| | | D synthase | | AAAAACCAGTGTGAGACCCGAA [symbol] | |
| | | | | CAT | |
| | | | | (SEQ ID NO: 575) | |
| M552 | PTGES | prostaglandin | NM_004878 | ACGTGGAACGCTGCCTCAGGGC [symbol] | 42 bp, e2-3, 64% GC |
| | | E synthase | | CACCGGAACGACATGGAGA | |
| | | | | (SEQ ID NO. 576) | |
| M553 | LYST | Chediak-Higas [symbol] | U84744 | CCTGTCCAAGAAGAAAAGGCAA [symbol] | 50 bp, e4-5, 46% GC |
| | | syndrome protein | | AGATTTTAACCTACCGCTCTCAG [symbol] | |
| | | short iso | | AGA | |
| | | | | (SEQ ID NO: 577) | |
| M554 | MITF | microphthalmia [symbol] | NM_000248 | AATGCTAGAATATAATCACTAT [symbol] | 50 bp, e1-2, 44% GC |
| | | associated | | GGTGCAGACCCACCTCGAAAAC [symbol] | |
| | | transcription | | CCA | |
| | | factor | | (SEQ ID NO: 578) | |
| M555 | OCA2 | oculocutaneous | NM_000275 | GTTTGCCTTTGTGGTGCTGTGTT [symbol] | 50 bp, e3-4, 44% GC |
| | | albinism II | | ATTTTGTTCAGCCTATATCCGGA [symbol] | |
| | | | | C | |
| | | | | (SEQ ID NO: 579) | |
| M556 | OMP | olfactory marke [symbol] | NM_006189 | TGGCCAAGATCCGCAAGGTCAT [symbol] | 46 bp, 54% GC |
| | | protein | | ACTTCCTCGTCACCTTTGGCGA | |
| | | | | (SEQ ID NO: 580) | |
| M557 | TRP-2 | tyrosinase- | AJ000503 | TCTTCCACCGGACCTGCAAGTG [symbol] | 50 bp, e1-2, 50% GC |
| | | related | NM_001922 | CAGGAAACTTTGCCGGCTATAA [symbol] | |
| | | protein 2 | | GT | |
| | | (dopachrome | | (SEQ ID NO: 581) | |
| | | tautomerase) | | | |
| M558 | | | | TATTCTGTTAGAGATACATTATT [symbol] | 50 bp, e2-3, 46% GC |
| | | | | GGACCAGGACGCCCCTACAGGG [symbol] | |
| | | | | CAT | |
| | | | | (SEQ ID NO: 582) | |
| M559 | TYR | tyrosinase | NM_000372 | AGCATCATTCTTCTCCTCTTGGC [symbol] | 50 bp, e1-2, 48% GC |
| | | | | GATTGTCTGTAGCCGATTGGAG [symbol] | |
| | | | | GT | |
| | | | | (SEQ ID NO: 583) | |
| M560 | TYRP1 | tyrosinase- | NM_000550 | GCCTGTGACCAGAGGGTTCTCAT [symbol] | 50 bp, e2-3, 46% GC |
| | | related | | GTCAGGAGAAATCTTCTGGACTT [symbol] | |
| | | protein 1 | | AG | |
| | | | | (SEQ ID NO: 584) | |
| M561 | PNMT | phenylethanola [symbol] | NM_002686 | TGCCTCATTTGAGGGCAAGGGGG [symbol] | 41 bp, e2-3, 56% GC |
| | | ne N-methyl- | | ATGCTGGCAGGATAAGGA | |
| | | transferase | | (SEQ ID NO: 585) | |
| M562 | HGF | hepatocyte | D14012 | GCAGTGCACACAGGAAGTGGTG [symbol] | 50 bp, e4-5, 46% GC |
| | | growth | | GCCACAACTCACAACTACGAC | |
| | | factor | | (SEQ ID NO: 586) | |
| M563 | MET | hepatocyte | NM_000245 | GATCTGTCTGCCTGCAATCTACA [symbol] | 50 bp, e5-6, 46% GC |
| | | growth | | GGTTTTCCCAAATAGTGCACCC [symbol] | |
| | | factor | | TG | |
| | | receptor | | (SEQ ID NO: 587) | |
| M564 | NPY | neuropeptide | NM_000905 | ACATCAACCTCATCACCAGGCA [symbol] | 50 bp, e1-2, 50% GC |
| | | Y | | AGATATGGAAAACGATCCAGCC [symbol] | |
| | | | | AGAG | |
| | | | | (SEQ ID NO: 588) | |
| M565 | NPY2R | neuropeptide | NM_000910 | GCAAAAACGCGAGGTCCAGGT [symbol] | 49 bp, e1-2, 53% GC |
| | | Y receptor Y2 | | GTTGTAGACTCTTGTGCTGGTTG [symbol] | |
| | | | | AG | |
| | | | | (SEQ ID NO: 589) | |
| M566 | | neuropeptide | M84755 | ATTTATTTGCTACTTCAAGATAT [symbol] | 50 bp, e1-2, 28% GC |
| | | y receptor | | TATACGCCTAAAAAGGAGAAA [symbol] | |
| | | | | ACA | |
| | | | | (SEQ ID NO: 590) | |
| M567 | BDNF | brain-derived | NM_001709 | TGATAGAAGAGCTGTTGGATGA [symbol] | 50 bp, 44% GC |
| | | neurotrophic | | GACCATAAAGTTCGGCCCAATG [symbol] | |
| | | factor | | AGAA | |
| | | | | (SEQ ID NO: 591) | |
| M568 | NTF3 | neurotrophin 3 | NM_002527 | TTGCCAGAAGACTCGCTCAATT [symbol] | 50 bp, 46% GC |
| | | | | CTCATTATTAAGCTGATCCAGG [symbol] | |
| | | | | GA | |
| | | | | (SEQ ID NO: 592) | |
| M569 | NTF5 | neurotrophin 5 | NM_006179 | TCCGCCAGTACTTCTTTGAAAC [symbol] | 50 bp, e4-5, 46% GC |
| | | | | GCTGCAAGGCTGATAACGGTGA [symbol] | |
| | | | | GAA | |
| | | | | (SEQ ID NO: 593) | |
| M570 | OGFR | opioid growth | NM_007346 | TTGAGGAGAATCACTCCTACAT [symbol] | 50 bp, e4-5, 50% GC |
| | | factor receptor | | AGTGGCTGTTTCCTCTGCGAGAA [symbol] | |
| | | | | CA | |
| | | | | (SEQ ID NO: 594) | |
| M571 | OPRD1 | opioid receptor | NM_000911 | CTTGTCATGTTCGGCATCGTCCG [symbol] | 50 bp, e1-2, 52% GC |
| | | delta 1 | | TACACTAAGATGAAGACGGCCA [symbol] | |
| | | | | CAA | |
| | | | | (SEQ ID NO: 595) | |
| M572 | OPRK1 | opioid receptor | NM_000912 | CGCTGGTCATGTTCGTGATCAT [symbol] | 50 bp, e1-2, 46% GC |
| | | kappa 1 | | GATACAGAAAGATGAAGACAG [symbol] | |
| | | | | ACC | |
| | | | | (SEQ ID NO: 596) | |
| M573 | OPRM1 | opioid receptor | NM_000914 | TCCTGGTCATGTATGTGATTGTC [symbol] | 50 bp, e1-2, 46% GC |
| | | mu 1 | | GATACACCAAGATGAAGACTGC [symbol] | |
| | | | | ACC | |
| | | | | (SEQ ID NO: 597) | |
| M574 | VIPR1 | vasoactive | NM_004624 | ATGACAAGGCAGCGAGTTTGGA [symbol] | 50 bp, e3-4, 50% GC |
| | | intestinal | | GAGCAGCAGACCATGTITCTACG [symbol] | |
| | | peptide | | TCT | |
| | | receptor 1 | | (SEQ ID NO: 598) | |
| M575 | VIPR2 | vasoactive | XM_036413 | CAGCGACCCGGAGGATGAGAG [symbol] | 50 bp, e4-5, 50% GC |
| | | intestinal | | AGATCACGTTTTATATTCTGGTG [symbol] | |
| | | peptide | | AGG | |
| | | receptor 2 | | (SEQ ID NO: 599) | |
| M576 | AKR1C2 | aldo-keto | XM_048859 | ATTTGGCACCTATGCGCCTGCA [symbol] | 50 bp, e1-2, 48% GC |
| | | reductase | | GGTTCCTAAAAGTAAAGCTCTA [symbol] | may also detect |
| | | family 1, | | GG | AKR1C1, DD1 |
| | | member C2 | | (SEQ ID NO: 600) | |
| M577 | AKR1C3 | aldo-keto | NM_003739 | TCATTCTCCAATGTCTCTAAAG [symbol] | 50 bp, e3-4, 44% GC |
| | | reductase | | AGGTGAGGAACTTTCACCAACA [symbol] | |
| | | family 1, | | ATG | |
| | | member C3 | | (SEQ ID NO: 601) | |
| M578 | CYP8B1 | cytochrome P4 [symbol] | XM_046266 | ATGTTTGAATTTCTGAAGCGCAT [symbol] | 50 bp, 46% GC |
| | | subfamily VIIII [symbol] | | AGGACCAAGGATGGGGATGTGT [symbol] | |
| | | | | GAG | |
| | | | | (SEQ ID NO: 602) | |
| M579 | EBRP | emopamil | NM_032565 | CCATAGTCAAAGAAAAATATTA [symbol] | 50 bp, e3-4, 44% GC |
| | | binding | | CGGCATTTCCTGCAGATCACCCT [symbol] | |
| | | related | | TGC | |
| | | protein | | (SEQ ID NO: 603) | |
| M580 | HSD3A | oxidative 3 | U89281 | GGAAGCCTTTTCAGATATTCTGA [symbol] | 50 bp, e1-2, 44% GC |
| | | alpha hydroxy- | | GCGTGAGATTCAACATTTTGGG [symbol] | |
| | | steroid | | GA | |
| | | dehydrogenase | | (SEQ ID NO: 604) | |
| M581 | HSD3B | 3 Beta- | X53321 | TCTATCATGAATGTCAATGTGAA [symbol] | 50 bp, e2-3, 46% GC |
| | | hydroxysteroid | | GGTACCCAGCTCCTGTTAGAGG [symbol] | |
| | | dehydrogenase | | TG | |
| | | | | (SEQ ID NO: 605) | |
| M582 | RDHL | retinol | NM_005771 | GTGAAGAACCAAGTTGGGGAGA [symbol] | 50 bp, e1-2, 48% GC |
| | | dehydrogenase | | AGGTCTCTGGGGTCTGATCAATA [symbol] | will also detect |
| | | homolog | | TGC | hydroxysteroid |
| | | | | (SEQ ID NO: 606) | epimerase |
| | | | | | (NM_003725) |
| M583 | | sterol/retinol | AF057034 | TGAAGGAGTGCGTGAGAGACAA [symbol] | 50 bp, e1-2; 52% GC |
| | | dehydrogenase | | GGACTCTGGGGGCTGGTGAATA [symbol] | |
| | | | | GCT | |
| | | | | (SEQ ID NO: 607) | |
| M584 | TAC1 | tachykinin, | NM_003182 | GGATTAATGGGCAAACGGGATG [symbol] | 50 bp, e3-4, 44% GC |
| | | precursor 1 | | TGATTCCTCAATTGAAAAACAA [symbol] | |
| | | | | GGC | |
| | | | | (SEQ ID NO: 608) | |
| M585 | TACR1 | tachykinin | NM_001058 | GAGCAAGTCTCTGCCAAGCGCA [symbol] | 50 bp, e3-4, 50% GC |
| | | receptor 1 | | GGTGGTCAAAATGATGATTGTC [symbol] | will detect both |
| | | | | GGT | isoforms |
| | | | | (SEQ ID NO: 609) | |
| M586 | TACR2 | tachykinin | NM_001057 | ATCTACTGCTGTCTCAACCACA [symbol] | 50 bp, e4-5, 54% GC |
| | | receptor 2 | | TTTCGCTCTGGGTTCCGGCTTGC [symbol] | |
| | | | | TT | |
| | | | | (SEQ ID NO: 610) | |
| M587 | TACR3 | Tachykinin | NM_001059 | TCATCTACTGCTGTCTGAATAAA [symbol] | 50 bp, e4-5, 42% GC |
| | | receptor 3 | | GATTTCGAGCTGGCTTCAAGAGA [symbol] | |
| | | | | CA | |
| | | | | (SEQ ID NO: 611) | |
| M588 | TBG | thyroxine- | M14091 | GAGGACAATGGTCTGAAACTTT [symbol] | 50 bp, e4-5, 50% GC = |
| | | binding | | AATGCTGCCCATAAGGCTGTGCT [symbol] | serine (or cysteine) |
| | | globulin | | CA | proteinase inhibitor |
| | | | | (SEQ ID NO: 612) | |
| M589 | TPO | thyroid | NM_000547 | CCGCCATGTACGCCACGATGCA [symbol] | 50 bp, e2-3, 50% GC |
| | | peroxidase | | AGAAACCTCAAGAAAAGAGGAA [symbol] | |
| | | | | CCTT | |
| | | | | (SEQ ID NO: 613) | |
| M590 | CALB2b | calbindin 2 | NM_007087 | GCTCCAGGAATACACCCAAACC [symbol] | 50 bp, e5-6, 46% GC |
| | | | | TACTACGGATGTTTGACTTGAA [symbol] | |
| | | | | GGG | |
| | | | | (SEQ ID NO: 614) | |
| M591 | CALB3 | calbindin 3 | NM_004057 | GCTGAATTCCCCAGTTTACTCAA [symbol] | 50 bp, e2-3, 44% GC |
| | | | | GGTCCAAACACCCTAGATGATCT [symbol] | |
| | | | | TT | |
| | | | | (SEQ ID NO: 615) | |
| M592 | NCOA2 | nuclear | NM_006540 | AAAAACCTGCTGCCAAAGTCTAGG [symbol] | 50 bp, e6-7, 46% GC |
| | | receptor | | GTAAATGGGGGATCTTGGTCTGGAA [symbol] | |
| | | coactivator 2 | | GA | |
| | | | | (SEQ ID NO: 616) | |
| M593 | VDR | vitamin D | NM_000376 | GTGGACATCGGCATGATGAAGG [symbol] | 50 bp, e3-4, 48% GC |
| | | receptor | | GTTCATTCTGACAGATGAGGAA [symbol] | |
| | | | | GCA | |
| | | | | (SEQ ID NO: 617) | |
| M594 | VDBP | vitamin D | NM_000583 | CTCTGAAGATTGCATGGCCAAA [symbol] | 50 bp, e7-8, 46% GC |
| | | binding | | AGCTGCCTGAACACACAGTAAA [symbol] | |
| | | protein | | CTCT | |
| | | | | (SEQ ID NO: 618) | |
| M595 | BMP1 | bone | NM_006128 | TCGTAAGTCCTCCATCAAAGCT [symbol] | 50 bp, e2-3, 50% GC |
| | | morphogenetic | | AGTTCCAGGAAACACTTCTACC [symbol] | |
| | | protein 1 | | CA | |
| | | | | (SEQ ID NO: 619) | |
| M596 | BMP10 | bone | NM_014482 | CATCATTAGGAGTTTCAAGAAT [symbol] | 50 bp, e1-2, 42% GC |
| | | morphogenetic | | AGATCTGTTTTCCCAGCCGGTCA [symbol] | detects almost all |
| | | protein 10 | | TT | splicing variants |
| | | | | (SEQ ID NO: 620) | |
| M597 | BMP11 | bone | AF100907 | GTCATTAGCATGGCCCAGGAGA [symbol] | 45 bp, e1-2, 55% GC |
| | | morphogenetic | | GGACCCAGCAGTACAGACAGAT [symbol] | |
| | | protein 11 | | (SEQ ID NO: 621) | |
| M598 | BMP15 | bone | NM_005448 | ACCAGTGTGGCAAGGCCTCACA [symbol] | 47 bp, e1-2, 53% GC |
| | | morphogenetic | | AGGTACCTGGCATATACAGATC [symbol] | |
| | | protein 15 | | (SEQ ID NO: 622) | |
| M599 | BMP2 | bone | NM_00120 | ACTGTGCGCAGCTTCCACCATGA [symbol] | 50 bp, e2-3, 46% GC |
| | | morphogenetic | | GAATCTTTGGAAGAACTACCAG [symbol] | |
| | | protein 2 | | AAC | |
| | | | | (SEQ ID NO: 623) | |
| M600 | BMP3 | bone | NM_001201 | CAGCTTTCGGGCGGCAGCAGCA [symbol] | 50 bp, e1-2, 48% GC |
| | | morphogenetic | | AAACTCTTGAAAGAAAAGGACT [symbol] | |
| | | protein 3 | | TATA | |
| | | | | (SEQ ID NO: 624) | |
| M601 | BMP4 | bone | NM_001202 | AGCTTCCACCACGAAGAACATC [symbol] | 50 bp, e3-4, 50% GC |
| | | morphogenetic | | GAGAACATCCCAGGGACCAGTG [symbol] | |
| | | protein 4 | | AAA | |
| | | | | (SEQ ID NO: 625) | |
| M602 | BMP5 | Bone | NM_021073 | CAGCTCTGTGCAGAAACAGGGG [symbol] | 50 bp, e1-2, 48% GC |
| | | morphogenetic | | TGGACGCAGTATCAACGTAAAA [symbol] | |
| | | protein 5 | | GTGC | |
| | | | | (SEQ ID NO: 626) | |
| M603 | BMP6 | bone | NM_001718 | ACATGGTCATGAGCTTTGTGAA [symbol] | 50 bp, e1-2, 50% GC |
| | | morphogenetic | | TGGTGGAGTACGACAAGGAGTT [symbol] | |
| | | protein 6 | | TCC | |
| | | | | (SEQ ID NO: 627) | |
| M604 | BMP7 | bone | NM_001719 | ATGGTCATGAGCTTGGTCAACCT [symbol] | 50 bp, e1-2, 46% GC = |
| | | morphogenetic | | GTGGAACATGACAAGGAATTCT [symbol] | TGF beta? |
| | | protein 7 | | CA | |
| | | | | (SEQ ID NO: 628) | |
| M605 | BMP8 | bone | NM_001720 | AGGTGGTCCAGGAGCAGTCCAA [symbol] | 50 bp, e3-4, 50% GC |
| | | morphogenetic | | AGGGAGTCTGACTTGTTCTTTTT [symbol] | |
| | | protein 8 | | GAT | |
| | | | | (SEQ ID NO: 629) | |
| M606 | BMP9 | bone | AF188285 | ATTGTGCGGAGCTFCAGCATGGA [symbol] | 50 bp, e1-2, 50% GC |
| | | morphogenetic | | GATGCCATCTCCATAACTGCCA [symbol] | |
| | | protein 9 | | GA | |
| | | | | (SEQ ID NO: 630) | |
| M607 | BMPR1A | bone | NM_004329 | AGTGGGTCTGGACTACCTTTATT [symbol] | 50 bp, e8-9, 44% GC |
| | | morphogenetic | | GTTCAGCGAACTATTGCCAAACA [symbol] | |
| | | protein | | AT | |
| | | receptor | | (SEQ ID NO: 631) | |
| | | type IA | | | |
| M608 | BMPR1B | bone | NM_001203 | GAAGGCTCAGATTTTCAGTGTC [symbol] | 50 bp, e5-6, 48% GC |
| | | morphogenetic | | GACACTCCCATTCCTCATCAAA [symbol] | |
| | | protein | | AG | |
| | | receptor | | (SEQ ID NO: 632) | |
| | | type IB | | | |
| M609 | BMPR2 | bone | NM_001204 | TGCTTTGGATACAGAATGTTGA [symbol] | 50 bp, e4-5, 44% GC |
| | | morphogenetic | | GGAGACCGTAAACAAGGTCTTC [symbol] | |
| | | protein | | CAG | |
| | | receptor | | (SEQ ID NO: 633) | |
| | | type 2 | | | |
| M610 | GDF1 | growth | NM_001492 | TCTTCTTTGACAACAGCGACAA [symbol] | 49 bp, e2, 51% GC |
| | | differentiation | | TGGTGCTGCGGCAGTATGAGGA [symbol] | |
| | | factor 1 | | AT | |
| | | | | (SEQ ID NO: 634) | |
| M611 | GDF1 | growth | NM_004962 | CATGTGAGTTCCCCATGCCTAA [symbol] | 48 bp, e3-4, 52% GC |
| | | differentiation | | TCGTTCGTCCATCCAACCATGC [symbol] | |
| | | factor 10 | | (SEQ ID NO: 635) | |
| M612 | GDF3 | Growth | NM_020634 | GGAATGTACTTCGCTTTCTCCCA [symbol] | 50 bp, e1-2, 48% GC |
| | | differentiation | | ACCAAGGTTTCTTTCTTTACCCA [symbol] | |
| | | factor 3 | | AG | |
| | | | | (SEQ ID NO: 636) | |
| M613 | GDF7 | Growth | NM_000557 | ACCAGCTTTATTGACAAAGGGC [symbol] | 50 bp, e1-2, 52% GC |
| | | differentiation | | AGATGACCGAGGTCCCGTGGTC [symbol] | |
| | | factor 7 | | GGAA | |
| | | | | (SEQ ID NO: 637) | |
| M614 | GDF8 | Growth | NM_005259 | CAGGACCAGGAGAAGATGGGCT [symbol] | 50 bp, e2-3, 48% GC |
| | | differentiation | | AATCCGTTTTTAGAGGTCAAGGT [symbol] | |
| | | factor 8 | | ACA | |
| | | | | (SEQ ID NO: 638) | |
| M615 | GDF9 | Growth | NM_005260 | AAGCAGGCTCCTGGAGACCAGG [symbol] | 48 bp, e1-2, 52% GC |
| | | differentiation | | AACAGGAATCCTTCCATCAGTG [symbol] | |
| | | factor 9 | | A | |
| | | | | (SEQ ID NO: 639) | |
| M616 | AQP1 | aquaporin 1 | NM_000385 | TGTAGCCCTTGGACACCTCCTG [symbol] | 50 bp, e2-3, 52% GC |
| | | | | TATTGACTACACTGGCTGTGGGA [symbol] | |
| | | | | TA | |
| | | | | (SEQ ID NO: 640) | |
| M617 | AQP10 | aquaporin 10 | NM_080429 | GGAGCCACCTATGTTCTCTACCA [symbol] | 50 bp, e3-4, 50% GC |
| | | | | GATGCCCTACAGAACTATACAG [symbol] | |
| | | | | GG | |
| | | | | (SEQ ID NO: 641) | |
| M618 | AQP2 | aquaporin 2 | NM_000486 | TGTCGTCACTGGCAAATTTGAT [symbol] | 48 bp, e3-4, 52% GC |
| | | | | CCACTGGGTCTTCTGGATCGGA [symbol] | |
| | | | | (SEQ ID NO:642) | |
| M619 | AQP3 | aquaporin 3 | NM_004925 | CTGGAATAGTTTTTGGGCTGTAT [symbol] | 50 bp, e3-4, 44% GC |
| | | | | ATGATGCAATCTGGCACTTTGC [symbol] | |
| | | | | AC | |
| | | | | (SEQ ID NO: 643) | |
| M620 | AQP4 | aquaporin 4 | NM_001650 | CAGTTATCATGGGAAATTGGGA [symbol] | 50 bp, e4-5, 48% GC |
| | | | | AACCAUGGATATATTGGGTTG [symbol] | |
| | | | | CCC | |
| | | | | (SEQ ID NO: 644) | |
| M621 | AQP5 | aquaporin 5 | NM_001651 | ACCCTGGGCCACCTTGTCGGAAT [symbol] | 47 bp, e4-5, 55% GC |
| | | | | TACTTCACTGGCTGCTCCATGAA [symbol] | |
| | | | | (SEQ ID NO: 645) | |
| M622 | AQP6 | aquaporin 6 | XM_113241 | ATCCGAGAGACCCTTGGGATCA [symbol] | 48 bp, e1-2, 54% GC |
| | | | | CGTGGTCCGGAACAGTGTCTCAA [symbol] | |
| | | | | T | |
| | | | | (SEQ ID NO: 646) | |
| M623 | AQP7 | aquaporin 7 | NM_001170 | AGTTCATGAGCACATATGTCAT [symbol] | 50 bp, e3-4, 50% GC |
| | | | | TGGTATTCGGCCTTGGTTCCGTT [symbol] | |
| | | | | CC | |
| | | | | (SEQ ID NO: 647) | |
| M624 | AQP8 | aquaporin 8 | NM_001169 | GCCATCTGATCCTGATGTCTGGA [symbol] | 50 bp, e1-2, 48% GC |
| | | | | AGATAGCCATGTGTGAGCCTGA [symbol] | |
| | | | | TT | |
| | | | | (SEQ ID NO: 648) | |
| M625 | AQP9 | aquaporin 9 | NM_020980 | CGTTCATCTTGATTGTCCTTGGA [symbol] | 50 bp, e1-2, 48% GC |
| | | | | GTGGCTGTGTTGCCCAAGCTATT [symbol] | |
| | | | | TC | |
| | | | | (SEQ ID NO: 649) | |
| M626 | SLC6A1 | neurotransmitter | NM_003042 | TACCTGTACAACTCCTTCACCA [symbol] | 50 bp, e4-5, 52% GC |
| | | transporter, | | ACACTGCCGTGGAAACAGTGCG [symbol] | |
| | | GABA | | CAA | |
| | | | | (SEQ ID NO: 650) | |
| M627 | SLC6A11 | neurotransmitter | NM_014229 | GGAGGAAAGTTTGCCCTTTATTT [symbol] | 50 bp, e2-3, 46% GC |
| | | transporter, | | AAGGCATTGGCTATGCAACACA [symbol] | |
| | | GABA | | GTG | |
| | | | | (SEQ ID NO: 651) | |
| M628 | SLC6A9 | neurotransmitter | | TGGAGGATCAGCCCCATGTTCAA [symbol] | 50 bp, e3-4, 52% GC |
| | | transporter 9 | | GGAGTGGGCTATGGTATGATGG [symbol] | |
| | | | | GGT | |
| | | | | (SEQ ID NO: 652) | |
| M629 | SLC6A13 | neurotransmitter | NM_016615 | TATCTCTGCTACAAAAATGGGG [symbol] | 50 bp, e1-2, 50% GC |
| | | transporter, | | GGTGCCTTCTTCATCCCCTACCT [symbol] | may detect also |
| | | GABA | | GT | SLC6A12 |
| | | | | (SEQ ID NO: 653) | |
| M630 | SLC6A14 | neurotransmitter | NM_007231 | GTTTCTTCAAGTGCAGGGAGAA [symbol] | 50 bp, e1-2, 46% GC |
| | | transporter | | GAGAAAGTGTCGGCTTCATCAG [symbol] | |
| | | | | GAAT | |
| | | | | (SEQ ID NO: 654) | |
| M631 | SLC6A2 | neurotransmitter | NM_001043 | TGGAAAATCTGCCCATTCTTCAA [symbol] | 50 bp, e2-3, 48% GC |
| | | transporter, | | GGCGTTGGCTATGCTGTCATCCT [symbol] | |
| | | noradrenalin | | AT | |
| | | | | (SEQ ID NO: 655) | |
| M632 | SLC6A3 | neurotransmitter | NM_001044 | AACAAGTTCACCAACAACTGCTA [symbol] | 50 bp, e7-8, 50% GC |
| | | transporter, | | AGGGACGCGATTGTCACCACCT [symbol] | |
| | | dotamine | | AT | |
| | | | | (SEQ ID NO: 656) | |
| M633 | SLC6A4 | neurotransmitter | NM_001045 | ACAACAAGTTCAACAACAACTG [symbol] | 50 bp, e6-7, 50% GC |
| | | transporter, | | TACCAAGATGCCCTGGTGACCA [symbol] | |
| | | serotonin | | GTG | |
| | | | | (SEQ ID NO: 657) | |
| M634 | SLC6A5 | neurotransmitter | NM_004211 | AGACTTCAGGAAAAGTGGTGTA [symbol] | 50 bp, e7-8, 50% GC |
| | | transporter, | | TTCACGGCCACGTTCCCGTATGT [symbol] | |
| | | glycine | | GTA | |
| | | | | (SEQ ID NO: 658) | |
| M635 | SLC6A6 | neurotransmitter | NM_003043 | ACAAGTACAAGTATAACTCGTA [symbol] | 50 bp, e3-4, 48% GC |
| | | transporter, | | AGGGACTGTATGCTGCTGGGAT [symbol] | |
| | | taurine | | CTG | |
| | | | | (SEQ ID NO: 659) | |
| M636 | SLC6A7 | neurotransmitter | NM_014228 | CCACCACTTGTTGTCTTCCAAGG [symbol] | 50 bp, e6-7, 48% GC |
| | | transporter, L- | | GTGGATTGAAGCTGCTCTTCAGA [symbol] | |
| | | proline | | CT | |
| | | | | (SEQ ID NO: 660) | |
| M637 | SLC6A8 | neurotransmitter | NM_005629 | CGCTTCAACAACAACTGCTACAA [symbol] | 50 bp, e6-7, 50% GC |
| | | transporter, | | GACGCCATCATCCTGGCTCTCAT [symbol] | |
| | | creatine | | AA | |
| | | | | (SEQ ID NO: 661) | |
| M638 | GABBR1 | gamma- | NM_001470 | CAATACCCGCAGCATTTCCAACA [symbol] | 50 bp, e9-10, 46% GC |
| | | aminobutyric a [symbol] | | GACATCCCAGGAATTTGTGGAG [symbol] | |
| | | B receptor, 1 | | AAC | |
| | | | | (SEQ ID NO: 662) | |
| M639 | GABRA1 | gamma- | NM_000806 | TTCTGAGCACACTGACTGGAAGA [symbol] | 50 bp, e2-3, 46% GC |
| | | aminobutyric a [symbol] | | AGCTATGGACAGCCGTCATTACA [symbol] | |
| | | A receptor, | | GAT | |
| | | alpha 1 | | (SEQ ID NO: 663) | |
| M640 | GABRA2 | gamma- | NM_000807 | GATAATCGGCTTAGACCAGGAC [symbol] | 50 bp, e3-4, 46% GC |
| | | aminobutyric a [symbol] | | GGGAGACAGTATTACTGAAGTC [symbol] | |
| | | A receptor, | | GAG | |
| | | alpha 2 | | (SEQ ID NO: 664) | |
| M641 | GABRA3 | gamma- | NM_000808 | CTGCGACCTGGGCTTGGAGATG [symbol] | 50 bp, e3-4, 52% GC |
| | | aminobutyric a [symbol] | | GTGACTGAAGTGAAGACTGACA [symbol] | |
| | | A receptor, | | CTA | |
| | | alpha 3 | | (SEQ ID NO: 665) | |
| M642 | GABRA4 | gamma- | NM_000809 | AGGCTGCGTCCTGGATTTGGGG [symbol] | 50 bp, e2-3, 50% GC |
| | | aminobutyric a [symbol] | | CCTGTTACAGAAGTGAAAACTG [symbol] | |
| | | A receptor, | | CAT | |
| | | alpha 4 | | (SEQ ID NO: 666) | |
| M643 | GABRA5 | gamma- | NM_000810 | ACTGAGAACATCAGCACCAGCA [symbol] | 50 bp, e1-2, 48% GC |
| | | aminobutyric a [symbol] | | AGGCGAATACACAATCATGACA [symbol] | |
| | | A receptor, | | CTCA | |
| | | alpha 5 | | (SEQ ID NO: 667) | |
| M644 | GABRA6 | gamma- | NM_000811 | ATGGAACCATTTTATACACCAT [symbol] | 50 bp, e4-5, 46% GC |
| | | aminobutyric a [symbol] | | GGCTTACCATCAATGGTGACTGT [symbol] | |
| | | A receptor, | | CC | |
| | | alpha 6 | | (SEQ ID NO: 668) | |
| M645 | GABRB1 | gamma- | NM_000812 | TGATGGAACAGTTCTCTATGGA [symbol] | 50 bp, e4-5, 46% GC |
| | | aminobutyric a [symbol] | | CCGAATCACAACCACAGCTGCA [symbol] | |
| | | A receptor, | | GTA | |
| | | beta | | (SEQ ID NO: 669) | |
| M646 | GABRB2 | gamma- | XM_011222 | ACCAAGAAGGTTGTTTTTTCCA [symbol] | 50 bp, e1-2, 48% GC |
| | | aminobutyric a [symbol] | | GGTTCCTATCCCAGGTTATCCCT [symbol] | |
| | | A receptor, | | AG | |
| | | beta | | (SEQ ID NO: 670) | |
| M647 | GABRB3 | gamma- | XM_007751 | GAACTGCACTCTGGAAATTGAA [symbol] | 50 bp, e4-5, 46% GC |
| | | aminobutyric a [symbol] | | GCTATGGCTACACCACGGATGA [symbol] | |
| | | A receptor, | | ATTG | |
| | | beta | | (SEQ ID NO: 671) | |
| M648 | GABRD | gamma- | NM_000815 | AGCATCGACCACATCTCAGAGG [symbol] | 50 bp, e2-3, 52% GC |
| | | aminobutyric a [symbol] | | CAACATGGAGTACACCATGACG [symbol] | |
| | | A receptor, | | TGTT | |
| | | delta | | (SEQ ID NO: 672) | |
| M649 | GABRE | gamma- | NM_004961 | GGATGGCAAGGTGTTGTACACA [symbol] | 50 bp, e4-5, 48% GC |
| | | aminobutyric a [symbol] | | TTAGGATGACCATTGATGCCGGA [symbol] | |
| | | A receptor, | | GCT | |
| | | epsilon | | (SEQ ID NO: 673) | |
| M650 | GABRG2 | gamma- | NM_000816 | TGGTCGAGTGCTCTACTCCCTAA [symbol] | 50 bp, e4-5, 48% GC |
| | | aminobutyric a [symbol] | | GTTGACAATTGATGCTGAGTGC [symbol] | |
| | | A receptor, | | AT | |
| | | gamma 2 | | (SEQ ID NO: 674) | |
| M651 | GABRG3 | gamma- | NM_033223 | AAAAGATGCTACGCCAGCAAGA [symbol] | 50 bp, e6-7, 50% GC |
| | | aminobutyric a [symbol] | | CAGCATTAGGCATCACCACGGT [symbol] | |
| | | A receptor, | | TGA | |
| | | gamma 3 | | (SEQ ID NO: 675) | |
| M652 | GABRP | gamma- | XM_003708 | AATTTCTCAGGCCCAATTTTGGT [symbol] | 50 bp, e3-4, 48% GC |
| | | aminobutyric a [symbol] | | GAGAACCCGTACAGATAGCGCT [symbol] | |
| | | A receptor, pi | | ACT | |
| | | | | (SEQ ID NO: 676) | |
| M653 | GABRR1 | gamma- | NM_002042 | ATGAAGATGCCCACAAGCAAGT [symbol] | 50 bp, e2-3, 50% GC |
| | | aminobutyric a [symbol] | | AGCCCAATTCTGAGACGAAGTC [symbol] | |
| | | A receptor, rho | | GAC | |
| | | | | (SEQ ID NO: 677) | |
| M654 | GABR2 | gamma- | NM_002043 | CAGACCTGTTCTTTGGAGCTGGA [symbol] | 50 bp, e1-2, 48% GC |
| | | aminobutyric a [symbol] | | AGCTATGCCTATACAGATGAAG [symbol] | |
| | | A receptor, rho | | CT | |
| | | | | (SEQ ID NO: 678) | |
| M655 | CRH-R1 | | | GGGCCCCATGATCCTGGTCCTG [symbol] | 50 bp, e10-11, 48% G [symbol] |
| | | | | GATCAATTTCATCTTCCTTTTCAA [symbol] | |
| | | | | A | |
| | | | | (SEQ ID NO: 679) | |
| M656 | | | | CCATGATCCTGGTCCTGCTGAT [symbol] | 40 bp, e1-2, 42% GC |
| | | | | ATTTCATCTTCCTTTT | |
| | | | | (SEQ ID NO: 680) | |
| M657 | | | | ATCCTGGTCCTGCTGATCAATTT [symbol] | 30 bp, e1-2, 42% GC |
| | | | | ATCTTC | |
| | | | | (SEQ ID NO: 681) | |
| M658 | | Insulin | | AGCTGGAGAACTACTGCAACTA [symbol] | 40 bp |
| | | | | ACGCAGCCCGCAGGCAG | |
| | | | | (SEQ ID NO: 682) | |
| M659 | | Insulin | | CACCACTTCTTCTGACATGTGGT [symbol] | 45 bp |
| | | receptor | | CTTTGGCGTGGTCCTTTGGGA | |
| | | | | (SEQ ID NO: 683) | |
| M660 | | Insulin | | TGTCACAAGCACCAGCCTTGCCA [symbol] | 45 bp |
| | | degradi [symbol] | | AACCTGAAGTGATTCAGAACA | |
| | | enzyme | | (SEQ ID NO: 684) | |
| M661 | | Proenkephalin | | GCCGACATCAACTTCCTGGCTT [symbol] | 45 bp |
| | | | | GTAATGGAATGTGAAGGTAAA | |
| | | | | (SEQ ID NO: 685) | |
| M662 | | Pituitary | | GGATCAGGCCAGAGGAAGAGG [symbol] | 40 bp |
| | | adenylate cycla [symbol] | | TACGGCGAGGACGGAAA | |
| | | activating | | (SEQ ID NO: 686) | |
| | | peptide | | | |
| M663 | | Pituitary | | GCAACCACTGCTTCATCTCCACT [symbol] | 45 bp Detects recepto [symbol] |
| | | adenylate cycla [symbol] | | TGGAATGTAAGGCCGTCATGG | A, B, B2, C |
| | | activating | | (SEQ ID NO: 687) | |
| | | peptide | | | |
| | | recetor A | | | |
| M664 | PC1 | PC1 (NEC1 | M90753 | AGAAGATGGTGGATCCAGGGGA [symbol] | 45 bp |
| | | | | GAGCAGCCCACACAAGAGAAC [symbol] | |
| | | | | (SEQ ID NO: 688) | |
| M665 | PC2 | proprotein | NM_002594 | AGGAAAAGGAACCCCGAGGCC [symbol] | |
| | | convertase | | TGTGGCAACCACAGATTTGTAC | |
| | | subtilisin/kexin | | (SEQ ID NO: 689) | |
| | | type 2 | | | |
| M666 | | polypeptide 7B | Y00757 | GAAGGGAGGAGAGAGACGAAA [symbol] | |
| | | | | CGGAGGAGTGTCAATCCATATC [symbol] | |
| | | | | (SEQ ID NO: 690) | |
| M667 | COL14A | collagen, type | XM_044622 | CAGTGTTTATACAAAGCTCCAG [symbol] | 50 bp, 42% GC |
| | | XIV, alpha 1 | | GATTGAAGGACCTAGTGTGAGC [symbol] | |
| | | | | TAA | |
| | | | | (SEQ ID NO: 691) | |
| M668 | COL1A1 | collagen, type | NM_000088 | ATGCCATCAAAGTCTTCTGCAA [symbol] | 50 bp, 50% GC |
| | | I alpha 1 | | TGGAGACTGGTGAGACCTGCGT [symbol] | |
| | | | | AC | |
| | | | | (SEQ ID NO: 692) | |
| M669 | COL1A2 | collagen, type | NM_000089 | TGAACTTGTTGCTGAGGGCAACA [symbol] | 50 bp, 46% GC |
| | | I alpha 2 | | CAGGTTCACTTACACTGTTCTTG [symbol] | |
| | | | | AG | |
| | | | | (SEQ ID NO: 693) | |
| M670 | COL3A1 | collagen, type | NM_000090 | GTAGATATTGCACCCTATGACAT [symbol] | 50 bp, 42% GC; dete [symbol] |
| | | I alpha 1 | | GGTGGTCCTGATCAAGAATTTG [symbol] | also COL5A2 |
| | | | | GT | |
| | | | | (SEQ ID NO: 694) | |
| M671 | COL5A1 | collagen, type [symbol] | NM_000093 | GGACATCATGTTCAATGACTTC [symbol] | 50 bp, 42% GC |
| | | alpha 1 | | TGAAGCGTCACAGAAATTTGGA [symbol] | |
| | | | | TG | |
| | | | | (SEQ ID NO: 695) | |
| M672 | COL7A1 | collagen, type | NM_000094 | AGTATTCTGTGGAGGAGTACCA [symbol] | 50 bp, 50% GC |
| | | VII, alpha 1 | | GACCCTGAAGCTCCTTGGGATA [symbol] | |
| | | | | GAT | |
| | | | | (SEQ ID NO: 696) | |
| M673 | FN1 | fibronectin 1 | NM_002026 | TGAGTGCTTCATGCCTTTAGATG [symbol] | |
| | | | | ACAGGCTGACAGAGAAGATTCC [symbol] | |
| | | | | GAG | |
| | | | | (SEQ ID NO: 697) | |
| M674 | LAMA2 | laminin, | NM_000426 | ATCAGATCCCTGAAGCTCACCAA [symbol] | |
| | | alpha 2 | | GGCACAGCAAGCCACTGGAGGT [symbol] | |
| | | (merosin) | | AAT | |
| | | | | (SEQ ID NO: 698) | |
| M675 | LAMB2 | laminin, | NM_002292 | GACCAGCATCTTGACTTGCTCAA [symbol] | |
| | | beta 2 | | CATTCAAACTTCCTGGGTGCCTA [symbol] | |
| | | | | GA | |
| | | | | (SEQ ID NO: 699) | |
| M676 | LAMB3 | laminin, | NM_000228 | GATTGGGTTGGAATGCTTTCCAT [symbol] | |
| | | beta 3 | | TCCAGGAGACTTTCATGCAGCCT [symbol] | |
| | | | | AA | |
| | | | | (SEQ ID NO: 700) | |
| M677 | LAMC2 | laminin, | NM_018891 | TTACAATCCAAGACACACTCAA [symbol] | |
| | | gamma | | CATTAGACGGCCTCCTGCATCT [symbol] | |
| | | | | TG | |
| | | | | (SEQ ID NO: 701) | |
| M678 | LOX | lysyl | NM_002317 | GACTATACCAACAATGTTGTGC [symbol] | |
| | | oxidase | | TGTGACATTCGCTACACAGGACA [symbol] | |
| | | | | CA | |
| | | | | (SEQ ID NO: 702) | |
| M679 | MMP1 | interstitial | NM_002421 | ATCTACAGCTCCTTTGGCTTCCC [symbol] | |
| | | collagenase | | AGAACTGTGAAGCATATCGATG [symbol] | |
| | | | | GC | |
| | | | | (SEQ ID NO: 703) | |
| M680 | MMP10 | stromelysin 2 | NM_002425 | TCAGTGGATCATCACAGTTTGA [symbol] | |
| | | | | TTGACCCCAATGCCAGGATGGT [symbol] | |
| | | | | CA | |
| | | | | (SEQ ID NO: 704) | |
| M681 | MMP11 | stromelysin 3 | NM_005940 | CCTCTACTGGAAGTTTGACCCT [symbol] | |
| | | | | GAAGGTGAAGGCTCTGGAAGG [symbol] | |
| | | | | T | |
| | | | | (SEQ ID NO: 705) | |
| M682 | MMP3 | stromelysin 1 | NM_002422 | AGGAAATCAATTCTGGGCCATC [symbol] | |
| | | | | GAGGAAATGAGGTACGAGCTG [symbol] | |
| | | | | TACC | |
| | | | | (SEQ ID NO: 706) | |
| M683 | MMP9 | gelatinase B | NM_004994 | AGTGAGTTGAACCAGGTGGACC [symbol] | |
| | | | | AGTGGGCTACGTGACCTATGAC [symbol] | |
| | | | | (SEQ ID NO: 707) | |
| M684 | PLOD | lysine | NM_000302 | AGAACGTGCCGACTATTGACAT [symbol] | |
| | | hydroxylase | | ACATGAACCAGATCGGCTTTGA [symbol] | |
| | | | | (SEQ ID NO: 708) | |
| M685 | | prolyl 4- | X05130 | AGGAGCCAGACATGGAGGAAGA [symbol] | |
| | | hydoxylase | | GATGACCAGAAAGCTGTGAAAG [symbol] | |
| | | beta subunit | | TGAA | |
| | | | | (SEQ ID NO: 709) | |
| M686 | | prolyl 4- | U12762 | GTCTTCACTGAGGCCAAGTCTA [symbol] | |
| | | hydroxylase | | ATTCTGCCCACCAAGAATGATG [symbol] | |
| | | alpha | | CT | |
| | | subunit | | (SEQ ID NO: 710) | |
| M687 | TNXB | tenascin XB | NM_019105 | ACTATGCCCATGGTTTTGGGAA [symbol] | |
| | | | | TCTCTGGAGAGTTCTGGCTGGG [symbol] | |
| | | | | AT | |
| | | | | (SEQ ID NO: 711) | |
| M688 | VTN | vitronectin | NM_000638 | CTTCTTCTTCTCTGGAGACAAGT [symbol] | |
| | | | | CTACCGAGTCAATCTTCGCACA [symbol] | |
| | | | | GC | |
| | | | | (SEQ ID NO: 712) | |
| M689 | FLG | filaggrin | XM_048104 | ATCATCCCATGAACAGGCAAGA [symbol] | |
| | | | | CAAGTGCAGGAGAGAGACATG [symbol] | |
| | | | | TCCC | |
| | | | | (SEQ ID NO: 713) | |
| M690 | IVL | involucrin | NM_005547 | AGAGCAGCAGGTAGGACAGCCA [symbol] | |
| | | | | AGCACCTGGAACAGCAGGAAA [symbol] | |
| | | | | CA | |
| | | | | (SEQ ID NO: 714) | |
| M691 | KRT1 | keratin 1 | NM_006121 | CAGTTCCAGCGTGAGGTTTGTTT [symbol] | |
| | | | | TACCACTTATTCCGGAGTAACCA [symbol] | |
| | | | | AT | |
| | | | | (SEQ ID NO: 715) | |
| M692 | KRT10 | keratin 10 | NM_000421 | CTGAAACCGAGTGCCAGAATAC [symbol] | |
| | | | | GAATACCAACAACTCACGGATA [symbol] | |
| | | | | AAG | |
| | | | | (SEQ ID NO: 716) | |
| M693 | KRT14 | keratin 14 | NM_000526 | TGACCAGTATGAGAAGATGGCA [symbol] | |
| | | | | AGAAGAACCGCAAGGATGCCGA [symbol] | |
| | | | | GAAT | |
| | | | | (SEQ ID NO: 717) | |
| M694 | KRT17 | keratin 17 | NM_000422 | GAGATGCGTGACCAGTATGAGA [symbol] | |
| | | | | GATGGCAGAGAAGAACCGCAA [symbol] | |
| | | | | ATG | |
| | | | | (SEQ ID NO: 718) | |
| M695 | KRT19 | keratin 19 | NM_002276 | TGCTCGAGGGACAGGAAGATCA [symbol] | |
| | | | | TACAACAATTTGTCTGCCTCCAA [symbol] | |
| | | | | GTC | |
| | | | | (SEQ ID NO: 719) | |
| M696 | KRT5 | keratin 5 | NM_000424 | GAAGAGCTTCAAGAGCTAAGAA [symbol] | |
| | | | | CTGCTGCAAGTCACTGCCTTCCA [symbol] | |
| | | | | GTG | |
| | | | | (SEQ ID NO: 720) | |
| M697 | KRTHA5 | keratin, | NM_002280 | TGGAGATTGAGCTGCAGGCTCA [symbol] | |
| | | hair, | | CACAGCATGAGAGATGCTTTGG [symbol] | |
| | | acidic, 5 | | AT | |
| | | | | (SEQ ID NO: 721) | |
| M698 | KRTHB5 | keratin, | NM_002283 | ATCACTGCACTGAATGGCATGT [symbol] | |
| | | hair, | | ATGGAAAATGTGTGCTTGCTTC [symbol] | |
| | | basic, 5 | | GA | |
| | | | | (SEQ ID NO: 722) | |
| M699 | LOR | loricrin | NM_000427 | TGCAAATCCTTCATGTCTTAAC [symbol] | |
| | | | | ACCTGGAAGAAGCCATTGAGCT [symbol] | |
| | | | | CC | |
| | | | | (SEQ ID NO: 723) | |
| M700 | TGMI | transgluta- | NM_000359 | ACAGTGAAACTGCACCTCTACCT [symbol] | |
| | | minase 1 | | TCAGTCACTTTCTATACTGGTGT [symbol] | |
| | | | | AG | |
| | | | | (SEQ ID NO: 724) | |
| M701 | TGM2 | transgluta- | NM_004613 | TCCTCTTTGCATCCTCTATGAGA [symbol] | |
| | | minase 2 | | ATACCGTGACTGCCTTACGGAGT [symbol] | |
| | | | | CA | |
| | | | | (SEQ ID NO: 725) | |
| M702 | TGM3 | transgluta- | NM_003245 | AAGAGGCAGAACATCCCATAAA [symbol] | |
| | | minase 3 | | ATCTCGTACGCTCAGTATGAGA [symbol] | |
| | | | | TAG | |
| | | | | (SEQ ID NO: 726) | |
| M703 | THH | trichohyalin | NM_007113 | CTTAAGTGATGTTGCCAATATCT [symbol] | |
| | | | | GACACCTGCCAAAGCTTCCAGCA [symbol] | |
| | | | | GG | |
| | | | | (SEQ ID NO: 727) | |
| M704 | TRHY | trichohyalin | L09190 | CAATACCGCCCTTAAGTGATGTT [symbol] | |
| | | | | CCAATATCTTGACACCTGCCAAA [symbol] | |
| | | | | CT | |
| | | | | (SEQ ID NO: 728) | |
| M705 | | agouti | U12770 | TATATAGGCTGCTCGAAGGTGT [symbol] | |
| | | | | GGCTGTTTCTGTAAAGGTCCCGA [symbol] | |
| | | | | AG | |
| | | | | (SEQ ID NO: 729) | |
| M706 | AGRP | agouti | NM_001138 | TTCTTCAATGCCTTCTGCTACTG [symbol] | |
| | | related | | GCAAGCTGGGTACTGCCATGAA [symbol] | |
| | | protein | | (SEQ ID NO: 730) | |
| M707 | CCS | copper | NM_005125 | CAGAATGGAGGATGAGCAGCTG [symbol] | |
| | | chapero [symbol] | | AGGTGTGGGATGTGATTGG | |
| | | for super- | | (SEQ ID NO: 731) | |
| | | oxide | | | |
| | | dismutase | | | |
| M708 | GCH1 | GTP | NM_000161 | GCTACCAGGAGACCATCTCAGA [symbol] | |
| | | cyclo- | | GTCCTAAACGATGCTATATTTGA [symbol] | |
| | | hydrolase | | GAA | |
| | | | | (SEQ ID NO: 732) | |
| M709 | GGT1 | gamma- | NM_005265 | ACAACCAGCTTCTGCCCAACGT [symbol] | |
| | | glutamyl- | | CGACAGTGGAGAGAAACATTGA [symbol] | |
| | | transferase 1 | | CA | |
| | | | | (SEQ ID NO: 733) | |
| M710 | GSR | glutathione | NM_000637 | CAGTGAAGATGGGAGCAACGAA [symbol] | |
| | | reductase | | GCAGACTTTGACAACACAGTCG [symbol] | |
| | | | | ATT | |
| | | | | (SEQ ID NO: 734) | |
| M711 | LAMP1 | lysosomal- | NM_005561 | CAAGGAATCCAGTTGAATACAA [symbol] | |
| | | associated | | CTTCCTGACGCCAGAGACCCTG [symbol] | |
| | | membrane | | TT | |
| | | protein 1 | | (SEQ ID NO: 735) | |
| M712 | Pmel17 | | S73003 | GCAGGTCTGAGTACTCTCATAT [symbol] | |
| | | | | TGCTGTGATTTTCCTGGAGTTGA [symbol] | |
| | | | | AG | |
| | | | | (SEQ ID NO: 736) | |
| M713 | TXNRD1 | thioredoxin | NM_003330 | TCAGAGGCTCTATGCAGGTTCCA [symbol] | |
| | | reductase 1 | | TGTCAAGTGTGACTATGAAAAT [symbol] | |
| | | | | TC | |
| | | | | (SEQ ID NO: 737) | |
| M714 | TXNRD2 | thioredoxin | NM_006440 | GCGAAGTTACTCAAGGATTTGCT [symbol] | |
| | | reductase 2 | | TGGGGATCAAGTGTGGGGCTTC [symbol] | |
| | | | | AT | |
| | | | | (SEQ ID NO: 738) | |
| M715 | BMAL1a | | D89722 | CAACGACCAAGGATCAAGTAGT [symbol] | |
| | | | | CCAGTAATGATGAGGCAGCAAT [symbol] | |
| | | | | GCTG | |
| | | | | (SEQ ID NO: 739) | |
| M716 | CLOCK | clock | NM_+ AF8- | ACAACAGCAACAGTCACAGACA [symbol] | |
| | | homolog | 004898 | TGTCAGTAACGCAGCAGCAGCA [symbol] | |
| | | | | CAG | |
| | | | | (SEQ ID NO: 740) | |
| M717 | CRY1 | cryptochrome | NM_004075 | CTATCAGCAGCTTTCACGATATA [symbol] | |
| | | 1 | | AGGACTAGGTCTTCTGGCATCA [symbol] | |
| | | | | AC | |
| | | | | (SEQ ID NO: 741) | |
| M718 | CRY2 | cryptochrome | XM_051030 | TATGGCGAGAGTTCTTCTACAC [symbol] | |
| | | 2 | | CAGCTACCAACAACCCCAGGTTT [symbol] | |
| | | | | A | |
| | | | | (SEQ ID NO: 742) | |
| M719 | CYP39A1 | cytochrome P4 [symbol] | NM_016593 | TGAATATAAACAAAGAATATGA [symbol] | |
| | | subfamily | | ATCTGTTGGGCCTCACAAGGAC [symbol] | |
| | | XXXIX | | GGG | |
| | | (oxysterol 7 | | (SEQ ID NO: 743) | |
| | | alpha- | | | |
| | | hydroxylase), | | | |
| | | polypeptide 1 | | | |
| M720 | CYP7A1 | cholesterol | NM_000780 | ATCCGGACAGCTAAGGAGGATT [symbol] | |
| | | 7alpha- | | CACTTTGCACCTTGAGGACGGTT [symbol] | |
| | | monooxygenase | | CTA | |
| | | | | (SEQ ID NO: 744) | |
| M721 | CYP7B1 | oxysterol 7 | NM_004820 | CCTACATGGTGACCCTGAAATCT [symbol] | |
| | | alpha | | TGAAGCTCCAGAGGAGTTTAGA [symbol] | |
| | | hydroxylase | | ATG | |
| | | | | (SEQ ID NO: 745) | |
| M722 | DHCR7 | 7-dehydro- | NM_001360 | TGCAGGCCATCTACGTGATTGA [symbol] | |
| | | cholesterol | | TCTTCTGGAACGAAACCTGGTA [symbol] | |
| | | reductase | | TG | |
| | | | | (SEQ ID NO: 746) | |
| M723 | PAH | phenylalanine | NM_000277 | GCCTCTCTGGGTGCACCTGATGA [symbol] | |
| | | hydroxylase | | TACATTGAAAAGCTCGCCACAA [symbol] | |
| | | | | TA | |
| | | | | (SEQ ID NO: 747) | |
| M724 | PER1 | period | NM_002616 | CAGTCCAGCCTTACCTACAGCA [symbol] | |
| | | homolog | | AAACTGCACCAGCTAGACTCCA [symbol] | |
| | | | | CT | |
| | | | | (SEQ ID NO: 748) | |
| M725 | PER2 | period | NM_022817 | CCTTCTCTGGGACTCAGCGAAGT [symbol] | |
| | | homolog | | TCGGACACCAAAGAAGACGAAA [symbol] | |
| | | | | TGG | |
| | | | | (SEQ ID NO: 749) | |
| M726 | PER3 | period | NM_+ AF8- | CTCATGGGCAAAAGGAGGAGCT [symbol] | |
| | | homolog | 016831 | GCTAAGGTGTATAATTGGATTCA [symbol] | |
| | | | | AGC | |
| | | | | (SEQ ID NO: 750) | |
| M727 | TH | tyrosine | NM_000360 | CTACCAAGACCAGACGTACCAG [symbol] | |
| | | hydroxylase | | CAGTCTACTTCGTGTCTGAGAG [symbol] | |
| | | | | TCA | |
| | | | | (SEQ ID NO: 751) | |
| M728 | GSTA1 | glutathione | NM_145740 | ACCAGAGCCATTCTCAACTACAT [symbol] | |
| | | S-transferase | | GCCAGCAAATACAACCTCTATG [symbol] | |
| | | A1 | | GAA | |
| | | | | (SEQ ID NO: 752) | |
| M729 | GSTA2 | glutathione | NM_000846 | TGAAAACCAGAATCAGTAACCT [symbol] | |
| | | S-transferase | | CCCACAGTGAAGAAGTTTCTACA [symbol] | |
| | | A2 | | CCT | |
| | | | | (SEQ ID NO: 753) | |
| M730 | GSTA3 | glutathione | NM_000847 | CATTGCCAGCAAATACAACCTCT [symbol] | |
| | | S-transferase | | CGGGAAAGACATAAAGGAGAGA [symbol] | |
| | | A3 | | CCC | |
| | | | | (SEQ ID NO: 754) | |
| M731 | GSTA4 | glutathione | NM_001512 | CAGACCCGAAGCATTCTCCACTA [symbol] | |
| | | S-transferase | | ATAGCAGACAAGCACAATCTCT [symbol] | |
| | | A4 | | GG | |
| | | | | (SEQ ID NO: 755) | |
| M732 | GSTM1 | glutathione | NM_000561 | AGTACTTGGAGGAACTCCCTGAA [symbol] | |
| | | S-transferase | | AAGCTAAAGCTCTACTCAGAGTT [symbol] | |
| | | M1 | | CTG | |
| | | | | (SEQ ID NO: 756) | |
| M733 | GSTM3 | glutathione | NM_000849 | AGCTCTGACCACGAAAAACTGA [symbol] | |
| | | S-transferase | | GCCTCAGTACTTGGAAGAGCTA [symbol] | |
| | | M3 (brain) | | TGG | |
| | | | | (SEQ ID NO: 757) | |
| M734 | GSTM4 | glutathione | NM_000850 | GCCAGAATACTTGGAGGAACTT [symbol] | |
| | | S-transferase | | TACAATGATGCAGCACTTCTCA [symbol] | |
| | | M4 | | GT | |
| | | | | (SEQ ID NO: 758) | |
| M735 | GSTM5 | glutathione | NM_000851 | TTTCCTTGCCTATGATGTCCTTGA [symbol] | |
| | | S-transferase | | ATGAAGCGTATATTITGAGCCCAA [symbol] | |
| | | M5 | | T | |
| | | | | (SEQ ID NO: 759) | |
| M736 | GSTP1 | glutathione | NM_000852 | GCTGCTGATCCATGAGGTCCTA [symbol] | |
| | | S-transferase | | CCCTGGCTGCCTGGATGCGTT | |
| | | pi | | (SEQ ID NO: 760) | |
| M737 | GSTT1 | glutathione | NM_000853 | AAACCTCACCCTTGCACCGTCCT [symbol] | |
| | | S-transferase | | AGCAGTCCACAAAGCATTTTCAT [symbol] | |
| | | theta | | TC | |
| | | | | (SEQ ID NO: 761) | |
| M738 | GSTZ1 | glutathione | NM_145870 | CATATACTGTGTAGGAGACGAG [symbol] | |
| | | transferase | | TGACCATGGCTGATCTGTGCTT [symbol] | |
| | | zeta | | T | |
| | | | | (SEQ ID NO: 762) | |
| M739 | MGST1 | microsomal | NM_145792 | GGAGTTACTCTTTCCATGGCTTA [symbol] | |
| | | glutathione | | AGGTTGCTGAAAAGTAAATTGTA [symbol] | |
| | | S-transferase | | CT | |
| | | 1 | | (SEQ ID NO: 763) | |
| M740 | MGST2 | microsomal | NM_002413 | CCAAGAAACTGAGGCGGCAATT [symbol] | |
| | | glutathione | | TAACTTTTTCTCTTCCCTTTAAT [symbol] | |
| | | S-transferase | | TT | |
| | | 2 | | (SEQ ID NO: 764) | |
| M741 | MGST3 | microsomal | NM_004528 | AAGTACAAAGTGGAGTATCCTA [symbol] | |
| | | glutathione | | ATGTACAGCACGGACCCTGAAA [symbol] | |
| | | S-transferase | | TGG | |
| | | 3 | | (SEQ ID NO: 765) | |
| M742 | AKR1A1 | Ido-keto | NM_153326 | ATCTGCATCCCCAAAAGTATCA [symbol] | |
| | | reducta | | CCTTCTCGAATCCTTCAGAACAT [symbol] | |
| | | family 1, | | AA | |
| | | member A1 | | (SEQ ID NO: 766) | |
| M743 | ALDH1A | aldehyde | NM_000689 | TGGTGGATTCAAGATGTCTGGAA [symbol] | |
| | | dehydrogenase | | TGGAAGAGAACTGGGAGAGTA [symbol] | |
| | | family, | | GTT | |
| | | member A1 | | (SEQ ID NO: 767) | |
| M744 | ALDH1A | aldehyde | NM_170697 | AAGCTGGGACTGTTTGGATCAAT [symbol] | |
| | | dehydrogenase | | GTTACAATGCCTTAAATGCCCA [symbol] | |
| | | family, | | GC | |
| | | member A2 | | (SEQ ID NO: 768) | |
| M745 | ALDH1A | aldehyde | NM_000693 | CAACATGCGGATTGCCAAAGAG [symbol] | |
| | | dehydrogenase | | AGATTTTCGGGCCAGTGCAACCA [symbol] | |
| | | family, | | TAC | |
| | | member A3 | | (SEQ ID NO: 769) | |
| M746 | ALDH1B | aldehyde | NM_000692 | AGAGGTAAAGACGGTCACCATC [symbol] | |
| | | dehydrogenase | | AGGTTCCTCAGAAGAACTCGTAA [symbol] | |
| | | family, | | GAGC | |
| | | member B1 | | (SEQ ID NO: 770) | |
| M747 | ALDH2 | aldehyde | NM_000690 | GTCAAAGTGCCTCAGAAGAACT [symbol] | |
| | | dehydrogenase | | ATAAGAATCATGCAAGCTTCCT [symbol] | |
| | | family | | CTC | |
| | | | | (SEQ ID NO: 771) | |
| M748 | ALDH3A | aldehyde | NM_000691 | CCTCTACATGTTCTCCAGCAAC [symbol] | |
| | | dehydrogenase | | CAAGGTGATTAAGAAGATGATT [symbol] | |
| | | family, | | CAG | |
| | | member A1 | | (SEQ ID NO: 772) | |
| M749 | ALDH3A | aldehyde | NM_000382 | GAATGATCCTGTTCAACCTCCTA [symbol] | |
| | | dehydrogenase | | TGCCTCTACTGAATTATTCCTCT [symbol] | |
| | | family, | | T | |
| | | member A2 | | (SEQ ID NO. 773) | |
| M750 | ALDH4A | aldehyde | NM_003748 | ATGCTGCCGGCAACTTCTACAT [symbol] | |
| | | dehydrogenase | | ACGACAAGTCCACTGGCTCGATA [symbol] | |
| | | family, | | GTG | |
| | | member A1 | | (SEQ ID NO: 774) | |
| M751 | ALDH5A | aldehyde | NM_170740 | ATGCTGTGCACTCATGAAGAGA [symbol] | |
| | | dehydrogenase | | TTCGGGCCTCTGGCACCAGTTAT [symbol] | |
| | | family, | | AA | |
| | | member A1 | | (SEQ ID NO: 775) | |
| M752 | ALDH6A | aldehyde | NM_005589 | AGGGCATCCAATFCTACACTCA [symbol] | |
| | | dehydrogenase | | TAAAGACCATTACTTCTCAGTG [symbol] | |
| | | family, | | AA | |
| | | member A1 | | (SEQ ID NO: 776) | |
| M753 | ALDH7A | aldehyde | NM_001182 | CTTGGACCTAAAGGATCAGACT [symbol] | |
| | | dehydrogenase | | GGCATTGTAAATGTCAACATTC [symbol] | |
| | | family, | | AC | |
| | | member A1 | | (SEQ ID NO: 777) | |
| M754 | ALDH8A | aldehyde | NM_022568 | CTGGAATAGGTAGAGAGGGAG [symbol] | |
| | | dehydrogenase | | AAGGACTCTTACGACTTCTTCA [symbol] | |
| | | family, | | GAG | |
| | | member A1 | | (SEQ ID NO: 778) | |
| M755 | ALDH9A | aldehyde | NM_000696 | TAGCTGAGCTTCAGGCTGGGAC [symbol] | |
| | | dehydrogenase | | GCTTCATTAACAACTATAACGT [symbol] | |
| | | family, | | GC | |
| | | member A1 | | (SEQ ID NO: 779) | |
| M756 | GPX1 | glutathione | NM_000581 | TACGAGGGAGGAACACCTTGAT [symbol] | |
| | | peroxidase 1 | | TTACAGAAAATACCACCTCGAG [symbol] | |
| | | | | GGG | |
| | | | | (SEQ ID NO: 780) | |
| M757 | GPX2 | glutathione | NM_002083 | AATGAGGAGATCCTGAACAGTC [symbol] | |
| | | peroxidase 2 | | CAAGTATGTCCGTCCTGGGGGT [symbol] | |
| | | | | ATA | |
| | | | | (SEQ ID NO: 781) | |
| M758 | GPX3 | glutathione | NM_002084 | CAACCAATTTGGAAAACAGGAA [symbol] | |
| | | peroxidase 3 | | CAGGAGAGAACTCAGAGATCCT [symbol] | |
| | | | | CCTA | |
| | | | | (SEQ ID NO: 782) | |
| M759 | GPX4 | glutathione | NM_002085 | TGTAACCAGTTCGGGAAGCAGG [symbol] | |
| | | peroxidase 4 | | GCCAGGGAGTAACGAAGAGAT [symbol] | |
| | | | | AAGA | |
| | | | | (SEQ ID NO: 783) | |
| M760 | GPX5 | glutathione | NM_001509 | CTGAACTAAATGCACTCCAGGA [symbol] | |
| | | peroxidase 5 | | GAGCTGAAGCCCTATGGTCTAGT [symbol] | |
| | | | | GTG | |
| | | | | (SEQ ID NO: 784) | |
| M761 | GPX6 | glutathione | NM_015696 | ACCTACAGTGTCTCATTCCCCAT [symbol] | |
| | | peroxidase 6 | | TTTAGCAAGATTGCAGTCACCG [symbol] | |
| | | | | AC | |
| | | | | (SEQ ID NO: 785) | |

### EXAMPLE 10

Analysis of Gene Expression

The molecular chip showed distinctive skin patterns of transcriptional expression of functional neuro-endocrine-immune and barrier forming pathways after ultraviolet light exposure. The full list of differentially expressed genes is presented in Table 2, classified into several major functionally-related groups: water and salt balance; prostaglandin metabolism; interleukins and growth factors; metalloproteinases; markers of keratinocyte differentiation and stress response. Genes from the same group that were similarly expressed presumably reflected a highly coordinated keratinocyte response to UV, which indicated genetic regulation. Each gene had a unique time response that was classified as early, when the maximum induction of expression had occurred by 4 h after treatment. A late response group was defined by expression maximum occurring at 24 h after irradiation.

### EXAMPLE 11

Genes Involved in Water and Salt Balance

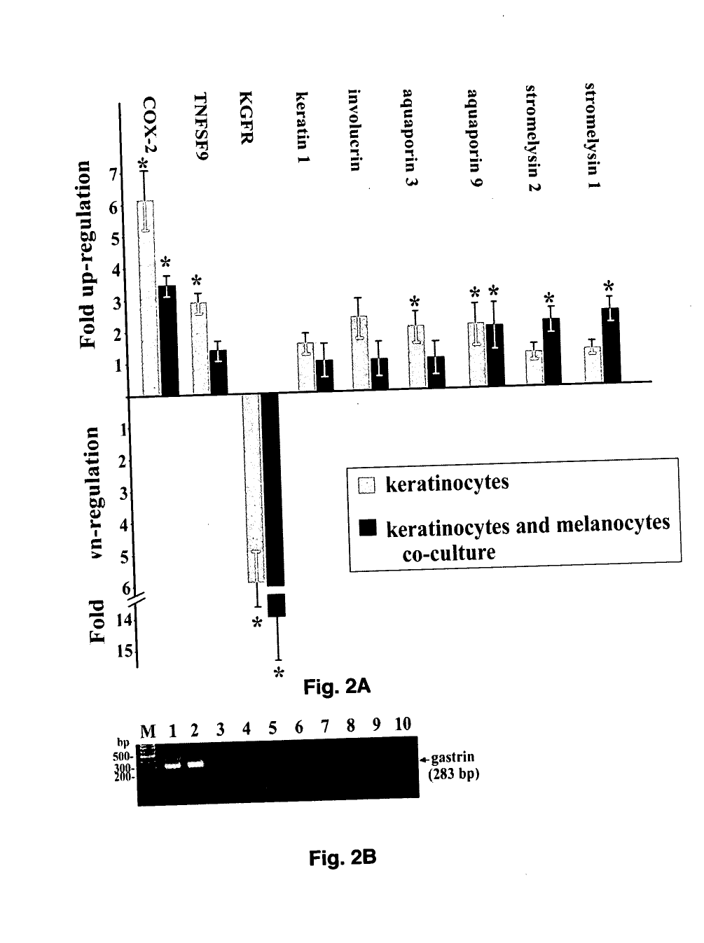

Up-regulation of gastrin, vasopressin and aquaporin genes was detected. All these genes are involved in the regulation of water and salt balance (Table 2, FIG. 2). The highest stimulation was observed at 12 to 24 h post-irradiation. Vasopressin is highly relevant to water metabolism by being involved in water resorption in the kidneys. Other genes related to water balance are those coding for aquaporin water channels (AQP). Aquaporins are a family of membrane proteins that facilitate water movement across biological membranes; they are important for osmotic water movement across cell membranes of epithelial and endothelial barriers. Aquaporins are also expressed in the skin. AQP3 knockout mice are remarkably polyuric and have dry skin (Verkman, 2002). The microarray data was verified by real-time quantitative RT-PCR, which showed 2-fold increases of aquaporin 3 mRNA (FIG. 2A, p<0.05).

The gastrin gene is related to salt balance since gastrin receptors are expressed in the kidneys on tubules and collecting ducts, where they mediate renal potassium and sodium absorption (von Schrenck et al., 2000). Gastrin has potent growth stimulatory effects on gastrointestinal tissues and human kidney cells (Stepan et al., 1999). Gastrin-releasing peptide may also accelerate wound healing in burns, injuries, chronic ulcers, and skin graft donor sites through enhancement of keratinocyte growth and spreading (Yamaguchi et al., 2002). Since the gastrin message was not previously detected in skin, this gene was amplified herein from several skin cell lines including human keratinocytes, melanocytes, fibroblasts and different melanomas (FIG. 2B). The band of correct length was detected only in normal and immortalized keratinocytes (FIG. 2B); after sequencing, the gene was showed perfectly matched with gastrin. Thus, gastrin is expressed in human keratinocytes and its expression is stimulated by UVR (Table 2).

TABLE 2
Genes differentially expressed in ultraviolet irradiated human epidermal keratinocytes

| UP-REGULATED GENES | | | | DOWN-REGULATED GENES | | | |
|---|---|---|---|---|---|---|---|
| | mean ± SD | | | | mean ± SD | | |
| gene | 4 h | 12 h | 24 h | gene | 4 h | 12 h | 24 h |
| Water and salt balance | | | | | | | |
| Gastrin | 1.17 ± 0.35 | 2.06 ± 0.32 | 1.46 ± 0.17 | | | | |
| Vasopressin precursor | 1.04 ± 0.49 | 1.56 ± 0.01 | 1.54 ± 0.01 | | | | |
| Aquaporin 1 | 1.53 ± 0.24 | 1.87 ± 0.37 | 4.11 ± 0.42 | | | | |
| Aquaporin 3 | 1.11 ± 0.10 | 1.49 ± 0.09 | 2.37 ± 0.19 | | | | |
| Aquaporin 9 | 1.04 ± 0.28 | 1.87 ± 0.42 | 2.05 ± 0.07 | | | | |
| Prostaglandin metabolism | | | | | | | |
| CYP4F8 (cytochrome P450, | 1.57 ± 0.02 | 2.41 ± 0.32 | 3.15 ± 0.72 | | | | |
| subfamily IVF, polypeptide 8) | | | | | | | |
| Prostaglandin-endoperoxide | 1.87 ± 0.25 | 3.61 ± 0.40 | 1.20 ± 0.16 | | | | |
| synlhase 2 (COX-2) | | | | | | | |
| Interleukins and growth factors | | | | | | | |
| Interferon-beta-2 | 0.94 ± 0.33 | 1.61 ± 0.12 | 0.98 ± 0.04 | Epidermal growth | 1.31 ± 0.71 | 0.50 ± 0.10 | 1.00 ± 0.3 |
| (interleukin 6) | | | | factor receptor | | | |
| Interleukin 1, beta | 2.14 ± 0.07 | 2.22 ± 0.48 | 0.98 ± 0.10 | Keratinocyte | 1.07 ± 0.10 | 0.49 ± 0.10 | 0.74 ± 0.1 |
| | | | | growth factor | | | |
| Interleukin 2 | 1.16 ± 0.25 | 1.13 ± 0.07 | 2.02 ± 0.10 | Keratinocyte | 0.78 ± 0.09 | 0.46 ± 0.04 | 0.59 ± 0.0 |
| growth factor | | | | | | | |
| Interleukin 8 | 2.44 ± 0.28 | 1.95 ± 0.12 | 1.70 ± 0.20 | Activin A recept | 0.58 ± 0.04 | 0.49 ± 0.07 | 0.99 ± 0.0 |
| | | | | type I | | | |
| | 1.76 ± 0.08 | 2.10 ± 0.10 | 1.28 ± 0.22 | TNFSF10 (TRAI | 0.51 ± 0.03 | 0.65 ± 0.03 | 0.73 ± 0.0 |
| TNFSF9 | | | | | | | |
| Bone morphogenetic | 1.16 ± 0.06 | 1.67 ± 0.03 | 1.10 ± 0.06 | | | | |
| protein 3 | | | | | | | |
| Insulin-like growth | 1.78 ± 0.24 | 0.98 ± 0.16 | 1.78 ± 0.42 | | | | |
| factor binding | | | | | | | |
| protein 6 | | | | | | | |
| Endothelin 1 | 2.30 ± 2.70 | 2.78 ± 4.10 | 2.10 ± 2.20 | | | | |
| Vascular endothelial growth factor | 1.10 ± 0.09 | 2.09 ± 0.08 | 1.85 ± 0.49 | | | | |
| Metalloproteinases | | | | | | | |
| Stromelysin 1^{a} | n/d | 2.02 ± 0.03 | n/d | | | | |
| Stromelysin 2^{a} | n/d | 2.78 ± 0.93 | n/d | | | | |
| Markers of keratinocyte | | | | | | | |
| differentiation | | | | | | | |
| Filaggrin | 1.04 ± 0.21 | 1.57 ± 0.12 | 1.14 ± 0.04 | Fibronectin 1 | 0.96 ± 0.04 | 0.62 ± 0.07 | 0.41 ± 0.0 |
| Involucrin | 1.05 ± 0.04 | 1.57 ± 0.08 | 1.46 ± 0.03 | | | | |
| Keratin 1 | 1.26 ± 0.18 | 1.80 ± 0.17 | 1.57 ± 0.04 | | | | |
| Keratin 10 | 1.18 ± 0.06 | 2.09 ± 0.43 | 0.98 ± 0.04 | | | | |
| Keratin 19 | 0.92 ± 0.38 | 0.85 ± 0.23 | 3.17 ± 0.31 | | | | |
| Transglutaminase 1 | 1.05 ± 0.15 | 1.18 ± 0.22 | 2.01 ± 0.23 | | | | |
| Loricrin | 1.29 ± 0.21 | 1.54 ± 0.24 | 0.51 ± 0.07 | | | | |
| Stress-related genes | | | | | | | |
| CRF-R2 gamma | 1.10 ± 0.08 | 1.99 ± 0.45 | 1.60 ± 0.07 | Urotensin 2 | 1.10 ± 0.08 | 0.81 ± 0.13 | 0.62 ± 0.0 |
| Aldo-keto reductase family 1, | 1.10 ± 0.08 | 1.25 ± 0.12 | 1.56 ± 0.05 | | | | |
| member C2 | | | | | | | |

^{a}effect was seen only when keratinocytes were co-cultivated with melanocytes; n/d—not done; data represents mean +/− SD (N = 4); underlined ratios (UV versus control) are significantly different from 1 (no difference), P < 0.05.

### EXAMPLE 12

Prostaglandin Synthesis

Up-regulation of several genes involved in prostaglandin metabolism including prostaglandin-endoperoxide synthase 2 (Cyclooxygenase 2 or COX-2 gene) (Table 2, FIG. 2); and, of cytochrome P450, subfamily IVF, polypeptide 8 (CYP4F8) (Table 2) were detected. This was consistent with the increase in prostaglandin E2 in UVR-induced inflammation in the skin (Gresham et al., 1996). Real-time PCR also showed a 6-fold increase of COX-2 message (FIG. 2), which was in fact the highest up-regulation ever detected by quantitative PCR. Stimulation of COX-2 expression by UVR had been previously reported (Brecher, 2002) and when taken together with findings presented here, indicated de novo synthesis of prostaglandins. Therefore, the increases in prostaglandin levels in inflammation would be due to increased prostaglandin production coupled with enhanced arachidonate deacylation from the membrane (Gresham et al., 1996).

Another prostaglandin-related gene CYP4F8 was also up-regulated in UV-irradiated keratinocytes (Table 2). That gene, expressed both in human seminal vesicles and skin, catalyzes the 19-hydroxylation of prostaglandins H(1) and H(2) (Stark et al., 2003). CYP4F8 immunoreactivity and mRNA are prominently induced in psoriasis suggesting a pathophysiological role in skin (Stark et al., 2003).

Prostaglandins function has been investigated on melanocytes and keratinocytes. Prostaglandins can induce melanin synthesis in cutaneous melanocytes. Uveal melanocytes also demonstrate increased tyrosinase activity upon exposure to prostaglandin E1 (Fuller and Meyskens, 1981). Prostaglandin E2 receptor agonist has stimulating effects on growth, melanogenesis and dendrification of iridal melanocytes (Hu et al., 2001). In keratinocytes prostaglandins increase vascular endothelial growth factor (VEGF) expression (Trompezinski et al., 2001), the latter a well-recognized effect of UV radiation (Mildner et al., 1999).

### EXAMPLE 13

Interleukins and Growth Factors

Ultraviolet B radiation generates cutaneous inflammation in part, by inducing oxidative stress and cytokine production in human keratinocytes. Expression of the cytokines interleukin (IL)-1, IL-6, IL-10, endothelin 1, VEGFB has already been reported to increase in epidermal keratinocytes upon UV radiation (Kirnbauer et al., 1991; Grewe et al., 1995; Kang et al., 1998; Petit-Frere et al., 1998; Mildner et al., 1999; Brink et al., 2000). An increase of message corresponding to these genes (Table 2) was detected. Additionally, the microarray showed stimulation of IL-2, IL-8, tumor necrosis factor superfamily member 9 (TNFSF9), bone morphogenetic protein 3 (BMP3) and insulin-like growth factor binding protein 6 (IGFBP6) (Table 2).

Decreased expression was noted for the following genes in UV-treated keratinocytes (Table 2): epidermal growth factor receptor (EGFR or erbB), keratinocyte growth factor (KGF or fibroblast growth factor 7), keratinocyte growth factor receptor (KGFR or fibroblast growth factor receptor 2), Activin A receptor type I and tumor necrosis factor superfamily member 10 (TNFSF10 or TRAIL).

The majority of the gene expression changes that were detected herein had been already reported in UV-irradiated skin cells. Therefore, real-time RT-PCR was used only to verify the novel TNFSF9, KGFR and KGF changes in gene expression. TNFSF9 increased 3-fold (FIG. 2A, p<0.05); KGFR gene expression was significantly inhibited in UV-irradiated keratinocytes and to even more significant degree in co-cultures of keratinocytes and melanocytes (FIG. 2A, p<0.05). KGF was also down-regulated (data not shown), but since original expression of this gene was low, it was technically difficult to amplify the amount of message to allow analysis of the data in a statistically meaningful manner.

The functional significance of the differential gene expression could be explained as follows. IL-8 is a neutrophil chemoattractant cytokine with proinflammatory and growth-promoting activities, and is involved in the pathogenesis of several inflammatory diseases. IL-8 is produced in response to ultraviolet B radiation (Countryman et al., 2000). Tumor necrosis factor superfamily member 9 (TNFSF9 or 4-1BBL) is also an important proinflammatory mediator binding CD137 receptors on activated CD4 and CD8 T cells. TNSF9 provides a costimulatory signal that induces a high level of IL-2 production by primary resting T cells (DeBenedette et al., 1999).

Within this context, it would be anticipated that IL-2 expression would increase after UVR, as was indeed found (Table 2). Investigation of infiltrating inflammatory cells and cytokine levels in UV-irradiated skin has shown that interferon-gamma and IL-2, together with the proinflammatory cytokine IL-8, become detectable at 6 h (Terui et al., 2001). In the present invention,-IL-2 was detected 24 h after radiation and most interestingly only after up-regulation of TNFSF9 (4, 12 h; Table 2). This might reflect autocrine stimulation of IL-2 by TNFSF9. The significance of this response is underlined by the effectiveness of interferons and IL-2 in the treatment of skin cancers that include metastatic melanomas (Garbe, 1995).

Stimulation of bone morphogenic protein 3 (BMP-3) and insulin-like growth factor binding protein 6 (IGFBP-6) was detected. Both these proteins inhibit proliferation and differentiation. Treatment of human bone marrow osteoprogenitors with BMP-3 causes dose- and time-dependent inhibition of DNA synthesis and cell proliferation. Simultaneously, BMP3 stimulates type I collagen synthesis (Amedee et al., 1994). IGFBP-6 is a negative effector of oligodendrocyte survival and differentiation (Kuhl et al., 2003), and is secreted by an immortalized human keratinocyte cell line (HaCaT) (Marinaro et al., 1999). Purified IGFBP-6 inhibits the growth of HaCaT and Balb/MK keratinocyte cell lines, as well as Mv1Lu cells (Kato et al., 1995).

EGFR overexpression is important for the expression of malignant behavior, since activation of the receptor stimulates tyrosine kinase activity, which initiates signal transduction cascade (including other kinases) leading to cell proliferation and protection from apoptosis (Lage et al., 2003). Conversely, anti-EGFR agents posses antitumor activity (Lage et al., 2003). Within this context, the known effect of EGF stimulating extracellular receptor-activated kinases (ERK) is prevented by UVR, in a dose-dependent manner. Therefore, the data presented herein on UVR-induced transient (12 hours) inhibition of EGFR expression, suggested that this effect on EGFR was the most likely explanation of the described ERK response inhibition.

Down-regulation of the tumor necrosis factor superfamily member 10 (TNFSF10 or TRAIL), which is a TNF-related apoptosis-inducing ligand was observed. Both up- and down-regulation of TRAIL and its receptors had been reported after exposure to UVB. Since TRAIL and TRAIL receptors function as important sensors in the human epidermis preserving skin integrity and preventing cell transformation, down-regulation of these molecules with sensor function increases the risk for less efficient elimination of aberrant cells. Supporting this concept is the low amount or absence of these molecules in precancerous states developing after long-term ultraviolet exposure (Bachmann et al., 2001).

Keratinocyte growth factor (KGF or fibroblast growth factor 7) is a potent mitogen for human keratinocytes, stimulating proliferation and migration of these cells and also affecting their differentiation process (Werner, 1998). Deletion of the KGF receptor (KGFR) gene results in severe functional defects of the anterior pituitary and also, in developmental abnormalities of the skin (De Moerlooze et al., 2000). IL-6, interferon-gamma, and ultraviolet B (UVB) treatment lead to substantial down-regulation of KGFR expression (Zhou et al., 1996). Down regulation of both KGF and KGFR (Table 2) was detected, confirmed by real-time PCR (FIG. 2A). The UV-induced down-regulation of KGF and KGFR synthesis might therefore represent an antiproliferative mechanism addressed at preventing cells from entering malignant transformation.

### EXAMPLE 14

Genes Involved in Extracellular Matrix Rearrangement

Involvement of matrix metalloproteinases (MMPs), the enzymes responsible for extracellular matrix rearrangement, UVR-induced skin aging and promotion of skin cancer (Brenneisen et al., 2002), was also observed. This was represented by a significant stimulation of stromelysins 1 and 2 expression, but only when keratinocytes were co-cultivated with melanocytes (Table 2). Real-time quantitative RT-PCR did confirm this observation (FIG. 2A, p<0.05), suggesting that either keratinocytes express UV-induced metalloproteinases only when they are in contact with melanocytes or, that the matrix metalloproteinases are produced by melanocytes.

### EXAMPLE 15

Stress Response Genes

CRH may be the primary mediator of autonomic, behavioral and neuroendocrine responses to stress. While CRH receptors type 1 were previously detected exclusively in human skin cells (Slominski et al., 2004), the CRH receptor type 2 gamma isoform, whose expression is increased in UVR-treated human keratinocytes were included in the present invention.

Aldo-keto reductase, another stress-response related gene, was observed to be up-regulated. Interestingly, the fungi S. cerevisiae that have aldo-keto reductases deleted show enhanced sensitivity to stress (Chang et al., 2003).

The following references were cited herein:
Amedee, J., et al: Differentiation 58 (1994) 157-64.
Bachrnann, F., et al: J Invest Dermatol 117 (2001) 59-66.
Bilban, M., et al: Curr Issues Mol Biol 4 (2002) 57-64.
Brecher, A. R.: J Drugs Dermatol 1 (2002) 44-7.
Brenneisen, P., et al: Ann N Y Acad Sci 973 (2002) 31-43.
Brink, N., et al: Inflamm Res 49 (2000) 290-6.
Chang, Q., et al: Chem Biol Interact 143-144 (2003) 325-32.
Countryman, N. B., et al: J Invest Dermatol 115 (2000) 267-72.
DeBenedette, M. A., et al: J Immunol 163 (1999) 4833-41.
De Moerlooze, L., et al: Development 127 (2000) 483-92.
Fuller, B. B. and Meyskens, F. L., Jr.: J Natl Cancer Inst 66 (1981) 799-802.
Garbe, C.: Recent Results Cancer Res 139 (1995) 349-69.
Gresham, A., et al: Am J Physiol 270 (1996) C1037-50.
Grewe, M., et al: J Invest Dermatol 104 (1995) 3-6.
Hu, D. N., et al: Exp Eye Res 73 (2001) 93-100.
Ichihashi, M., et al: Toxicology 189 (2003) 21-39.
Kang, K., et al: J Invest Dermatol 111 (1998) 31-8.
Kato, M., et al: J Biol Chem 270 (1995) 12373-9.
Kirnbauer, R, et al: J Invest Dermatol 96 (1991) 484-9.
Kuhl, N. M., et al: Glia 44 (2003) 91-101.
Lage, A., et al: Ann Med 35 (2003) 327-36.
Maeda, T., et al: J Cutan Med Surg 5 (2001) 294-8.
Marinaro, J. A., et al: Am J Physiol 276 (1999) E536-42.
Maziere, C., et al: Free Radic Biol Med 34 (2003) 629-36.
Mildner, M., et al: Photochem Photobiol 70 (1999) 674-9.
Petit-Frere, C., et al: J Invest Dermatol 111 (1998) 354-9.
Pisarchik, A. and Slominski, A. T.: Faseb J 15 (2001) 2754-6.
Slominski, A. and Pawelek, J.: Clin Dermatol 16 (1998) 503-15.
Slominski, A., et al: Endocrinology 145 (2004) 941-50.
Slominski, A. and Wortsman, J.: Endocr Rev 21 (2000) 457-87.
Stark, K., et al: Arch Biochem Biophys 409 (2003) 188-96.
Stepan, V. M., et al: Am J Physiol 277 (1999) R572-81.
Terui, T., et al: Acta Derm Venereol 81 (2001) 8-13.
Trompezinski, S., et al: Inflamm Res 50 (2001) 422-7.
Verkmnan, A. S.: Ann Med 34 (2002) 192-200.
von Schrenck, T., et al: Kidney Int 58 (2000) 995-1003.
Werner, S.: Cytokine Growth Factor Rev 9 (1998) 153-65.
Yamaguchi, Y., et al: Dermatol Surg 28 (2002) 314-9.
Zhou, Y., et al: Exp Dermatol 5 (1996) 138-44.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A DNA microarray chip for measuring transcriptional responses to stress in a cell, said chip comprising oligonucleotides for genes related to production and metabolism of hormones, neurotransmitters, neuropeptides, cytokines, biological modulators, growth factors, corresponding receptors and normal skin metabolism genes.

2. The DNA microarray chip of claim 1, wherein said chip comprising about 761 oligonucleotide sequences.

3. The DNA microarray chip of claim 1, wherein said oligonucleotides for genes have SEQ ID Nos. shown in SEQ ID No. 25-785.

4. A method of detecting transcriptional responses to stress in a cell, comprising the steps of:
challenging said cell with stress;
isolating nucleic acid samples from said cell;
measuring gene expression levels; and
analyzing said gene expression levels statistically as compared to those without stress, wherein a statistically significant upregulation of said gene expression indicates transcriptional responses to stress.

5. The method of claim 4, wherein said gene expression is measured by DNA microarray.

6. The method of claim 5, wherein said gene expression is further verified by RT-PCR.

7. The method of claim 4, wherein said stress comprises ultraviolet radiation, thermal stress, chemical stress, and immune stress.

8. The method of claim 4, wherein said cell comprises epidermal keratinocyte, melanocyte, fibroblast, and immune cell.

9. The method of claim 4, wherein said statistical analysis is one-way analysis of variance or post-hoc test or student t test.

10. The method of claim 4, wherein said statistically significant upregulation is at least a 1.5-fold increase of expression of said genes.

11. The method of claim 4, wherein said genes comprises genes involved in water and salt balance, genes involved in prostaglandin metabolism, interleukin genes, growth factor genes, markers of keratinocyte differentiation genes, metalloproteinases genes, and stress response genes.

12. The method of claim 4, wherein said upregulated genes are selected from the group of genes encoding for gastrin, vasopressin, aquaporin, prostaglandin-endoperoxide synthase 2, CYP4F8, IL-1, IL-2, IL-6, IL-8, tumor necrosis factor superfamily member 9, bone morphogenetic protein 3, insulin-like growth factor binding protein 6, vascular endothelial growth factor B, stromelysins 1 and 2, corticotropin releasing factor receptor type 1 and 2, and aldo-keto reductase.

13. A method of detecting transcriptional responses to stress in a cell, comprising the steps of:
challenging said cell with stress;
isolating nucleic acid samples from said cell;
measuring gene expression levels; and
analyzing said gene expression levels statistically as compared to those without stress, wherein a statistically significant downregulation of said gene expression indicates transcriptional responses to stress.

14. The method of claim 13, wherein said gene expression is measured by DNA microarray.

15. The method of claim 14, wherein said gene expression is further verified by RT-PCR.

16. The method of claim 13, wherein said stress comprises ultraviolet radiation, thermal stress, chemical stress, and immune stress.

17. The method of claim 13, wherein said cell comprises epidermal keratinocyte, melanocyte, fibroblast, and immune cell.

18. The method of claim 13, wherein said statistical analysis is one-way analysis of variance or post-hoc test or student t test.

19. The method of claim 13, wherein said statistically significant downregulation is at least a 1.5-fold decrease of expression of said genes.

20. The method of claim 13, wherein said a group of genes comprises genes involved in water and salt balance, genes involved in prostaglandin metabolism, interleukin genes, growth factor genes, markers of keratinocyte differentiation genes, metalloproteinases genes, and stress response genes.

21. The method of claim 13, wherein said downregulated genes are selected from the group of genes encoding for epidermal growth factor receptor, keratinocyte growth factor, keratinocyte growth factor receptor, activin A receptor type I, and tumor necrosis factor superfamily member 10.


SEQUENCE LISTING (785 sequences)

SEQ ID NO: 1 - DNA, 21 bp, artificial sequence
  forward primer P898 for prostaglandin- endoperoxide synthase 2
atgagtgtgggatttgaccag

SEQ ID NO: 2 - DNA, 21 bp, artificial sequence
  reverse primer P899 for prostaglandin- endoperoxide synthase 2
ttccggtgttgagcagttttc

SEQ ID NO: 3 - DNA, 21 bp, artificial sequence
  forward primer p900 for tnrsf9
agctacaaagaggacacgaag

SEQ ID NO: 4 - DNA, 21 bp, artificial sequence
  reverse primer p901 for tnrsf9
ttccggtgttgagcagttttc

SEQ ID NO: 5 - DNA, 21 bp, artificial sequence
  forward primer p904 for keratinocyte growth factor receptor
gatggtgcggaagattttgtc

SEQ ID NO: 6 - DNA, 21 bp, artificial sequence
  reverse primer p905 for keratinocyte growth factor receptor
gccgcttttccatcttttctg

SEQ ID NO: 7 - DNA, 21 bp, artificial sequence
  forward primer p908 for stromelysin 2
attttggccctctcttccatc

SEQ ID NO: 8 - DNA, 21 bp, artificial sequence
  reverse primer p909 for stromelysin 2
tgatggcccagaactcatttc

SEQ ID NO: 9 - DNA, 21 bp, artificial sequence
  forward primer p910 for stromelysin 1
ggaaatcaattctgggccatc

SEQ ID NO: 10 - DNA, 21 bp, artificial sequence
  reverse primer p911 for stromelysin 1
catcgattttcctcacggttg

SEQ ID NO: 11 - DNA, 21 bp, artificial sequence
  forward primer p912 for aquaporin 3
tttggctttgctgtcactctg

SEQ ID NO: 12 - DNA, 20 bp, artificial sequence
  reverse primer p913 for aquaporin 3
aagcacatggcaaaggtcac

SEQ ID NO: 13 - DNA, 21 bp, artificial sequence
  forward primer p916 for aquaporin 9
tttgctggtggaaaactgctg

SEQ ID NO: 14 - DNA, 21 bp, artificial sequence
  reverse primer p917 for aquaporin 9
aaatgcgttcgccagagatag

SEQ ID NO: 15 - DNA, 20 bp, artificial sequence
  forward primer k1-1 for keratin 1
tgaccaaggtggaccttcag

SEQ ID NO: 16 - DNA, 20 bp, artificial sequence
  reverse primer k1-r for keratin 1
atgatgctgtccaggtcgag

SEQ ID NO: 17 - DNA, 21 bp, artificial sequence
  forward primer ivl-1 for involucrin
tcagccttactgtgagtctgg

SEQ ID NO: 18 - DNA, 20 bp, artificial sequence
  reverse primer ivl-r for involucrin
agtcttgaggagctcctgac

SEQ ID NO: 19 - DNA, 25 bp, artificial sequence
  forward primer g-1 for gadph
tgatgacatcaagaaggtggtgaag

SEQ ID NO: 20 - DNA, 23 bp, artificial sequence
  reverse primer g-r for gadph
tccttggaggccatgtaggccat

SEQ ID NO: 21 - DNA, 20 bp, artificial sequence
  forward primer ba-1 for ?-actin
gccctggcacccagcacaat

SEQ ID NO: 22 - DNA, 20 bp, artificial sequence
  reverse primer ba-r for ?-actin
tcatagtccgcctagaagca

SEQ ID NO: 23 - DNA, 20 bp, artificial sequence
  forward primer P432 for human gastrin
tgcagacgagatgcagcgac

SEQ ID NO: 24 - DNA, 21 bp, artificial sequence
  reverse primer P433 for human gastrin
gtccatccatccataggcttc

SEQ ID NO: 25 - DNA, 50 bp, artificial sequence
  primer M001 for GCG
ttcaagacacagaggagaaatccagatcattctcagcttcccaggcagac

SEQ ID NO: 26 - DNA, 50 bp, artificial sequence
  primer M002 for GCG
tggtacctgccttggcaccacaaagtgcaacaccgcttcgtgttcaagag

SEQ ID NO: 27 - DNA, 50 bp, artificial sequence
  primer M003 for CRH-R1
gtgccaggagatcctcaatgaggagaaaaaaagcaaggtgcactaccatg

SEQ ID NO: 28 - DNA, 40 bp, artificial sequence
  primer M004 for CRH-R1
aggagatcctcaatgaggagaaaaaaagcaaggtgcacta

SEQ ID NO: 29 - DNA, 30 bp, artificial sequence
  primer M005 for CRH-R1
atcctcaatgaggagaaaaaaagcaaggtg

SEQ ID NO: 30 - DNA, 50 bp, artificial sequence
  primer M006 for CRF-R1h
gtgccaggagatcctcaatgaggaggagcctgggactcaggcacagcctg

SEQ ID NO: 31 - DNA, 40 bp, artificial sequence
  primer M007 for CRF-R1h
aggagatcctcaatgaggaggagcctgggactcaggcaca

SEQ ID NO: 32 - DNA, 30 bp, artificial sequence
  primer M008 for CRF-R1h
atcctcaatgaggaggagcctgggactcag

SEQ ID NO: 33 - DNA, 50 bp, artificial sequence
  primer M009 for CRF-R1h
gttctactgtttcctcaatagtgaggtccgttctgccatccggaagaggt

SEQ ID NO: 34 - DNA, 40 bp, artificial sequence
  primer M010 for CRF-R1h
actgtttcctcaatagtgaggtccgttctgccatccggaa

SEQ ID NO: 35 - DNA, 50 bp, artificial sequence
  primer M011 for CRF-R1h
caactccttcctggaatccttccaggtccgttctgccatccggaagaggt

SEQ ID NO: 36 - DNA, 40 bp, artificial sequence
  primer M012 for CRF-R1h
ccttcctggaatccttccaggtccgttctgccatccggaa

SEQ ID NO: 37 - DNA, 50 bp, artificial sequence
  primer M013 for 5HTR2B
attgccctcttgacaataatgtttgaggctatgtggcccctcccacttgt

SEQ ID NO: 38 - DNA, 40 bp, artificial sequence
  primer M014 for 5HTR2B
cctcttgacaataatgtttgaggctatgtggcccctccca

SEQ ID NO: 39 - DNA, 30 bp, artificial sequence
  primer M015 for 5HTR2B
tgacaataatgtttgaggctatgtggcccc

SEQ ID NO: 40 - DNA, 50 bp, artificial sequence
  primer M016 for 5HTR2Bb
attgccctcttgacaataatgtttggcattgccattccagtccctattaa

SEQ ID NO: 41 - DNA, 40 bp, artificial sequence
  primer M017 for 5HTR2Bb
cctcttgacaataatgtttggcattgccattccagtccct

SEQ ID NO: 42 - DNA, 30 bp, artificial sequence
  primer M018 for 5HTR2Bb
tgacaataatgtttggcattgccattccag

SEQ ID NO: 43 - DNA, 50 bp, artificial sequence
  primer M019 for 5HTR7
ccctgtgcgtgatcagcattgacaggtaccttgggatcacaaggcccctc

SEQ ID NO: 44 - DNA, 40 bp, artificial sequence
  primer M020 for 5HTR7
tgcgtgatcagcattgacaggtaccttgggatcacaaggc

SEQ ID NO: 45 - DNA, 30 bp, artificial sequence
  primer M021 for 5HTR7
gatcagcattgacaggtaccttgggatcac

SEQ ID NO: 46 - DNA, 40 bp, artificial sequence
  primer M022 for CRF-R2 alpha
cacccctggaccccgagggtccctactcctactgcaacac

SEQ ID NO: 47 - DNA, 50 bp, artificial sequence
  primer M023 for CRF-R2 beta
catgaccctcaccaacctctcaggtccctactcctactgcaacacgacct

SEQ ID NO: 48 - DNA, 40 bp, artificial sequence
  primer M024 for CRF-R2 beta
ccctcaccaacctctcaggtccctactcctactgcaacac

SEQ ID NO: 49 - DNA, 50 bp, artificial sequence
  primer M025 for CRF-R2 gamma
ctttcctcagctcttctgccaaggtccctactcctactgcaacacgacct

SEQ ID NO: 50 - DNA, 50 bp, artificial sequence
  primer M026 for CRF
cgagcagttagcacagcaagctcacagcaacaggaaactcatggagatta

SEQ ID NO: 51 - DNA, 50 bp, artificial sequence
  primer M027 for Uro
cttctctgtccattgacctcacctttcacctgctgcggaccctgctggag

SEQ ID NO: 52 - DNA, 50 bp, artificial sequence
  primer M028 for CRFBP
ataaagacagaccccaacctctttccttgcaatgtcatttctcagactcc

SEQ ID NO: 53 - DNA, 49 bp, artificial sequence
  primer M029 for FSH beta
tgtgctggctactgctacaccagggatctggtgtataaggacccagcca

SEQ ID NO: 54 - DNA, 50 bp, artificial sequence
  primer M030 for FSHR
gaaccttcccaaccttcaatatctgttaatatccaacacaggtattaagc

SEQ ID NO: 55 - DNA, 48 bp, artificial sequence
  primer M031 for CGB
caccaaggatggagatgttccaggggctgctgctgttgctgctgctga

SEQ ID NO: 56 - DNA, 50 bp, artificial sequence
  primer M032 for GLYCA
ctccattccgctcctgatgtgcaggattgcccagaatgcacgctacagga

SEQ ID NO: 57 - DNA, 50 bp, artificial sequence
  primer M033 for GLYCA
ctgtgtagctaaatcatataacagggtcacagtaatggggggtttcaaag

SEQ ID NO: 58 - DNA, 47 bp, artificial sequence
  primer M034 for LHB
tgtgccggctactgccccaccatgatgcgcgtgctgcaggcggtcct

SEQ ID NO: 59 - DNA, 50 bp, artificial sequence
  primer M035 for LHCGR
tcaatgggacgacactgacttcactggagctaaaggaaaacgtacatctg

SEQ ID NO: 60 - DNA, 50 bp, artificial sequence
  primer M036 for GNRH1
cctccgagacctgaaaggagctctggaaagtctgattgaagaggaaactg

SEQ ID NO: 61 - DNA, 47 bp, artificial sequence
  primer M037 for GNRH2
cccagaatgcccttaggcccccaggaagggccctggacactgcagca

SEQ ID NO: 62 - DNA, 50 bp, artificial sequence
  primer M038 for GNRHR
cgggtccttcatcaggacccccacgaactacaactgaatcagtccaagaa

SEQ ID NO: 63 - DNA, 50 bp, artificial sequence
  primer M039 for GNRHR2
tagtttcctgcttgccttcccccagctgttcctgttccacacggtccact

SEQ ID NO: 64 - DNA, 50 bp, artificial sequence
  primer M040 for GH2
ttccaacagggtgaaaacgcagcagaaatctaacctagagctgctccgca

SEQ ID NO: 65 - DNA, 50 bp, artificial sequence
  primer M041 for GH1, CSH1, CSH2 and GH2
cagggaggaaacacaacagaaatccaacctagagctgctccgcatctccc

SEQ ID NO: 66 - DNA, 47 bp, artificial sequence
  primer M042 for GH1
agcctggtgtacggcgcctctgacagcaacgtctatgacctcctaaa

SEQ ID NO: 67 - DNA, 50 bp, artificial sequence
  primer M043 for GHR
aagtgtttctctgttgatgaaatagtgcaaccagatccacccattgccct

SEQ ID NO: 68 - DNA, 47 bp, artificial sequence
  primer M044 for GHRH
ccacctccccctttgaccctcaggatgcggcggtatgcagatgccat

SEQ ID NO: 69 - DNA, 50 bp, artificial sequence
  primer M045 for GHRHR
ttctctcacttcagctcagagtcaggggctgtgaaacgggattgtactat

SEQ ID NO: 70 - DNA, 50 bp, artificial sequence
  primer M046 for ghrelin
gagccctgaacaccagagagtccagcagagaaaggagtcgaagaagccac

SEQ ID NO: 71 - DNA, 50 bp, artificial sequence
  primer M047 for MC1R
aacctctttctcgccctcatcatctgcaatgccatcatcgaccccctcat

SEQ ID NO: 72 - DNA, 50 bp, artificial sequence
  primer M048 for MC2R
gatcgtcctgctggctgtgttcaagaataagaatctccaggcacccatgt

SEQ ID NO: 73 - DNA, 46 bp, artificial sequence
  primer M049 for POMC
ctcaccacggaaagcaacctgctggagtgcatccgggcctgcaagc

SEQ ID NO: 74 - DNA, 46 bp, artificial sequence
  primer M050 for POMC
tcagagagcagcctcccgagacagagcctcagcctgcctggaagat

SEQ ID NO: 75 - DNA, 48 bp, artificial sequence
  primer M051 for LOC51
actccatggagatccgcacccctgacatcaatcctgcctggtacgcca

SEQ ID NO: 76 - DNA, 49 bp, artificial sequence
  primer M052 for PrRPR
tgctgctggtcacctacctgctccctctgctggtcatcctcctgtctta

SEQ ID NO: 77 - DNA, 50 bp, artificial sequence
  primer M053 for PRL
gggcatggagctgatagtcagccaggttcatcctgaaaccaaagaaaatg

SEQ ID NO: 78 - DNA, 50 bp, artificial sequence
  primer M054 for PRLR
ggatttgatgctcatctgttggagaagggcaagtctgaagaactactgag

SEQ ID NO: 79 - DNA, 47 bp, artificial sequence
  primer M055 for SST
tggctgctgccgcggggaagcaggaactggccaagtacttcttggca

SEQ ID NO: 80 - DNA, 50 bp, artificial sequence
  primer M056 for SSTR1
cctctatggctttctctcagacaacttcaagcgctctttccaacgcatcc

SEQ ID NO: 81 - DNA, 50 bp, artificial sequence
  primer M057 for SSTR2
ggctatccattccatttgacctcaatggctctgtggtgtcaaccaacacc

SEQ ID NO: 82 - DNA, 50 bp, artificial sequence
  primer M058 for SSTR2, SSTR1,SSTR3, and SSTR5
tcaatcagttcaccagcatcttctgcctgacagtcatgagcatcgaccga

SEQ ID NO: 83 - DNA, 50 bp, artificial sequence
  primer M059 for SSTR3
cgctcttcgtgctctgctggatgcccttctacgtgctcaacatcgtcaac

SEQ ID NO: 84 - DNA, 47 bp, artificial sequence
  primer M060 for SSTR4
gtcttcgtggtctacactttcctgctgggcttcctgctgcccgtgct

SEQ ID NO: 85 - DNA, 50 bp, artificial sequence
  primer M061 for SSTR5
tcaccaacatctacattctcaacctggcagtggccgacgtcctgtacatg

SEQ ID NO: 86 - DNA, 48 bp, artificial sequence
  primer M062 for TRH
aaggggaccagggtgagcactccgcgtcccagatctttcaatctgact

SEQ ID NO: 87 - DNA, 50 bp, artificial sequence
  primer M063 for TRHR
cagcacagtatcttcaaggaagcaggtcaccaagatgctggcagtggttg

SEQ ID NO: 88 - DNA, 50 bp, artificial sequence
  primer M064 for TSHb
ctgtgctggatattgtatgacacgggatatcaatggcaaactgtttcttc

SEQ ID NO: 89 - DNA, 50 bp, artificial sequence
  primer M065 for TSHR
gcttaccgcccagtacgcagactctgaagcttattgagactcacctgaga

SEQ ID NO: 90 - DNA, 50 bp, artificial sequence
  primer M066 for DIO1
tgcaacatttgggagtttatgcaaggtaataggccactggtgctgaattt

SEQ ID NO: 91 - DNA, 50 bp, artificial sequence
  primer M067 for DIO2
ctggaagagcttcctcctcgatgcctacaaacaggtgaaattgggtgagg

SEQ ID NO: 92 - DNA, 50 bp, artificial sequence
  primer M068 for DIO3
gctaccaggtctctgagctgcgcacttggttggaacgctatgatgagcaa

SEQ ID NO: 93 - DNA, 50 bp, artificial sequence
  primer M069 for SLC26A4
agagcactggaggaaagacacaggttgctggcatcatctctgctgcgatt

SEQ ID NO: 94 - DNA, 50 bp, artificial sequence
  primer M070 for THRA
acttgtgagggctgcaagggcttctttcgccgcacaatccagaagaacct

SEQ ID NO: 95 - DNA, 50 bp, artificial sequence
  primer M071 for THRB
atctatgttggcatggcaacagatttggtgctggatgacagcaagaggct

SEQ ID NO: 96 - DNA, 50 bp, artificial sequence
  primer M072 for TG
tagggaagcccaagaaatgccccacgccctgtcaattacaggctgagcaa

SEQ ID NO: 97 - DNA, 50 bp, artificial sequence
  primer M073 for TG
caagggcgggaactggctgagacaggtttggagttgttactggatgaaat

SEQ ID NO: 98 - DNA, 50 bp, artificial sequence
  primer M074 for AG
tatgaagcagggatgactctgggagcccggaagctgaagaaacttggtaa

SEQ ID NO: 99 - DNA, 50 bp, artificial sequence
  primer M075 for SBP
agggactcaggcagaattcaatctccgagacattccccagcctcatgcag

SEQ ID NO: 100 - DNA, 50 bp, artificial sequence
  primer M076 for ESR1
tgctacgaagtgggaatgatgaaaggtgggatacgaaaagaccgaagagg

SEQ ID NO: 101 - DNA, 50 bp, artificial sequence
  primer M077 for ESRRB or ESR2
ttacgaagtgggaatggtgaagtgtggctcccggagagagagatgtgggt

SEQ ID NO: 102 - DNA, 50 bp, artificial sequence
  primer M078 for ESSRA
atcagctgggccaagagcatcccaggcttctcatcgctgtcgctgtctga

SEQ ID NO: 103 - DNA, 50 bp, artificial sequence
  primer M079 for ESSRA
gactctgttggatcagaaggtcaaggtggtctatgttgcccgaaacccaa

SEQ ID NO: 104 - DNA, 50 bp, artificial sequence
  primer M080 for REA
gagcagcccagaatatctccaagacgatcgccacatcacagaatcgtatc

SEQ ID NO: 105 - DNA, 50 bp, artificial sequence
  primer M081 for REA
tgaaatgggcaaaggcaataccaggtttcaggaacttacacctggatgac

SEQ ID NO: 106 - DNA, 50 bp, artificial sequence
  primer M082 for REA
gaccatgagtattgaattccccgagatgttagctgaaatcatcaccaatc

SEQ ID NO: 107 - DNA, 50 bp, artificial sequence
  primer M083 for LOC14
acaaagcagggaagagaaacttgaggtggcagcatgtgtcagtcttcccc

SEQ ID NO: 108 - DNA, 50 bp, artificial sequence
  primer M084 for MR
cttcaagctggaatgaatttaggagcacgaaagtcaaagaagttgggaaa

SEQ ID NO: 109 - DNA, 50 bp, artificial sequence
  primer M085 for MR
cttcaagctggaatgaatttaggagagagaagatgcatcagtctgccatg

SEQ ID NO: 110 - DNA, 50 bp, artificial sequence
  primer M086 for BZRP
ttctttggtgcccgacaaatgggctgggccttggtggatctcctgctggt

SEQ ID NO: 111 - DNA, 50 bp, artificial sequence
  primer M087 for NR1H2
cctcctgaaggcatccactatcgagatcatgctgctagagacagccaggc

SEQ ID NO: 112 - DNA, 50 bp, artificial sequence
  primer M088 for NR113
gtgagggctgcaagggtttcttcaggagaacagtcagcaaaagcattggt

SEQ ID NO: 113 - DNA, 50 bp, artificial sequence
  primer M089 for NR2C1
tgatctgtctgcacaacacctgcagctcctaacagataattctccagacc

SEQ ID NO: 114 - DNA, 50 bp, artificial sequence
  primer M090 for NR2C2
ttcagacacacacgtcacatttaagctaacaatgcccagtccaatgccag

SEQ ID NO: 115 - DNA, 50 bp, artificial sequence
  primer M091 for NR2F2
aaggccatagtcctgttcacctcagatgcctgtggtctctctgatgtagc

SEQ ID NO: 116 - DNA, 47 bp, artificial sequence
  primer M092 for NR4A1
tggcggtgggcatggtgaaggaagttgtccgaacagacagcctgaag

SEQ ID NO: 117 - DNA, 46 bp, artificial sequence
  primer M93 for NR0B1
cgcttctgtaccgctgctgcttttgcggtgaagaccacccgcagca

SEQ ID NO: 118 - DNA, 47 bp, artificial sequence
  primer M094 for NR2F1
tcaaagtgggcatgaggcgggaagcggttcagcgaggaagaatgcct

SEQ ID NO: 119 - DNA, 50 bp, artificial sequence
  primer M095 for NR1H4
gcactgacctgtgaggggtgtaaaggtttcttcaggagaagcattaccaa

SEQ ID NO: 120 - DNA, 50 bp, artificial sequence
  primer M096 for NR2E1
tgagcaagccagccggatcaacaagccgcattttagatatcccctgcaaa

SEQ ID NO: 121 - DNA, 50 bp, artificial sequence
  primer M097 for NR4A2
tgtttgtccttcgattagcatacaggtccaacccagtggagggtaaactc

SEQ ID NO: 122 - DNA, 50 bp, artificial sequence
  primer M098 for NR4A3
ctcagtgttggaatggtaaaagaagttgtccgtacagatagtctgaaagg

SEQ ID NO: 123 - DNA, 46 bp, artificial sequence
  primer M099 for NR5A1
tgctcacgtgtgagagctgcaagggcttcttcaagcgcacggtgca

SEQ ID NO: 124 - DNA, 50 bp, artificial sequence
  primer M100 for NR5A1
aagttcatcatcctcttcagcctggatttgaagttcctgaataaccacat

SEQ ID NO: 125 - DNA, 50 bp, artificial sequence
  primer M101 for NR6A1
cgccctccgatgaagaactacacagatttagtgatgaagggatggaggtg

SEQ ID NO: 126 - DNA, 50 bp, artificial sequence
  primer M102 for PGR
cttctttaagagggcaatggaagggcagcacaactacttatgtgctggaa

SEQ ID NO: 127 - DNA, 50 bp, artificial sequence
  primer M103 for PGR
gctgtcaggctggcatggtccttggaggttttcgaaacttacatattgat

SEQ ID NO: 128 - DNA, 50 bp, artificial sequence
  primer M104 for PGRMC1
agtgactgggagtctcagttcactttcaagtatcatcacgtgggcaaact

SEQ ID NO: 129 - DNA, 50 bp, artificial sequence
  primer M105 for PGRMC2
cgagaatgggaaatgcagtttaaagaaaaatatgattatgtaggcagact

SEQ ID NO: 130 - DNA, 50 bp, artificial sequence
  primer M106 for RARA
tttgaagtgggcatgtccaaggagtctgtgagaaacgaccgaaacaagaa

SEQ ID NO: 131 - DNA, 50 bp, artificial sequence
  primer M107 for RARBv1
ggtcagcgcctgtgagggatgtaagggctttttccgcagaagtattcaga

SEQ ID NO: 132 - DNA, 49 bp, artificial sequence
  primer M108 for RARG
acctccggggcatcagcactaagggagctgaaagggccattactctgaa

SEQ ID NO: 133 - DNA, 46 bp, artificial sequence
  primer M109 for RXRA
atctgcggggaccgctcctcaggcaagcactatggagtgtacagct

SEQ ID NO: 134 - DNA, 48 bp, artificial sequence
  primer M110 for RXRB
caatctgcggggacagaagctcaggcaaacactacggggtttacagct

SEQ ID NO: 135 - DNA, 50 bp, artificial sequence
  primer M111 for ADR1D1
tggtacctactcagaacctaaatcgacccctaagggagcctgtgcaacat

SEQ ID NO: 136 - DNA, 48 bp, artificial sequence
  primer M112 for CYP11A
tccagaagtatggcccgatttacagggagaagctcggcaacgtggagt

SEQ ID NO: 137 - DNA, 50 bp, artificial sequence
  primer M113 for CYP11B1
aaatgtggcgtgttcttgctgaatgggcctgaatggcgcttcaaccgatt

SEQ ID NO: 138 - DNA, 48 bp, artificial sequence
  primer M114 for CYP17
acttctctgggcggcctcaaatggcaactctagacatcgcgtccaaca

SEQ ID NO: 139 - DNA, 50 bp, artificial sequence
  primer M115 for CYP19
gctcttcttgaggatccctttggacgaaagtgctatcgtggttaaaatcc

SEQ ID NO: 140 - DNA, 50 bp, artificial sequence
  primer M116 for CYP21A2
tgtggacgtgattccctttctcaggttcttccccaatccaggtctccgga

SEQ ID NO: 141 - DNA, 50 bp, artificial sequence
  primer M117 for CYP24
ctcaagaaacagcacgacaccctggtggagtaccacaagaagtatggcaa

SEQ ID NO: 142 - DNA, 50 bp, artificial sequence
  primer M118 for CYP27A1
ggttggaatgccatcttttcctttgggaagaagctgattgatgagaagct

SEQ ID NO: 143 - DNA, 48 bp, artificial sequence
  primer M119 for CYP2C9
ctgctgaagcacccagaggtcacagctaaagtccaggaagagattgaa

SEQ ID NO: 144 - DNA, 50 bp, artificial sequence
  primer M120 for CYP3A4
agtcgcctcgaagatacacaaaagcaccgagtggatttccttcagctgat

SEQ ID NO: 145 - DNA, 49 bp, artificial sequence
  primer M121 for HSD17B10
agtgtggctgccttcgagggtcaggttggacaagctgcatactctgctt

SEQ ID NO: 146 - DNA, 50 bp, artificial sequence
  primer M122 for HSD17B3
cctgaacgcaccggatgaaatccagagcctcatccattgtaacatcacct

SEQ ID NO: 147 - DNA, 49 bp, artificial sequence
  primer M123 for HSD17B8
aacatcagtagcatcgtaggaaaggtggggaacgtggggcagacaaact

SEQ ID NO: 148 - DNA, 50 bp, artificial sequence
  primer M124 for HSD17B8
ttcgtgctctgctggttccccttcttcttcagctacagcctgtacggcat

SEQ ID NO: 149 - DNA, 46 bp, artificial sequence
  primer M125 for HSD17B8
aggctttgccaacggctcgacggggcttcttggggagtttcttagg

SEQ ID NO: 150 - DNA, 46 bp, artificial sequence
  primer M126 for PNMT
aacatgcctgcctcattgagggcaagggggaatgctggcaggataa

SEQ ID NO: 151 - DNA, 50 bp, artificial sequence
  primer M127 for SLC6A
ttctaccgcttgaaagaggccacggtatggattgatgccgcaactcagat

SEQ ID NO: 152 - DNA, 50 bp, artificial sequence
  primer M128 for ADM
tgtcgcgtcggagtttcgaaagaagtggaataagtgggctctgagtcgtg

SEQ ID NO: 153 - DNA, 46 bp, artificial sequence
  primer M129 for ADMR
tcccaagggaaactcaggcgtgtgctggtcccaatgtcagtgaaac

SEQ ID NO: 154 - DNA, 50 bp, artificial sequence
  primer M130 for ANGPT1
caaccttgtcaatctttgcactaaagaaggtgttttactaaagggaggaa

SEQ ID NO: 155 - DNA, 50 bp, artificial sequence
  primer M131 for ANGPT2
atcccagtccacctgaggaactgtctcgaactattttcaaagacttaagc

SEQ ID NO: 156 - DNA, 47 bp, artificial sequence
  primer M132 for ANGPT4
aagaaagggctaacgcctcggccccggccttcataatggcaggtgag

SEQ ID NO: 157 - DNA, 47 bp, artificial sequence
  primer M133 for CDT6
agacggtcactcagacctccgcagatgccatctacgactgctcttcc

SEQ ID NO: 158 - DNA, 50 bp, artificial sequence
  primer M134 for LOC16
aactggatctgagcgcaaaaaggaagacacagacggtcaaaaacgacccc

SEQ ID NO: 159 - DNA, 50 bp, artificial sequence
  primer M135 for TEK
tgtggagtcagcttgctcctttctggaactgtggaaggtgccatggactt

SEQ ID NO: 160 - DNA, 50 bp, artificial sequence
  primer M136 for BDKRB1
ctgctgcacagagtgctgccaacatttatcatctccatctgtttcttcgg

SEQ ID NO: 161 - DNA, 46 bp, artificial sequence
  primer M137 for BDKRB2
tccgtgcccaccacggcctctttcagcgccgacatgctcaatgtca

SEQ ID NO: 162 - DNA, 50 bp, artificial sequence
  primer M138 for PTGIS
tgatgaaaaggccaggatgaaactgactcttctccacagagagctccagg

SEQ ID NO: 163 - DNA, 50 bp, artificial sequence
  primer M138 for CT
tcatgttagcatgccccagaatgccaactaaactcctccctttccttcct

SEQ ID NO: 164 - DNA, 49 bp, artificial sequence
  primer M140 for CALCA
atcattgcccagaagagagcctgtgacactgccacctgtgtgactcatc

SEQ ID NO: 165 - DNA, 50 bp, artificial sequence
  primer M141 for CALCB
tcaagcctgagcagatgaatgactccaggaagaaggttatcatgaaactg

SEQ ID NO: 166 - DNA, 50 bp, artificial sequence
  primer M142 for CALCR
agggccgtgtacttcaatgacaactgctggctgagtgtggaaacccattt

SEQ ID NO: 167 - DNA, 49 bp, artificial sequence
  primer M143 for ECE2
agccactgagagacctcattgagaagattggtggttggaacattacggg

SEQ ID NO: 168 - DNA, 47 bp, artificial sequence
  primer M144 for ECE2
ggctgccttgtggccctaggggtgtatgggatggattatttgcagca

SEQ ID NO: 169 - DNA, 50 bp, artificial sequence
  primer M145 for ECE1
ctctcatcaacaccaccgacagatgcctgctcaacaactacatgatctgg

SEQ ID NO: 170 - DNA, 50 bp, artificial sequence
  primer M146 for EDN1
acatcatttgggtcaacactcccgagcacgttgttccgtatggacttgga

SEQ ID NO: 171 - DNA, 50 bp, artificial sequence
  primer M147 for EDN2
acatcatctgggtgaacactcctgaacagacagctccttacggcctggga

SEQ ID NO: 172 - DNA, 49 bp, artificial sequence
  primer M148 for EDN3
acatcatttggatcaacactcccgaacagacggtgccctatggactgtc

SEQ ID NO: 173 - DNA, 50 bp, artificial sequence
  primer M149 for EDNRA
tgatctccctatcaatgtatttaagctgctggctgggcgctggccttttg

SEQ ID NO: 174 - DNA, 50 bp, artificial sequence
  primer M150 for EDNRB
tgacatccctatcaatgtctacaagctgctggcagaggactggccatttg

SEQ ID NO: 175 - DNA, 50 bp, artificial sequence
  primer M151 for EDNRB
gtctatgtgctctgagtattgacagatatcgagctgttgcttcttggagt

SEQ ID NO: 176 - DNA, 50 bp, artificial sequence
  primer M152 for CCK
agatacatccagcaggcccggaaagctccttctggacgaatgtccatcgt

SEQ ID NO: 177 - DNA, 47 bp, artificial sequence
  primer M153 for CCKBR
tgcaaggcggtttcctacctcatgggggtgtctgtgagtgtgtccac

SEQ ID NO: 178 - DNA, 41 bp, artificial sequence
  primer M154 for GAS
cccacacctcgtggcagacccgtccaagaagcagggaccat

SEQ ID NO: 179 - DNA, 49 bp, artificial sequence
  primer M155 for GAS
cgactatgtgtgtatgtgctgatctttgcactggctctggccgccttct

SEQ ID NO: 180 - DNA, 50 bp, artificial sequence
  primer M156 for GRP
aaagatgtaggttcaaaaggcaaagttggtagactctctgctccaggttc

SEQ ID NO: 181 - DNA, 50 bp, artificial sequence
  primer M157 for GRPR
agggaatatacatgtcaagaagcagattgaatcccggaagcgacttgcca

SEQ ID NO: 182 - DNA, 50 bp, artificial sequence
  primer M158 for LEP
caggatcaatgacatttcacacacgcagtcagtctcctccaaacagaaag

SEQ ID NO: 183 - DNA, 50 bp, artificial sequence
  primer M159 for LEPR
accttttcatggcctatgagcaaagtaaatatcgtgcagtcactcagtgc

SEQ ID NO: 184 - DNA, 50 bp, artificial sequence
  primer M160 for MCH-R2
tcatgactgtaatgagtgtggacaggtactttgccctcgtccaaccattt

SEQ ID NO: 185 - DNA, 50 bp, artificial sequence
  primer M161 for PMCH
cgaagaggaaaataaagtttcaaagaacacaggctccaaacataatttct

SEQ ID NO: 186 - DNA, 50 bp, artificial sequence
  primer M162 for PMCH
catgctcagatgtatgctgggaagagtctaccgaccttgttggcaagtct

SEQ ID NO: 187 - DNA, 49 bp, artificial sequence
  primer M163 for gene similar to PMCH on chromosome 5
gtagcgttaagatagatcagtaaccaaaatcataggctttttctgtgca

SEQ ID NO: 188 - DNA, 50 bp, artificial sequence
  primer M164 for CABP1
ggaactgcgagatgctttccgagagtttgacaccaatggtgatggggaaa

SEQ ID NO: 189 - DNA, 50 bp, artificial sequence
  primer M165 for CAPB1 variant 2
tccactgagaaatctctcaaggaaggatagatcactgcgaccagaggaaa

SEQ ID NO: 190 - DNA, 47 bp, artificial sequence
  primer M166 for CABP2
gcagcataaaggactcaaggaccacagcctctgcccaccagcatgtt

SEQ ID NO: 191 - DNA, 50 bp, artificial sequence
  primer M167 for CAPB3
ctcaagaaatgcttcccgcagctcttcaatctcatcttgtcccagtggtc

SEQ ID NO: 192 - DNA, 50 bp, artificial sequence
  primer M168 for CABP5
cactgggacaagatgagattgaagagctgcgggaagcatttcttgagttc

SEQ ID NO: 193 - DNA, 50 bp, artificial sequence
  primer M169 for CALM1
aactgtcatgaggtcactgggtcagaacccaacagaagctgaattgcagg

SEQ ID NO: 194 - DNA, 50 bp, artificial sequence
  primer M170 for CALM2
agaagcattccgtgtgtttgataaggatggcaatggctatattagtgctg

SEQ ID NO: 195 - DNA, 52 bp, artificial sequence
  primer M171 for CALM3
atatgatcaatgaggtggatgcagatgaccttccagggaacgggaccattga

SEQ ID NO: 196 - DNA, 50 bp, artificial sequence
  primer M172 for CALN1
cacgatagacagcatattctggcagtttgacatgcaaaggataactctgg

SEQ ID NO: 197 - DNA, 49 bp, artificial sequence
  primer M173 for CAMKK2
cacgtctccatcacgggtatgcaggactgtgtgcagctgaatcagtata

SEQ ID NO: 198 - DNA, 50 bp, artificial sequence
  primer M173 for PDE1A
gaaacagtttgcagcagcctgaagggattgacagagccaaaaccatgtcc

SEQ ID NO: 199 - DNA, 50 bp, artificial sequence
  primer M173 for MLN
ctatggcgaactccagaggatgcaggaaaaggaacggaataaagggcaaa

SEQ ID NO: 200 - DNA, 46 bp, artificial sequence
  primer M176 for NPPB
acttggaaacgtccgggttacaggagcagcgcaaccatttgcaggg

SEQ ID NO: 201 - DNA, 48 bp, artificial sequence
  primer M177 for NPPC
caaatacaaaggagccaacaagaagggcttgtccaagggctgcttcgg

SEQ ID NO: 202 - DNA, 50 bp, artificial sequence
  primer M178 for NPR1
cggatgtggaaccgaagctttcaaggtgtgacaggatacctgaaaattga

SEQ ID NO: 203 - DNA, 50 bp, artificial sequence
  primer M179 for NPR1
ggatcccgagaatggtgccttcagggttgtactgaactacaatgggactt

SEQ ID NO: 204 - DNA, 50 bp, artificial sequence
  primer M180 for NPR2
aaagatgcagggacgaagatatcacggtgtaactgggctggttgtcatgg

SEQ ID NO: 205 - DNA, 50 bp, artificial sequence
  primer M181 for NPR2
ttggacgacccatcctgtgataaaactccactttcaaccctggcaattgt

SEQ ID NO: 206 - DNA, 48 bp, artificial sequence
  primer M182 for NPR3
caatatccaggccagtgagagagtggtgatcatgtgtgcgagcagtga

SEQ ID NO: 207 - DNA, 50 bp, artificial sequence
  primer M183 for KIAA0976
ccctcctgagacgttctgtgcaatgggcaatccctacatgtgcaataatg

SEQ ID NO: 208 - DNA, 48 bp, artificial sequence
  primer M184 for KIAA1857
ccctgagaggttctgctcccatgagaatccctacctatgcagcaacga

SEQ ID NO: 209 - DNA, 44 bp, artificial sequence
  primer M185 for NTN1
cagcagcgtggaggagcctgaagactgcgattcctactgcaagg

SEQ ID NO: 210 - DNA, 49 bp, artificial sequence
  primer M186 for NTN4
ttctcagctccagatgcttgcaaaccgtgttcctgccatccagtaggat

SEQ ID NO: 211 - DNA, 50 bp, artificial sequence
  primer M187 for iNOS
caggatgaccctaagagtcacaagcatcaaaatggtttcccccagttcct

SEQ ID NO: 212 - DNA, 50 bp, artificial sequence
  primer M188 for NOS1
caactctgtgcaggaagaaaggaagagctacaaggtccgattcaacagcg

SEQ ID NO: 213 - DNA, 50 bp, artificial sequence
  primer M189 for NOS2
tgacacaggatgaccttcagtatcacaacctcagcaagcagcagaatgag

SEQ ID NO: 214 - DNA, 50 bp, artificial sequence
  primer M190 for NOS3
cacaggaaatgttcacctacatctgcaaccacatcaagtatgccaccaac

SEQ ID NO: 215 - DNA, 50 bp, artificial sequence
  primer M191 for PEDF
gcctcccggatcgtctttgagaagaagctgcgcataaaatccagctttgt

SEQ ID NO: 216 - DNA, 50 bp, artificial sequence
  primer M192 for NPY5R
gctactgtctggacactaggttttgccatctgttctccccttccagtgtt

SEQ ID NO: 217 - DNA, 50 bp, artificial sequence
  primer M193 for neuropeptidey/peptide YY receptor type 2
aatgggtccaataggtgcagaggctgatgagaaccagacagtggaagaaa

SEQ ID NO: 218 - DNA, 44 bp, artificial sequence
  primer M194 for PPY2
ttggagccattgtacccaggggacaataccacaccggagcagat

SEQ ID NO: 219 - DNA, 50 bp, artificial sequence
  primer M195 for PPYR1
tacaccatcatggactactggatctttggagagaccctctgcaagatgtc

SEQ ID NO: 220 - DNA, 41 bp, artificial sequence
  primer M196 for PYY
actacctcaacctggtcacccggcagcggtatgggaaaaga

SEQ ID NO: 221 - DNA, 46 bp, artificial sequence
  primer M197 for PYY2
tacgcctatcctcgccactacctcatcctggtcactcagccgtcgt

SEQ ID NO: 222 - DNA, 50 bp, artificial sequence
  primer M198 for VIP
tttacagggcaccttctgctctcaggttgggtgacagaataccctttgag

SEQ ID NO: 223 - DNA, 50 bp, artificial sequence
  primer M199 for VIP
agtctcttatgggaaaacgtgttagcagtaacatctcagaagaccctgta

SEQ ID NO: 224 - DNA, 50 bp, artificial sequence
  primer M200 for PTH
aaatcggatgggaaatctgttaagaagagatctgtgagtgaaatacagct

SEQ ID NO: 225 - DNA, 50 bp, artificial sequence
  primer M201 for PTHR1
ggtgcccatcctggcctccattgtgctcaacttcatcctcttcatcaata

SEQ ID NO: 226 - DNA, 50 bp, artificial sequence
  primer M202 for PTHR1
catatactgtttctgcaatggcgaggtacaagctgagatcaagaaatctt

SEQ ID NO: 227 - DNA, 50 bp, artificial sequence
  primer M203 for PTHR2
ggcatgacacaaggaagcaatacaggaaactggccaaatcgacactggtc

SEQ ID NO: 228 - DNA, 50 bp, artificial sequence
  primer M204 for PTHR2
ggcaacttctgtggacaaatcacaatatatcgggtgcaagattgctgttg

SEQ ID NO: 229 - DNA, 50 bp, artificial sequence
  primer M205 for CYP4F8
gtccgatctgtcatcaatacctcagatgccattacagacaaggacatagt

SEQ ID NO: 230 - DNA, 50 bp, artificial sequence
  primer M206 for PTGDR
cactatgtgttctctgcccgtaatttatcgcgcttactatggagcattta

SEQ ID NO: 231 - DNA, 47 bp, artificial sequence
  primer M207 for CYP4F11
gcctatcaccagtgcctcagctgctgtcgcacccaaggatatgattt

SEQ ID NO: 232 - DNA, 50 bp, artificial sequence
  primer M208 for CYP4F12
aacaagagtgcaaacatcatgcttgacaagtggcagcacctggcctcaga

SEQ ID NO: 233 - DNA, 50 bp, artificial sequence
  primer M209 for CYP4F2
atccggtctgtcatcaacgcctcagctgccattgcaccaaaggacaagtt

SEQ ID NO: 234 - DNA, 50 bp, artificial sequence
  primer M210 for CYP4F3
cattgatgtactcctgctgagcaaggatgaagatgggaagaagttgtccg

SEQ ID NO: 235 - DNA, 40 bp, artificial sequence
  primer M211 for PTGER1
cgtgcatctgctggagcccaatgctggtgttggtggcgct

SEQ ID NO: 236 - DNA, 50 bp, artificial sequence
  primer M212 for PTGER2
cgccgtctgctccttgcctttcacgatttttgcatatatgaatgaaacct

SEQ ID NO: 237 - DNA, 50 bp, artificial sequence
  primer M213 for PTGER3
aggacttggtgcagttctcatgatagagaaccctgcagtgtccagctaag

SEQ ID NO: 238 - DNA, 48 bp, artificial sequence
  primer M214 for PTGER4
tcatctgctccatcccgctcgtggtgcgagtattcgtcaaccagttat

SEQ ID NO: 239 - DNA, 50 bp, artificial sequence
  primer M215 for PTGFR
tcctgtatttgttggagcccatttctggttacaatggccaacattggaat

SEQ ID NO: 240 - DNA, 37 bp, artificial sequence
  primer M216 for PTGIR
tgtgctccctgcctctcacgatccgctgcttcaccca

SEQ ID NO: 241 - DNA, 50 bp, artificial sequence
  primer M217 for PTGS2
ttctatggagaaaactgctcaacaccggaatttttgacaagaataaaatt

SEQ ID NO: 242 - DNA, 50 bp, artificial sequence
  primer M218 for PTGS2
cagacaagcaggctaatactgataggagagactattaagattgtgattga

SEQ ID NO: 243 - DNA, 44 bp, artificial sequence
  primer M219 for TBXA2R
tctctgtcgcttcatgggcgtcgtcatgatcttcttcggcctgt

SEQ ID NO: 244 - DNA, 50 bp, artificial sequence
  primer M220 for TBXAS1
tttcagtcctgaaaagctgaacgagatggttcccctcatcagccaagcct

SEQ ID NO: 245 - DNA, 49 bp, artificial sequence
  primer M221 for TBXAS1
ggtagacgtttttaaggagaaacacatggcccctgagttctgcagcctc

SEQ ID NO: 246 - DNA, 50 bp, artificial sequence
  primer M222 for AGTR1
tgtacgctagtgtgtttctactcacgtgtctcagcattgatcgatacctg

SEQ ID NO: 247 - DNA, 50 bp, artificial sequence
  primer M223 for AGTR2
atttactccttttggctactcttcctctatgggcaacctattattcttat

SEQ ID NO: 248 - DNA, 50 bp, artificial sequence
  primer M224 for ACE
cttctacaacaggaaagacttcaggatcaagcagtgcacacgggtcacga

SEQ ID NO: 249 - DNA, 50 bp, artificial sequence
  primer M225 for AGT
ttcaacacctacgtccacttccaagggaagatgaagggcttctccctgct

SEQ ID NO: 250 - DNA, 50 bp, artificial sequence
  primer M226 for REN
cgacagacaccaccacctttaaacggatcttcctcaagagaatgccctca

SEQ ID NO: 251 - DNA, 49 bp, artificial sequence
  primer M227 for REN
ggagccaagaagaggctgtttgattatgtcgtgaagtgtaacgagggcc

SEQ ID NO: 252 - DNA, 49 bp, artificial sequence
  primer M228 for RENBP
aagtatgtgtggctgcaggggaggcaggtatggatgtattgtcgcctgt

SEQ ID NO: 253 - DNA, 50 bp, artificial sequence
  primer M229 for ACE2
agaactgaagttgaaaaggccatcaggatgtcccggagccgtatcaatga

SEQ ID NO: 254 - DNA, 37 bp, artificial sequence
  primer M230 for SCT
ctggtggggaagcgcagcgagcaggacgcagagaaca

SEQ ID NO: 255 - DNA, 49 bp, artificial sequence
  primer M231 for SCTR
ggatgctcaccagcagaaatggttccttgttccgaaactgcacacagga

SEQ ID NO: 256 - DNA, 50 bp, artificial sequence
  primer M232 for UTS2
atgctgggtgcagaaagaggggatattctcaggaaagcagactcaagtac

SEQ ID NO: 257 - DNA, 50 bp, artificial sequence
  primer M233 for AQP4
cagttatcatgggaaattgggaaaaccattggatatattgggttgggccc

SEQ ID NO: 258 - DNA, 50 bp, artificial sequence
  primer M234 for AVPR1A
acagaacattgtcttacttgatcttcccgatgacctcaacaacaggaaag

SEQ ID NO: 259 - DNA, 50 bp, artificial sequence
  primer M235 for AVPR1A
agatttcaagtccagcatctcaacgtgacaaccttggtaactctgcatga

SEQ ID NO: 260 - DNA, 41 bp, artificial sequence
  primer M236 for AVPR1B
cggtgaccatgctcacggcctgctacagcctcatctgccat

SEQ ID NO: 261 - DNA, 50 bp, artificial sequence
  primer M237 for AVPR1B
ccaccaatgtggctttcaccatctctatgcttttgggcaacctcaacagc

SEQ ID NO: 262 - DNA, 40 bp, artificial sequence
  primer M238 for AVPR2
atgctcatggcgtccaccacttccgctgtgcctgggcatc

SEQ ID NO: 263 - DNA, 43 bp, artificial sequence
  primer M239 for OXTR
atgccaacgcgcccaaggaagcctcggccttcatcatcgtcat

SEQ ID NO: 264 - DNA, 39 bp, artificial sequence
  primer M240 for OXT
aaaggccgctgcttcgggcccaatatctgctgcgcggaa

SEQ ID NO: 265 - DNA, 50 bp, artificial sequence
  primer M241 for oxytocinase
caccgctttatcaaatatgcctaagaagtcatcagtcgttctagatgatg

SEQ ID NO: 266 - DNA, 50 bp, artificial sequence
  primer M242 for RLN1
cctcagacacctagaccagtggcagaaattgtaccatccttcatcaacaa

SEQ ID NO: 267 - DNA, 50 bp, artificial sequence
  primer M243 for RLN1
ctatctgagaggcaaccatcattaccagagctacagcagtatgtacctgc

SEQ ID NO: 268 - DNA, 50 bp, artificial sequence
  primer M244 for RLN2
gagagttccttggtgccctttccaaattgtgccatccttcatcaacaaag

SEQ ID NO: 269 - DNA, 45 bp, artificial sequence
  primer M245 for RLN3
atcctggcccacgaggctatgggagataccttcccggatgcagat

SEQ ID NO: 270 - DNA, 36 bp, artificial sequence
  primer M246 for vassopressin precursor
atgtccgacctggagctgagacagtgcctcccctgc

SEQ ID NO: 271 - DNA, 34 bp, artificial sequence
  primer M247 for vassopressi precursor
tttgctgcaacgacgagagctgcgtgaccgagcc

SEQ ID NO: 272 - DNA, 50 bp, artificial sequence
  primer M248 for CHRNA1
gacaaccaatgtgcgtctgaaacagcaatgggtggattacaacctaaaat

SEQ ID NO: 273 - DNA, 50 bp, artificial sequence
  primer M249 for CHRNA1
tattctacctgcccacagactcaggggagaagatgactctgagcatctct

SEQ ID NO: 274 - DNA, 49 bp, artificial sequence
  primer M250 for CHRNA2
accaccaacgtctggctaaaacaggagtggagcgactacaaactgcgct

SEQ ID NO: 275 - DNA, 50 bp, artificial sequence
  primer M251 for CHRNA3
tcatggagaccaacctgtggctcaagcaaatctggaatgactacaagctg

SEQ ID NO: 276 - DNA, 50 bp, artificial sequence
  primer M252 for CHRNA4
gtccatcgctcagctcattgacgtggatgagaagaaccagatgatgacca

SEQ ID NO: 277 - DNA, 50 bp, artificial sequence
  primer M253 for CHRNA5
caccagacatcgttttgtttgataatgcagatggacgttttgaagggacc

SEQ ID NO: 278 - DNA, 50 bp, artificial sequence
  primer M254 for CHRNA6
ggaaaccaatttgtggctgcgtcacatctggaatgattataaattgcgct

SEQ ID NO: 279 - DNA, 50 bp, artificial sequence
  primer M255 for CHRNA7
gagcctcctgcagatcatggacgtggatgagaagaaccaagttttaacca

SEQ ID NO: 280 - DNA, 49 bp, artificial sequence
  primer M256 for CHRNB1
atcctggcgcaactcatcagcctgaacgagaaggatgaagagatgagca

SEQ ID NO: 281 - DNA, 50 bp, artificial sequence
  primer M257 for CHRNB2
agcagatcatgaccaccaatgtctggctgacccaggagtgggaagattat

SEQ ID NO: 282 - DNA, 50 bp, artificial sequence
  primer M258 for CHRNB3
ttcctgacatagttctctttgaaaatgctgacggccgcttcgaaggctcc

SEQ ID NO: 283 - DNA, 50 bp, artificial sequence
  primer M259 for CHRNB4
agaatgacgatgaagaccagagtgtcgttgaggactggaagtacgtggct

SEQ ID NO: 284 - DNA, 50 bp, artificial sequence
  primer M260 for DRD1
agactttgccctgcgacgaataatgccatagagacggtgagtatcaataa

SEQ ID NO: 285 - DNA, 50 bp, artificial sequence
  primer M261 for DRD2
ctcctcttcggactcaataacgcagaccagaacgagtgcatcattgccaa

SEQ ID NO: 286 - DNA, 49 bp, artificial sequence
  primer M262 for DRD3
ttctgtttggctttaataccacaggggaccccactgtctgctccatctc

SEQ ID NO: 287 - DNA, 36 bp, artificial sequence
  primer M263 for DRD4
tggtgctgccgctcttcgtctactccgaggtccagg

SEQ ID NO: 288 - DNA, 44 bp, artificial sequence
  primer M264 for AANAT
ctttgagatcgagcgtgaagccttcatctccgtcttgggcgtct

SEQ ID NO: 289 - DNA, 39 bp, artificial sequence
  primer M265 for AANAT
acaaggagagactcatgcaggagtcactgacgctgcaca

SEQ ID NO: 290 - DNA, 50 bp, artificial sequence
  primer M266 for TPH
gatctgaactagatgcagaccatcctggcttcaaagacaatgtctaccgt

SEQ ID NO: 291 - DNA, 49 bp, artificial sequence
  primer M267 for deletion of exon 2
ataaaagccctgaaaatctttcaggtatggaaactgttccttggtttcc

SEQ ID NO: 292 - DNA, 50 bp, artificial sequence
  primer M268 for TPH
cagatcccttctataccccagagccagatacctgccatgaactcttaggt

SEQ ID NO: 293 - DNA, 50 bp, artificial sequence
  primer M269 for HIOMT
aagaacagattgacttccaggaaggggatttcttcaaagaccctcttccg

SEQ ID NO: 294 - DNA, 50 bp, artificial sequence
  primer M270 for ABAT
ggaagtcccagggcctagatctcaggagttaatgaaacagctgaatataa

SEQ ID NO: 295 - DNA, 50 bp, artificial sequence
  primer M271 for AOX1
gccagaatttgaggaaggaagtaagacaagtccaaaactcttcgcagaag

SEQ ID NO: 296 - DNA, 48 bp, artificial sequence
  primer M272 for DBH
taccacaacccactggtgatagaaggacgaaacgactcctcaggcatc

SEQ ID NO: 297 - DNA, 50 bp, artificial sequence
  primer M273 for CHATvM
cgtcatcgtagcctgctgcaatcagttctttgtcttggatgttgtcatta

SEQ ID NO: 298 - DNA, 50 bp, artificial sequence
  primer M274 for Variant N1
tgaggctttgagaaaggagtaggagcctagcattccggcagaggaagaaa

SEQ ID NO: 299 - DNA, 42 bp, artificial sequence
  primer M275 for COMT
agagggctggagcctgctcagaggtgctttgaagatgccgga

SEQ ID NO: 300 - DNA, 46 bp, artificial sequence
  primer M276 for COMT
catgaacgtgggcgacaagaaaggcaagatcgtggacgccgtgatt

SEQ ID NO: 301 - DNA, 49 bp, artificial sequence
  primer M277 for DDC
aaaggactgcaggcttatatccgcaagcatgtccagctgtcccatgagt

SEQ ID NO: 302 - DNA, 50 bp, artificial sequence
  primer M278 for GAD2
ctctgctctcctggttagagaagagggattgatgcagaattgcaaccaaa

SEQ ID NO: 303 - DNA, 50 bp, artificial sequence
  primer M279 for GAD2
gacttctctaatctgtttgctagagatctgcttccggctaagaacggtga

SEQ ID NO: 304 - DNA, 50 bp, artificial sequence
  primer M280 for GAD2
gacagtgagcccaagtgtgtggaagagttgcctgagtggaatttcgatgg

SEQ ID NO: 305 - DNA, 50 bp, artificial sequence
  primer M281 for GAD2
catgcgggaggagaatggtctgaagtacatcgaggaggccattgagaaac

SEQ ID NO: 306 - DNA, 50 bp, artificial sequence
  primer M282 for HNMT
aagatgctcattattgttgtgtcaggaagcagtggctgggacaagctgtg

SEQ ID NO: 307 - DNA, 50 bp, artificial sequence
  primer M283 for MAOA
ctgggtacaagaacctgaatcaaaggacgttccagcggtagaaatcaccc

SEQ ID NO: 308 - DNA, 50 bp, artificial sequence
  primer M284 for MAOB
gggaggcaggacttacactcttaggaaccaaaaggttaaatatgtggacc

SEQ ID NO: 309 - DNA, 50 bp, artificial sequence
  primer M285 for HRH1
ccgagaggacaagtgtgagacagacttctatgatgtcacctggttcaagg

SEQ ID NO: 310 - DNA, 50 bp, artificial sequence
  primer M286 for HRH2
aagggcaatcataccacctctaagtgcaaagtccaggtcaatgaagtgta

SEQ ID NO: 311 - DNA, 43 bp, artificial sequence
  primer M287 for HRH3
tcaacctcgccatctccgacttcctcgtcggcgccttctgcat

SEQ ID NO: 312 - DNA, 50 bp, artificial sequence
  primer M288 for HRH4
ggccatctctgacttctttgtgggtgtgatctccattcctttgtacatcc

SEQ ID NO: 313 - DNA, 50 bp, artificial sequence
  primer M289 for MTNR1A
gtatcggaacaagaagctcaggaacgcaggaaacatctttgtggtgagct

SEQ ID NO: 314 - DNA, 48 bp, artificial sequence
  primer M290 for MTNR1B
aggaaccgcaagctccggaacgcaggtaatttgttcttggtgagtctg

SEQ ID NO: 315 - DNA, 50 bp, artificial sequence
  primer M291 for NTS
ttcagctcctggagtctgtgctcagattcagaagaggaaatgaaagcatt

SEQ ID NO: 316 - DNA, 50 bp, artificial sequence
  primer M292 for NTSR1
catctcggatgagcagtggactccgttcctctatgacttctaccactact

SEQ ID NO: 317 - DNA, 47 bp, artificial sequence
  primer M293 for NTSR2
accgcgctccaagtctttatccaggtgaatgtgctggtgtccttcgt

SEQ ID NO: 318 - DNA, 50 bp, artificial sequence
  primer M294 for ADRA1A
ttttcttagtcatgcccattgggtctttcttccctgatttcaagccctct

SEQ ID NO: 319 - DNA, 48 bp, artificial sequence
  primer M295 for ADRA1B
tacccttcttcatcgctctaccgcttggctccttgttctccaccctga

SEQ ID NO: 320 - DNA, 45 bp, artificial sequence
  primer M296 for ADRA1D
tgctggttccctttcttctttgtcctgccgctcggctccttgttc

SEQ ID NO: 321 - DNA, 50 bp, artificial sequence
  primer M297 for ADRA2A
tctccctactctctcccgccgcttagaaataaaacttggctgtattagga

SEQ ID NO: 322 - DNA, 50 bp, artificial sequence
  primer M298 for ADRA2B
ttcctcattctctttaccatcttcggcaacgctctggtcatcctggctgt

SEQ ID NO: 323 - DNA, 48 bp, artificial sequence
  primer M299 for ADRA2C
caacagctcgctcaacccggtcatctacacggtcttcaaccaggattt

SEQ ID NO: 324 - DNA, 50 bp, artificial sequence
  primer M300 for ADRB1
taagaccgatagcaggtgaactcgaagcccacaatcctcgtctgaatcat

SEQ ID NO: 325 - DNA, 49 bp, artificial sequence
  primer M301 for ADRB2
gagacctgctgtgacttcttcacgaaccaagcctatgccattgcctctt

SEQ ID NO: 326 - DNA, 50 bp, artificial sequence
  primer M302 for ADRB3
cctgaaggacaagaagcaacaactctgttgatcagaacctgtggaaaacc

SEQ ID NO: 327 - DNA, 50 bp, artificial sequence
  primer M303 for HTR1A
aattggctgggctactccaactctctgcttaaccccgtcatttacgcata

SEQ ID NO: 328 - DNA, 46 bp, artificial sequence
  primer M304 for HTR1B
tggaaaagaagaaactcatggccgctagggagcgcaaagccaccaa

SEQ ID NO: 329 - DNA, 49 bp, artificial sequence
  primer M305 for HTR1D
ccaaatcttgtgtgacatctggctgtcctctgacatcacgtgctgcaca

SEQ ID NO: 330 - DNA, 50 bp, artificial sequence
  primer M306 for HTR1E
ggaaacatgaacatcacaaactgtaccacagaggccagcatggctataag

SEQ ID NO: 331 - DNA, 50 bp, artificial sequence
  primer M307 for HTR1F
attgtgtatattgtgagagagagctggattatggggcaagtggtctgtga

SEQ ID NO: 332 - DNA, 45 bp, artificial sequence
  primer M308 for HTR3A
catgttaaccatcctgtatgggtaccggtggcctctgccgagcaa

SEQ ID NO: 333 - DNA, 50 bp, artificial sequence
  primer M309 for HTR2C
ctgtctctcctggcaatcctttatgattatgtctggccactacctagata

SEQ ID NO: 334 - DNA, 50 bp, artificial sequence
  primer M310 for HTR3A
tcccggacattctcatcaatgagttcgtggatgtggggaagtctccaaat

SEQ ID NO: 335 - DNA, 50 bp, artificial sequence
  primer M311 for HTR3B
accttcaagagcattctgcatacagtggaagacgtagacctggcctttct

SEQ ID NO: 336 - DNA, 50 bp, artificial sequence
  primer M312 for HTR4
ttcctgtaatggacaaacttgatgctaatgtgagttctgaggagggtttc

SEQ ID NO: 337 - DNA, 43 bp, artificial sequence
  primer M313 for HTR5A
acccatatccgaagctgtggaggtgaaggactctgccaaacag

SEQ ID NO: 338 - DNA, 40 bp, artificial sequence
  primer M314 for HTR6
tttgtggccaacatagtccaggccgtgtgcgactgcatct

SEQ ID NO: 339 - DNA, 50 bp, artificial sequence
  primer M315 for EGF
agagagatgggaaaacatgtagcggttgttcctcacccgataatggtgga

SEQ ID NO: 340 - DNA, 50 bp, artificial sequence
  primer M316 for EGFR
aagtgcgaagggccttgccgcaaagtgtgtaacggaataggtattggtga

SEQ ID NO: 341 - DNA, 49 bp, artificial sequence
  primer M317 for EGFR
atacgcggcaggaccaagcaacatggtcagttttctcttgcagtcgtca

SEQ ID NO: 342 - DNA, 49 bp, artificial sequence
  primer M318 for ERBB2
tgaactggtgtatgcagattgccaaggggatgagctacctggaggatgt

SEQ ID NO: 343 - DNA, 50 bp, artificial sequence
  primer M319 for FGF1
caccgacgggcttttatacggctcacagacaccaaatgaggaatgtttgt

SEQ ID NO: 344 - DNA, 50 bp, artificial sequence
  primer M320 for FGF2
gtccgggagaagagcgaccctcacatcaagctacaacttcaagcagaaga

SEQ ID NO: 345 - DNA, 46 bp, artificial sequence
  primer M321 for FGF3
tggccatgaacaagaggggacgactctatgcttcggagcactacag

SEQ ID NO: 346 - DNA, 45 bp, artificial sequence
  primer M322 for FGF4
ggcaagctctatggctcgcccttcttcaccgatgagtgcacgttc

SEQ ID NO: 347 - DNA, 50 bp, artificial sequence
  primer M323 for FGF5
gatcccacgaagccaatatgttaagtgttttggaaatatttgctgtgtct

SEQ ID NO: 348 - DNA, 50 bp, artificial sequence
  primer M324 for FGF6
cagtaaaggaagattgtacgcaacgcccagcttccaagaagaatgcaagt

SEQ ID NO: 349 - DNA, 50 bp, artificial sequence
  primer M325 for FGF7
acccaagagatgaagaataattacaatatcatggaaatcaggacagtggc

SEQ ID NO: 350 - DNA, 45 bp, artificial sequence
  primer M326 for FGF8
agaaggggaagctgatcgccaagagcaacggcaaaggcaaggact

SEQ ID NO: 351 - DNA, 50 bp, artificial sequence
  primer M327 for FGF10
agaaggggaaactctatggctcaaaagaatttaacaatgactgtaagctg

SEQ ID NO: 352 - DNA, 48 bp, artificial sequence
  primer M328 for FGF11
atgctgagggactgctctacagttcgccgcatttcacagctgagtgtc

SEQ ID NO: 353 - DNA, 50 bp, artificial sequence
  primer M329 for FGF12
accaaggacgaaaacagcgactacactctcttcaatctaattcccgtggg

SEQ ID NO: 354 - DNA, 50 bp, artificial sequence
  primer M330 for FGF13
aagaagaggcgcagaagaagaccagagcctcagcttaagggtatagttac

SEQ ID NO: 355 - DNA, 50 bp, artificial sequence
  primer M331 for FGF14
accaaggatgacagcactaattctacactcttcaacctcataccagtggg

SEQ ID NO: 356 - DNA, 50 bp, artificial sequence
  primer M332 for FGF16
acctaggaatgaatgagcgaggagaactctatgggtcgaagaaactcaca

SEQ ID NO: 357 - DNA, 50 bp, artificial sequence
  primer M333 for FGF17
ttctctgctgtcaaactcagggggagaatcacccgtctcctaattttaac

SEQ ID NO: 358 - DNA, 46 bp, artificial sequence
  primer M334 for FGF18
cctccgcctgcacttgcctgtgtttacacttcctgctgctgtgctt

SEQ ID NO: 359 - DNA, 45 bp, artificial sequence
  primer M335 for FGF19
gacggcaagatgcaggggctgcttcagtactcggaggaagactgt

SEQ ID NO: 360 - DNA, 50 bp, artificial sequence
  primer M336 for FGF20
gaatgacaaaggagaactctatggatcagagaaacttacttccgaatgca

SEQ ID NO: 361 - DNA, 45 bp, artificial sequence
  primer M337 for FGF21
tgctgaccagagccccgaaagtctcctgcagctgaaagccttgaa

SEQ ID NO: 362 - DNA, 40 bp, artificial sequence
  primer M338 for FGF22
ccacggccaggacagcatcctggagatccgctctgtacac

SEQ ID NO: 363 - DNA, 50 bp, artificial sequence
  primer M339 for FGF23
tttcagaggcaacatttttggatcacactatttcgacccggagaactgca

SEQ ID NO: 364 - DNA, 50 bp, artificial sequence
  primer M340 for FGFR1
cgactactataaaaagacaaccaacggccgactgcctgtgaagtggatgg

SEQ ID NO: 365 - DNA, 50 bp, artificial sequence
  primer M341 for FGFR2
ttcttggagcctgcacacaggatgggcctctctatgtcatagttgagtat

SEQ ID NO: 366 - DNA, 49 bp, artificial sequence
  primer M342 for FGFR3
accgtagccgtgaagatgctgaaagacgatgccactgacaaggacctgt

SEQ ID NO: 367 - DNA, 48 bp, artificial sequence
  primer M343 for FGFR4
actgtggccgtcaagatgctcaaagacaacgcctctgacaaggacctg

SEQ ID NO: 368 - DNA, 50 bp, artificial sequence
  primer M344 for FIBP
cactactcatcgagaggtactatgcctttgatgaggcctttgttcgggag

SEQ ID NO: 369 - DNA, 50 bp, artificial sequence
  primer M345 for SPRY1
ctgttcacaatcacactgctgctctagatacctgtgtatgggagccatgt

SEQ ID NO: 370 - DNA, 50 bp, artificial sequence
  primer M346 for SPRY2
cccagaacgtgattgactatgggacttgtgtatgctgtgtgaaaggtctc

SEQ ID NO: 371 - DNA, 50 bp, artificial sequence
  primer M347 for IGF1R
taccggcacaattactgctccaaagacaaaatccccatcaggaagtatgc

SEQ ID NO: 372 - DNA, 41 bp, artificial sequence
  primer M348 for IGF2
tcgacccctccgaccgtgcttccggacaacttccccagata

SEQ ID NO: 373 - DNA, 50 bp, artificial sequence
  primer M349 for IGF1
gacatgcccaagacccagaaggaagtacatttgaagaacgcaagtagagg

SEQ ID NO: 374 - DNA, 51 bp, artificial sequence
  primer M350 for IGFBP1
gaatggattttatcacagcagacagtgtgagacatccatggatggagaggc

SEQ ID NO: 375 - DNA, 50 bp, artificial sequence
  primer M351 for IGFBP2
tcataccatgaccggaagtcaaaagttgacctggataggctcaatgatga

SEQ ID NO: 376 - DNA, 50 bp, artificial sequence
  primer M352 for IGFBP3
aagcgggagacagaatatggtccctgccgtagagaaatggaagacacact

SEQ ID NO: 377 - DNA, 42 bp, artificial sequence
  primer M353 for IGFBP4
aacggcaacttccaccccaagcagtgtcacccagctctggat

SEQ ID NO: 378 - DNA, 49 bp, artificial sequence
  primer M354 for IGFBP5
caaaggattctacaagagaaagcagtgcaaaccttcccgtggccgcaag

SEQ ID NO: 379 - DNA, 42 bp, artificial sequence
  primer M355 for IGFBP6
gcgcgcctgctgttgcagaggagaatcctaaggagagtaaac

SEQ ID NO: 380 - DNA, 50 bp, artificial sequence
  primer M356 for IMP-1
atggtacagtagagaactgtgagcaagtgaacaccgagagtgagacggca

SEQ ID NO: 381 - DNA, 50 bp, artificial sequence
  primer M357 for IMP-2
ggacagtggagaatgtggaacaagtcaacacagacacagaaaccgccgtt

SEQ ID NO: 382 - DNA, 50 bp, artificial sequence
  primer M358 for IMP-3
tatcccgcctcatttacagtgggaggtgctggatagtttactagtccagt

SEQ ID NO: 383 - DNA, 50 bp, artificial sequence
  primer M359 for NGFB
gttctacactctgatcacagcttttctgatcggcatacaggcggaaccac

SEQ ID NO: 384 - DNA, 50 bp, artificial sequence
  primer M360 for NGF-2
caagctctccaagcagatggtggacgttaaggaaaattaccagagcaccc

SEQ ID NO: 385 - DNA, 50 bp, artificial sequence
  primer M361 for NGFR
ttgtggcctacatagccttcaagaggtggaacagctgcaagcagaacaag

SEQ ID NO: 386 - DNA, 44 bp, artificial sequence
  primer M362 for TDGF1
tctttgaactgggattagttgccgggctgggccatcaggaattt

SEQ ID NO: 387 - DNA, 48 bp, artificial sequence
  primer M363 for PDGFA
agatagactccgtagggagtgaggattctttggacaccagcctgagag

SEQ ID NO: 388 - DNA, 47 bp, artificial sequence
  primer M364 for PDGFB
gacctgtccaggtgagaaagatcgagattgtgcggaagaagccaatc

SEQ ID NO: 389 - DNA, 50 bp, artificial sequence
  primer M365 for PDGFC
tttccagcaacaaggaacagaacggagtacaagatcctcagcatgagaga

SEQ ID NO: 390 - DNA, 50 bp, artificial sequence; identical to SEQ ID NO: 375
  primer M366 for PDGFD

SEQ ID NO: 391 - DNA, 50 bp, artificial sequence
  primer M367 for PDGFRA
tcaatggacttaccctggagaagtgaaaggcaaaggcatcacaatgctgg

SEQ ID NO: 392 - DNA, 50 bp, artificial sequence
  primer M368 for PDGFRB
tgcctactatgtctacagactccaggtgtcatccatcaacgtctctgtga

SEQ ID NO: 393 - DNA, 50 bp, artificial sequence
  primer M369 for VEGF
tgaggagtccaacatcaccatgcagattatgcggatcaaacctcaccaag

SEQ ID NO: 394 - DNA, 50 bp, artificial sequence
  primer M370 for VEGFB
accagaggaaagtggtgtcatggatagatgtgtatactcgcgctacctgc

SEQ ID NO: 395 - DNA, 50 bp, artificial sequence
  primer M371 for VEGFC
cagcacgagctacctcagcaagacgttatttgaaattacagtgcctctct

SEQ ID NO: 396 - DNA, 50 bp, artificial sequence
  primer M372 for VEGFD
cagtaatgaacatggaccagtgaagcgatcatctcagtccacattggaac

SEQ ID NO: 397 - DNA, 50 bp, artificial sequence
  primer M373 for VEGFD
tttgcagaacttgtggaaaaactagaaaatagtgggtttacatactcaac

SEQ ID NO: 398 - DNA, 48 bp, artificial sequence
  primer M374 for ADAR
aaagaaggccctgcccatgaacccaagttccaatactgtgttgcagtg

SEQ ID NO: 399 - DNA, 48 bp, artificial sequence
  primer M375 for SPS
atcgtagacgacccttacatgatgggcaggatagcgtgtgccaatgtc

SEQ ID NO: 400 - DNA, 50 bp, artificial sequence
  primer M376 for SPS2
atgttactcagcgtcagccagagtatgagtgaggaggaacgcgaaaaggt

SEQ ID NO: 401 - DNA, 50 bp, artificial sequence
  primer M377 for LOC123574
gaactgaagggcacaggctgcaaagagaaccacttccaagaagatgagca

SEQ ID NO: 402 - DNA, 50 bp, artificial sequence
  primer M378 for LOC129579
ttaaagatgcagccgaggaagcaggaatatgtagagttgacataccaaga

SEQ ID NO: 403 - DNA, 50 bp, artificial sequence
  primer M379 for LOC150537
aaaatatgaatcccacacctgggccacctcttaatctagacagaaatagc

SEQ ID NO: 404 - DNA, 50 bp, artificial sequence
  primer M380 for LOC158861
cagaatgtgacaatatgctgatgctccttggagtcagtaataaaatgacc

SEQ ID NO: 405 - DNA, 50 bp, artificial sequence
  primer M381 for LOC167038
ttacagtgcttttgctgaaaatgtgaatgcaggaactgagaccatcatta

SEQ ID NO: 406 - DNA, 50 bp, artificial sequence; identical to SEQ ID NO: 404
  primer M382 for LOC168474

SEQ ID NO: 407 - DNA, 50 bp, artificial sequence
  primer M383 for LOC202064
atatcacaggctttggcattctaggacactctcagaacctcgtgaagcaa

SEQ ID NO: 408 - DNA, 44 bp, artificial sequence
  primer M384 for SCYA21
aaaggaaagggctccaaaggctgcaagaggactgagcggtcaca

SEQ ID NO: 409 - DNA, 50 bp, artificial sequence
  primer M385 for ACVR1
ccaatgttggagacagcactttagcagatttattggatcattcgtgtaca

SEQ ID NO: 410 - DNA, 50 bp, artificial sequence
  primer M386 for ACVR1B
agattgctcgaagatgcaattctggaggagtccatgaagaatatcagctg

SEQ ID NO: 411 - DNA, 50 bp, artificial sequence
  primer M387 for ACVR2
gctgatcacagcatttcatgaaaagggttcactatcagactttcttaagg

SEQ ID NO: 412 - DNA, 45 bp, artificial sequence
  primer M388 for ACVR2B
tgctggctagatgacttcaactgctacgataggcaggagtgtgtg

SEQ ID NO: 413 - DNA, 47 bp, artificial sequence
  primer M389 for INHBA
atcatcacgtttgccgagtcaggaacagccaggaagacgctgcactt

SEQ ID NO: 414 - DNA, 40 bp, artificial sequence
  primer M390 for INHB
aatcatcagcttcgccgagacagatggcctcgcctcctcc

SEQ ID NO: 415 - DNA, 49 bp, artificial sequence
  primer M391 for INHBC
gaaatcatcagctttgctgagacaggcctctccaccatcaaccagactc

SEQ ID NO: 416 - DNA, 50 bp, artificial sequence
  primer M392 for BLC
atggttgtccaagaaaagaaatcatagtctggaagaagaacaagtcaatt

SEQ ID NO: 417 - DNA, 50 bp, artificial sequence
  primer M393 for TNFSF13B
gccgttcagggtccagaagaaacagtcactcaagactgcttgcaactgat

SEQ ID NO: 418 - DNA, 50 bp, artificial sequence
  primer M394 for TNFRSF5 (CD40L)
aaacagtcagtgctgttctttgtgccagccaggacagaaactggtgagtg

SEQ ID NO: 419 - DNA, 50 bp, artificial sequence
  primer M395 for CNTF
ctgactgctcttacggaatcctatgtgaagcatcagggcctgaacaagaa

SEQ ID NO: 420 - DNA, 50 bp, artificial sequence
  primer M396 for CNTFR
atcacctttgacgagttcaccattgtgaagcctgatcctccagaaaatgt

SEQ ID NO: 421 - DNA, 50 bp, artificial sequence
  primer M397 for CSF1
acatttgagtttgtagaccaggaacagttgaaagatccagtgtgctacct

SEQ ID NO: 422 - DNA, 50 bp, artificial sequence
  primer M398 for CSF1R
cttcctccaacacaacaacactaagctcgcaatccctcaacaatctgact

SEQ ID NO: 423 - DNA, 50 bp, artificial sequence
  primer M399 for CSF2
tgaacctgagtagagacactgctgctgagatgaatgaaacagtagaagtc

SEQ ID NO: 424 - DNA, 50 bp, artificial sequence
  primer M400 for CSF2RA
ctagtctcaatgtgaggtttgactccaggacgatgaatttaagctgggac

SEQ ID NO: 425 - DNA, 49 bp, artificial sequence
  primer M401 for CSF2RB
ggcagagaaacacataaagagctcagtgaacatccagatggcccctcca

SEQ ID NO: 426 - DNA, 43 bp, artificial sequence
  primer M402 for CSF3
aggagaagctggtgagtgagtgtgccacctacaagctgtgcca

SEQ ID NO: 427 - DNA, 50 bp, artificial sequence
  primer M403 for CTF1
catgtgtacatctcagccttatctcaaggaggtgacaccttctctccttg

SEQ ID NO: 428 - DNA, 42 bp, artificial sequence
  primer M404 for CLC
aagcctcaatgacaaactgcggctgacccagaactacgaggc

SEQ ID NO: 429 - DNA, 50 bp, artificial sequence
  primer M405 for EMAPII (SCYE1)
caggcagaaattcaaaatggagtgaagcaaatagcatttccatctggtac

SEQ ID NO: 430 - DNA, 49 bp, artificial sequence
  primer M406 for Epo
ccgagaatatcacgacgggctgtgctgaacactgcagcttgaatgagaa

SEQ ID NO: 431 - DNA, 50 bp, artificial sequence
  primer M407 for CCR3
catggcatgtgtaagctcctctcagggttttatcacacaggcttgtacag

SEQ ID NO: 432 - DNA, 50 bp, artificial sequence
  primer M408 for eotaxin
tggcaaatgtccccagaaagctgtgatcttcaagaccaaactggccaagg

SEQ ID NO: 433 - DNA, 50 bp, artificial sequence
  primer M409 for osteopontin
ctaggcatcacctgtgccataccagttaaacaggctgattctggaagttc

SEQ ID NO: 434 - DNA, 50 bp, artificial sequence
  primer M410 for GSN
aagatgggaaaatctttgtctggaaaggcaagcaggcaaacacggaggag

SEQ ID NO: 435 - DNA, 50 bp, artificial sequence
  primer M411 for GZMB
agagattaaaaagacttcctttaagggggactctggaggccctcttgtgt

SEQ ID NO: 436 - DNA, 50 bp, artificial sequence
  primer M412 for CD44
gacaggacctctttcaatgacaacgcagcagagtaattctcagagcttct

SEQ ID NO: 437 - DNA, 50 bp, artificial sequence
  primer M413 for CD44
agcacagacagaatccctcgtaccaatatggactccagtcatagtacaac

SEQ ID NO: 438 - DNA, 50 bp, artificial sequence
  primer M414 for IFN-alpha D
attctgcaccgaactctaccagcagctgaatgacttggaagcctgtgtga

SEQ ID NO: 439 - DNA, 50 bp, artificial sequence
  primer M415 for IFN-alpha 14
tgcctgaaggacagacatgactttgaatttccccaggaggaatttgatgg

SEQ ID NO: 440 - DNA, 50 bp, artificial sequence
  primer M416 for IFN-alpha 16
acaaaggattcatctgctgcttgggatgagaccctcctagacaaattcta

SEQ ID NO: 441 - DNA, 50 bp, artificial sequence
  primer M417 for IFN-alpha 4b
aatacagcccttgtgcctgggaggttgtcagagcagaaatcatgagatcc

SEQ ID NO: 442 - DNA, 50 bp, artificial sequence
  primer M418 for IFN-alpha 5
gtaacaggaggactttgatgataatggcacaaatgggaagaatctctcct

SEQ ID NO: 443 - DNA, 50 bp, artificial sequence
  primer M419 for IFN-alpha 6, 5, 16 and F
atgacttcagatttccccaggaggagtttgatggcaaccagttccagaag

SEQ ID NO: 444 - DNA, 50 bp, artificial sequence
  primer M420 for IFN-alpha 7 and IFN-alpha 21
agcctgcgtaataggagggccttgatactcctggcacaaatgggaagaat

SEQ ID NO: 445 - DNA, 50 bp, artificial sequence
  primer M421 for IFN-alpha 1B and IFN-alpha 8
gagatgatccagcagaccttcaacctcttcagcacaaaggactcatctgc

SEQ ID NO: 446 - DNA, 50 bp, artificial sequence
  primer M422 for IFNAR1
gactcatttacaccatttcgcaaagctcagattggtcctccagaagtaca

SEQ ID NO: 447 - DNA, 50 bp, artificial sequence
  primer M423 for soluble and membrane bound IFNAR2
cacttaatttggttctcatggtgtatatcagcctcgtgtttggtatttca

SEQ ID NO: 448 - DNA, 50 bp, artificial sequence
  primer M424 for IFN-beta
gatcaagcaactccagcaattccagaaagaagacgcagctctgactatct

SEQ ID NO: 449 - DNA, 50 bp, artificial sequence
  primer M425 for IFN-beta2a(IL6)
atgcttccaatctggattcaatgaggagacttgcctggtgaaaatcatca

SEQ ID NO: 450 - DNA, 50 bp, artificial sequence; identical to SEQ ID NO: 446
  primer M426 for IFMBR

SEQ ID NO: 451 - DNA, 50 bp, artificial sequence
  primer M427 for IFN-gamma antagonist cytokine
accttctaagtcacgtcaccatgagatgccaagggagtacaatgaggatg

SEQ ID NO: 452 - DNA, 50 bp, artificial sequence
  primer M428 for IFNG
cttaggcattttgaagaattggaaagaggagagtgacagaaaaataatgc

SEQ ID NO: 453 - DNA, 50 bp, artificial sequence
  primer M429 for IFNGR1
gtatgtgagaatgaacggaagtgagatccagtataaaatactcacgcaga

SEQ ID NO: 454 - DNA, 50 bp, artificial sequence
  primer M430 for IFNGR2
ttgtctaccgagtgcagtttaaatacaccgacagtaaatggttcacggcc

SEQ ID NO: 455 - DNA, 50 bp, artificial sequence
  primer M431 for IL18
gatatgactgattctgactgtagagataatgcaccccggaccatatttat

SEQ ID NO: 456 - DNA, 50 bp, artificial sequence
  primer M432 for IL1A
gcattacataatctggatgaagcagtgaaatttgacatgggtgcttataa

SEQ ID NO: 457 - DNA, 50 bp, artificial sequence
  primer M433 for IL1B
ccagtgaaatgatggcttattacagtggcaatgaggatgacttgttcttt

SEQ ID NO: 458 - DNA, 50 bp, artificial sequence
  primer M434 for IL1E
gatggtggaggaagggccgtctatcaatcaatgtgtaaacctattactgg

SEQ ID NO: 459 - DNA, 50 bp, artificial sequence
  primer M435 for IL1R1
gtaatagaatttattactctagaggaaaacaaacccacaaggcctgtgat

SEQ ID NO: 460 - DNA, 50 bp, artificial sequence
  primer M436 for IL1R2
ctggcacctacgtctgcactactagaaatgcttcttactgtgacaaaatg

SEQ ID NO: 461 - DNA, 50 bp, artificial sequence
  primer M437 for IL10
cttcagcagagtgaagactttctttcaaatgaaggatcagctggacaact

SEQ ID NO: 462 - DNA, 50 bp, artificial sequence
  primer M438 for IL10RA
tacctgctatgaagtggcgctcctgaggtatggaatagagtcctggaact

SEQ ID NO: 463 - DNA, 50 bp, artificial sequence
  primer M439 for IL10RB
ttctcttccacagcacctgaaagagtttttgggccatcctcatcataaca

SEQ ID NO: 464 - DNA, 40 bp, artificial sequence
  primer M440 for IL11
gacatgaactgtgtttgccgcctggtcctggtcgtgctga

SEQ ID NO: 465 - DNA, 49 bp, artificial sequence
  primer M441 for IL10RA
tatgagaacttctcttgcacttggagtcccagccagatcagcggtttac

SEQ ID NO: 466 - DNA, 50 bp, artificial sequence
  primer M442 for IL12
ggccgtcagcaacatgctccagaaggccagacaaactctagaattttacc

SEQ ID NO: 467 - DNA, 49 bp, artificial sequence
  primer M443 for IL12RB1
tgaccctgcagctctacaactcagttaaatatgagcctcctctgggaga

SEQ ID NO: 468 - DNA, 50 bp, artificial sequence
  primer M444 for IL12RB2
agtaacagcaggctctggaatatggttaatgttacaaaggccaaaggaag

SEQ ID NO: 469 - DNA, 46 bp, artificial sequence
  primer M445 for IL13
atcacccagaaccagaaggctccgctctgcaatggcagcatggtat

SEQ ID NO: 470 - DNA, 50 bp, artificial sequence
  primer M446 for IL13RA1
ccagaatttgagagaaatgtggagaatacatcttgtttcatggtccctgg

SEQ ID NO: 471 - DNA, 50 bp, artificial sequence
  primer M447 for IL13RA2
tggagtgataaacaatgctgggaaggtgaagacctatcgaagaaaacttt

SEQ ID NO: 472 - DNA, 50 bp, artificial sequence
  primer M448 for IL15
cattcatgtcttcattttgggctgtttcagtgcagggcttcctaaaacag

SEQ ID NO: 473 - DNA, 47 bp, artificial sequence
  primer M449 for IL15RA
ttctggaaaagagcccgcagcttcatctcccagctcaaacaacacag

SEQ ID NO: 474 - DNA, 50 bp, artificial sequence
  primer M450 for IL2
atttaagttttacatgcccaagaaggccacagaactgaaacagcttcagt

SEQ ID NO: 475 - DNA, 50 bp, artificial sequence
  primer M451 for IL2RA
aaccaatgtcaatgcacaagctctgccactcggaacacaacgaaacaagt

SEQ ID NO: 476 - DNA, 50 bp, artificial sequence
  primer M452 for IL2RB
aacctgatcctcggagccccagattctcagaaactgaccacagttgacat

SEQ ID NO: 477 - DNA, 50 bp, artificial sequence
  primer M453 for IL2RG
tgggaatgaagacaccacagctgatttcttcctgaccactatgcccactg

SEQ ID NO: 478 - DNA, 50 bp, artificial sequence
  primer M454 for IL3
atcagcaattgagagcattcttaaaaatctcctgccatgtctgcccctgg

SEQ ID NO: 479 - DNA, 50 bp, artificial sequence
  primer M455 for IL3RA
ttgccaacaggcgtcaacagtacgagtgtcttcactacaaaacggatgct

SEQ ID NO: 480 - DNA, 50 bp, artificial sequence
  primer M456 for IL4
taacagacatctttgctgcctccaagaacacaactgagaaggaaaccttc

SEQ ID NO: 481 - DNA, 50 bp, artificial sequence
  primer M457 for IL4R
ttggagtgaaaacgacccggcagatttcagaatctataacgtgacctacc

SEQ ID NO: 482 - DNA, 50 bp, artificial sequence
  primer M458 for IL5
tcgaactctgctgatagccaatgagactctgaggattcctgttcctgtac

SEQ ID NO: 483 - DNA, 50 bp, artificial sequence
  primer M459 for IL5RA
acacgcagtattttctctactataggtatggctcttggactgaagaatgc

SEQ ID NO: 484 - DNA, 50 bp, artificial sequence
  primer M460 for IFNB1
agtgtctcctccaaattgctctcctgttgtgcttctccactacagctctt

SEQ ID NO: 485 - DNA, 50 bp, artificial sequence
  primer M461 for IL6R
tcaaagacattcacaacatggatggtcaaggacctccagcatcactgtgt

SEQ ID NO: 486 - DNA, 50 bp, artificial sequence
  primer M462 for IL7
taatggtcagcatcgatcaattattggacagcatgaaagaaattggtagc

SEQ ID NO: 487 - DNA, 50 bp, artificial sequence
  primer M463 for IL7R
ggagaaagtggctatgctcaaaatggagacttggaagatgcagaactgga

SEQ ID NO: 488 - DNA, 50 bp, artificial sequence
  primer M464 for IL8
ctgatttctgcagctctgtgtgaaggtgcagttttgccaaggagtgctaa

SEQ ID NO: 489 - DNA, 50 bp, artificial sequence
  primer M465 for IL8RA
cttggtcaagtttgtttgtcttggctgctggggactgtctatgaatctgt

SEQ ID NO: 490 - DNA, 50 bp, artificial sequence
  primer M466 for IL8RB
ggaagattttaacatggagagtgacagctttgaagatttctggaaaggtg

SEQ ID NO: 491 - DNA, 50 bp, artificial sequence
  primer M467 for IL9
actaaagaacaacaagtgtccatatttttcctgtgaacagccatgcaacc

SEQ ID NO: 492 - DNA, 46 bp, artificial sequence
  primer M468 for IL9R
tgttcaagctgtcgcccagggtgaagagaatcttctaccagaacgt

SEQ ID NO: 493 - DNA, 48 bp, artificial sequence
  primer M469 for LIF
tgccaatgccctctttattctctattacacagcccagggggagccgtt

SEQ ID NO: 494 - DNA, 50 bp, artificial sequence
  primer M470 for LIFR
ggagccctgtgaagaacatttcttggatacctgattctcagactaaggtt

SEQ ID NO: 495 - DNA, 50 bp, artificial sequence
  primer M471 for BLT1
tcactaggtgtagagttcatctctctgctggctatcatcctgctgtcagt

SEQ ID NO: 496 - DNA, 44 bp, artificial sequence
  primer M472 for BLT2
ttggccttcttcagttctagcgtcaacccggtgctctacgtctt

SEQ ID NO: 497 - DNA, 50 bp, artificial sequence
  primer M473 for CYSLT1
caggaaatctgacagtatcttctgccacatgccatgacactattgatgac

SEQ ID NO: 498 - DNA, 50 bp, artificial sequence
  primer M474 for LTA4H
tgtgaaattaacctatactgcagaggtgtctgtccctaaagaactggtgg

SEQ ID NO: 499 - DNA, 36 bp, artificial sequence
  primer M475 for LTC4S
gtctaccgagcccaggtgaactgcagcgagtacttc

SEQ ID NO: 500 - DNA, 50 bp, artificial sequence
  primer M476 for LTB4DH
atgatggggcagcaagtggccaaagttgtggaaagtaaaaatgtagccct

SEQ ID NO: 501 - DNA, 50 bp, artificial sequence
  primer M477 for SELL
tcatctccagaaccaacctgtcaagtgattcagtgtgagcctctatcagc

SEQ ID NO: 502 - DNA, 50 bp, artificial sequence
  primer M478 for SCYC1
tctctcactgcatacattgtggaaggtgtagggagtgaagtctcagataa

SEQ ID NO: 503 - DNA, 50 bp, artificial sequence
  primer M479 for CCXCR1
caaactcctcaatatgatcttctccatcagcctctacagcagcatcttct

SEQ ID NO: 504 - DNA, 41 bp, artificial sequence
  primer M480 for SCYA22=MDC
ttcaagcaactgaggcaggcccctacggcgccaacatggaa

SEQ ID NO: 505 - DNA, 50 bp, artificial sequence
  primer M481 for both isoforms of CCR2 and for CCR5
agtgcttgactgacatttacctgctcaacctggccatctctgatctgctt

SEQ ID NO: 506 - DNA, 41 bp, artificial sequence
  primer M482 for SCYA3
aagcccggtgtcatcttcctaaccaagcgaagccggcaggt

SEQ ID NO: 507 - DNA, 50 bp, artificial sequence
  primer M483 for SCYA4
gctcccagccagctgtggtattccaaaccaaaagaagcaagcaagtctgt

SEQ ID NO: 508 - DNA, 30 bp, artificial sequence
  primer M484 for AMH
agccccgcggagaggactcccggctgagta

SEQ ID NO: 509 - DNA, 48 bp, artificial sequence
  primer M485 for AMHR2
atgaggagcgctggcagaatggccaatataaaccaggtattgcccacc

SEQ ID NO: 510 - DNA, 47 bp, artificial sequence
  primer M486 for SCYD1
tgcggcaaacgcgcaatcatcttggagacgagacagcacaggctgtt

SEQ ID NO: 511 - DNA, 40 bp, artificial sequence
  primer M487 for NRTN
tcagtgctctgcagctccgtgctgtccatctggatgtgtc

SEQ ID NO: 512 - DNA, 50 bp, artificial sequence
  primer M488 for BMP-8
aggtggtccaggagcagtccaacagggagtctgacttgttctttttggat

SEQ ID NO: 513 - DNA, 49 bp, artificial sequence
  primer M489 for FAAH
tgctcttcacctatgtgggaaaggcctgggaagtgaacaaagggaccaa

SEQ ID NO: 514 - DNA, 50 bp, artificial sequence
  primer M490 for OSM
atctcatgcaggacaccagcagactcctggacccctatatacgtatccaa

SEQ ID NO: 515 - DNA, 50 bp, artificial sequence
  primer M491 for OSMR
tccttgaggacttaccagagtgaagtcttggctgaacgtttaccattgac

SEQ ID NO: 516 - DNA, 44 bp, artificial sequence
  primer M492 for SCYA5
aagtgctccaacccagcagtcgtctttgtcacccgaaagaaccg

SEQ ID NO: 517 - DNA, 50 bp, artificial sequence
  primer M493 for RANTES Receptor
tgtactccttggtatttgtcattggcctggttggaaacatcctggtggtc

SEQ ID NO: 518 - DNA, 50 bp, artificial sequence; identical to SEQ ID NO: 266
  primer M494 for relaxin H1

SEQ ID NO: 519 - DNA, 50 bp, artificial sequence
  primer M495 for SDF1
ctccaaactgtgcccttcagattgtagcccggctgaagaacaacaacaga

SEQ ID NO: 520 - DNA, 50 bp, artificial sequence
  primer M496 for SDF2
cttcacctctttctggaaaccaggaagtgagtgcttttggtgaggaaggt

SEQ ID NO: 521 - DNA, 50 bp, artificial sequence
  primer M497 for SDFR1
agaatgccagcaacatggagtacaggatcaataagccgagagctgaggat

SEQ ID NO: 522 - DNA, 48 bp, artificial sequence
  primer M498 for TGF-alpha
tttttggtgcacgaggacaagccagcatgtgtctgccattctgggtac

SEQ ID NO: 523 - DNA, 50 bp, artificial sequence
  primer M499 for TGFB1
gtgctaatggtggaaacccacaacgaaatctatgacaagttcaagcagag

SEQ ID NO: 524 - DNA, 50 bp, artificial sequence
  primer M500 for TGFB2
gaagaactagaagcaagatttgcaggtattgatggcacctccacatatac

SEQ ID NO: 525 - DNA, 50 bp, artificial sequence
  primer M501 for TGFB3
gaggtgatggaaatcaaattcaaaggcgtggacaatgaggatgaccatgg

SEQ ID NO: 526 - DNA, 50 bp, artificial sequence
  primer M502 for TGFBR1
atgacaacgtcaggttctggctcaggtttaccattgcttgttcagagaac

SEQ ID NO: 527 - DNA, 50 bp, artificial sequence
  primer M503 for TGFBR2
cacaggaagtctgtgtggctgtatggagaaagaatgacgagaacataaca

SEQ ID NO: 528 - DNA, 50 bp, artificial sequence
  primer M504 for TGFBR3
tggggtctccagactgtttttggtgtctgagggttctgtggtccagtttt

SEQ ID NO: 529 - DNA, 50 bp, artificial sequence
  primer M505 for THPO
tttagcttgggagaatggaaaacccagatggaggagaccaaggcacagga

SEQ ID NO: 530 - DNA, 46 bp, artificial sequence
  primer M506 for TNFTNFSF2
cgagtgacaagcctgtagcccatgttgtagcaaaccctcaagctga

SEQ ID NO: 531 - DNA, 45 bp, artificial sequence
  primer M507 for TNFSF1 (LTA)
aaacctgctgctcacctcattggagaccccagcaagcagaactca

SEQ ID NO: 532 - DNA, 39 bp, artificial sequence
  primer M508 for TNFSF3 (LTB)
cttagtgccccaggatcagggaggactggtaacggagac

SEQ ID NO: 533 - DNA, 50 bp, artificial sequence
  primer M509 for TNFSF4
tgcctgcacttctctgctcttcaggtatcacatcggtatcctcgaattca

SEQ ID NO: 534 - DNA, 50 bp, artificial sequence
  primer M510 for TNFSF5
gaggccagcagtaaaacaacatctgtgttacagtgggctgaaaaaggata

SEQ ID NO: 535 - DNA, 50 bp, artificial sequence
  primer M511 for TNFSF6
gagctgaggaaagtggcccatttaacaggcaagtccaactcaaggtccat

SEQ ID NO: 536 - DNA, 43 bp, artificial sequence
  primer M512 for TNFSF7
tgcagctgaatcacacaggacctcagcaggaccccaggctata

SEQ ID NO: 537 - DNA, 50 bp, artificial sequence
  primer M513 for TNFSF8
attatggtgttggtcgttcagaggacggactccattcccaactcacctga

SEQ ID NO: 538 - DNA, 43 bp, artificial sequence
  primer M514 for TNFSF9
agggcatgtttgcgcagctggtggcccaaaatgttctgctgat

SEQ ID NO: 539 - DNA, 50 bp, artificial sequence
  primer M515 for TNFSF10
gtactttaccaacgagctgaagcagatgcaggacaagtactccaaaagtg

SEQ ID NO: 540 - DNA, 50 bp, artificial sequence
  primer M516 for TNFSF11
gcctttcaaggagctgtgcaaaaggaattacaacatatcgttggatcaca

SEQ ID NO: 541 - DNA, 47 bp, artificial sequence
  primer M517 for TNFSF12
ctctactacctgtactgtcaggtgcactttgatgaggggaaggctgt

SEQ ID NO: 542 - DNA, 50 bp, artificial sequence
  primer M518 for TNFSF13
tggagtttatctgctgtatagccaggtcctgtttcaagacgtgactttca

SEQ ID NO: 543 - DNA, 42 bp, artificial sequence
  primer M519 for TNFSF14
agctgatacaagagcgaaggtctcacgaggtcaacccagcag

SEQ ID NO: 544 - DNA, 50 bp, artificial sequence
  primer M520 for TNFSF15 and transmembrane light mRNA
tcaccaagaaccgaatgaactataccaacaaattcctgctgatcccagag

SEQ ID NO: 545 - DNA, 50 bp, artificial sequence
  primer M521 for TNFSF18
aaggagccctgtatggctaagtttggaccattaccctcaaaatggcaaat

SEQ ID NO: 546 - DNA, 50 bp, artificial sequence
  primer M522 for TNFRSF10B
ttttctgcttgcgctgcaccaggtgtgattcaggtgaagtggagctaagt

SEQ ID NO: 547 - DNA, 50 bp, artificial sequence
  primer M523 for TNFRSF11A
acaaatgcagaccctggaccaactgtaccttccttggaaagagagtagaa

SEQ ID NO: 548 - DNA, 50 bp, artificial sequence
  primer M524 for TNFRSF11B
tggatttggagtggtgcaagctggaaccccagagcgaaatacagtttgca

SEQ ID NO: 549 - DNA, 43 bp, artificial sequence
  primer M525 for TNFRSF12
cacacacggctactctgttcccgcagagatactgactgtggga

SEQ ID NO: 550 - DNA, 50 bp, artificial sequence
  primer M526 for TNFRSF14
gtgaaaagaagaaagccaaggggtgatgtagtcaaggtgatcgtctccgt

SEQ ID NO: 551 - DNA, 50 bp, artificial sequence
  primer M527 for TNFRSF17
catgtcagcgttattgtaatgcaagtgtgaccaattcagtgaaaggaacg

SEQ ID NO: 552 - DNA, 49 bp, artificial sequence
  primer M528 for TNFRSF18
actgcaaaccttggacagactgcacccagttcgggtttctcactgtgtt

SEQ ID NO: 553 - DNA, 50 bp, artificial sequence
  primer M529 for TNFRSF19
gtattactaggctatttgtcatgtaaagtgacttgtgaatcaggagactg

SEQ ID NO: 554 - DNA, 50 bp, artificial sequence
  primer M530 for TNFRSF21
acttatgttcccaaaggcatgaactcaacagaatccaactcttctgcctc

SEQ ID NO: 555 - DNA, 50 bp, artificial sequence
  primer M531 for TNFRSF3
actgaagccgagctcaaagatgaagttgggaagggtaacaaccactgcgt

SEQ ID NO: 556 - DNA, 41 bp, artificial sequence
  primer M532 for TNFRSF4
tgcacgtggtgtaacctcagaagtgggagtgagcggaagca

SEQ ID NO: 557 - DNA, 50 bp, artificial sequence
  primer M533 for TNFRSF6
agattgtgtgatgaaggacatggcttagaagtggaaataaactgcacccg

SEQ ID NO: 558 - DNA, 38 bp, artificial sequence
  primer M534 for TNFRSF6B
tcagcaccagggtaccaggagctgaggagtgtgagcgt

SEQ ID NO: 559 - DNA, 50 bp, artificial sequence
  primer M535 for TNFRSF7
catcaacgaaggaaatatagatcaaacaaaggagaaagtcctgtggagcc

SEQ ID NO: 560 - DNA, 50 bp, artificial sequence
  primer M536 for TNFRSF8
cacggagcacaccaataacaagattgagaaaatctacatcatgaaggctg

SEQ ID NO: 561 - DNA, 50 bp, artificial sequence
  primer M537 for TNFRSF9
tgtgacatatgcaggcagtgtaaaggtgttttcaggaccaggaaggagtg

SEQ ID NO: 562 - DNA, 50 bp, artificial sequence
  primer M538 for GAPD
cccttcattgacctcaactacatggtttacatgttccaatatgattccac

SEQ ID NO: 563 - DNA, 40 bp, artificial sequence
  primer M539 for GAPD
cattgacctcaactacatggtttacatgttccaatatgat

SEQ ID NO: 564 - DNA, 30 bp, artificial sequence
  primer M540 for GAPD
acctcaactacatggtttacatgttccaat

SEQ ID NO: 565 - DNA, 50 bp, artificial sequence
  primer M541 for GAPD
agtatgacaacagcctcaagatcatcagcaatgcctcctgcaccaccaac

SEQ ID NO: 566 - DNA, 40 bp, artificial sequence
  primer M542 for GAPD
gacaacagcctcaagatcatcagcaatgcctcctgcacca

SEQ ID NO: 567 - DNA, 30 bp, artificial sequence
  primer M543 for GAPD
cagcctcaagatcatcagcaatgcctcctg

SEQ ID NO: 568 - DNA, 50 bp, artificial sequence
  primer M544 for GAPD
aactttggtatcgtggaaggactcatgaccacagtccatgccatcactgc

SEQ ID NO: 569 - DNA, 50 bp, artificial sequence
  primer M545 for ACTB
agatcactgccctggcacccagcacaatgaagatcaagatcattgctcct

SEQ ID NO: 570 - DNA, 40 bp, artificial sequence
  primer M546 for ACTB
gccctggcacccagcacaatgaagatcaagatcattgctc

SEQ ID NO: 571 - DNA, 30 bp, artificial sequence
  primer M547 for ACTB
tggcacccagcacaatgaagatcaagatca

SEQ ID NO: 572 - DNA, 50 bp, artificial sequence
  primer M548 for negative control
atgacaacagcctcaagatcatcaggaagatcaagatcattgctcctcct

SEQ ID NO: 573 - DNA, 40 bp, artificial sequence
  primer M549 for negative control
aacagcctcaagatcatcaggaagatcaagatcattgctc

SEQ ID NO: 574 - DNA, 30 bp, artificial sequence
  primer M550 for negative control
cctcaagatcatcaggaagatcaagatcat

SEQ ID NO: 575 - DNA, 50 bp, artificial sequence
  primer M551 for PTGDS
aacctgacctccaccttcctcaggaaaaaccagtgtgagacccgaaccat

SEQ ID NO: 576 - DNA, 42 bp, artificial sequence
  primer M552 for PTGES
acgtggaacgctgcctcagggcccaccggaacgacatggaga

SEQ ID NO: 577 - DNA, 50 bp, artificial sequence
  primer M553 for LYST
cctgtccaagaagaaaaggcaacagattttaacctaccgctctcagcaga

SEQ ID NO: 578 - DNA, 50 bp, artificial sequence
  primer M554 for MITF
aatgctagaatataatcactatcaggtgcagacccacctcgaaaacccca

SEQ ID NO: 579 - DNA, 50 bp, artificial sequence
  primer M555 for OCA2
gtttgcctttgtggtgctgtgttctattttgttcagcctatatccggatc

SEQ ID NO: 580 - DNA, 46 bp, artificial sequence
  primer M556 for OMP
tggccaagatccgcaaggtcatgtacttcctcgtcacctttggcga

SEQ ID NO: 581 - DNA, 50 bp, artificial sequence
  primer M557 for TRP-2
tcttccaccggacctgcaagtgcacaggaaactttgccggctataattgt

SEQ ID NO: 582 - DNA, 50 bp, artificial sequence
  primer M558 for TRP-2
tattctgttagagatacattattaggaccaggacgcccctacagggccat

SEQ ID NO: 583 - DNA, 50 bp, artificial sequence
  primer M559 for TYR
agcatcattcttctcctcttggcagattgtctgtagccgattggaggagt

SEQ ID NO: 584 - DNA, 50 bp, artificial sequence
  primer M560 for TYRP1
gcctgtgaccagagggttctcatagtcaggagaaatcttctggacttaag

SEQ ID NO: 585 - DNA, 41 bp, artificial sequence
  primer M561 for PNMT
tgcctcattgagggcaagggggaatgctggcaggataagga

SEQ ID NO: 586 - DNA, 44 bp, artificial sequence
  primer M562 for HGF
gcagtgcacacaggaagtggtgtgccacaactcacaactacgac

SEQ ID NO: 587 - DNA, 50 bp, artificial sequence
  primer M563 for MET
gatctgtctgcctgcaatctacaaggttttcccaaatagtgcaccccttg

SEQ ID NO: 588 - DNA, 50 bp, artificial sequence
  primer M564 for NPY
acatcaacctcatcaccaggcagagatatggaaaacgatccagcccagag

SEQ ID NO: 589 - DNA, 49 bp, artificial sequence
  primer M565 for NPY2R
gcaaaaacgcgaggtccaggtcagttgtagactcttgtgctggttgcag

SEQ ID NO: 590 - DNA, 50 bp, artificial sequence
  primer M566 for neuropeptide y receptor
atttatttgctacttcaagatatatatacgcctaaaaaggagaaacaaca

SEQ ID NO: 591 - DNA, 50 bp, artificial sequence
  primer M567 for BDNF
tgatagaagagctgttggatgaggaccataaagttcggcccaatgaagaa

SEQ ID NO: 592 - DNA, 50 bp, artificial sequence
  primer M568 for NTF3
ttgccagaagactcgctcaattccctcattattaagctgatccaggcaga

SEQ ID NO: 593 - DNA, 50 bp, artificial sequence
  primer M569 for NTF5
tccgccagtacttctttgaaacccgctgcaaggctgataacgctgaggaa

SEQ ID NO: 594 - DNA, 50 bp, artificial sequence
  primer M570 for OGFR
ttgaggacaatcactcctacatccagtggctgtttcctctgcgagaacca

SEQ ID NO: 595 - DNA, 50 bp, artificial sequence
  primer M571 for OPRD1
cttgtcatgttcggcatcgtccggtacactaagatgaagacggccaccaa

SEQ ID NO: 596 - DNA, 50 bp, artificial sequence
  primer M572 for OPRK1
cgctggtcatgttcgtgatcatccgatacacaaagatgaagacagcaacc

SEQ ID NO: 597 - DNA, 50 bp, artificial sequence
  primer M573 for OPRM1
tcctggtcatgtatgtgattgtcagatacaccaagatgaagactgccacc

SEQ ID NO: 598 - DNA, 50 bp, artificial sequence
  primer M574 for VIPR1
atgacaaggcagcgagtttggatgagcagcagaccatgttctacggttct

SEQ ID NO: 599 - DNA, 50 bp, artificial sequence
  primer M575 for VIPR2
cagcgacccggaggatgagagcaagatcacgttttatattctggtgaagg

SEQ ID NO: 600 - DNA, 50 bp, artificial sequence
  primer M576 for AKR1C2
atttggcacctatgcgcctgcagaggttcctaaaagtaaagctctagagg

SEQ ID NO: 601 - DNA, 50 bp, artificial sequence
  primer M577 for AKR1C3
tcattctccaatgtctctaaagccaggtgaggaactttcaccaacagatg

SEQ ID NO: 602 - DNA, 50 bp, artificial sequence
  primer M578 for CYP8B1
atgtttgaatttctgaagcgcatgaggaccaagcatggggatgtgttcac

SEQ ID NO: 603 - DNA, 50 bp, artificial sequence
  primer M579 for EBRP
ccatagtcaaagaaaaatattaccggcatttcctgcagatcaccctgtgc

SEQ ID NO: 604 - DNA, 50 bp, artificial sequence
  primer M580 for HSD3A
ggaagccttttcagatattctgaggcgtgagattcaacattttggggtga

SEQ ID NO: 605 - DNA, 50 bp, artificial sequence
  primer M581 for HSD3B
tctatcatgaatgtcaatgtgaaaggtacccagctcctgttagaggcctg

SEQ ID NO: 606 - DNA, 50 bp, artificial sequence
  primer M582 for RDHL and hydroxysteroid epimerase
gtgaagaaccaagttggggagaaaggtctctggggtctgatcaataatgc

SEQ ID NO: 607 - DNA, 50 bp, artificial sequence
  primer M583 for sterol/retinol dehydrogenase
tgaaggagtgcgtgagagacaaaggactctggggcctggtgaataatgct

SEQ ID NO: 608 - DNA, 50 bp, artificial sequence
  primer M584 for TAC1
ggattaatgggcaaacgggatgctgattcctcaattgaaaaacaagtggc

SEQ ID NO: 609 - DNA, 50 bp, artificial sequence
  primer M585 for both isoforms of TACR1
gagcaagtctctgccaagcgcaaggtggtcaaaatgatgattgtcgtggt

SEQ ID NO: 610 - DNA, 50 bp, artificial sequence
  primer M586 for TACR2
atctactgctgtctcaaccacaggtttcgctctgggttccggcttgcctt

SEQ ID NO: 611 - DNA, 50 bp, artificial sequence
  primer M587 for TACR3
tcatctactgctgtctgaataaaagatttcgagctggcttcaagagagca

SEQ ID NO: 612 - DNA, 50 bp, artificial sequence
  primer M588 for TBG
gaggacaatggtctgaaactttccaatgctgcccataaggctgtgctgca

SEQ ID NO: 613 - DNA, 50 bp, artificial sequence
  primer M589 for TPO
ccgccatgtacgccacgatgcagagaaacctcaagaaaagaggaatcctt

SEQ ID NO: 614 - DNA, 50 bp, artificial sequence
  primer M590 for CALB2b
gctccaggaatacacccaaaccatactacggatgtttgacttgaacgggg

SEQ ID NO: 615 - DNA, 50 bp, artificial sequence
  primer M591 for CALB3
gctgaattccccagtttactcaaaggtccaaacaccctagatgatctctt

SEQ ID NO: 616 - DNA, 50 bp, artificial sequence
  primer M592 for NCOA2
aaaaacctgctgccaaagtctatagtaaatgggggatcttggtctggcga

SEQ ID NO: 617 - DNA, 50 bp, artificial sequence
  primer M593 for VDR
gtggacatcggcatgatgaaggagttcattctgacagatgaggaagtgca

SEQ ID NO: 618 - DNA, 50 bp, artificial sequence
  primer M594 for VDBP
ctctgaagattgcatggccaaagagctgcctgaacacacagtaaaactct

SEQ ID NO: 619 - DNA, 50 bp, artificial sequence
  primer M595 for BMP1
tcgtaagtcctccatcaaagctgcagttccaggaaacacttctaccccca

SEQ ID NO: 620 - DNA, 50 bp, artificial sequence
  primer M596 for BMP10
catcattaggagtttcaagaatgaagatctgttttcccagccggtcagtt

SEQ ID NO: 621 - DNA, 45 bp, artificial sequence
  primer M597 for BMP11
gtcattagcatggcccaggagacggacccagcagtacagacagat

SEQ ID NO: 622 - DNA, 47 bp, artificial sequence
  primer M598 for BMP15
accagtgtggcaaggcctcacagaggtacctggcatatacagatcct

SEQ ID NO: 623 - DNA, 50 bp, artificial sequence
  primer M599 for BMP2
actgtgcgcagcttccaccatgaagaatctttggaagaactaccagaaac

SEQ ID NO: 624 - DNA, 50 bp, artificial sequence
  primer M600 for BMP3
cagctttcgggcggcagcagcagaaactcttgaaagaaaaggactgtata

SEQ ID NO: 625 - DNA, 50 bp, artificial sequence
  primer M601 for BMP4
agcttccaccacgaagaacatctggagaacatcccagggaccagtgaaaa

SEQ ID NO: 626 - DNA, 50 bp, artificial sequence
  primer M602 for BMP5
cagctctgtgcagaaacaggggatggacgcagtatcaacgtaaaatctgc

SEQ ID NO: 627 - DNA, 50 bp, artificial sequence
  primer M603 for BMP6
acatggtcatgagctttgtgaacctggtggagtacgacaaggagttctcc

SEQ ID NO: 628 - DNA, 50 bp, artificial sequence
  primer M604 for BMP7
atggtcatgagcttcgtcaacctcgtggaacatgacaaggaattcttcca

SEQ ID NO: 629 - DNA, 50 bp, artificial sequence; identical to SEQ ID NO: 512
  primer M605 for BMP8

SEQ ID NO: 630 - DNA, 50 bp, artificial sequence
  primer M606 for BMP9
attgtgcggagcttcagcatggaagatgccatctccataactgccacaga

SEQ ID NO: 631 - DNA, 50 bp, artificial sequence
  primer M607 for BMPR1A
agtgggtctggactacctttattggttcagcgaactattgccaaacagat

SEQ ID NO: 632 - DNA, 50 bp, artificial sequence
  primer M608 for BMPR1B
gaaggctcagattttcagtgtcgggacactcccattcctcatcaaagaag

SEQ ID NO: 633 - DNA, 50 bp, artificial sequence
  primer M609 for BMPR2
tgctttggatacagaatgttgacaggagaccgtaaacaaggtcttcacag

SEQ ID NO: 634 - DNA, 49 bp, artificial sequence
  primer M610 for GDF1
tcttctttgacaacagcgacaacgtggtgctgcggcagtatgaggacat

SEQ ID NO: 635 - DNA, 48 bp, artificial sequence
  primer M611 for GDF10
catgtgagttccccatgcctaagatcgttcgtccatccaaccatgcca

SEQ ID NO: 636 - DNA, 50 bp, artificial sequence
  primer M612 for GDF3
ggaatgtacttcgctttctcccagaccaaggtttctttctttacccaaag

SEQ ID NO: 637 - DNA, 50 bp, artificial sequence
  primer M613 for GDF7
accagctttattgacaaagggcaagatgaccgaggtcccgtggtcaggaa

SEQ ID NO: 638 - DNA, 50 bp, artificial sequence
  primer M614 for GDF8
caggaccaggagaagatgggctgaatccgtttttagaggtcaaggtaaca

SEQ ID NO: 639 - DNA, 48 bp, artificial sequence
  primer M615 for GDF9
aagcaggctcctggagaccaggtaacaggaatccttccatcagtggaa

SEQ ID NO: 640 - DNA, 50 bp, artificial sequence
  primer M616 for AQP1
tgtagcccttggacacctcctggctattgactacactggctgtgggatta

SEQ ID NO: 641 - DNA, 50 bp, artificial sequence
  primer M617 for AQP10
ggagccacctatgttctctaccatgatgccctacagaactatacaggtgg

SEQ ID NO: 642 - DNA, 48 bp, artificial sequence
  primer M618 for AQP2
tgtcgtcactggcaaatttgatgaccactgggtcttctggatcggacc

SEQ ID NO: 643 - DNA, 50 bp, artificial sequence
  primer M619 for AQP3
ctggaatagtttttgggctgtattatgatgcaatctggcactttgccgac

SEQ ID NO: 644 - DNA, 50 bp, artificial sequence; identical to SEQ ID NO: 257
  primer M620 for AQP4

SEQ ID NO: 645 - DNA, 47 bp, artificial sequence
  primer M621 for AQP5
accctgggccaccttgtcggaatctacttcactggctgctccatgaa

SEQ ID NO: 646 - DNA, 48 bp, artificial sequence
  primer M622 for AQP6
atccgagagacccttgggatcaacgtggtccggaacagtgtctcaact

SEQ ID NO: 647 - DNA, 50 bp, artificial sequence
  primer M623 for AQP7
agttcatgagcacatatgtcatgatggtattcggccttggttccgtggcc

SEQ ID NO: 648 - DNA, 50 bp, artificial sequence
  primer M624 for AQP8
gccatctgatcctgatgtctggagagatagccatgtgtgagcctgaattt

SEQ ID NO: 649 - DNA, 50 bp, artificial sequence
  primer M625 for AQP9
cgttcatcttgattgtccttggatgtggctgtgttgcccaagctattctc

SEQ ID NO: 650 - DNA, 50 bp, artificial sequence
  primer M626 for SLC6A1
tacctgtacaactccttcaccacgacactgccgtggaaacagtgcgacaa

SEQ ID NO: 651 - DNA, 50 bp, artificial sequence
  primer M627 for SLC6A11
ggaggaaagtttgccctttatttgaaggcattggctatgcaacacaggtg

SEQ ID NO: 652 - DNA, 50 bp, artificial sequence
  primer M628 for SLC6A9
tggaggatcagccccatgttcaaaggagtgggctatggtatgatggtggt

SEQ ID NO: 653 - DNA, 50 bp, artificial sequence
  primer M629 for SLC6A13 and SLC6A12
tatctctgctacaaaaatgggggaggtgccttcttcatcccctacctcgt

SEQ ID NO: 654 - DNA, 50 bp, artificial sequence
  primer M630 for SLC6A14
gtttcttcaagtgcagggagaaggagaaagtgtcggcttcatcagagaat

SEQ ID NO: 655 - DNA, 50 bp, artificial sequence
  primer M631 for SLC6A2
tggaaaatctgcccattcttcaaaggcgttggctatgctgtcatcctgat

SEQ ID NO: 656 - DNA, 50 bp, artificial sequence
  primer M632 for SLC6A3
aacaagttcaccaacaactgctacagggacgcgattgtcaccacctccat

SEQ ID NO: 657 - DNA, 50 bp, artificial sequence
  primer M633 for SLC6A4
acaacaagttcaacaacaactgctaccaagatgccctggtgaccagcgtg

SEQ ID NO: 658 - DNA, 50 bp, artificial sequence
  primer M634 for SLC6A5
agacttcaggaaaagtggtgtacttcacggccacgttcccgtatgtcgta

SEQ ID NO: 659 - DNA, 50 bp, artificial sequence
  primer M635 for SLC6A6
acaagtacaagtataactcgtacagggactgtatgctgctgggatgcctg

SEQ ID NO: 660 - DNA, 50 bp, artificial sequence
  primer M636 for SLC6A7
ccaccacttgttgtcttccaaggtgtggattgaagctgctcttcagatct

SEQ ID NO: 661 - DNA, 50 bp, artificial sequence
  primer M637 for SLC6A8
cgcttcaacaacaactgctacaaggacgccatcatcctggctctcatcaa

SEQ ID NO: 662 - DNA, 50 bp, artificial sequence
  primer M638 for GABBR1
caatacccgcagcatttccaacatgacatcccaggaatttgtggagaaac

SEQ ID NO: 663 - DNA, 50 bp, artificial sequence
  primer M639 for GABRA1
ttctgagcacactgactggaagaagctatggacagccgtcattacaagat

SEQ ID NO: 664 - DNA, 50 bp, artificial sequence
  primer M640 for GABRA2
gataatcggcttagaccaggactgggagacagtattactgaagtcttcac

SEQ ID NO: 665 - DNA, 50 bp, artificial sequence
  primer M641 for GABRA3
ctgcgacctgggcttggagatgcagtgactgaagtgaagactgacatcta

SEQ ID NO: 666 - DNA, 50 bp, artificial sequence
  primer M642 for GABRA4
aggctgcgtcctggatttgggggtcctgttacagaagtgaaaactgacat

SEQ ID NO: 667 - DNA, 50 bp, artificial sequence
  primer M643 for GABRA5
actgagaacatcagcaccagcacaggcgaatacacaatcatgacagctca

SEQ ID NO: 668 - DNA, 50 bp, artificial sequence
  primer M644 for GABRA6
atggaaccattttatacaccatgaggcttaccatcaatgctgactgtccc

SEQ ID NO: 669 - DNA, 50 bp, artificial sequence
  primer M645 for GABRB1
tgatggaacagttctctatggactccgaatcacaaccacagctgcatgta

SEQ ID NO: 670 - DNA, 50 bp, artificial sequence
  primer M646 for GABRB2
accaagaaggttgttttttccacaggttcctatcccaggttatccctcag

SEQ ID NO: 671 - DNA, 50 bp, artificial sequence
  primer M647 for GABRB3
gaactgcactctggaaattgaaagctatggctacaccacggatgacattg

SEQ ID NO: 672 - DNA, 50 bp, artificial sequence
  primer M648 for GABRD
agcatcgaccacatctcagaggccaacatggagtacaccatgacggtgtt

SEQ ID NO: 673 - DNA, 50 bp, artificial sequence
  primer M649 for GABRE
ggatggcaaggtgttgtacacaattaggatgaccattgatgccggatgct

SEQ ID NO: 674 - DNA, 50 bp, artificial sequence
  primer M650 for GABRG2
tggtcgagtgctctactccctaaggttgacaattgatgctgagtgccaat

SEQ ID NO: 675 - DNA, 50 bp, artificial sequence
  primer M651 for GABRG3
aaaagatgctacgccagcaagaacagcattaggcatcaccacggtgctga

SEQ ID NO: 676 - DNA, 50 bp, artificial sequence
  primer M652 for GABRP
aatttctcaggcccaattttggtggagaacccgtacagatagcgctgact

SEQ ID NO: 677 - DNA, 50 bp, artificial sequence
  primer M653 for GABRR1
atgaagatgcccacaagcaagtcagcccaattctgagacgaagtcctgac

SEQ ID NO: 678 - DNA, 50 bp, artificial sequence
  primer M654 for GABRR2
cagacctgttctttggagctggagagctatgcctatacagatgaagatct

SEQ ID NO: 679 - DNA, 50 bp, artificial sequence
  primer M655 for CRH-R1
gggccccatgatcctggtcctgctgatcaatttcatcttccttttcaaca

SEQ ID NO: 680 - DNA, 40 bp, artificial sequence
  primer M656 for CRH-R1
ccatgatcctggtcctgctgatcaatttcatcttcctttt

SEQ ID NO: 681 - DNA, 30 bp, artificial sequence
  primer M657 for CRH-R1
atcctggtcctgctgatcaatttcatcttc

SEQ ID NO: 682 - DNA, 40 bp, artificial sequence
  primer M658 for insulin
agctggagaactactgcaactagacgcagcccgcaggcag

SEQ ID NO: 683 - DNA, 45 bp, artificial sequence
  primer M659 for insulin receptor
caccacttcttctgacatgtggtcctttggcgtggtcctttggga

SEQ ID NO: 684 - DNA, 45 bp, artificial sequence
  primer M660 for insulin degrading enzyme
tgtcacaagcaccagccttgccacaacctgaagtgattcagaaca

SEQ ID NO: 685 - DNA, 45 bp, artificial sequence
  primer M661 for Proenkephalin
gccgacatcaacttcctggcttgcgtaatggaatgtgaaggtaaa

SEQ ID NO: 686 - DNA, 40 bp, artificial sequence
  primer M662 for pituitary adenylate cyclase activating peptide
ggatcaggccagaggaagaggcgtacggcgaggacggaaa

SEQ ID NO: 687 - DNA, 45 bp, artificial sequence
  primer M663 for pituitary adenylate cyclase activating peptide receptors A, B, B2, C
gcaaccactgcttcatctccactgtggaatgtaaggccgtcatgg

SEQ ID NO: 688 - DNA, 45 bp, artificial sequence
  primer M664 for PC1
agaagatggtggatccaggggaggagcagcccacacaagagaacc

SEQ ID NO: 689 - DNA, 45 bp, artificial sequence
  primer M665 for PC2
aggaaaaggaaccccgaggccggtgtggcaaccacagatttgtac

SEQ ID NO: 690 - DNA, 45 bp, artificial sequence
  primer M666 for polypeptide 7B2
gaagggaggagagagacgaaagcggaggagtgtcaatccatatct

SEQ ID NO: 691 - DNA, 50 bp, artificial sequence
  primer M667 for COL14A1
cagtgtttatacaaagctccaggagattgaaggacctagtgtgagcataa

SEQ ID NO: 692 - DNA, 50 bp, artificial sequence
  primer M668 for COL1A1
atgccatcaaagtcttctgcaacatggagactggtgagacctgcgtgtac

SEQ ID NO: 693 - DNA, 50 bp, artificial sequence
  primer M669 for COL1A2
tgaacttgttgctgagggcaacagcaggttcacttacactgttcttgtag

SEQ ID NO: 694 - DNA, 50 bp, artificial sequence
  primer M670 for COL3A1 and COL5A2
gtagatattgcaccctatgacattggtggtcctgatcaagaatttggtgt

SEQ ID NO: 695 - DNA, 50 bp, artificial sequence
  primer M671 for COL5A1
ggacatcatgttcaatgacttcggtgaagcgtcacagaaatttggatttg

SEQ ID NO: 696 - DNA, 50 bp, artificial sequence
  primer M672 for COL7A1
agtattctgtggaggagtaccaggaccctgaagctccttgggatagtgat

SEQ ID NO: 697 - DNA, 50 bp, artificial sequence
  primer M673 for FN1
tgagtgcttcatgcctttagatgtacaggctgacagagaagattcccgag

SEQ ID NO: 698 - DNA, 50 bp, artificial sequence
  primer M674 for LAMA2
atcagatccctgaagctcaccaaaggcacagcaagccactggaggttaat

SEQ ID NO: 699 - DNA, 50 bp, artificial sequence
  primer M675 for LAMB2
gaccagcatcttgacttgctcaaacattcaaacttcctgggtgcctatga

SEQ ID NO: 700 - DNA, 50 bp, artificial sequence
  primer M676 for LAMB3
gattgggttggaatgctttccatctccaggagactttcatgcagcctaaa

SEQ ID NO: 701 - DNA, 50 bp, artificial sequence
  primer M677 for LAMC2
ttacaatccaagacacactcaacacattagacggcctcctgcatctgatg

SEQ ID NO: 702 - DNA, 50 bp, artificial sequence
  primer M678 for LOX
gactataccaacaatgttgtgcgctgtgacattcgctacacaggacatca

SEQ ID NO: 703 - DNA, 50 bp, artificial sequence
  primer M679 for MMP1
atctacagctcctttggcttccctagaactgtgaagcatatcgatgctgc

SEQ ID NO: 704 - DNA, 50 bp, artificial sequence
  primer M680 for MMP10
tcagtggatcatcacagtttgagtttgaccccaatgccaggatggtgaca

SEQ ID NO: 705 - DNA, 48 bp, artificial sequence
  primer M681 for MMP11
cctctactggaagtttgaccctgtgaaggtgaaggctctggaaggctt

SEQ ID NO: 706 - DNA, 50 bp, artificial sequence
  primer M682 for MMP3
aggaaatcaattctgggccatcagaggaaatgaggtacgagctggatacc

SEQ ID NO: 707 - DNA, 47 bp, artificial sequence
  primer M683 for MMP9
agtgagttgaaccaggtggaccaagtgggctacgtgacctatgacat

SEQ ID NO: 708 - DNA, 48 bp, artificial sequence
  primer M684 for PLOD
agaacgtgccgactattgacatccacatgaaccagatcggctttgagc

SEQ ID NO: 709 - DNA, 50 bp, artificial sequence
  primer M685 for prolyl 4-hydroxylase beta subunit
aggagccagacatggaggaagacgatgaccagaaagctgtgaaagatgaa

SEQ ID NO: 710 - DNA, 50 bp, artificial sequence
  primer M686 for prolyl 4-hydroxylase alpha subunit
gtcttcactgaggccaagtctacaattctgcccaccaagaatgatgctct

SEQ ID NO: 711 - DNA, 50 bp, artificial sequence
  primer M687 for TNXB
actatgcccatggttttgggaacatctctggagagttctggctgggcaat

SEQ ID NO: 712 - DNA, 50 bp, artificial sequence
  primer M688 for VTN
cttcttcttctctggagacaagtactaccgagtcaatcttcgcacacggc

SEQ ID NO: 713 - DNA, 50 bp, artificial sequence
  primer M689 for FLG
atcatcccatgaacaggcaagatcaagtgcaggagagagacatggatccc

SEQ ID NO: 714 - DNA, 48 bp, artificial sequence
  primer M690 for IVL
agagcagcaggtaggacagccaaagcacctggaacagcaggaaaagca

SEQ ID NO: 715 - DNA, 50 bp, artificial sequence
  primer M691 for KRT1
cagttccagcgtgaggtttgtttctaccacttattccggagtaaccagat

SEQ ID NO: 716 - DNA, 50 bp, artificial sequence
  primer M692 for KRT10
ctgaaaccgagtgccagaatactgaataccaacaactcacggatattaag

SEQ ID NO: 717 - DNA, 50 bp, artificial sequence
  primer M693 for KRT14
tgaccagtatgagaagatggcagagaagaaccgcaaggatgccgaggaat

SEQ ID NO: 718 - DNA, 49 bp, artificial sequence
  primer M694 for KRT17
gagatgcgtgaccagtatgagaagatggcagagaagaaccgcaaggatg

SEQ ID NO: 719 - DNA, 50 bp, artificial sequence
  primer M695 for KRT19
tgctcgagggacaggaagatcactacaacaatttgtctgcctccaaggtc

SEQ ID NO: 720 - DNA, 50 bp, artificial sequence
  primer M696 for KRT5
gaagagcttcaagagctaagaacctgctgcaagtcactgccttccaagtg

SEQ ID NO: 721 - DNA, 48 bp, artificial sequence
  primer M697 for KRTHA5
tggagattgagctgcaggctcagcacagcatgagagatgctttggaat

SEQ ID NO: 722 - DNA, 50 bp, artificial sequence
  primer M698 for KRTHB5
atcactgcactgaatggcatgtgaatggaaaatgtgtgcttgcttccaga

SEQ ID NO: 723 - DNA, 50 bp, artificial sequence
  primer M699 for LOR
tgcaaatccttcatgtcttaacctacctggaagaagccattgagctctcc

SEQ ID NO: 724 - DNA, 50 bp, artificial sequence
  primer M700 for TGM1
acagtgaaactgcacctctacctctcagtcactttctatactggtgtcag

SEQ ID NO: 725 - DNA, 50 bp, artificial sequence
  primer M701 for TGM2
tcctctttgcatcctctatgagaaataccgtgactgccttacggagtcca

SEQ ID NO: 726 - DNA, 50 bp, artificial sequence
  primer M702 for TGM3
aagaggcagaacatcccataaagatctcgtacgctcagtatgagaggtac

SEQ ID NO: 727 - DNA, 50 bp, artificial sequence
  primer M703 for THH
cttaagtgatgttgccaatatcttgacacctgccaaagcttccagcacgg

SEQ ID NO: 728 - DNA, 50 bp, artificial sequence
  primer M704 for TRHY
caataccgcccttaagtgatgttgccaatatcttgacacctgccaaagct

SEQ ID NO: 729 - DNA, 50 bp, artificial sequence
  primer M705 for agouti
tatataggctgctcgaaggtgtgcggctgtttctgtaaaggtcccgaaag

SEQ ID NO: 730 - DNA, 48 bp, artificial sequence
  primer M706 for AGRP
ttcttcaatgccttctgctactgccgcaagctgggtactgccatgaat

SEQ ID NO: 731 - DNA, 42 bp, artificial sequence
  primer M707 for CCS
cagaatggaggatgagcagctgaaggtgtgggatgtgattgg

SEQ ID NO: 732 - DNA, 50 bp, artificial sequence
  primer M708 for GCH1
gctaccaggagaccatctcagatgtcctaaacgatgctatatttgatgaa

SEQ ID NO: 733 - DNA, 49 bp, artificial sequence
  primer M709 for GGT1
acaaccagcttctgcccaacgtcacgacagtggagagaaacattgacca

SEQ ID NO: 734 - DNA, 50 bp, artificial sequence
  primer M710 for GSR
cagtgaagatgggagcaacgaaggcagactttgacaacacagtcgccatt

SEQ ID NO: 735 - DNA, 50 bp, artificial sequence
  primer M711 for LAMP1
caaggaatccagttgaatacaattcttcctgacgccagagaccctgcctt

SEQ ID NO: 736 - DNA, 50 bp, artificial sequence
  primer M712 for Pme17
gcaggtctgagtactctcatatgatgctgtgattttcctggagttgacag

SEQ ID NO: 737 - DNA, 50 bp, artificial sequence
  primer M713 for TXNRD1
tcagaggctctatgcaggttccactgtcaagtgtgactatgaaaatgttc

SEQ ID NO: 738 - DNA, 50 bp, artificial sequence
  primer M714 for TXNRD2
gcgaagttactcaaggatttgctctggggatcaagtgtggggcttcctat

SEQ ID NO: 739 - DNA, 50 bp, artificial sequence
  primer M715 for BMAL1a
caacgaccaaggatcaagtagtcccagtaatgatgaggcagcaatggctg

SEQ ID NO: 740 - DNA, 49 bp, artificial sequence
  primer M716 for CLOCK
acaacagcaacagtcacagacattgtcagtaacgcagcagcagcagcag

SEQ ID NO: 741 - DNA, 50 bp, artificial sequence
  primer M717 for CRY1
ctatcagcagctttcacgatatagaggactaggtcttctggcatcagtac

SEQ ID NO: 742 - DNA, 49 bp, artificial sequence
  primer M718 for CRY2
tatggcgagagttcttctacacggcagctaccaacaaccccaggtttga

SEQ ID NO: 743 - DNA, 50 bp, artificial sequence
  primer M719 for CYP39A1
tgaatataaacaaagaatatgacatctgttgggcctcacaaggaccaggg

SEQ ID NO: 744 - DNA, 50 bp, artificial sequence
  primer M720 for CYP7A1
atccggacagctaaggaggatttcactttgcaccttgaggacggttccta

SEQ ID NO: 745 - DNA, 50 bp, artificial sequence
  primer M721 for CYP7B1
cctacatggtgaccctgaaatctttgaagctccagaggagtttagatatg

SEQ ID NO: 746 - DNA, 50 bp, artificial sequence
  primer M722 for DHCR7
tgcaggccatctacgtgattgacttcttctggaacgaaacctggtacctg

SEQ ID NO: 747 - DNA, 50 bp, artificial sequence
  primer M723 for PAH
gcctctctgggtgcacctgatgaatacattgaaaagctcgccacaattta

SEQ ID NO: 748 - DNA, 50 bp, artificial sequence
  primer M724 for PER1
cagtccagccttacctacagcaggaaactgcaccagctagactccattct

SEQ ID NO: 749 - DNA, 50 bp, artificial sequence
  primer M725 for PER2
ccttctctgggactcagcgaagtgtcggacaccaaagaagacgaaaatgg

SEQ ID NO: 750 - DNA, 50 bp, artificial sequence
  primer M726 for PER3
ctcatgggcaaaaggaggagctggctaaggtgtataattggattcaaagc

SEQ ID NO: 751 - DNA, 50 bp, artificial sequence
  primer M727 for TH
ctaccaagaccagacgtaccagtcagtctacttcgtgtctgagagcttca

SEQ ID NO: 752 - DNA, 50 bp, artificial sequence
  primer M728 for GSTA1
accagagccattctcaactacattgccagcaaatacaacctctatgggaa

SEQ ID NO: 753 - DNA, 50 bp, artificial sequence
  primer M729 for GSTA2
tgaaaaccagaatcagtaacctgcccacagtgaagaagtttctacagcct

SEQ ID NO: 754 - DNA, 50 bp, artificial sequence
  primer M730 for GSTA3
cattgccagcaaatacaacctctacgggaaagacataaaggagagagccc

SEQ ID NO: 755 - DNA, 50 bp, artificial sequence
  primer M731 for GSTA4
cagacccgaagcattctccactacatagcagacaagcacaatctctttgg

SEQ ID NO: 756 - DNA, 50 bp, artificial sequence
  primer M732 for GSTM1
agtacttggaggaactccctgaaaagctaaagctctactcagagtttctg

SEQ ID NO: 757 - DNA, 50 bp, artificial sequence
  primer M733 for GSTM3
agctctgaccacgaaaaactgaagcctcagtacttggaagagctacctgg

SEQ ID NO: 758 - DNA, 50 bp, artificial sequence
  primer M734 for GSTM4
gccagaatacttggaggaacttcctacaatgatgcagcacttctcacagt

SEQ ID NO: 759 - DNA, 50 bp, artificial sequence
  primer M735 for GSTM5
tttccttgcctatgatgtccttgacatgaagcgtatatttgagcccaagt

SEQ ID NO: 760 - DNA, 45 bp, artificial sequence
  primer M736 for GSTP1
gctgctgatccatgaggtcctagcccctggctgcctggatgcgtt

SEQ ID NO: 761 - DNA, 50 bp, artificial sequence
  primer M737 for GSTT1
aaacctcacccttgcaccgtcctcagcagtccacaaagcattttcatttc

SEQ ID NO: 762 - DNA, 48 bp, artificial sequence
  primer M738 for GSTZ1
catatactgtgtaggagacgaggtgaccatggctgatctgtgcttggt

SEQ ID NO: 763 - DNA, 50 bp, artificial sequence
  primer M739 for MGST1
ggagttactctttccatggcttacaggttgctgaaaagtaaattgtacct

SEQ ID NO: 764 - DNA, 50 bp, artificial sequence
  primer M740 for MGST2
ccaagaaactgaggcggcaattctaactttttctcttccctttaatgctt

SEQ ID NO: 765 - DNA, 50 bp, artificial sequence
  primer M741 for MGST3
aagtacaaagtggagtatcctatcatgtacagcacggaccctgaaaatgg

SEQ ID NO: 766 - DNA, 50 bp, artificial sequence
  primer M742 for AKR1A1
atctgcatccccaaaagtatcactccttctcgaatccttcagaacatcaa

SEQ ID NO: 767 - DNA, 50 bp, artificial sequence
  primer M743 for ALDH1A1
tggtggattcaagatgtctggaaatggaagagaactgggagagtacggtt

SEQ ID NO: 768 - DNA, 50 bp, artificial sequence
  primer M744 for ALDH1A2
aagctgggactgtttggatcaattgttacaatgccttaaatgcccagagc

SEQ ID NO: 769 - DNA, 50 bp, artificial sequence
  primer M745 for ALDH1A3
caacatgcggattgccaaagaggagattttcgggccagtgcaaccaatac

SEQ ID NO: 770 - DNA, 50 bp, artificial sequence
  primer M746 for ALDH1B1
agaggtaaagacggtcaccatcaaggttcctcagaagaactcgtaagagc

SEQ ID NO: 771 - DNA, 50 bp, artificial sequence
  primer M747 for ALDH2
gtcaaagtgcctcagaagaactcataagaatcatgcaagcttcctccctc

SEQ ID NO: 772 - DNA, 50 bp, artificial sequence
  primer M748 for ALDH3A1
cctctacatgttctccagcaacgacaaggtgattaagaagatgattgcag

SEQ ID NO: 773 - DNA, 50 bp, artificial sequence
  primer M749 for ALDH3A2
gaatgatcctgttcaacctcctagtgcctctactgaattattcctctttt

SEQ ID NO: 774 - DNA, 50 bp, artificial sequence
  primer M750 for ALDH4A1
atgctgccggcaacttctacatcaacgacaagtccactggctcgatagtg

SEQ ID NO: 775 - DNA, 50 bp, artificial sequence
  primer M751 for ALDH5A1
atgctgtgcactcatgaagagactttcgggcctctggcaccagttatcaa

SEQ ID NO: 776 - DNA, 50 bp, artificial sequence
  primer M752 for ALDH6A1
agggcatccaattctacactcagttaaagaccattacttctcagtggaaa

SEQ ID NO: 777 - DNA, 50 bp, artificial sequence
  primer M753 for ALDH7A1
cttggacctaaaggatcagactgtggcattgtaaatgtcaacattccaac

SEQ ID NO: 778 - DNA, 50 bp, artificial sequence
  primer M754 for ALDH8A1
ctggaataggtagagagggagccaaggactcttacgacttcttcactgag

SEQ ID NO: 779 - DNA, 50 bp, artificial sequence
  primer M755 for ALDH9A1
tagctgagcttcaggctgggacgtgcttcattaacaactataacgtcagc

SEQ ID NO: 780 - DNA, 50 bp, artificial sequence
  primer M756 for GPX1
tacgagggaggaacaccttgatcttacagaaaataccacctcgagatggg

SEQ ID NO: 781 - DNA, 50 bp, artificial sequence
  primer M757 for GPX2
aatgaggagatcctgaacagtctcaagtatgtccgtcctgggggtggata

SEQ ID NO: 782 - DNA, 50 bp, artificial sequence
  primer M758 for GPX3
caaccaatttggaaaacaggaaccaggagagaactcagagatccttccta

SEQ ID NO: 783 - DNA, 50 bp, artificial sequence
  primer M759 for GPX4
tgtaaccagttcgggaagcaggagccagggagtaacgaagagatcaaaga

SEQ ID NO: 784 - DNA, 50 bp, artificial sequence
  primer M760 for GPX5
ctgaactaaatgcactccaggaggagctgaagccctatggtctagttgtg

SEQ ID NO: 785 - DNA, 50 bp, artificial sequence
  primer M761 for GPX6
acctacagtgtctcattccccatgtttagcaagattgcagtcaccggtac